SEQUENCE LISTING (829 sequences)

SEQ ID NO: 1 - protein, 22 aa, Artificial sequence
  synthetic
  X (16)..(16): X = Nal
PVLDLFRELLNELLEXLKQKLK

SEQ ID NO: 2 - protein, 23 aa, Artificial sequence
  synthetic
GVLDLFRELLNELLEALKQKLKK

SEQ ID NO: 3 - protein, 22 aa, Artificial sequence
  synthetic
PVLDLFRELLNELLEWLKQKLK

SEQ ID NO: 4 - protein, 22 aa, Artificial sequence
  synthetic
PVLDLFRELLNELLEALKQKLK

SEQ ID NO: 5 - protein, 23 aa, Artificial sequence
  synthetic
PVLDLFRELLNELLEALKQKLKK

SEQ ID NO: 6 - protein, 22 aa, Artificial sequence
  synthetic
  X (13)..(13): X = Aib
PVLDLFRELLNEXLEALKQKLK

SEQ ID NO: 7 - protein, 22 aa, Artificial sequence
  synthetic
PVLDLFKELLNELLEALKQKLK

SEQ ID NO: 8 - protein, 22 aa, Artificial sequence
  synthetic
PVLDLFRELLNEGLEALKQKLK

SEQ ID NO: 9 - protein, 22 aa, Artificial sequence
  synthetic
PVLDLFRELGNELLEALKQKLK

SEQ ID NO: 10 - protein, 22 aa, Artificial sequence
  synthetic
  X (17)..(17): X = Nal
PVLDLFRELLNELLEAXKQKLK

SEQ ID NO: 11 - protein, 22 aa, Artificial sequence
  synthetic
PVLDLFKELLQELLEALKQKLK

SEQ ID NO: 12 - protein, 22 aa, Artificial sequence
  synthetic
PVLDLFRELLNELLEAGKQKLK

SEQ ID NO: 13 - protein, 22 aa, Artificial sequence
  synthetic
GVLDLFRELLNEGLEALKQKLK

SEQ ID NO: 14 - protein, 22 aa, Artificial sequence
  synthetic
  X (18)..(18): Xaa = Orn
  X (20)..(20): Xaa = Orn
  X (22)..(22): Xaa = Orn
PVLDLFRELLNELLEALXQXLX

SEQ ID NO: 15 - protein, 22 aa, Artificial sequence
  synthetic
PVLDLFRELWNELLEALKQKLK

SEQ ID NO: 16 - protein, 22 aa, Artificial sequence
  synthetic
PVLDLLRELLNELLEALKQKLK

SEQ ID NO: 17 - protein, 22 aa, Artificial sequence
  synthetic
PVLELFKELLQELLEALKQKLK

SEQ ID NO: 18 - protein, 22 aa, Artificial sequence
  synthetic
GVLDLFRELLNELLEALKQKLK

SEQ ID NO: 19 - protein, 22 aa, Artificial sequence
  synthetic
PVLDLFRELLNEGLEALKQKLK

SEQ ID NO: 20 - protein, 22 aa, Artificial sequence
  synthetic
PVLDLFREGLNELLEALKQKLK

SEQ ID NO: 21 - protein, 22 aa, Artificial sequence
  synthetic
PVLDLFRELLNELLEALKQKLK

SEQ ID NO: 22 - protein, 22 aa, Artificial sequence
  synthetic
PVLDLFRELLNELLEGLKQKLK

SEQ ID NO: 23 - protein, 22 aa, Artificial sequence
  synthetic
PLLELFKELLQELLEALKQKLK

SEQ ID NO: 24 - protein, 22 aa, Artificial sequence
  synthetic
PVLDLFRELLNELLEALQKKLK

SEQ ID NO: 25 - protein, 22 aa, Artificial sequence
  synthetic
  X (13)..(13): X = Aib
PVLDFFRELLNEXLEALKQKLK

SEQ ID NO: 26 - protein, 22 aa, Artificial sequence
  synthetic
PVLDLFRELLNELLELLKQKLK

SEQ ID NO: 27 - protein, 22 aa, Artificial sequence
  synthetic
  X (14)..(14): X = Nal
PVLDLFRELLNELXEALKQKLK

SEQ ID NO: 28 - protein, 22 aa, Artificial sequence
  synthetic
PVLDLFRELLNELWEALKQKLK

SEQ ID NO: 29 - protein, 22 aa, Artificial sequence
  synthetic
AVLDLFRELLNELLEALKQKLK

SEQ ID NO: 30 - protein, 22 aa, Artificial sequence
  synthetic
PVLDLPRELLNELLEALKQKLK

SEQ ID NO: 31 - protein, 22 aa, Artificial sequence
  synthetic
  X (13)..(13): X = Aib
PVLDLFLELLNEXLEALKQKLK

SEQ ID NO: 32 - protein, 22 aa, Artificial sequence
  synthetic
  X (1)..(1): X = Aib
XVLDLFRELLNELLEALKQKLK

SEQ ID NO: 33 - protein, 22 aa, Artificial sequence
  synthetic
PVLDLFREKLNELLEALKQKLK

SEQ ID NO: 34 - protein, 22 aa, Artificial sequence
  synthetic
  X (5)..(5): X = Nal
PVLDXFRELLNELLEALKQKLK

SEQ ID NO: 35 - protein, 22 aa, Artificial sequence
  synthetic
PVLDWFRELLNELLEALKQKLK

SEQ ID NO: 36 - protein, 22 aa, Artificial sequence
  synthetic
PLLELLKELLQELLEALKQKLK

SEQ ID NO: 37 - protein, 22 aa, Artificial sequence
  synthetic
PVLDLFREWLNELLEALKQKLK

SEQ ID NO: 38 - protein, 22 aa, Artificial sequence
  synthetic
  X (13)..(13): X = Aib
PVLDLFRELLNEXLEAWKQKLK

SEQ ID NO: 39 - protein, 22 aa, Artificial sequence
  synthetic
PVLDLFRELLEELLKALKKKLK

SEQ ID NO: 40 - protein, 22 aa, Artificial sequence
  synthetic
PVLDLFNELLRELLEALQKKLK

SEQ ID NO: 41 - protein, 22 aa, Artificial sequence
  synthetic
  X (13)..(13): X = Aib
PVLDLWRELLNEXLEALKQKLK

SEQ ID NO: 42 - protein, 22 aa, Artificial sequence
  synthetic
  X (13)..(13): X = Aib
PVLDEFREKLNEXWEALKQKLK

SEQ ID NO: 43 - protein, 22 aa, Artificial sequence
  synthetic
  X (13)..(13): X = Aib
PVLDEFREKLWEXLEALKQKLK

SEQ ID NO: 44 - protein, 22 aa, Artificial sequence
  synthetic
  X (13)..(13): X = Aib
PVLDEFREKLNEXLEALKQKLK

SEQ ID NO: 45 - protein, 22 aa, Artificial sequence
  synthetic
  X (13)..(13): X = Aib
PVLDEFREKLNEXLEALKQKLK

SEQ ID NO: 46 - protein, 22 aa, Artificial sequence
  synthetic
  X (13)..(13): X = Aib
PVLDLFREKLNEXLEALKQKLK

SEQ ID NO: 47 - protein, 21 aa, Artificial sequence
  synthetic
VLDLFRELLNEGLEALKQKLK

SEQ ID NO: 48 - protein, 22 aa, Artificial sequence
  synthetic
PVLDLFRELLNELLEALKQKLK

SEQ ID NO: 49 - protein, 22 aa, Artificial sequence
  synthetic
PVLDLFRNLLEKLLEALEQKLK

SEQ ID NO: 50 - protein, 22 aa, Artificial sequence
  synthetic
  X (13)..(13): X = Aib
PVLDLFRELLWEXLEALKQKLK

SEQ ID NO: 51 - protein, 22 aa, Artificial sequence
  synthetic
  X (13)..(13): X = Aib
PVLDLFWELLNEXLEALKQKLK

SEQ ID NO: 52 - protein, 22 aa, Artificial sequence
  synthetic
  X (13)..(13): X = Aib
PVWDEFREKLNEXLEALKQKLK

SEQ ID NO: 53 - protein, 22 aa, Artificial sequence
  synthetic
VVLDLFRELLNELLEALKQKLK

SEQ ID NO: 54 - protein, 22 aa, Artificial sequence
  synthetic
PVLDLFRELLNEWLEALKQKLK

SEQ ID NO: 55 - protein, 19 aa, Artificial sequence
  synthetic
PLFRELLNELLEALKQKLK

SEQ ID NO: 56 - protein, 22 aa, Artificial sequence
  synthetic
PVLDLFRELLNELLEALKQKKK

SEQ ID NO: 57 - protein, 22 aa, Artificial sequence
  synthetic
PVLDLFRNLLEELLKALEQKLK

SEQ ID NO: 58 - protein, 21 aa, Artificial sequence
  synthetic
  X (13)..(13): X = Aib
PVLDEFREKLNEXLEALKQKL

SEQ ID NO: 59 - protein, 22 aa, Artificial sequence
  synthetic
LVLDLFRELLNELLEALKQKLK

SEQ ID NO: 60 - protein, 19 aa, Artificial sequence
  synthetic
PVLDLFRELLNELLEALKQ

SEQ ID NO: 61 - protein, 22 aa, Artificial sequence
  synthetic
  X (13)..(13): X = Aib
PVLDEFRWKLNEXLEALKQKLK

SEQ ID NO: 62 - protein, 22 aa, Artificial sequence
  synthetic
  X (13)..(13): X = Aib
PVLDEWREKLNEXLEALKQKLK

SEQ ID NO: 63 - protein, 22 aa, Artificial sequence
  synthetic
  X (13)..(13): X = Aib
PVLDFFREKLNEXLEALKQKLK

SEQ ID NO: 64 - protein, 22 aa, Artificial sequence
  synthetic
  X (13)..(13): X = Aib
PWLDEFREKLNEXLEALKQKLK

SEQ ID NO: 65 - protein, 21 aa, Artificial sequence
  synthetic
  X (12)..(12): X = Aib
VLDEFREKLNEXLEALKQKLK

SEQ ID NO: 66 - protein, 22 aa, Artificial sequence
  synthetic
PVLDLFRNLLEELLEALQKKLK

SEQ ID NO: 67 - protein, 21 aa, Artificial sequence
  synthetic
VLDLFRELLNELLEALKQKLK

SEQ ID NO: 68 - protein, 22 aa, Artificial sequence
  synthetic
  X (13)..(13): X = Aib
PVLDEFRELLKEXLEALKQKLK

SEQ ID NO: 69 - protein, 22 aa, Artificial sequence
  synthetic
  X (13)..(13): X = Aib
PVLDEFRKKLNEXLEALKQKLK

SEQ ID NO: 70 - protein, 22 aa, Artificial sequence
  synthetic
  X (13)..(13): X = Aib
PVLDEFRELLYEXLEALKQKLK

SEQ ID NO: 71 - protein, 22 aa, Artificial sequence
  synthetic
  X (14)..(14): X = Aib
PVLDEFREKLNELXEALKQKLK

SEQ ID NO: 72 - protein, 22 aa, Artificial sequence
  synthetic
  misc_feature (13)..(13): Xaa can be any naturally occurring amino acid
PVLDLFRELLNEXLWALKQKLK

SEQ ID NO: 73 - protein, 22 aa, Artificial sequence
  synthetic
  X (13)..(13): X = Aib
PVLDEFWEKLNEXLEALKQKLK

SEQ ID NO: 74 - protein, 22 aa, Artificial sequence
  synthetic
  X (13)..(13): X = Aib
PVLDKFREKLNEXLEALKQKLK

SEQ ID NO: 75 - protein, 22 aa, Artificial sequence
  synthetic
PVLDEFREKLNEELEALKQKLK

SEQ ID NO: 76 - protein, 22 aa, Artificial sequence
  synthetic
  X (13)..(13): X = Aib
PVLDEFRELLFEXLEALKQKLK

SEQ ID NO: 77 - protein, 22 aa, Artificial sequence
  synthetic
  X (13)..(13): X = Aib
PVLDEFREKLNKXLEALKQKLK

SEQ ID NO: 78 - protein, 22 aa, Artificial sequence
  synthetic
  X (13)..(13): X = Aib
PVLDEFRDKLNEXLEALKQKLK

SEQ ID NO: 79 - protein, 22 aa, Artificial sequence
  synthetic
PVLDEFRELLNELLEALKQKLK

SEQ ID NO: 80 - protein, 22 aa, Artificial sequence
  synthetic
PVLDLFERLLNELLEALQKKLK

SEQ ID NO: 81 - protein, 22 aa, Artificial sequence
  synthetic
  X (13)..(13): X = Aib
PVLDEFREKLNWXLEALKQKLK

SEQ ID NO: 82 - protein, 20 aa, Artificial sequence
  synthetic
  X (11)..(11): X = Aib
LDEFREKLNEXLEALKQKLK

SEQ ID NO: 83 - protein, 22 aa, Artificial sequence
  synthetic
  X (13)..(13): X = Aib
PVLDEFREKLNEXLEALWQKLK

SEQ ID NO: 84 - protein, 22 aa, Artificial sequence
  synthetic
PVLDEFREKLNELLEALKQKLK

SEQ ID NO: 85 - protein, 21 aa, Artificial sequence
  synthetic
PLDLFRELLNELLEALKQKLK

SEQ ID NO: 86 - protein, 22 aa, Artificial sequence
  synthetic
PVLELFERLLDELLNALQKKLK

SEQ ID NO: 87 - protein, 22 aa, Artificial sequence
  synthetic
PLLELLKELLQELLEALKQKLK

SEQ ID NO: 88 - protein, 22 aa, Artificial sequence
  synthetic
  X (13)..(13): X = Aib
PVLDKFRELLNEXLEALKQKLK

SEQ ID NO: 89 - protein, 22 aa, Artificial sequence
  synthetic
  X (13)..(13): X = Aib
PVLDEFREKLNEXLWALKQKLK

SEQ ID NO: 90 - protein, 19 aa, Artificial sequence
  synthetic
  misc_feature (10)..(10): Xaa can be any naturally occurring amino acid
DEFREKLNEXLEALKQKLK

SEQ ID NO: 91 - protein, 22 aa, Artificial sequence
  synthetic
  X (13)..(13): X = Aib
PVLDEFRELLNEXLEALKQKLK

SEQ ID NO: 92 - protein, 22 aa, Artificial sequence
  synthetic
  X (13)..(13): X = Aib
PVLDEFRELYNEXLEALKQKLK

SEQ ID NO: 93 - protein, 22 aa, Artificial sequence
  synthetic
  X (13)..(13): X = Aib
PVLDEFREKLNEXLKALKQKLK

SEQ ID NO: 94 - protein, 22 aa, Artificial sequence
  synthetic
PVLDEFREKLNEALEALKQKLK

SEQ ID NO: 95 - protein, 22 aa, Artificial sequence
  synthetic
  X (13)..(13): X = Aib
PVLDLFRELLNLXLEALKQKLK

SEQ ID NO: 96 - protein, 22 aa, Artificial sequence
  synthetic
  X (13)..(13): X = Aib
PVLDLFRELLNEXLEALKQKLK

SEQ ID NO: 97 - protein, 15 aa, Artificial sequence
  synthetic
PVLDLFRELLNELLE

SEQ ID NO: 98 - protein, 22 aa, Artificial sequence
  synthetic
PVLDLFRELLNEELEALKQKLK

SEQ ID NO: 99 - protein, 22 aa, Artificial sequence
  synthetic
KLKQKLAELLENLLERFLDLVP

SEQ ID NO: 100 - protein, 22 aa, Artificial sequence
  synthetic
PVLDLFRELLNELLEALKQKLK

SEQ ID NO: 101 - protein, 22 aa, Artificial sequence
  synthetic
  X (13)..(13): X = Aib
PVLDLFRELLNWXLEALKQKLK

SEQ ID NO: 102 - protein, 22 aa, Artificial sequence
  synthetic
  X (13)..(13): X = Aib
PVLDLFRELLNLXLEALKEKLK

SEQ ID NO: 103 - protein, 22 aa, Artificial sequence
  synthetic
PVLDEFRELLNEELEALKQKLK

SEQ ID NO: 104 - protein, 15 aa, Artificial sequence
  synthetic
PLLNELLEALKQKLK

SEQ ID NO: 105 - protein, 22 aa, Artificial sequence
  synthetic
PAADAFREAANEAAEAAKQKAK

SEQ ID NO: 106 - protein, 22 aa, Artificial sequence
  synthetic
PVLDLFREKLNEELEALKQKLK

SEQ ID NO: 107 - protein, 22 aa, Artificial sequence
  synthetic
KLKQKLAELLENLLERFLDLVP

SEQ ID NO: 108 - protein, 22 aa, Artificial sequence
  synthetic
  X (13)..(13): X = Aib
PVLDLFRWLLNEXLEALKQKLK

SEQ ID NO: 109 - protein, 22 aa, Artificial sequence
  synthetic
PVLDEFREKLNERLEALKQKLK

SEQ ID NO: 110 - protein, 22 aa, Artificial sequence
  synthetic
  X (13)..(14): X = Aib
PVLDEFREKLNEXXEALKQKLK

SEQ ID NO: 111 - protein, 22 aa, Artificial sequence
  synthetic
  X (13)..(13): X = Aib
PVLDEFREKLWEXWEALKQKLK

SEQ ID NO: 112 - protein, 22 aa, Artificial sequence
  synthetic
  X (13)..(13): X = Aib
PVLDEFREKLNEXSEALKQKLK

SEQ ID NO: 113 - protein, 22 aa, Artificial sequence
  synthetic
PVLDEFREKLNEPLEALKQKLK

SEQ ID NO: 114 - protein, 22 aa, Artificial sequence
  synthetic
  X (13)..(13): X = Aib
PVLDEFREKLNEXMEALKQKLK

SEQ ID NO: 115 - protein, 22 aa, Artificial sequence
  synthetic
  X (13)..(13): X = Aib
PKLDEFREKLNEXLEALKQKLK

SEQ ID NO: 116 - protein, 22 aa, Artificial sequence
  synthetic
  X (13)..(13): X = Aib
PHLDEFREKLNEXLEALKQKLK

SEQ ID NO: 117 - protein, 22 aa, Artificial sequence
  synthetic
  X (13)..(13): X = Aib
PELDEFREKLNEXLEALKQKLK

SEQ ID NO: 118 - protein, 22 aa, Artificial sequence
  synthetic
  X (13)..(13): X = Aib
PVLDEFREKLNEXLEALEQKLK

SEQ ID NO: 119 - protein, 22 aa, Artificial sequence
  synthetic
  X (17)..(17): X = Aib
PVLDEFREKLNEELEAXKQKLK

SEQ ID NO: 120 - protein, 22 aa, Artificial sequence
  synthetic
  X (16)..(16): X = Aib
PVLDEFREKLNEELEXLKQKLK

SEQ ID NO: 121 - protein, 22 aa, Artificial sequence
  synthetic
PVLDEFREKLNEELEALWQKLK

SEQ ID NO: 122 - protein, 22 aa, Artificial sequence
  synthetic
PVLDEFREKLNEELEWLKQKLK

SEQ ID NO: 123 - protein, 22 aa, Artificial sequence
  synthetic
QVLDLFRELLNELLEALKQKLK

SEQ ID NO: 124 - protein, 22 aa, Artificial sequence
  synthetic
  X (7)..(7): Xaa = Orn
  X (18)..(18): Xaa = Orn
  X (20)..(20): Xaa = Orn
  X (22)..(22): Xaa = Orn
PVLDLFXELLNELLEALXQXLX

SEQ ID NO: 125 - protein, 22 aa, Artificial sequence
  synthetic
NVLDLFRELLNELLEALKQKLK

SEQ ID NO: 126 - protein, 22 aa, Artificial sequence
  synthetic
PVLDLFRELLNELGEALKQKLK

SEQ ID NO: 127 - protein, 22 aa, Artificial sequence
  synthetic
PVLDLFRELLNELLELLKQKLK

SEQ ID NO: 128 - protein, 22 aa, Artificial sequence
  synthetic
PVLDLFRELLNELLEFLKQKLK

SEQ ID NO: 129 - protein, 22 aa, Artificial sequence
  synthetic
PVLELFNDLLRELLEALQKKLK

SEQ ID NO: 130 - protein, 22 aa, Artificial sequence
  synthetic
PVLELFNDLLRELLEALKQKLK

SEQ ID NO: 131 - protein, 22 aa, Artificial sequence
  synthetic
PVLELFKELLNELLDALRQKLK

SEQ ID NO: 132 - protein, 22 aa, Artificial sequence
  synthetic
PVLDLFRELLENLLEALQKKLK

SEQ ID NO: 133 - protein, 22 aa, Artificial sequence
  synthetic
PVLELFERLLEDLLQALNKKLK

SEQ ID NO: 134 - protein, 22 aa, Artificial sequence
  synthetic
  X (19)..(19): Xaa = Orn
PVLELFERLLEDLLKALNXKLK

SEQ ID NO: 135 - protein, 22 aa, Artificial sequence
  synthetic
DVLDLFRELLNELLEALKQKLK

SEQ ID NO: 136 - protein, 22 aa, Artificial sequence
  synthetic
PALELFKDLLQELLEALKQKLK

SEQ ID NO: 137 - protein, 22 aa, Artificial sequence
  synthetic
  X (17)..(17): X = Nal
PVLDLFRELLNEGLEAXKQKLK

SEQ ID NO: 138 - protein, 22 aa, Artificial sequence
  synthetic
PVLDLFRELLNEGLEWLKQKLK

SEQ ID NO: 139 - protein, 22 aa, Artificial sequence
  synthetic
PVLDLFRELWNEGLEALKQKLK

SEQ ID NO: 140 - protein, 22 aa, Artificial sequence
  synthetic
  X (18)..(18): Xaa = Orn
  X (20)..(20): Xaa = Orn
  X (22)..(22): Xaa = Orn
PVLDLFRELLNEGLEALXQXLX

SEQ ID NO: 141 - protein, 22 aa, Artificial sequence
  synthetic
PVLDFFRELLNEGLEALKQKLK

SEQ ID NO: 142 - protein, 22 aa, Artificial sequence
  synthetic
PVLELFRELLNEGLEALKQKLK

SEQ ID NO: 143 - protein, 22 aa, Artificial sequence
  synthetic
PVLDLFRELLNEGLEALKQKLK

SEQ ID NO: 144 - protein, 22 aa, Artificial sequence
  synthetic
PVLELFENLLERLLDALQKKLK

SEQ ID NO: 145 - protein, 22 aa, Artificial sequence
  synthetic
GVLELFENLLERLLDALQKKLK

SEQ ID NO: 146 - protein, 22 aa, Artificial sequence
  synthetic
PVLELFENLLERLLDALQKKLK

SEQ ID NO: 147 - protein, 22 aa, Artificial sequence
  synthetic
PVLELFENLLERLFDALQKKLK

SEQ ID NO: 148 - protein, 22 aa, Artificial sequence
  synthetic
PVLELFENLLERLGDALQKKLK

SEQ ID NO: 149 - protein, 22 aa, Artificial sequence
  synthetic
PVLELFENLWERLLDALQKKLK

SEQ ID NO: 150 - protein, 22 aa, Artificial sequence
  synthetic
PLLELFENLLERLLDALQKKLK

SEQ ID NO: 151 - protein, 22 aa, Artificial sequence
  synthetic
PVLELFENLGERLLDALQKKLK

SEQ ID NO: 152 - protein, 22 aa, Artificial sequence
  synthetic
PVFELFENLLERLLDALQKKLK

SEQ ID NO: 153 - protein, 22 aa, Artificial sequence
  synthetic
AVLELFENLLERLLDALQKKLK

SEQ ID NO: 154 - protein, 22 aa, Artificial sequence
  synthetic
PVLELFENLLERGLDALQKKLK

SEQ ID NO: 155 - protein, 22 aa, Artificial sequence
  synthetic
PVLELFLNLWERLLDALQKKLK

SEQ ID NO: 156 - protein, 22 aa, Artificial sequence
  synthetic
PVLELFLNLLERLLDALQKKLK

SEQ ID NO: 157 - protein, 22 aa, Artificial sequence
  synthetic
PVLEFFENLLERLLDALQKKLK

SEQ ID NO: 158 - protein, 22 aa, Artificial sequence
  synthetic
PVLELFLNLLERLLDWLQKKLK

SEQ ID NO: 159 - protein, 22 aa, Artificial sequence
  synthetic
PVLDLFENLLERLLDALQKKLK

SEQ ID NO: 160 - protein, 22 aa, Artificial sequence
  synthetic
PVLELFENLLERLLDWLQKKLK

SEQ ID NO: 161 - protein, 22 aa, Artificial sequence
  synthetic
PVLELFENLLERLLEALQKKLK

SEQ ID NO: 162 - protein, 22 aa, Artificial sequence
  synthetic
PVLELFENWLERLLDALQKKLK

SEQ ID NO: 163 - protein, 22 aa, Artificial sequence
  synthetic
PVLELFENLLERLWDALQKKLK

SEQ ID NO: 164 - protein, 22 aa, Artificial sequence
  synthetic
PVLELFENLLERLLDAWQKKLK

SEQ ID NO: 165 - protein, 22 aa, Artificial sequence
  synthetic
PVLELFENLLERLLDLLQKKLK

SEQ ID NO: 166 - protein, 22 aa, Artificial sequence
  synthetic
PVLELFLNLLEKLLDALQKKLK

SEQ ID NO: 167 - protein, 22 aa, Artificial sequence
  synthetic
PVLELFENGLERLLDALQKKLK

SEQ ID NO: 168 - protein, 22 aa, Artificial sequence
  synthetic
PVLELFEQLLEKLLDALQKKLK

SEQ ID NO: 169 - protein, 22 aa, Artificial sequence
  synthetic
PVLELFENLLEKLLDALQKKLK

SEQ ID NO: 170 - protein, 22 aa, Artificial sequence
  synthetic
  X (12)..(12): Xaa = Orn
  X (19)..(20): Xaa = Orn
  X (22)..(22): Xaa = Orn
PVLELFENLLEXLLDALQXXLX

SEQ ID NO: 171 - protein, 22 aa, Artificial sequence
  synthetic
PVLELFENLLEKLLDLLQKKLK

SEQ ID NO: 172 - protein, 22 aa, Artificial sequence
  synthetic
PVLELFLNLLERLGDALQKKLK

SEQ ID NO: 173 - protein, 22 aa, Artificial sequence
  synthetic
PVLDLFDNLLDRLLDLLNKKLK

SEQ ID NO: 174 - protein, 22 aa, Artificial sequence
  synthetic
PVLELFENLLERLLDALQKKLK

SEQ ID NO: 175 - protein, 22 aa, Artificial sequence
  synthetic
PVLELFENLLERLLELLNKKLK

SEQ ID NO: 176 - protein, 22 aa, Artificial sequence
  synthetic
PVLELWENLLERLLDALQKKLK

SEQ ID NO: 177 - protein, 22 aa, Artificial sequence
  synthetic
GVLELFLNLLERLLDALQKKLK

SEQ ID NO: 178 - protein, 22 aa, Artificial sequence
  synthetic
PVLELFDNLLEKLLEALQKKLR

SEQ ID NO: 179 - protein, 22 aa, Artificial sequence
  synthetic
PVLELFDNLLERLLDALQKKLK

SEQ ID NO: 180 - protein, 22 aa, Artificial sequence
  synthetic
PVLELFDNLLDKLLDALQKKLR

SEQ ID NO: 181 - protein, 22 aa, Artificial sequence
  synthetic
PVLELFENLLERWLDALQKKLK

SEQ ID NO: 182 - protein, 22 aa, Artificial sequence
  synthetic
PVLELFENLLEKLLEALQKKLK

SEQ ID NO: 183 - protein, 22 aa, Artificial sequence
  synthetic
PLLELFENLLEKLLDALQKKLK

SEQ ID NO: 184 - protein, 22 aa, Artificial sequence
  synthetic
PVLELFLNLLERLLDAWQKKLK

SEQ ID NO: 185 - protein, 22 aa, Artificial sequence
  synthetic
  X (19)..(20): Xaa = Orn
  X (22)..(22): Xaa = Orn
PVLELFENLLERLLDALQXXLX

SEQ ID NO: 186 - protein, 22 aa, Artificial sequence
  synthetic
PVLELFEQLLERLLDALQKKLK

SEQ ID NO: 187 - protein, 22 aa, Artificial sequence
  synthetic
PVLELFENLLERLLDALNKKLK

SEQ ID NO: 188 - protein, 22 aa, Artificial sequence
  synthetic
PVLELFENLLDRLLDALQKKLK

SEQ ID NO: 189 - protein, 22 aa, Artificial sequence
  synthetic
DVLELFENLLERLLDALQKKLK

SEQ ID NO: 190 - protein, 22 aa, Artificial sequence
  synthetic
PVLEFWDNLLDKLLDALQKKLR

SEQ ID NO: 191 - protein, 18 aa, Artificial sequence
  synthetic
PVLDLLRELLEELKQKLK

SEQ ID NO: 192 - protein, 18 aa, Artificial sequence
  synthetic
PVLDLFKELLEELKQKLK

SEQ ID NO: 193 - protein, 18 aa, Artificial sequence
  synthetic
PVLDLFRELLEELKQKLK

SEQ ID NO: 194 - protein, 18 aa, Artificial sequence
  synthetic
PVLELFRELLEELKQKLK

SEQ ID NO: 195 - protein, 18 aa, Artificial sequence
  synthetic
PVLELFKELLEELKQKLK

SEQ ID NO: 196 - protein, 18 aa, Artificial sequence
  synthetic
PVLDLFRELLEELKNKLK

SEQ ID NO: 197 - protein, 18 aa, Artificial sequence
  synthetic
PLLDLFRELLEELKQKLK

SEQ ID NO: 198 - protein, 18 aa, Artificial sequence
  synthetic
GVLDLFRELLEELKQKLK

SEQ ID NO: 199 - protein, 18 aa, Artificial sequence
  synthetic
PVLDLFRELWEELKQKLK

SEQ ID NO: 200 - protein, 18 aa, Artificial sequence
  synthetic
NVLDLFRELLEELKQKLK

SEQ ID NO: 201 - protein, 18 aa, Artificial sequence
  synthetic
PLLDLFKELLEELKQKLK

SEQ ID NO: 202 - protein, 18 aa, Artificial sequence
  synthetic
PALELFKDLLEELRQKLR

SEQ ID NO: 203 - protein, 18 aa, Artificial sequence
  synthetic
AVLDLFRELLEELKQKLK

SEQ ID NO: 204 - protein, 18 aa, Artificial sequence
  synthetic
PVLDFFRELLEELKQKLK

SEQ ID NO: 205 - protein, 18 aa, Artificial sequence
  synthetic
PVLDLFREWLEELKQKLK

SEQ ID NO: 206 - protein, 18 aa, Artificial sequence
  synthetic
PLLELLKELLEELKQKLK

SEQ ID NO: 207 - protein, 18 aa, Artificial sequence
  synthetic
PVLELLKELLEELKQKLK

SEQ ID NO: 208 - protein, 18 aa, Artificial sequence
  synthetic
PALELFKDLLEELRQRLK

SEQ ID NO: 209 - protein, 18 aa, Artificial sequence
  synthetic
PVLDLFRELLNELLQKLK

SEQ ID NO: 210 - protein, 18 aa, Artificial sequence
  synthetic
PVLDLFRELLEELKQKLK

SEQ ID NO: 211 - protein, 18 aa, Artificial sequence
  synthetic
  X (14)..(14): Xaa = Orn
  X (16)..(16): Xaa = Orn
  X (18)..(18): Xaa = Orn
PVLDLFRELLEELXQXLX

SEQ ID NO: 212 - protein, 18 aa, Artificial sequence
  synthetic
  X (7)..(7): Xaa = Orn
  X (14)..(14): Xaa = Orn
  X (16)..(16): Xaa = Orn
PVLDLFXELLEELXQXLK

SEQ ID NO: 213 - protein, 18 aa, Artificial sequence
  synthetic
PALELFKDLLEEFRQRLK

SEQ ID NO: 214 - protein, 18 aa, Artificial sequence
  synthetic
PVLDLFRELLEELKQKLK

SEQ ID NO: 215 - protein, 18 aa, Artificial sequence
  synthetic
PVLDLFRELLEEWKQKLK

SEQ ID NO: 216 - protein, 18 aa, Artificial sequence
  synthetic
PVLELFKELLEELKQKLK

SEQ ID NO: 217 - protein, 18 aa, Artificial sequence
  synthetic
PVLDLFRELLELLKQKLK

SEQ ID NO: 218 - protein, 18 aa, Artificial sequence
  synthetic
PVLDLFRELLNELLQKLK

SEQ ID NO: 219 - protein, 18 aa, Artificial sequence
  synthetic
PVLDLFRELLNELWQKLK

SEQ ID NO: 220 - protein, 18 aa, Artificial sequence
  synthetic
PVLDLFRELLEELQKKLK

SEQ ID NO: 221 - protein, 18 aa, Artificial sequence
  synthetic
DVLDLFRELLEELKQKLK

SEQ ID NO: 222 - protein, 18 aa, Artificial sequence
  synthetic
PVLDAFRELLEALLQLKK

SEQ ID NO: 223 - protein, 18 aa, Artificial sequence
  synthetic
PVLDAFRELLEALAQLKK

SEQ ID NO: 224 - protein, 18 aa, Artificial sequence
  synthetic
PVLDLFREGWEELKQKLK

SEQ ID NO: 225 - protein, 18 aa, Artificial sequence
  synthetic
PVLDAFRELAEALAQLKK

SEQ ID NO: 226 - protein, 18 aa, Artificial sequence
  synthetic
PVLDAFRELGEALLQLKK

SEQ ID NO: 227 - protein, 18 aa, Artificial sequence
  synthetic
PVLDLFRELGEELKQKLK

SEQ ID NO: 228 - protein, 18 aa, Artificial sequence
  synthetic
PVLDLFREGLEELKQKLK

SEQ ID NO: 229 - protein, 18 aa, Artificial sequence
  synthetic
PVLDLFRELLEEGKQKLK

SEQ ID NO: 230 - protein, 18 aa, Artificial sequence
  synthetic
PVLELFERLLEDLQKKLK

SEQ ID NO: 231 - protein, 18 aa, Artificial sequence
  synthetic
PVLDLFRELLEKLEQKLK

SEQ ID NO: 232 - protein, 18 aa, Artificial sequence
  synthetic
PLLELFKELLEELKQKLK

SEQ ID NO: 233 - protein, 18 aa, Artificial sequence
  synthetic
LDDLLQKWAEAFNQLLKK

SEQ ID NO: 234 - protein, 18 aa, Artificial sequence
  synthetic
EWLKAFYEKVLEKLKELF

SEQ ID NO: 235 - protein, 18 aa, Artificial sequence
  synthetic
EWLEAFYKKVLEKLKELF

SEQ ID NO: 236 - protein, 18 aa, Artificial sequence
  synthetic
DWLKAFYDKVAEKLKEAF

SEQ ID NO: 237 - protein, 18 aa, Artificial sequence
  synthetic
DWFKAFYDKVFEKFKEFF

SEQ ID NO: 238 - protein, 18 aa, Artificial sequence
  synthetic
GIKKFLGSIWKFIKAFVG

SEQ ID NO: 239 - protein, 18 aa, Artificial sequence
  synthetic
DWFKAFYDKVAEKFKEAF

SEQ ID NO: 240 - protein, 18 aa, Artificial sequence
  synthetic
DWLKAFYDKVAEKLKEAF

SEQ ID NO: 241 - protein, 18 aa, Artificial sequence
  synthetic
DWLKAFYDKVFEKFKEFF

SEQ ID NO: 242 - protein, 18 aa, Artificial sequence
  synthetic
EWLEAFYKKVLEKLKELP

SEQ ID NO: 243 - protein, 18 aa, Artificial sequence
  synthetic
DWFKAFYDKFFEKFKEFF

SEQ ID NO: 244 - protein, 18 aa, Artificial sequence
  synthetic
EWLKAFYEKVLEKLKELF

SEQ ID NO: 245 - protein, 18 aa, Artificial sequence
  synthetic
EWLKAEYEKVEEKLKELF

SEQ ID NO: 246 - protein, 18 aa, Artificial sequence
  synthetic
EWLKAEYEKVLEKLKELF

SEQ ID NO: 247 - protein, 18 aa, Artificial sequence
  synthetic
EWLKAFYKKVLEKLKELF

SEQ ID NO: 248 - protein, 15 aa, Artificial sequence
  synthetic
PVLDLFRELLEQKLK

SEQ ID NO: 249 - protein, 16 aa, Artificial sequence
  synthetic
PVLDLFRELLEELKQK

SEQ ID NO: 250 - protein, 16 aa, Artificial sequence
  synthetic
PVLDLFRELLEKLKQK

SEQ ID NO: 251 - protein, 15 aa, Artificial sequence
  synthetic
PVLDLFRELLEKLQK

SEQ ID NO: 252 - protein, 16 aa, Artificial sequence
  synthetic
PVLDLFRELLEALKQK

SEQ ID NO: 253 - protein, 16 aa, Artificial sequence
  synthetic
PVLDLFENLLERLKQK

SEQ ID NO: 254 - protein, 16 aa, Artificial sequence
  synthetic
PVLDLFRELLNELKQK

SEQ ID NO: 255 - protein, 18 aa, Artificial sequence
  synthetic
DWLKAFYDKVAEKLKEAF

SEQ ID NO: 256 - protein, 18 aa, Artificial sequence
  synthetic
DWLKAFYDKVAEKLKEAF

SEQ ID NO: 257 - protein, 18 aa, Artificial sequence
  synthetic
DWLKAFYDKVAEKLKEAF

SEQ ID NO: 258 - protein, 18 aa, Artificial sequence
  synthetic
DWFKAFYDKVAEKLKEAF

SEQ ID NO: 259 - protein, 18 aa, Artificial sequence
  synthetic
DWLKAFYDKVAEKFKEAF

SEQ ID NO: 260 - protein, 18 aa, Artificial sequence
  synthetic
DWFKAFYDKVAEKFKEAF

SEQ ID NO: 261 - protein, 18 aa, Artificial sequence
  synthetic
DWLKAFYDKVFEKFKEFF

SEQ ID NO: 262 - protein, 18 aa, Artificial sequence
  synthetic
DWLKAFYDKFFEKFKEFF

SEQ ID NO: 263 - protein, 18 aa, Artificial sequence
  synthetic
DWFKAFYDKFFEKFKEFF

SEQ ID NO: 264 - protein, 18 aa, Artificial sequence
  synthetic
DWLKAFYDKVAEKLKEFF

SEQ ID NO: 265 - protein, 18 aa, Artificial sequence
  synthetic
DWLKAFYDKVFEKFKEAF

SEQ ID NO: 266 - protein, 18 aa, Artificial sequence
  synthetic
DWLKAFYDKVFEKLKEFF

SEQ ID NO: 267 - protein, 18 aa, Artificial sequence
  synthetic
DWLKAFYDKVAEKFKEFF

SEQ ID NO: 268 - protein, 18 aa, Artificial sequence
  synthetic
DWLKAFYDKVFEKFKEFF

SEQ ID NO: 269 - protein, 18 aa, Artificial sequence
  synthetic
EWLKLFYEKVLEKFKEAF

SEQ ID NO: 270 - protein, 18 aa, Artificial sequence
  synthetic
EWLKAFYDKVAEKFKEAF

SEQ ID NO: 271 - protein, 18 aa, Artificial sequence
  synthetic
EWLKAFYDKVAEKLKEFF

SEQ ID NO: 272 - protein, 18 aa, Artificial sequence
  synthetic
EWLKAFYDKVFEKFKEAF

SEQ ID NO: 273 - protein, 18 aa, Artificial sequence
  synthetic
EWLKAFYDKVFEKLKEFF

SEQ ID NO: 274 - protein, 18 aa, Artificial sequence
  synthetic
EWLKAFYDKVAEKFKEFF

SEQ ID NO: 275 - protein, 18 aa, Artificial sequence
  synthetic
EWLKAFYDKVFEKFKEFF

SEQ ID NO: 276 - protein, 14 aa, Artificial sequence
  synthetic
AFYDKVAEKLKEAF

SEQ ID NO: 277 - protein, 14 aa, Artificial sequence
  synthetic
AFYDKVAEKFKEAF

SEQ ID NO: 278 - protein, 14 aa, Artificial sequence
  synthetic
AFYDKVAEKFKEAF

SEQ ID NO: 279 - protein, 14 aa, Artificial sequence
  synthetic
AFYDKFFEKFKEFF

SEQ ID NO: 280 - protein, 14 aa, Artificial sequence
  synthetic
AFYDKFFEKFKEFF

SEQ ID NO: 281 - protein, 14 aa, Artificial sequence
  synthetic
AFYDKVAEKFKEAF

SEQ ID NO: 282 - protein, 14 aa, Artificial sequence
  synthetic
AFYDKVAEKLKEFF

SEQ ID NO: 283 - protein, 14 aa, Artificial sequence
  synthetic
AFYDKVFEKFKEAF

SEQ ID NO: 284 - protein, 14 aa, Artificial sequence
  synthetic
AFYDKVFEKLKEFF

SEQ ID NO: 285 - protein, 14 aa, Artificial sequence
  synthetic
AFYDKVAEKFKEFF

SEQ ID NO: 286 - protein, 14 aa, Artificial sequence
  synthetic
KAFYDKVFEKFKEF

SEQ ID NO: 287 - protein, 14 aa, Artificial sequence
  synthetic
LFYEKVLEKFKEAF

SEQ ID NO: 288 - protein, 14 aa, Artificial sequence
  synthetic
AFYDKVAEKFKEAF

SEQ ID NO: 289 - protein, 14 aa, Artificial sequence
  synthetic
AFYDKVAEKLKEFF

SEQ ID NO: 290 - protein, 14 aa, Artificial sequence
  synthetic
AFYDKVFEKFKEAF

SEQ ID NO: 291 - protein, 14 aa, Artificial sequence
  synthetic
AFYDKVFEKLKEFF

SEQ ID NO: 292 - protein, 14 aa, Artificial sequence
  synthetic
AFYDKVAEKFKEFF

SEQ ID NO: 293 - protein, 14 aa, Artificial sequence
  synthetic
AFYDKVFEKFKEFF

SEQ ID NO: 294 - protein, 18 aa, Artificial sequence
  synthetic
DWLKALYDKVAEKLKEAL

SEQ ID NO: 295 - protein, 18 aa, Artificial sequence
  synthetic
DWFKAFYEKVAEKLKEFF

SEQ ID NO: 296 - protein, 18 aa, Artificial sequence
  synthetic
DWFKAFYEKFFEKFKEFF

SEQ ID NO: 297 - protein, 18 aa, Artificial sequence
  synthetic
EWLKALYEKVAEKLKEAL

SEQ ID NO: 298 - protein, 18 aa, Artificial sequence
  synthetic
EWLKAFYEKVAEKLKEAF

SEQ ID NO: 299 - protein, 18 aa, Artificial sequence
  synthetic
EWFKAFYEKVAEKLKEFF

SEQ ID NO: 300 - protein, 18 aa, Artificial sequence
  synthetic
EWLKAFYEKVFEKFKEFF

SEQ ID NO: 301 - protein, 18 aa, Artificial sequence
  synthetic
EWLKAFYEKFFEKFKEFF

SEQ ID NO: 302 - protein, 18 aa, Artificial sequence
  synthetic
EWFKAFYEKFFEKFKEFF

SEQ ID NO: 303 - protein, 18 aa, Artificial sequence
  synthetic
DFLKAWYDKVAEKLKEAW

SEQ ID NO: 304 - protein, 18 aa, Artificial sequence
  synthetic
EFLKAWYEKVAEKLKEAW

SEQ ID NO: 305 - protein, 18 aa, Artificial sequence
  synthetic
DFWKAWYDKVAEKLKEWW

SEQ ID NO: 306 - protein, 18 aa, Artificial sequence
  synthetic
EFWKAWYEKVAEKLKEWW

SEQ ID NO: 307 - protein, 18 aa, Artificial sequence
  synthetic
DKLKAFYDKVFEWAKEAF

SEQ ID NO: 308 - protein, 18 aa, Artificial sequence
  synthetic
DKWKAVYDKFAEAFKEFL

SEQ ID NO: 309 - protein, 18 aa, Artificial sequence
  synthetic
EKLKAFYEKVFEWAKEAF

SEQ ID NO: 310 - protein, 18 aa, Artificial sequence
  synthetic
EKWKAVYEKFAEAFKEFL

SEQ ID NO: 311 - protein, 18 aa, Artificial sequence
  synthetic
DWLKAFVDKFAEKFKEAY

SEQ ID NO: 312 - protein, 18 aa, Artificial sequence
  synthetic
EKWKAVYEKFAEAFKEFL

SEQ ID NO: 313 - protein, 18 aa, Artificial sequence
  synthetic
DWLKAFVYDKVFKLKEFF

SEQ ID NO: 314 - protein, 18 aa, Artificial sequence
  synthetic
EWLKAFVYEKVFKLKEFF

SEQ ID NO: 315 - protein, 18 aa, Artificial sequence
  synthetic
DWLRAFYDKVAEKLKEAF

SEQ ID NO: 316 - protein, 18 aa, Artificial sequence
  synthetic
EWLRAFYEKVAEKLKEAF

SEQ ID NO: 317 - protein, 18 aa, Artificial sequence
  synthetic
DWLKAFYDRVAEKLKEAF

SEQ ID NO: 318 - protein, 18 aa, Artificial sequence
  synthetic
EWLKAFYERVAEKLKEAF

SEQ ID NO: 319 - protein, 18 aa, Artificial sequence
  synthetic
DWLKAFYDKVAERLKEAF

SEQ ID NO: 320 - protein, 18 aa, Artificial sequence
  synthetic
EWLKAFYEKVAERLKEAF

SEQ ID NO: 321 - protein, 18 aa, Artificial sequence
  synthetic
DWLKAFYDKVAEKLREAF

SEQ ID NO: 322 - protein, 18 aa, Artificial sequence
  synthetic
EWLKAFYEKVAEKLREAF

SEQ ID NO: 323 - protein, 18 aa, Artificial sequence
  synthetic
DWLKAFYDRVAERLKEAF

SEQ ID NO: 324 - protein, 18 aa, Artificial sequence
  synthetic
EWLKAFYERVAERLKEAF

SEQ ID NO: 325 - protein, 18 aa, Artificial sequence
  synthetic
DWLRAFYDKVAEKLREAF

SEQ ID NO: 326 - protein, 18 aa, Artificial sequence
  synthetic
EWLRAFYEKVAEKLREAF

SEQ ID NO: 327 - protein, 18 aa, Artificial sequence
  synthetic
DWLRAFYDRVAEKLKEAF

SEQ ID NO: 328 - protein, 18 aa, Artificial sequence
  synthetic
EWLRAFYERVAEKLKEAF

SEQ ID NO: 329 - protein, 18 aa, Artificial sequence
  synthetic
DWLKAFYDKVAERLREAF

SEQ ID NO: 330 - protein, 18 aa, Artificial sequence
  synthetic
EWLKAFYEKVAERLREAF

SEQ ID NO: 331 - protein, 18 aa, Artificial sequence
  synthetic
DWLRAFYDKVAERLKEAF

SEQ ID NO: 332 - protein, 18 aa, Artificial sequence
  synthetic
EWLRAFYEKVAERLKEAF

SEQ ID NO: 333 - protein, 18 aa, Artificial sequence
  synthetic
FAEKFKEAVKDYFAKFWD

SEQ ID NO: 334 - protein, 18 aa, Artificial sequence
  synthetic
DWFKAFYDKVAEKFKEAF

SEQ ID NO: 335 - protein, 18 aa, Artificial sequence
  synthetic
DWLKAFYDKVAEKLKEAF

SEQ ID NO: 336 - protein, 34 aa, Artificial sequence
  synthetic
PALEDLRQGLLPVLESFKVFLSALEEYTKKLNTQ

SEQ ID NO: 337 - protein, 26 aa, Artificial sequence
  synthetic
WDRVKDLATVYVDVLKDSGRDYVSQF

SEQ ID NO: 338 - protein, 21 aa, Artificial sequence
  synthetic
  misc_feature (20)..(20): Xaa can be any naturally occurring amino acid
LKLLDNWDSVTSTFSKLREXL

SEQ ID NO: 339 - protein, 22 aa, Artificial sequence
  synthetic
  misc_feature (4)..(4): Xaa can be any naturally occurring amino acid
  misc_feature (19)..(19): Xaa can be any naturally occurring amino acid
PVTXEFWDNLEKETEGLRXEMS

SEQ ID NO: 340 - protein, 11 aa, Artificial sequence
  synthetic
KDLEEVKAKVQ

SEQ ID NO: 341 - protein, 11 aa, Artificial sequence
  synthetic
  misc_feature (11)..(11): Xaa can be any naturally occurring amino acid
KDLEEVKAKVX

SEQ ID NO: 342 - protein, 22 aa, Artificial sequence
  synthetic
PYLDDFQKKWQEEMELYRQKVE

SEQ ID NO: 343 - protein, 22 aa, Artificial sequence
  synthetic
  misc_feature (18)..(18): Xaa can be any naturally occurring amino acid
PLRAELQEGARQKLHELXEKLS

SEQ ID NO: 344 - protein, 22 aa, Artificial sequence
  synthetic
PLGEEMRDRARAHVDALRTHLA

SEQ ID NO: 345 - protein, 22 aa, Artificial sequence
  synthetic
PYSDELRQRLAARLEALKENGG

SEQ ID NO: 346 - protein, 22 aa, Artificial sequence
  synthetic
ARLAEYHAKATEHLSTLSEKAK

SEQ ID NO: 347 - protein, 11 aa, Artificial sequence
  synthetic
  misc_feature (8)..(8): Xaa can be any naturally occurring amino acid
PALEDLRXGLL

SEQ ID NO: 348 - protein, 22 aa, Artificial sequence
  synthetic
PVLESFKVSFLSALEEYTKKLN

SEQ ID NO: 349 - protein, 21 aa, Artificial sequence
  synthetic
PVLESFVSFLSALEEYTKKLN

SEQ ID NO: 350 - protein, 24 aa, Artificial sequence
  synthetic
TVLLLTICSLEGALVRRQAKEPCV

SEQ ID NO: 351 - protein, 12 aa, Artificial sequence
  synthetic
QTVTDYGKDLME

SEQ ID NO: 352 - protein, 20 aa, Artificial sequence
  synthetic
  misc_feature (8)..(8): Xaa can be any naturally occurring amino acid
KVKSPELXAEAKSYFEKSKE

SEQ ID NO: 353 - protein, 24 aa, Artificial sequence
  synthetic
  misc_feature (20)..(20): Xaa can be any naturally occurring amino acid
VLTLALVAVAGARAEVSADXVATV

SEQ ID NO: 354 - protein, 22 aa, Artificial sequence
  synthetic
  misc_feature (11)..(11): Xaa can be any naturally occurring amino acid
  misc_feature (17)..(18): Xaa can be any naturally occurring amino acid
NNAKEAVEHLXKSELTXXLNAL

SEQ ID NO: 355 - protein, 25 aa, Artificial sequence
  synthetic
  misc_feature (18)..(18): Xaa can be any naturally occurring amino acid
LPVLVWLSIVLEGPAPAXGTPDVSS

SEQ ID NO: 356 - protein, 26 aa, Artificial sequence
  synthetic
LPVLVVVLSIVLEGPAPAQGTPDVSS

SEQ ID NO: 357 - protein, 21 aa, Artificial sequence
  synthetic
ALDKLKEFGNTLEDKARELIS

SEQ ID NO: 358 - protein, 22 aa, Artificial sequence
  synthetic
VVALLALLASARASEAEDASLL

SEQ ID NO: 359 - protein, 25 aa, Artificial sequence
  synthetic
  misc_feature (24)..(24): Xaa can be any naturally occurring amino acid
HLRKLRKRLLRDADDLQKRLAVYXA

SEQ ID NO: 360 - protein, 22 aa, Artificial sequence
  synthetic
AQAWGERLRARMEEMGSRTRDR

SEQ ID NO: 361 - protein, 20 aa, Artificial sequence
  synthetic
LDEVKEQVAEVRAKLEEQAQ

SEQ ID NO: 362 - protein, 37 aa, Artificial sequence
  synthetic
DWLKAFYDKVAEKLKEAFPDWAKAAYDKAAEKAKEAA

SEQ ID NO: 363 - protein, 21 aa, Artificial sequence
  synthetic
PVLDLFRELLNELLEALKQKL

SEQ ID NO: 364 - protein, 22 aa, Artificial sequence
  synthetic
PVLDLFRELLNELLEALKQKLA

SEQ ID NO: 365 - protein, 22 aa, Artificial sequence
  synthetic
PVLDLFRELLNELLEALKQKLA

SEQ ID NO: 366 - protein, 22 aa, Artificial sequence
  synthetic
PVLDLFRELLNELLEALKKLLK

SEQ ID NO: 367 - protein, 22 aa, Artificial sequence
  synthetic
PVLDLFRELLNELLEALKKLLA

SEQ ID NO: 368 - protein, 22 aa, Artificial sequence
  synthetic
PLLDLFRELLNELLEALKKLLA

SEQ ID NO: 369 - protein, 26 aa, Artificial sequence
  synthetic
EVRSKLEEWFAAFREFAEEFLARLKS

SEQ ID NO: 370 - protein, 33 aa, Artificial sequence
  synthetic
LQLQPFPQPELPYPQPELPYPQPELPYPQPQPF

SEQ ID NO: 371 - protein, 9 aa, Artificial sequence
  synthetic
PFPQPELPY

SEQ ID NO: 372 - protein, 9 aa, Artificial sequence
  synthetic
PYPQPELPY

SEQ ID NO: 373 - protein, 9 aa, Artificial sequence
  synthetic
PQPELPYPQ

SEQ ID NO: 374 - protein, 9 aa, Artificial sequence
  synthetic
FRPEQPYPQ

SEQ ID NO: 375 - protein, 9 aa, Artificial sequence
  synthetic
PQQSFPEQQ

SEQ ID NO: 376 - protein, 9 aa, Artificial sequence
  synthetic
IQPEQPAQL

SEQ ID NO: 377 - protein, 9 aa, Artificial sequence
  synthetic
QQPEQPYPQ

SEQ ID NO: 378 - protein, 9 aa, Artificial sequence
  synthetic
SQPEQEFPQ

SEQ ID NO: 379 - protein, 9 aa, Artificial sequence
  synthetic
PQPEQEFPQ

SEQ ID NO: 380 - protein, 9 aa, Artificial sequence
  synthetic
QQPEQPFPQ

SEQ ID NO: 381 - protein, 9 aa, Artificial sequence
  synthetic
PQPEQPFCQ

SEQ ID NO: 382 - protein, 9 aa, Artificial sequence
  synthetic
QQPFPEQPQ

SEQ ID NO: 383 - protein, 9 aa, Artificial sequence
  synthetic
PFPQPEQPF

SEQ ID NO: 384 - protein, 9 aa, Artificial sequence
  synthetic
PQPEQPFPW

SEQ ID NO: 385 - protein, 9 aa, Artificial sequence
  synthetic
PFSEQEQPV

SEQ ID NO: 386 - protein, 9 aa, Artificial sequence
  synthetic
FSQQQESPF

SEQ ID NO: 387 - protein, 9 aa, Artificial sequence
  synthetic
QQPIPEQPQ

SEQ ID NO: 388 - protein, 9 aa, Artificial sequence
  synthetic
PQPEQPFPQ

SEQ ID NO: 389 - protein, 9 aa, Artificial sequence
  synthetic
PIPEQPQPY

SEQ ID NO: 390 - protein, 9 aa, Artificial sequence
  synthetic
EQPIPEQPQ

SEQ ID NO: 391 - protein, 9 aa, Artificial sequence
  synthetic
PQPEQPFPQ

SEQ ID NO: 392 - protein, 9 aa, Artificial sequence
  synthetic
PYPEQEEPF

SEQ ID NO: 393 - protein, 9 aa, Artificial sequence
  synthetic
PYPEQEQPF

SEQ ID NO: 394 - protein, 9 aa, Artificial sequence
  synthetic
PFSEQEQPV

SEQ ID NO: 395 - protein, 9 aa, Artificial sequence
  synthetic
EGSFQPSQE

SEQ ID NO: 396 - protein, 9 aa, Artificial sequence
  synthetic
EQPQQPFPQ

SEQ ID NO: 397 - protein, 9 aa, Artificial sequence
  synthetic
EQPQQPYPE

SEQ ID NO: 398 - protein, 9 aa, Artificial sequence
  synthetic
QGYYPTSPQ

SEQ ID NO: 399 - protein, 9 aa, Artificial sequence
  synthetic
EGSFQPSQE

SEQ ID NO: 400 - protein, 9 aa, Artificial sequence
  synthetic
PQQSFPEQE

SEQ ID NO: 401 - protein, 9 aa, Artificial sequence
  synthetic
QGYYPTSPQ

SEQ ID NO: 402 - protein, 12 aa, Artificial sequence
  synthetic
QPFPQPQQPFPW

SEQ ID NO: 403 - protein, 12 aa, Artificial sequence
  synthetic
QPFPQPQQPIPV

SEQ ID NO: 404 - protein, 12 aa, Artificial sequence
  synthetic
QPFPQPEQPFPW

SEQ ID NO: 405 - protein, 11 aa, Artificial sequence
  synthetic
PFPQPEQPIPV

SEQ ID NO: 406 - protein, 12 aa, Artificial sequence
  synthetic
QPFPQPELPFPQ

SEQ ID NO: 407 - protein, 12 aa, Artificial sequence
  synthetic
LPYPQPQLPYPQ

SEQ ID NO: 408 - protein, 12 aa, Artificial sequence
  synthetic
LPYPQPELPYPQ

SEQ ID NO: 409 - protein, 12 aa, Artificial sequence
  synthetic
QPFPQPQLPYPQ

SEQ ID NO: 410 - protein, 12 aa, Artificial sequence
  synthetic
QPFPQPELPYPQ

SEQ ID NO: 411 - protein, 12 aa, Artificial sequence
  synthetic
QPFPQPQQPFSQ

SEQ ID NO: 412 - protein, 12 aa, Artificial sequence
  synthetic
QPFPQPEQPFSQ

SEQ ID NO: 413 - protein, 12 aa, Artificial sequence
  synthetic
QPFPQPQQPFCQ

SEQ ID NO: 414 - protein, 12 aa, Artificial sequence
  synthetic
QPFPQPEQPFCQ

SEQ ID NO: 415 - protein, 12 aa, Artificial sequence
  synthetic
QPFPQPQLPYSQ

SEQ ID NO: 416 - protein, 12 aa, Artificial sequence
  synthetic
QPFPQPELPYSQ

SEQ ID NO: 417 - protein, 16 aa, Artificial sequence
  synthetic
LQQQCSPVAMPQRLAR

SEQ ID NO: 418 - protein, 12 aa, Artificial sequence
  synthetic
QPFPQPQLPYLQ

SEQ ID NO: 419 - protein, 12 aa, Artificial sequence
  synthetic
QPFPQPELPYLQ

SEQ ID NO: 420 - protein, 12 aa, Artificial sequence
  synthetic
QQFIQPQQPFPQ

SEQ ID NO: 421 - protein, 12 aa, Artificial sequence
  synthetic
QQFIQPEQPFPQ

SEQ ID NO: 422 - protein, 12 aa, Artificial sequence
  synthetic
LERPWQQQPLPP

SEQ ID NO: 423 - protein, 12 aa, Artificial sequence
  synthetic
LERPWQEQPLPP

SEQ ID NO: 424 - protein, 12 aa, Artificial sequence
  synthetic
PIPQQPEQPFPL

SEQ ID NO: 425 - protein, 16 aa, Artificial sequence
  synthetic
QGQQGYYPISPQQSGQ

SEQ ID NO: 426 - protein, 16 aa, Artificial sequence
  synthetic
QGQPGYYPTSPQQIGQ

SEQ ID NO: 427 - protein, 16 aa, Artificial sequence
  synthetic
PGQGQSGYYPTSPQQS

SEQ ID NO: 428 - protein, 12 aa, Artificial sequence
  synthetic
PQQTFPQQPQLP

SEQ ID NO: 429 - protein, 12 aa, Artificial sequence
  synthetic
PQQTFPEQPQLP

SEQ ID NO: 430 - protein, 16 aa, Artificial sequence
  synthetic
GQGQSGYYPTSPQQSG

SEQ ID NO: 431 - protein, 16 aa, Artificial sequence
  synthetic
QYEVIRSLVLRTLPNM

SEQ ID NO: 432 - protein, 12 aa, Artificial sequence
  synthetic
QVDPSGQVQWPQ

SEQ ID NO: 433 - protein, 12 aa, Artificial sequence
  synthetic
QVDPSGEVQWPQ

SEQ ID NO: 434 - protein, 12 aa, Artificial sequence
  synthetic
QPFPQPQQPFPL

SEQ ID NO: 435 - protein, 12 aa, Artificial sequence
  synthetic
QPFPQPEQPFPL

SEQ ID NO: 436 - protein, 12 aa, Artificial sequence
  synthetic
QPFPQPQQPIPY

SEQ ID NO: 437 - protein, 12 aa, Artificial sequence
  synthetic
QPFPQPEQPIPY

SEQ ID NO: 438 - protein, 12 aa, Artificial sequence
  synthetic
PQQPVPQQPQPY

SEQ ID NO: 439 - protein, 12 aa, Artificial sequence
  synthetic
PQQPVPEQPQPY

SEQ ID NO: 440 - protein, 16 aa, Artificial sequence
  synthetic
PQPFPQQPIPQQPQPY

SEQ ID NO: 441 - protein, 11 aa, Artificial sequence
  synthetic
QQPIPQQPQPY

SEQ ID NO: 442 - protein, 11 aa, Artificial sequence
  synthetic
QQPIPEQPQPY

SEQ ID NO: 443 - protein, 12 aa, Artificial sequence
  synthetic
QQFPQPQQPFPQ

SEQ ID NO: 444 - protein, 12 aa, Artificial sequence
  synthetic
QQFPQPEQPFPQ

SEQ ID NO: 445 - protein, 16 aa, Artificial sequence
  synthetic
PQQPIPQQPQPYPQQP

SEQ ID NO: 446 - protein, 16 aa, Artificial sequence
  synthetic
QQPFPQQPFPQQPQPY

SEQ ID NO: 447 - protein, 12 aa, Artificial sequence
  synthetic
QPFPQPQQPFSW

SEQ ID NO: 448 - protein, 12 aa, Artificial sequence
  synthetic
QPFPQPEQPFSW

SEQ ID NO: 449 - protein, 16 aa, Artificial sequence
  synthetic
PQQPFPQQPQPYPQQP

SEQ ID NO: 450 - protein, 12 aa, Artificial sequence
  synthetic
QPFPQPQQPIPQ

SEQ ID NO: 451 - protein, 12 aa, Artificial sequence
  synthetic
QPFPQPEQPIPQ

SEQ ID NO: 452 - protein, 12 aa, Artificial sequence
  synthetic
QPFPQPQQPFPQ

SEQ ID NO: 453 - protein, 12 aa, Artificial sequence
  synthetic
QPFPQPEQPFPQ

SEQ ID NO: 454 - protein, 12 aa, Artificial sequence
  synthetic
QPFPQPQQPTPI

SEQ ID NO: 455 - protein, 12 aa, Artificial sequence
  synthetic
QPFPQPEQPTPI

SEQ ID NO: 456 - protein, 12 aa, Artificial sequence
  synthetic
PAPIQPQQPFPQ

SEQ ID NO: 457 - protein, 12 aa, Artificial sequence
  synthetic
PAPIQPEQPFPQ

SEQ ID NO: 458 - protein, 12 aa, Artificial sequence
  synthetic
PQQPFPQQPEQI

SEQ ID NO: 459 - protein, 12 aa, Artificial sequence
  synthetic
PQQPFPEQPEQI

SEQ ID NO: 460 - protein, 12 aa, Artificial sequence
  synthetic
PQQPFPQQPQQI

SEQ ID NO: 461 - protein, 12 aa, Artificial sequence
  synthetic
PQQPFPEQPQQI

SEQ ID NO: 462 - protein, 12 aa, Artificial sequence
  synthetic
PFPQQPEQIISQ

SEQ ID NO: 463 - protein, 12 aa, Artificial sequence
  synthetic
PFPQQPEQIISQ

SEQ ID NO: 464 - protein, 12 aa, Artificial sequence
  synthetic
PFPQQPEQIIPQ

SEQ ID NO: 465 - protein, 12 aa, Artificial sequence
  synthetic
PFPQQPEQIIPQ

SEQ ID NO: 466 - protein, 12 aa, Artificial sequence
  synthetic
QPFPQPQQQLPL

SEQ ID NO: 467 - protein, 12 aa, Artificial sequence
  synthetic
QPFPQPEQQLPL

SEQ ID NO: 468 - protein, 11 aa, Artificial sequence
  synthetic
LFPLPQQPFPQ

SEQ ID NO: 469 - protein, 11 aa, Artificial sequence
  synthetic
LFPLPEQPFPQ

SEQ ID NO: 470 - protein, 15 aa, Artificial sequence
  synthetic
LQPFPQPELPYPQPQ

SEQ ID NO: 471 - protein, 14 aa, Artificial sequence
  synthetic
QPFPQPEQPFPWQP

SEQ ID NO: 472 - protein, 15 aa, Artificial sequence
  synthetic
PEQPIPEQPQPYPQQ

SEQ ID NO: 473 - protein, 16 aa, Artificial sequence
  synthetic
PQQPFPQPEQPFPWQP

SEQ ID NO: 474 - protein, 16 aa, Artificial sequence
  synthetic
FPEQPIPEQPQPYPQQ

SEQ ID NO: 475 - protein, 15 aa, Artificial sequence
  synthetic
PEQPIPEQPQPYPQQ

SEQ ID NO: 476 - protein, 12 aa, Artificial sequence
  synthetic
PQPFLPQLPYPQ

SEQ ID NO: 477 - protein, 12 aa, Artificial sequence
  synthetic
QAFPQPQQTFPH

SEQ ID NO: 478 - protein, 11 aa, Artificial sequence
  synthetic
TPIQPQQPFPQ

SEQ ID NO: 479 - protein, 12 aa, Artificial sequence
  synthetic
PFPLQPQQPFPQ

SEQ ID NO: 480 - protein, 11 aa, Artificial sequence
  synthetic
PFTQPQQPTPI

SEQ ID NO: 481 - protein, 12 aa, Artificial sequence
  synthetic
QPFPQLQQPQQP

SEQ ID NO: 482 - protein, 16 aa, Artificial sequence
  synthetic
VAHAIIMHQQQQQQQE

SEQ ID NO: 483 - protein, 12 aa, Artificial sequence
  synthetic
SYPVQPQQPFPQ

SEQ ID NO: 484 - protein, 16 aa, Artificial sequence
  synthetic
PQQPQPFPQQPVPQQP

SEQ ID NO: 485 - protein, 12 aa, Artificial sequence
  synthetic
PFPWQPQQPFPQ

SEQ ID NO: 486 - protein, 12 aa, Artificial sequence
  synthetic
PFPLQPQQPFPQ

SEQ ID NO: 487 - protein, 12 aa, Artificial sequence
  synthetic
QQPFQPQQPFPQ

SEQ ID NO: 488 - protein, 16 aa, Artificial sequence
  synthetic
NPLQPQQPFPLQPQPP

SEQ ID NO: 489 - protein, 16 aa, Artificial sequence
  synthetic
PLQPQQPFPLQPQPPQ

SEQ ID NO: 490 - protein, 13 aa, Artificial sequence
  synthetic
PNPLQPQQPFPLQ

SEQ ID NO: 491 - protein, 12 aa, Artificial sequence
  synthetic
TIPQQPQQPFPL

SEQ ID NO: 492 - protein, 12 aa, Artificial sequence
  synthetic
SFSQQPQQPFPL

SEQ ID NO: 493 - protein, 12 aa, Artificial sequence
  synthetic
SFSEQPQQPFPL

SEQ ID NO: 494 - protein, 12 aa, Artificial sequence
  synthetic
YSPYQPQQPFPQ

SEQ ID NO: 495 - protein, 12 aa, Artificial sequence
  synthetic
QLPLQPQQPFPQ

SEQ ID NO: 496 - protein, 16 aa, Artificial sequence
  synthetic
QQPQQPFPLQPQQPVP

SEQ ID NO: 497 - protein, 12 aa, Artificial sequence
  synthetic
IIPQQPQQPFPL

SEQ ID NO: 498 - protein, 12 aa, Artificial sequence
  synthetic
PEQIIPQQPQQP

SEQ ID NO: 499 - protein, 11 aa, Artificial sequence
  synthetic
FLLQPQQPFSQ

SEQ ID NO: 500 - protein, 12 aa, Artificial sequence
  synthetic
IISQQPQQPFPL

SEQ ID NO: 501 - protein, 12 aa, Artificial sequence
  synthetic
PFPQRPQQPFPQ

SEQ ID NO: 502 - protein, 16 aa, Artificial sequence
  synthetic
ELQPFPQPELPYPQPQ

SEQ ID NO: 503 - protein, 15 aa, Artificial sequence
  synthetic
EQPFPQPEQPFPWQP

SEQ ID NO: 504 - protein, 16 aa, Artificial sequence
  synthetic
EPEQPIPEQPQPYPQQ

SEQ ID NO: 505 - protein, 16 aa, Artificial sequence
  synthetic
QLQPFPQPELPYPQPQ

SEQ ID NO: 506 - protein, 15 aa, Artificial sequence
  synthetic
QQPFPQPEQPFPWQP

SEQ ID NO: 507 - protein, 16 aa, Artificial sequence
  synthetic
FPEQPIPEQPQPYPQQ

SEQ ID NO: 508 - protein, 4 aa, Artificial sequence
  synthetic
PELP

SEQ ID NO: 509 - protein, 7 aa, Artificial sequence
  synthetic
QPELPYP

SEQ ID NO: 510 - protein, 7 aa, Artificial sequence
  synthetic
PQPELPY

SEQ ID NO: 511 - protein, 7 aa, Artificial sequence
  synthetic
FPQPELP

SEQ ID NO: 512 - protein, 8 aa, Artificial sequence
  synthetic
PELPYPQP

SEQ ID NO: 513 - protein, 8 aa, Artificial sequence
  synthetic
QPELPYPQ

SEQ ID NO: 514 - protein, 8 aa, Artificial sequence
  synthetic
PQPELPYP

SEQ ID NO: 515 - protein, 8 aa, Artificial sequence
  synthetic
FPQPELPY

SEQ ID NO: 516 - protein, 8 aa, Artificial sequence
  synthetic
PFPQPELP

SEQ ID NO: 517 - protein, 9 aa, Artificial sequence
  synthetic
PELPYPQPQ

SEQ ID NO: 518 - protein, 9 aa, Artificial sequence
  synthetic
QPELPYPQP

SEQ ID NO: 519 - protein, 9 aa, Artificial sequence
  synthetic
FPQPELPYP

SEQ ID NO: 520 - protein, 9 aa, Artificial sequence
  synthetic
PFPQPELPY

SEQ ID NO: 521 - protein, 9 aa, Artificial sequence
  synthetic
QPFPQPELP

SEQ ID NO: 522 - protein, 10 aa, Artificial sequence
  synthetic
QPELPYPQPQ

SEQ ID NO: 523 - protein, 10 aa, Artificial sequence
  synthetic
PQPELPYPQP

SEQ ID NO: 524 - protein, 10 aa, Artificial sequence
  synthetic
FPQPELPYPQ

SEQ ID NO: 525 - protein, 10 aa, Artificial sequence
  synthetic
PFPQPELPYP

SEQ ID NO: 526 - protein, 10 aa, Artificial sequence
  synthetic
QPFPQPELPY

SEQ ID NO: 527 - protein, 10 aa, Artificial sequence
  synthetic
LQPFPQPELP

SEQ ID NO: 528 - protein, 11 aa, Artificial sequence
  synthetic
PQPELPYPQPQ

SEQ ID NO: 529 - protein, 11 aa, Artificial sequence
  synthetic
FPQPELPYPQP

SEQ ID NO: 530 - protein, 11 aa, Artificial sequence
  synthetic
PFPQPELPYPQ

SEQ ID NO: 531 - protein, 11 aa, Artificial sequence
  synthetic
QPFPQPELPYP

SEQ ID NO: 532 - protein, 11 aa, Artificial sequence
  synthetic
LQPFPQPELPY

SEQ ID NO: 533 - protein, 12 aa, Artificial sequence
  synthetic
FPQPELPYPQPQ

SEQ ID NO: 534 - protein, 12 aa, Artificial sequence
  synthetic
PFPQPELPYPQP

SEQ ID NO: 535 - protein, 12 aa, Artificial sequence
  synthetic
LQPFPQPELPYP

SEQ ID NO: 536 - protein, 13 aa, Artificial sequence
  synthetic
PFPQPELPYPQPQ

SEQ ID NO: 537 - protein, 13 aa, Artificial sequence
  synthetic
LQPFPQPELPYPQ

SEQ ID NO: 538 - protein, 6 aa, Artificial sequence
  synthetic
QPEQPF

SEQ ID NO: 539 - protein, 7 aa, Artificial sequence
  synthetic
QPEQPFP

SEQ ID NO: 540 - protein, 7 aa, Artificial sequence
  synthetic
PQPEQPF

SEQ ID NO: 541 - protein, 8 aa, Artificial sequence
  synthetic
QPEQPFPW

SEQ ID NO: 542 - protein, 8 aa, Artificial sequence
  synthetic
PQPEQPFP

SEQ ID NO: 543 - protein, 8 aa, Artificial sequence
  synthetic
FPQPEQPF

SEQ ID NO: 544 - protein, 9 aa, Artificial sequence
  synthetic
QPEQPFPWQ

SEQ ID NO: 545 - protein, 9 aa, Artificial sequence
  synthetic
FPQPEQPFP

SEQ ID NO: 546 - protein, 10 aa, Artificial sequence
  synthetic
QPEQPFPWQP

SEQ ID NO: 547 - protein, 10 aa, Artificial sequence
  synthetic
PQPEQPFPWQ

SEQ ID NO: 548 - protein, 10 aa, Artificial sequence
  synthetic
FPQPEQPFPW

SEQ ID NO: 549 - protein, 10 aa, Artificial sequence
  synthetic
PFPQPEQPFP

SEQ ID NO: 550 - protein, 10 aa, Artificial sequence
  synthetic
QPFPQPEQPF

SEQ ID NO: 551 - protein, 11 aa, Artificial sequence
  synthetic
PQPEQPFPWQP

SEQ ID NO: 552 - protein, 11 aa, Artificial sequence
  synthetic
FPQPEQPFPWQ

SEQ ID NO: 553 - protein, 11 aa, Artificial sequence
  synthetic
PFPQPEQPFPW

SEQ ID NO: 554 - protein, 11 aa, Artificial sequence
  synthetic
QPFPQPEQPFP

SEQ ID NO: 555 - protein, 12 aa, Artificial sequence
  synthetic
FPQPEQPFPWQP

SEQ ID NO: 556 - protein, 12 aa, Artificial sequence
  synthetic
PFPQPEQPFPWQ

SEQ ID NO: 557 - protein, 13 aa, Artificial sequence
  synthetic
PFPQPEQPFPWQP

SEQ ID NO: 558 - protein, 13 aa, Artificial sequence
  synthetic
QPFPQPEQPFPWQ

SEQ ID NO: 559 - protein, 7 aa, Artificial sequence
  synthetic
PIPEQPQ

SEQ ID NO: 560 - protein, 8 aa, Artificial sequence
  synthetic
PIPEQPQP

SEQ ID NO: 561 - protein, 8 aa, Artificial sequence
  synthetic
QPIPEQPQ

SEQ ID NO: 562 - protein, 9 aa, Artificial sequence
  synthetic
QPIPEQPQP

SEQ ID NO: 563 - protein, 10 aa, Artificial sequence
  synthetic
PIPEQPQPYP

SEQ ID NO: 564 - protein, 10 aa, Artificial sequence
  synthetic
QPIPEQPQPY

SEQ ID NO: 565 - protein, 10 aa, Artificial sequence
  synthetic
EQPIPEQPQP

SEQ ID NO: 566 - protein, 10 aa, Artificial sequence
  synthetic
PEQPIPEQPQ

SEQ ID NO: 567 - protein, 12 aa, Artificial sequence
  synthetic
PIPEQPQPYPQQ

SEQ ID NO: 568 - protein, 12 aa, Artificial sequence
  synthetic
QPIPEQPQPYPQ

SEQ ID NO: 569 - protein, 12 aa, Artificial sequence
  synthetic
EQPIPEQPQPYP

SEQ ID NO: 570 - protein, 12 aa, Artificial sequence
  synthetic
PEQPIPEQPQPY

SEQ ID NO: 571 - protein, 13 aa, Artificial sequence
  synthetic
QPIPEQPQPYPQQ

SEQ ID NO: 572 - protein, 13 aa, Artificial sequence
  synthetic
EQPIPEQPQPYPQ

SEQ ID NO: 573 - protein, 13 aa, Artificial sequence
  synthetic
PEQPIPEQPQPYP

SEQ ID NO: 574 - protein, 14 aa, Artificial sequence
  synthetic
EQPIPEQPQPYPQQ

SEQ ID NO: 575 - protein, 14 aa, Artificial sequence
  synthetic
PEQPIPEQPQPYPQ

SEQ ID NO: 576 - protein, 4 aa, Artificial sequence
  synthetic
PDLP

SEQ ID NO: 577 - protein, 7 aa, Artificial sequence
  synthetic
PELPYPQ

SEQ ID NO: 578 - protein, 13 aa, Artificial sequence
  synthetic
QPFPQPELPYPQP

SEQ ID NO: 579 - protein, 14 aa, Artificial sequence
  synthetic
QPFPQPELPYPQPQ

SEQ ID NO: 580 - protein, 14 aa, Artificial sequence
  synthetic
LQPFPQPELPYPQP

SEQ ID NO: 581 - protein, 11 aa, Artificial sequence
  synthetic
PIPEQPQPYPQ

SEQ ID NO: 582 - protein, 11 aa, Artificial sequence
  synthetic
QPIPEQPQPYP

SEQ ID NO: 583 - protein, 11 aa, Artificial sequence
  synthetic
EQPIPEQPQPY

SEQ ID NO: 584 - protein, 11 aa, Artificial sequence
  synthetic
PEQPIPEQPQP

SEQ ID NO: 585 - protein, 9 aa, Artificial sequence
  synthetic
SQQPYLQLQ

SEQ ID NO: 586 - protein, 9 aa, Artificial sequence
  synthetic
ALALVRMLI

SEQ ID NO: 587 - protein, 10 aa, Artificial sequence
  synthetic
YLLPRRGPRL

SEQ ID NO: 588 - protein, 9 aa, Artificial sequence
  synthetic
AISPRTLNA

SEQ ID NO: 589 - protein, 9 aa, Artificial sequence
  synthetic
VMAPRTLIL

SEQ ID NO: 590 - protein, 9 aa, Artificial sequence
  synthetic
SQAPLPCVL

SEQ ID NO: 591 - protein, 9 aa, Artificial sequence
  synthetic
QMRPVSRVL

SEQ ID NO: 592 - protein, 16 aa, Artificial sequence
  synthetic
HAVSEGTKAVTKYTSS

SEQ ID NO: 593 - protein, 16 aa, Artificial sequence
  synthetic
PAETATPAPVEKSPAK

SEQ ID NO: 594 - protein, 16 aa, Artificial sequence
  synthetic
AYVRLAPDYDALDVAN

SEQ ID NO: 595 - protein, 16 aa, Artificial sequence
  synthetic
HAVSEGTKAVTKYTSA

SEQ ID NO: 596 - protein, 16 aa, Artificial sequence
  synthetic
AVSDGVIKVFNDMKVR

SEQ ID NO: 597 - protein, 16 aa, Artificial sequence
  synthetic
HAVSEGTKAVTKYTSS

SEQ ID NO: 598 - protein, 16 aa, Artificial sequence
  synthetic
QLLQANPILEAFGNAK

SEQ ID NO: 599 - protein, 16 aa, Artificial sequence
  synthetic
KSADTLWDIQKDLKDL

SEQ ID NO: 600 - protein, 16 aa, Artificial sequence
  synthetic
AYVRLAPDYDALDVAN

SEQ ID NO: 601 - protein, 16 aa, Artificial sequence
  synthetic
HAVSEGTKAVTKYTSA

SEQ ID NO: 602 - protein, 16 aa, Artificial sequence
  synthetic
TGLIKGSGTAEVELKK

SEQ ID NO: 603 - protein, 16 aa, Artificial sequence
  synthetic
VSDGVIKVFNDMKVRK

SEQ ID NO: 604 - protein, 16 aa, Artificial sequence
  synthetic
ASGNYATVISHNPETK

SEQ ID NO: 605 - protein, 16 aa, Artificial sequence
  synthetic
TAEILELAGNAARDNK

SEQ ID NO: 606 - protein, 16 aa, Artificial sequence
  synthetic
HAVSEGTKAVTKYTSA

SEQ ID NO: 607 - protein, 15 aa, Artificial sequence
  synthetic
PAPVEKSPAKKKATK

SEQ ID NO: 608 - protein, 15 aa, Artificial sequence
  synthetic
SADTLWDIQKDLKDL

SEQ ID NO: 609 - protein, 15 aa, Artificial sequence
  synthetic
TGLIKGSGTAEVELK

SEQ ID NO: 610 - protein, 15 aa, Artificial sequence
  synthetic
KSADTLWGIQKELQF

SEQ ID NO: 611 - protein, 15 aa, Artificial sequence
  synthetic
HGSYEDAVHSGALND

SEQ ID NO: 612 - protein, 15 aa, Artificial sequence
  synthetic
SDGVIKVFNDMKVRK

SEQ ID NO: 613 - protein, 15 aa, Artificial sequence
  synthetic
AGNLGGGVVTIERSK

SEQ ID NO: 614 - protein, 15 aa, Artificial sequence
  synthetic
AQAAAPASVPAQAPK

SEQ ID NO: 615 - protein, 15 aa, Artificial sequence
  synthetic
PRKIEEIKDFLLTAR

SEQ ID NO: 616 - protein, 14 aa, Artificial sequence
  synthetic
SEGTKAVTKYTSSK

SEQ ID NO: 617 - protein, 14 aa, Artificial sequence
  synthetic
VLKQVHPDTGISSK

SEQ ID NO: 618 - protein, 14 aa, Artificial sequence
  synthetic
SWTAADTAAQITQR

SEQ ID NO: 619 - protein, 14 aa, Artificial sequence
  synthetic
FISVGYVDDTQFVR

SEQ ID NO: 620 - protein, 14 aa, Artificial sequence
  synthetic
NIDDGTSDRPYSHA

SEQ ID NO: 621 - protein, 14 aa, Artificial sequence
  synthetic
VLKQVHPDTGISSK

SEQ ID NO: 622 - protein, 14 aa, Artificial sequence
  synthetic
RKTVTAMDVVYALK

SEQ ID NO: 623 - protein, 14 aa, Artificial sequence
  synthetic
SADTLWGIQKELQF

SEQ ID NO: 624 - protein, 14 aa, Artificial sequence
  synthetic
ASAETVDPASLWEY

SEQ ID NO: 625 - protein, 13 aa, Artificial sequence
  synthetic
TVVNKDVFRDPAL

SEQ ID NO: 626 - protein, 13 aa, Artificial sequence
  synthetic
KTVTAMDVVYALK

SEQ ID NO: 627 - protein, 13 aa, Artificial sequence
  synthetic
EGIPALDNFLDKL

SEQ ID NO: 628 - protein, 13 aa, Artificial sequence
  synthetic
RVTIMPKDIQLAR

SEQ ID NO: 629 - protein, 13 aa, Artificial sequence
  synthetic
PVAVMAESAFSFK

SEQ ID NO: 630 - protein, 13 aa, Artificial sequence
  synthetic
QTVAVGVIKAVDK

SEQ ID NO: 631 - protein, 13 aa, Artificial sequence
  synthetic
ILELAGNAARDNK

SEQ ID NO: 632 - protein, 12 aa, Artificial sequence
  synthetic
GTGASGSFKLNK

SEQ ID NO: 633 - protein, 12 aa, Artificial sequence
  synthetic
KQVHPDTGISSK

SEQ ID NO: 634 - protein, 12 aa, Artificial sequence
  synthetic
VGGTSDVEVNEK

SEQ ID NO: 635 - protein, 12 aa, Artificial sequence
  synthetic
NSVVEASEAAYK

SEQ ID NO: 636 - protein, 12 aa, Artificial sequence
  synthetic
ALRYPMAVGLNK

SEQ ID NO: 637 - protein, 12 aa, Artificial sequence
  synthetic
SLVSKGTLVQTK

SEQ ID NO: 638 - protein, 11 aa, Artificial sequence
  synthetic
PELAKSAPAPK

SEQ ID NO: 639 - protein, 11 aa, Artificial sequence
  synthetic
SEMEVQDAELK

SEQ ID NO: 640 - protein, 11 aa, Artificial sequence
  synthetic
QTYSTEPNNLK

SEQ ID NO: 641 - protein, 11 aa, Artificial sequence
  synthetic
PMFIVNTNVPR

SEQ ID NO: 642 - protein, 10 aa, Artificial sequence
  synthetic
AGFAGDDAPR

SEQ ID NO: 643 - protein, 10 aa, Artificial sequence
  synthetic
RVNAGTLAVL

SEQ ID NO: 644 - protein, 9 aa, Artificial sequence
  synthetic
IGQSKVFFR

SEQ ID NO: 645 - protein, 16 aa, Artificial sequence
  synthetic
TAEILELAGNAARDNK

SEQ ID NO: 646 - protein, 13 aa, Artificial sequence
  synthetic
ILELAGNAARDNK

SEQ ID NO: 647 - protein, 17 aa, Artificial sequence
  synthetic
ALAGCHLEDTQRKLQKG

SEQ ID NO: 648 - protein, 16 aa, Artificial sequence
  synthetic
MQLITRGKGAGTPNLI

SEQ ID NO: 649 - protein, 15 aa, Artificial sequence
  synthetic
KMKLRNTVHLSYLTV

SEQ ID NO: 650 - protein, 15 aa, Artificial sequence
  synthetic
CRASQTISSYLDWYQ

SEQ ID NO: 651 - protein, 13 aa, Artificial sequence
  synthetic
PAALTNKGNTVFA

SEQ ID NO: 652 - protein, 13 aa, Artificial sequence
  synthetic
WTPGPSAGVTGIA

SEQ ID NO: 653 - protein, 10 aa, Artificial sequence
  synthetic
ILRTIGKEAF

SEQ ID NO: 654 - protein, 9 aa, Artificial sequence
  synthetic
RSCGYACTA

SEQ ID NO: 655 - protein, 9 aa, Artificial sequence
  synthetic
FPNGFSFIH

SEQ ID NO: 656 - protein, 9 aa, Artificial sequence
  synthetic
SHGPYIKLI

SEQ ID NO: 657 - protein, 15 aa, Artificial sequence
  synthetic
AQAAAPASVPAQAPK

SEQ ID NO: 658 - protein, 17 aa, Artificial sequence
  synthetic
AYVRLAPDYDALDVANK

SEQ ID NO: 659 - protein, 16 aa, Artificial sequence
  synthetic
AYVRLAPDYDALDVAN

SEQ ID NO: 660 - protein, 16 aa, Artificial sequence
  synthetic
ASGNYATVISHNPETK

SEQ ID NO: 661 - protein, 15 aa, Artificial sequence
  synthetic
AGNLGGGVVTIERSK

SEQ ID NO: 662 - protein, 15 aa, Artificial sequence
  synthetic
PRKIEEIKDFLLTAR

SEQ ID NO: 663 - protein, 14 aa, Artificial sequence
  synthetic
NIDDGTSDRPYSHA

SEQ ID NO: 664 - protein, 13 aa, Artificial sequence
  synthetic
TVVNKDVFRDPAL

SEQ ID NO: 665 - protein, 12 aa, Artificial sequence
  synthetic
ALRYPMAVGLNK

SEQ ID NO: 666 - protein, 11 aa, Artificial sequence
  synthetic
QTYSTEPNNLK

SEQ ID NO: 667 - protein, 11 aa, Artificial sequence
  synthetic
PELAKSAPAPK

SEQ ID NO: 668 - protein, 12 aa, Artificial sequence
  synthetic
KQVHPDTGISSK

SEQ ID NO: 669 - protein, 14 aa, Artificial sequence
  synthetic
VLKQVHPDTGISSK

SEQ ID NO: 670 - protein, 14 aa, Artificial sequence
  synthetic
SEGTKAVTKYTSSK

SEQ ID NO: 671 - protein, 16 aa, Artificial sequence
  synthetic
HAVSEGTKAVTKYTSA

SEQ ID NO: 672 - protein, 16 aa, Artificial sequence
  synthetic
HAVSEGTKAVTKYTSS

SEQ ID NO: 673 - protein, 17 aa, Artificial sequence
  synthetic
HAVSEGTKAVTKYTSAK

SEQ ID NO: 674 - protein, 17 aa, Artificial sequence
  synthetic
HAVSEGTKAVTKYTSSK

SEQ ID NO: 675 - protein, 16 aa, Artificial sequence
  synthetic
HAVSEGTKAVTKYTSA

SEQ ID NO: 676 - protein, 10 aa, Artificial sequence
  synthetic
AGFAGDDAPR

SEQ ID NO: 677 - protein, 9 aa, Artificial sequence
  synthetic
SQAPLPCVL

SEQ ID NO: 678 - protein, 9 aa, Artificial sequence
  synthetic
VMAPRTLFL

SEQ ID NO: 679 - protein, 13 aa, Artificial sequence
  synthetic
PKKTESHHKAKGK

SEQ ID NO: 680 - protein, 7 aa, Artificial sequence
  synthetic
AAVLEYL

SEQ ID NO: 681 - protein, 25 aa, Artificial sequence
  synthetic
AQAAAPASVPAQAPKRTQAPTKASE

SEQ ID NO: 682 - protein, 19 aa, Artificial sequence
  synthetic
KLEKEEEEGISQESSEEEQ

SEQ ID NO: 683 - protein, 19 aa, Artificial sequence
  synthetic
GDRSEDFGVNEDLADSDAR

SEQ ID NO: 684 - protein, 18 aa, Artificial sequence
  synthetic
VAPEEHPVLLTEAPLNPK

SEQ ID NO: 685 - protein, 16 aa, Artificial sequence
  synthetic
STAGDTHLGGEDFDNR

SEQ ID NO: 686 - protein, 15 aa, Artificial sequence
  synthetic
KVPQVSTPTLVEVSR

SEQ ID NO: 687 - protein, 15 aa, Artificial sequence
  synthetic
PDPAKSAPAPKKGSK

SEQ ID NO: 688 - protein, 12 aa, Artificial sequence
  synthetic
LQAEIEGLKGQR

SEQ ID NO: 689 - protein, 11 aa, Artificial sequence
  synthetic
PDPAKSAPAPK

SEQ ID NO: 690 - protein, 11 aa, Artificial sequence
  synthetic
PELAKSAPAPK

SEQ ID NO: 691 - protein, 11 aa, Artificial sequence
  synthetic
PEPVKSAPVPK

SEQ ID NO: 692 - protein, 9 aa, Artificial sequence
  synthetic
AAPATRAAL

SEQ ID NO: 693 - protein, 9 aa, Artificial sequence
  synthetic
SAPSRATAL

SEQ ID NO: 694 - protein, 8 aa, Artificial sequence
  synthetic
ILNFPPPP

SEQ ID NO: 695 - protein, 8 aa, Artificial sequence
  synthetic
IAPTGHSL

SEQ ID NO: 696 - protein, 8 aa, Artificial sequence
  synthetic
ISPHGNAL

SEQ ID NO: 697 - protein, 15 aa, Artificial sequence
  synthetic
PDPAKSAPAPKKGSK

SEQ ID NO: 698 - protein, 11 aa, Artificial sequence
  synthetic
PDPAKSAPAPK

SEQ ID NO: 699 - protein, 11 aa, Artificial sequence
  synthetic
PELAKSAPAPK

SEQ ID NO: 700 - protein, 11 aa, Artificial sequence
  synthetic
PEPVKSAPVPK

SEQ ID NO: 701 - protein, 18 aa, Artificial sequence
  synthetic
VAPEEHPVLLTEAPLNPK

SEQ ID NO: 702 - protein, 13 aa, Artificial sequence
  synthetic
PLLALLALWGPDP

SEQ ID NO: 703 - protein, 11 aa, Artificial sequence
  synthetic
PKTRREAEVGQ

SEQ ID NO: 704 - protein, 11 aa, Artificial sequence
  synthetic
RREAEDLQGSL

SEQ ID NO: 705 - protein, 11 aa, Artificial sequence
  synthetic
RREAEDLEGSL

SEQ ID NO: 706 - protein, 13 aa, Artificial sequence
  synthetic
DLQVGQVELGGGP

SEQ ID NO: 707 - protein, 13 aa, Artificial sequence
  synthetic
DLQVGEVELGGGP

SEQ ID NO: 708 - protein, 13 aa, Artificial sequence
  synthetic
CGSHLVEALYLVC

SEQ ID NO: 709 - protein, 11 aa, Artificial sequence
  synthetic
MNILLEYVVKS

SEQ ID NO: 710 - protein, 17 aa, Artificial sequence
  synthetic
NMFTYEIAPVFVLLEYV

SEQ ID NO: 711 - protein, 17 aa, Artificial sequence
  synthetic
NVCFWYIPPSLRTLEDN

SEQ ID NO: 712 - protein, 17 aa, Artificial sequence
  synthetic
FNQLSTGLDMVGLAADW

SEQ ID NO: 713 - protein, 17 aa, Artificial sequence
  synthetic
GRTGTYILIDMVLNRMA

SEQ ID NO: 714 - protein, 17 aa, Artificial sequence
  synthetic
PKAARPPVTPVLLEKKS

SEQ ID NO: 715 - protein, 11 aa, Artificial sequence
  synthetic
LLALLALWGPD

SEQ ID NO: 716 - protein, 21 aa, Artificial sequence
  synthetic
KKKKYVSIDVTLQQLESHKKK

SEQ ID NO: 717 - protein, 15 aa, Artificial sequence
  synthetic
GLKMFPDLTKVYSTD

SEQ ID NO: 718 - protein, 15 aa, Artificial sequence
  synthetic
PRHRDTGILDSIGRF

SEQ ID NO: 719 - protein, 17 aa, Artificial sequence
  synthetic
ENPVVHFFKNIVTPRTP

SEQ ID NO: 720 - protein, 15 aa, Artificial sequence
  synthetic
ASDYKSAHKGFKGVD

SEQ ID NO: 721 - protein, 15 aa, Artificial sequence
  synthetic
GFKGVDAQGTLSKIF

SEQ ID NO: 722 - protein, 14 aa, Artificial sequence
  synthetic
TQQIRLQAEIFQAR

SEQ ID NO: 723 - protein, 14 aa, Artificial sequence
  synthetic
AQQIRLQAEAFQAR

SEQ ID NO: 724 - protein, 14 aa, Artificial sequence
  synthetic
TAQIRLQAEIFQAR

SEQ ID NO: 725 - protein, 14 aa, Artificial sequence
  synthetic
TQAIRLQAEIFQAR

SEQ ID NO: 726 - protein, 14 aa, Artificial sequence
  synthetic
TQQARLQAEIFQAR

SEQ ID NO: 727 - protein, 14 aa, Artificial sequence
  synthetic
TQQIALQAEIFQAR

SEQ ID NO: 728 - protein, 15 aa, Artificial sequence
  synthetic
QTQQIRLQAEIFQAR

SEQ ID NO: 729 - protein, 13 aa, Artificial sequence
  synthetic
QQIRLQAEIFQAR

SEQ ID NO: 730 - protein, 9 aa, Artificial sequence
  synthetic
NIDALNENK

SEQ ID NO: 731 - protein, 11 aa, Artificial sequence
  synthetic
NVLVLDTDYKK

SEQ ID NO: 732 - protein, 15 aa, Artificial sequence
  synthetic
NTPEVDDEALEKFDK

SEQ ID NO: 733 - protein, 15 aa, Artificial sequence
  synthetic
LEDARRLKAIYEKKK

SEQ ID NO: 734 - protein, 13 aa, Artificial sequence
  synthetic
HSLGKWLGHPDKF

SEQ ID NO: 735 - protein, 14 aa, Artificial sequence
  synthetic
NTWTTCQSIAFPSK

SEQ ID NO: 736 - protein, 16 aa, Artificial sequence
  synthetic
IAATYNFAVLKLMGRG

SEQ ID NO: 737 - protein, 13 aa, Artificial sequence
  synthetic
VHFFKNIVTPRTP

SEQ ID NO: 738 - protein, 15 aa, Artificial sequence
  synthetic
DEGGYTCFFRDHSYQ

SEQ ID NO: 739 - protein, 9 aa, Artificial sequence
  synthetic
HLVEALYLV

SEQ ID NO: 740 - protein, 9 aa, Artificial sequence
  synthetic
LNIDLLWSV

SEQ ID NO: 741 - protein, 9 aa, Artificial sequence
  synthetic
VLFGLGFAI

SEQ ID NO: 742 - protein, 9 aa, Artificial sequence
  synthetic
VYLKTNVFL

SEQ ID NO: 743 - protein, 9 aa, Artificial sequence
  synthetic
KYNKANAFL

SEQ ID NO: 744 - protein, 9 aa, Artificial sequence
  synthetic
KYNIANVFL

SEQ ID NO: 745 - protein, 9 aa, Artificial sequence
  synthetic
KYNKANVFL

SEQ ID NO: 746 - protein, 9 aa, Artificial sequence
  synthetic
FQDENYLYL

SEQ ID NO: 747 - protein, 9 aa, Artificial sequence
  synthetic
LYLVCGERG

SEQ ID NO: 748 - protein, 10 aa, Artificial sequence
  synthetic
VMNILLQYVV

SEQ ID NO: 749 - protein, 10 aa, Artificial sequence
  synthetic
RMMEYGTTMV

SEQ ID NO: 750 - protein, 10 aa, Artificial sequence
  synthetic
NLAQTDLATV

SEQ ID NO: 751 - protein, 9 aa, Artificial sequence
  synthetic
QLARQQVHV

SEQ ID NO: 752 - protein, 9 aa, Artificial sequence
  synthetic
SLSPLQAEL

SEQ ID NO: 753 - protein, 9 aa, Artificial sequence
  synthetic
SLAAGVKLL

SEQ ID NO: 754 - protein, 9 aa, Artificial sequence
  synthetic
VIVMLTPLV

SEQ ID NO: 755 - protein, 9 aa, Artificial sequence
  synthetic
KLQVFLIVL

SEQ ID NO: 756 - protein, 9 aa, Artificial sequence
  synthetic
FLIVLSVAL

SEQ ID NO: 757 - protein, 9 aa, Artificial sequence
  synthetic
FLWSVFMLI

SEQ ID NO: 758 - protein, 9 aa, Artificial sequence
  synthetic
NLFLFLFAV

SEQ ID NO: 759 - protein, 9 aa, Artificial sequence
  synthetic
FLFAVGFYL

SEQ ID NO: 760 - protein, 9 aa, Artificial sequence
  synthetic
YLLLRVLNI

SEQ ID NO: 761 - protein, 9 aa, Artificial sequence
  synthetic
RLLCALTSL

SEQ ID NO: 762 - protein, 9 aa, Artificial sequence
  synthetic
ALWMRLLPL

SEQ ID NO: 763 - protein, 9 aa, Artificial sequence
  synthetic
LWMRLLPLL

SEQ ID NO: 764 - protein, 9 aa, Artificial sequence
  synthetic
RLLPLLALL

SEQ ID NO: 765 - protein, 10 aa, Artificial sequence
  synthetic
HLCGSHLVEA

SEQ ID NO: 766 - protein, 9 aa, Artificial sequence
  synthetic
ALYLVCGER

SEQ ID NO: 767 - protein, 10 aa, Artificial sequence
  synthetic
LYLVCGERGF

SEQ ID NO: 768 - protein, 9 aa, Artificial sequence
  synthetic
LVCGERGFF

SEQ ID NO: 769 - protein, 10 aa, Artificial sequence
  synthetic
VCGERGFFYT

SEQ ID NO: 770 - protein, 8 aa, Artificial sequence
  synthetic
GERGFFYT

SEQ ID NO: 771 - protein, 9 aa, Artificial sequence
  synthetic
ERGFFYTPK

SEQ ID NO: 772 - protein, 9 aa, Artificial sequence
  synthetic
FYTPKTRRE

SEQ ID NO: 773 - protein, 11 aa, Artificial sequence
  synthetic
TPKTRREAEDL

SEQ ID NO: 774 - protein, 9 aa, Artificial sequence
  synthetic
SLQPLALEG

SEQ ID NO: 775 - protein, 9 aa, Artificial sequence
  synthetic
ALEGSLQKR

SEQ ID NO: 776 - protein, 10 aa, Artificial sequence
  synthetic
SLQKRGIVEQ

SEQ ID NO: 777 - protein, 10 aa, Artificial sequence
  synthetic
GIVEQCCTSI

SEQ ID NO: 778 - protein, 9 aa, Artificial sequence
  synthetic
IVEQCCTSI

SEQ ID NO: 779 - protein, 9 aa, Artificial sequence
  synthetic
SLYQLENYC

SEQ ID NO: 780 - protein, 9 aa, Artificial sequence
  synthetic
SLLLELEEV

SEQ ID NO: 781 - protein, 9 aa, Artificial sequence
  synthetic
LMWAKIGPV

SEQ ID NO: 782 - protein, 9 aa, Artificial sequence
  synthetic
VLFSSDFRI

SEQ ID NO: 783 - protein, 9 aa, Artificial sequence
  synthetic
SLSRFSWGA

SEQ ID NO: 784 - protein, 9 aa, Artificial sequence
  synthetic
KVEDPFYWV

SEQ ID NO: 785 - protein, 10 aa, Artificial sequence
  synthetic
RTFDPHFLRV

SEQ ID NO: 786 - protein, 9 aa, Artificial sequence
  synthetic
FLRVPCWKI

SEQ ID NO: 787 - protein, 10 aa, Artificial sequence
  synthetic
KITLFVIVPV

SEQ ID NO: 788 - protein, 9 aa, Artificial sequence
  synthetic
VLGPLVALI

SEQ ID NO: 789 - protein, 9 aa, Artificial sequence
  synthetic
TLFVIVPVL

SEQ ID NO: 790 - protein, 10 aa, Artificial sequence
  synthetic
RLAGQFLEEL

SEQ ID NO: 791 - protein, 9 aa, Artificial sequence
  synthetic
FLYGALLLA

SEQ ID NO: 792 - protein, 15 aa, Artificial sequence
  synthetic
FIEWNKLRFRQGLEW

SEQ ID NO: 793 - protein, 5 aa, Artificial sequence
  synthetic
GGGGS

SEQ ID NO: 794 - protein, 4 aa, Artificial sequence
  synthetic
GGSG

SEQ ID NO: 795 - protein, 4 aa, Artificial sequence
  synthetic
SGGG

SEQ ID NO: 796 - protein, 4 aa, Artificial sequence
  synthetic
GSGS

SEQ ID NO: 797 - protein, 6 aa, Artificial sequence
  synthetic
GSGSGS

SEQ ID NO: 798 - protein, 8 aa, Artificial sequence
  synthetic
GSGSGSGS

SEQ ID NO: 799 - protein, 10 aa, Artificial sequence
  synthetic
GSGSGSGSGS

SEQ ID NO: 800 - protein, 12 aa, Artificial sequence
  synthetic
GSGSGSGSGSGS

SEQ ID NO: 801 - protein, 6 aa, Artificial sequence
  synthetic
GGSGGS

SEQ ID NO: 802 - protein, 9 aa, Artificial sequence
  synthetic
GGSGGSGGS

SEQ ID NO: 803 - protein, 12 aa, Artificial sequence
  synthetic
GGSGGSGGSGGS

SEQ ID NO: 804 - protein, 4 aa, Artificial sequence
  synthetic
GGSG

SEQ ID NO: 805 - protein, 8 aa, Artificial sequence
  synthetic
GGSGGGSG

SEQ ID NO: 806 - protein, 12 aa, Artificial sequence
  synthetic
GGSGGGSGGGSG

SEQ ID NO: 807 - protein, 15 aa, Artificial sequence
  synthetic
GGGGSGGGGSGGGGS

SEQ ID NO: 808 - protein, 20 aa, Artificial sequence
  synthetic
SGGGSGGGSGGGSGGGSGGG

SEQ ID NO: 809 - protein, 4 aa, Artificial sequence
  synthetic
GGGG

SEQ ID NO: 810 - protein, 8 aa, Artificial sequence
  synthetic
GGGGGGGG

SEQ ID NO: 811 - protein, 12 aa, Artificial sequence
  synthetic
GGGGGGGGGGGG

SEQ ID NO: 812 - protein, 16 aa, Artificial sequence
  synthetic
GGGGGGGGGGGGGGGG

SEQ ID NO: 813 - protein, 20 aa, Artificial sequence
  synthetic
GGGGGGGGGGGGGGGGGGGG

SEQ ID NO: 814 - protein, 5 aa, Artificial sequence
  synthetic
GGGGG

SEQ ID NO: 815 - protein, 10 aa, Artificial sequence
  synthetic
GGGGGGGGGG

SEQ ID NO: 816 - protein, 15 aa, Artificial sequence
  synthetic
GGGGGGGGGGGGGGG

SEQ ID NO: 817 - protein, 20 aa, Artificial sequence
  synthetic
GGGGGGGGGGGGGGGGGGGG

SEQ ID NO: 818 - protein, 20 aa, Artificial sequence
  synthetic
GGGGGGGGGGGGGGGGGGGG

SEQ ID NO: 819 - protein, 10 aa, Artificial sequence
  synthetic
GENLYFQSGG

SEQ ID NO: 820 - protein, 5 aa, Artificial sequence
  synthetic
RSIAT

SEQ ID NO: 821 - protein, 17 aa, Artificial sequence
  synthetic
RPACKIPNDLKQKVMNH

SEQ ID NO: 822 - protein, 36 aa, Artificial sequence
  synthetic
GGSAGGSGSGSSGGSSGASGTGTAGGTGSGSGTGSG

SEQ ID NO: 823 - protein, 17 aa, Artificial sequence
  synthetic
AAANSSIDLISVPVDSR

SEQ ID NO: 824 - protein, 36 aa, Artificial sequence
  synthetic
GGSGGGSEGGGSEGGGSEGGGSEGGGSEGGGSGGGS

SEQ ID NO: 825 - protein, 16 aa, Artificial sequence
  synthetic
CSSGWYRSPFSRVVHL

SEQ ID NO: 826 - protein, 21 aa, Artificial sequence
  synthetic
MEVGWYRSPFSRVVHLYRNGK

SEQ ID NO: 827 - protein, 17 aa, Artificial sequence
  synthetic
ASFEAQGALANIAVDKA

SEQ ID NO: 828 - protein, 17 aa, Artificial sequence
  synthetic
ISQAVHAAHAEINEAGR

SEQ ID NO: 829 - protein, 21 aa, Artificial sequence
  synthetic
GPKGQTGKPGIAGFKGEQGPK

(19) United States
(12) Patent Application Publication
(10) Pub. No.: US 2023/0001010 A1
(43) Pub. Date: Jan. 5, 2023

(54) COMPOSITIONS AND METHODS FOR TREATING AUTOIMMUNE DISORDERS

(71) Applicant: The Regents of the University of Michigan, Ann Arbor, MI (US)

(72) Inventors: James J. Moon, Ann Arbor, MI (US); Anna A. Schwendeman, Ann Arbor, MI (US); Sayed Alireza Hassani Najafabadi, Ann Arbor, MI (US)

(21) Appl. No.: 17/628,418

(22) PCT Filed: Jul. 17, 2020
(86) PCT No.: PCT/US2020/042545

§ 371 Date: Jan. 19, 2022

Related U.S. Application Data
(60) Provisional application No. 62/876,419, filed on Jul. 19, 2019.
(60) Provisional application No. 63/017,444, filed on Apr. 29, 2020.

(51) Int. Cl. A61K 47/69; A61K 39/00

(52) U.S. Cl. CPC A61K 47/6917; A61K 39/0008; A61K 2039/55555

(57) ABSTRACT

The present invention relates to compositions comprising nanoparticles associated with a plurality of tolerogenic antigens (e.g., between 1-30 tolerogenic 5 antigens per nanoparticle) in such a manner that facilitates strong immune tolerance upon administration to a subject (e.g., a human subject suffering from or at risk of suffering from an autoimmune disorder e.g., MS or celiac disease). The present invention further relates to methods for utilizing such nanoparticles to treat autoimmune disorders (e.g., MS or celiac disease).

### SEQUENCE LISTING

The instant application contains a Sequence Listing which has been submitted electronically in ASCII format and is hereby incorporated by reference in its entirety. Said ASCII copy, created on Jul. 17, 2020 is named 37921-601_ST25.txt and is 203,000 bytes in size.

### FIELD OF THE INVENTION

The present invention relates to nanoparticles associated with a plurality of tolerogenic antigens (e.g., between 1-30 tolerogenic antigens (e.g., 4-30, 5-30, 6-30, 7-30, or 8-30 tolerogenic antigens per nanoparticle)) in such a manner that facilitates strong immune tolerance upon administration to a subject (e.g., a human subject suffering from or at risk of suffering from an autoimmune disorder e.g., MS or celiac disease). The present invention further relates to methods for synthesizing such nanoparticles associated with tolerogenic antigens implicated in celiac disease, as well as systems and methods utilizing such nanoparticles modified with tolerogenic antigens for the treatment of celiac disease.

### BACKGROUND OF THE INVENTION

Autoimmune disorders are diseases that occur when the body's immune system attacks its own normal tissues, organs or other in vivo components due to immune system abnormalities whose cause cannot be found. These autoimmune disorders are systemic diseases that can occur in almost all parts of the body, including the nervous system, the gastrointestinal system, the endocrine system, the skin, the skeletal system, and the vascular tissue. It is known that autoimmune disorders affect about 5-8% of the world population, but the reported prevalence of autoimmune disorders is lower than the actual level due to limitations in the understanding of autoimmune disorders and a method for diagnosing these diseases.

Improved compositions and methods for treating autoimmune conditions are needed.

Celiac disease is an autoimmune disorder triggered by the presence of gluten, specifically, the proteins classified as gliadin and glutenin proteins found in cereals from the genus Triticum including wheat, rye, and barely. About three million people suffer from celiac disease in the United States alone, but 83% of people living with celiac disease are undiagnosed. Since the 1950s, the number of people suffering from celiac disease has doubled about every 20 years, creating an increased need for effective methods of diagnosis and treatment. Currently, there are no therapeutics approved by the Federal Drug Administration for the treatment of celiac disease. Therefore, subjects suffering from celiac disease must observe a strict, gluten-free diet. However, a strict, gluten-free diet may be challenging for a subject to implement given that as little as 50 mg of gluten, the equivalent of 1/70^{th} of a slice of bread, per day can cause intestinal damage for a person suffering from celiac disease. Additionally, some subjects have been diagnosed with refractory celiac disease, meaning that despite adherence of a strict, gluten-free diet, the disease remains unresponsive, with symptoms and damage to the small intestines persisting.

The hallmark symptoms associated with celiac disease include diarrhea, bloating, gas, fatigue, weight loss, Fe deficiency anemia, constipation, an itchy rash, and depression. If left untreated, celiac disease can lead to osteoporosis, anemia, thyroid disease, and the development of certain types of cancer. While the pathogenesis of celiac disease is complex, it is known to be primarily triggered by the presence of a 33 amino acid gliadin polypeptide, which is naturally formed during gastrointestinal digestion of gliadin. When gliadin is present, the enzyme tissue transglutaminase 2 (TG2) deamidates specific glutamine residues on the gliadin peptide to form glutamic acid reside. If an individual carries the human leukocyte antigen (HLA) DQ2 or DQ8 haplotypes, the antigen presenting cells expressing HLA-DQ2 or HLA-DQ8 have a greater affinity for deamidated peptides and will bind the deamidated gliadin to form an HLA-DQ2/8-gliadin complex. This complex can then activate host-gluten-specific CD₄⁺ T-cells, which stimulates B-cells, resulting in production of anti-gliadin and anti-TG2 antibodies. Additionally, T-cell activation causes cytokine production, causing inflammation and damage to small intestine. The HLA-DG2/8-gliadin complex also increases production of IFNγ, which may lead to mucosal intestinal lesions. In order to combat the life-altering symptoms and damaging effects of celiac disease, there remains a need to develop therapeutics to effectively treat this autoimmune disease.

### SUMMARY OF THE INVENTION

The present disclosure provides nanoparticles associated with a population of tolerogenic antigens (e.g., between 1-30 (e.g., 8-30, e.g., 9 to 15, 12 to 18, 15 to 22, 18 to 25, 20 to 27, 22 to 28, or 25 to 30, tolerogenic antigens per nanoparticle)) in such a manner that the resulting composition is capable of facilitating strong immune tolerance to antigens associated with an autoimmune disorder (e.g., multiple sclerosis (MS), celiac disease, rheumatoid arthritis, diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, and inflammatory bowel disease) upon administration to a subject (e.g., a human subject suffering from or at risk of suffering from an autoimmune disorder (e.g., (MS or celiac disease)). The present invention further relates to methods for synthesizing such nanoparticles associated with tolerogenic antigens implicated in celiac disease, as well as systems and methods utilizing such nanoparticles to treat a subject suffering from celiac disease.

In a first aspect, the disclosure provides a composition including an sHDL nanoparticle associated with a plurality of tolerogenic antigens in such a manner that the resulting composition is capable of facilitating strong immune tolerance to antigens associated with an autoimmune disease (e.g., MS, celiac disease, rheumatoid arthritis, diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, and inflammatory bowel disease) upon administration to a subject, wherein the sHDL nanoparticle includes a mixture of at least one phospholipid and at least one HDL apolipoprotein or apolipoprotein mimetic. Such compositions are not limited to a particular nanoparticle. In some embodiments, the nanoparticle is a sHDL nanoparticle. In some embodiments, the average size of the nanoparticle is between 6 to 500 nm (e.g., 7 to 20 nm, 21 to 50 nm, 51 to 100 nm, 101 to 200 nm, 201 to 300 nm, 301 to 400 nm, and 401 to 500 nm). In some embodiments, the average particle size of the sHDL nanoparticle is between 6-70 nm (e.g., 7 to 10 nm, 11 to 20 nm, 21 to 30 nm, 31 to 40 nm, 41 to 50 nm, 51 to 60 nm, and 61 to 70 nm).

In some embodiments, the phospholipid is selected from the group consisting of 1,2-dilauroyl-sn-glycero-3-phosphocholine; 1,2-dimyristoyl-sn-glycero-3-phosphocholine; 1,2-dipalmitoyl-sn-glycero-3-phosphocholine; 1,2-distearoyl-sn-glycero-3-phosphocholine; 1,2-diarachidoyl-sn-glycero-3-phosphocholine; 1,2-dibehenoyl-sn-glycero-3-phosphocholine; 1,2-dilignoceroyl-sn-glycero-3-phosphocholine; 1,2-dimyristoleoyl-sn-glycero-3-phosphocholine; 1,2-dimyristelaidoyl-sn-glycero-3-phosphocholine; 1,2-dipalmitoleoyl-sn-glycero-3-phosphocholine; 1,2-dipalmitelaidoyl-sn-glycero-3-phosphocholine; 1,2-dipetroselenoyl-sn-glycero-3-phosphocholine; 1,2-dioleoyl-sn-glycero-3-phosphocholine; 1,2-dielaidoyl-sn-glycero-3-phosphocholine; 1,2-dieicosenoyl-sn-glycero-3-phosphocholine; 1,2-dinervonoyl-sn-glycero-3-phosphocholine; 1,2-dilauroyl-sn-glycero-3-phosphoethanolamine; 1,2-dimyristoyl-sn-glycero-3-phosphoethanolamine; 1,2-dipentadecanoyl-sn-glycero-3-phosphoethanolamine; 1,2-dipalmitoyl-sn-glycero-3-phosphoethanolamine; 1,2-distearoyl-sn-glycero-3-phosphoethanolamine; 1,2-dipalmitoleoyl-sn-glycero-3-phosphoethanolamine; 1,2-dielaidoyl-sn-glycero-3-phosphoethanolamine; 1,2-dioleoyl-sn-glycero-3-phosphoethanolamine; dioleoyl-sn-glycero-3-phosphoethanolamine-N-[3-(2-pyridyldithio) propionate]; 1,2-dipalmitoyl-sn-glycero-3-phosphothioethanol; 1,2-di-(9Z-octadecenoyl)-sn-glycero-3-phosphoethanolamine-N-[4-(p-maleimidophenyl)butyramide]; 1,2-dihexadecanoyl-sn-glycero-3-phosphoethanolamine-N-[4-(p-maleimidophenyl)butyramide]; 1,2-dihexadecanoyl-sn-glycero-3-phosphoethanolamine-N-[4-(p-maleimidomethyl)cyclohexane-carboxamide]; 1,2-di-(9Z-octadecenoyl)-sn-glycero-3-phosphoethanolamine-N-[4-(p-maleimidomethyl)cyclohexane-carboxamide]; N-[(3-Maleimide-1-oxopropyl)aminopropyl polyethyleneglycol-carbamyl] distearoylphosphatidyl-ethanolamine; N-[(3-Maleimide-1-oxopropyl)aminopropyl polyethyleneglycol-carbamyl] distearoylphosphatidyl-ethanolamine; N-(3-Maleimide-1-oxopropyl)-L-a-phosphatidylethanolamine, Distearoyl; N-[(3-Maleimide-1-oxopropyl)aminopropyl polyethyleneglycol-carbamyl] distearoylphosphatidyl-ethanolamine; N-(3-Maleimide-1-oxopropyl)-L-a-phosphatidylethanolamine, Dimyristoy; N-(3-Maleimide-1-oxopropyl)-L-a-phosphatidylethanolamine, Dioleoyl; N-(3-Maleimide-1-oxopropyl)-L-a-phosphatidylethanolamine, Dipalmitoyl; N-(3-Maleimide-1-oxopropyl)-L-a-phosphatidylethanolamine, 1-Palmitoyl-2-oleoyl; phosphatidylcholine; phosphatidylinositol; phosphatidylserine; phosphatidylethanolamine; N-(Succinimidyloxy-glutaryl)-L-a-phosphatidylethanolamine, Distearoyl; N-(Succinimidyloxy-glutaryl)-L-a-phosphatidylethanolamine, Dioleoyl; N-(Succinimidyloxy-glutaryl)-L-a-phosphatidylethanolamine, 1-Palmitoyl-2-oleoyl; N-(Succinimidyloxy-glutaryl)-L-a-phosphatidylethanolamine, Dipalmitoyl; N-(Succinimidyloxy-glutaryl)-L-a-phosphatidylethanolamine, Dimyristoyl; 3-(N-succinimidyloxyglutaryl)aminopropyl, and polyethyleneglycol-carbamyl distearoylphosphatidyl-ethanolamine; N-(3-oxopropoxy polyethyleneglycol)carbamyl-distearoyl-ethanolamine.

In some embodiments, the HDL apolipoprotein component is selected from the group consisting of apolipoprotein A-I (apo A-I), apolipoprotein A-II (apo A-II), apolipoprotein A-II xxx (apo A-II-xxx), apolipoprotein A4 (apo A4), apolipoprotein Cs (apo Cs), apolipoprotein E (apo E), apolipoprotein A-1 milano (apo A-I-milano), apolipoprotein A-I paris (apo A-I-paris), apolipoprotein M (apo M), an HDL apolipoprotein mimetic, preproapoliprotein, preproApoA-I, proApoA I, preproApoA-II, proApoA II, preproApoA-IV, proApoA-IV, ApoA-V, preproApoE, proApoE, preproApoA I_{Milano}, proApoA-I_{Milano}, preproApoA-I_{Paris}, proApoA-I_{Paris}, and mixtures thereof.

In some embodiments, the ApoA-I mimetic is described by any of SEQ ID NOs: 1-336 and

(SEQ ID NO: 341)
WDRVKDLATVYVDVLKDSGRDYVSQF,
(SEQ ID NO: 342)
LKLLDNWDSVTSTFSKLREOL,
(SEQ ID NO: 343)
PVTOEFWONLEKETEGLROEMS,
(SEQ ID NO: 344)
KDLEEVKAKVQ,
(SEQ ID NO: 345)
KDLEEVKAKVO,
(SEQ ID NO: 346)
PYLDDFQKKWQEEMELYRQKVE,
(SEQ ID NO: 347)
PLRAELQEGARQKLHELOEKLS,
(SEQ ID NO: 348)
PLGEEMRDRARAHVDALRTHLA,
(SEQ ID NO: 349)
PYSDELRQRLAARLEALKENGG,
(SEQ ID NO: 350)
ARLAEYHAKATEHLSTLSEKAK,
(SEQ ID NO: 351)
PALEDLROGLL,
(SEQ ID NO: 352)
PVLESFKVSFLSALEEYTKKLN,
(SEQ ID NO: 353)
PVLESFVSFLSALEEYTKKLN,
(SEQ ID NO: 352)
PVLESFKVSFLSALEEYTKKLN,
(SEQ ID NO: 354)
TVLLLTICSLEGALVRRQAKEPCV,
(SEQ ID NO: 355)
QTVTDYGKDLME,
(SEQ ID NO: 356)
KVKSPELOAEAKSYFEKSKE,
(SEQ ID NO: 357)
VLTLALVAVAGARAEVSADOVATV,
(SEQ ID NO: 358)
NNAKEAVEHLOKSELTOOLNAL,
(SEQ ID NO: 359)
LPVLVWLSIVLEGPAPAOGTPDVSS,
(SEQ ID NO: 360)
LPVLWVVLSIVLEGPAPAQGTPDVSS,
(SEQ ID NO: 361)
ALDKLKEFGNTLEDKARELIS,
(SEQ ID NO: 362)
VALLALLASARASEAEDASLL,
(SEQ ID NO: 363)
HLRKLRKRLLRDADDLQKRLAVYOA,
(SEQ ID NO: 364)
AQAWGERLRARMEEMGSRTRDR,
(SEQ ID NO: 365)
LDEVKEQVAEVRAKLEEQAQ,
(SEQ ID NO: 236)
DWLKAFYDKVAEKLKEAF,
(SEQ ID NO: 366)
DWLKAFYDKVAEKLKEAFPDWAKAAYDKAAEKAKEAA,
(SEQ ID NO: 367)
PVLDLFRELLNELLEALKQKL,
(SEQ ID NO: 368)
PVLDLFRELLNELLEALKQKLA,
(SEQ ID NO: 4)
PVLDLFRELLNELLEALKQKLK,
(SEQ ID NO: 369)
PVLDLFRELLNELLEALKQKLA,
(SEQ ID NO: 370)
PVLDLFRELLNELLEALKKLLK,
(SEQ ID NO: 371)
PVLDLFRELLNELLEALKKLLA,
(SEQ ID NO: 372)
PLLDLFRELLNELLEALKKLLA,
and
(SEQ ID NO: 373)
EVRSKLEEWFAAFREFAEEFLARLKS.

In some embodiments, the plurality of tolerogenic antigens are tolerogenic antigens including between 3 amino acids and 50 amino acids in length (e.g., e.g., about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, or about 50 amino acids in length).

In some embodiments, the plurality of tolerogenic antigens are tolerogenic antigens including a polypeptide including a nucleic acid sequence of any one of SEQ ID NOs: 375-796.

In some embodiments, the plurality of tolerogenic antigens are human allograft transplantation antigens. In some embodiments, the human allograft transplantation antigens are selected from subunits of the various MHC class I and MHC class II haplotype proteins, and single-amino-acid polymorphisms on minor blood group antigens including RhCE, Kell, Kidd, Duffy and Ss.

In some embodiments, the plurality of tolerogenic antigens are specific for type 1 diabetes mellitus. In some embodiments, the type 1 diabetes mellitus tolerogenic antigens are selected from insulin, proinsulin, preproinsulin, glutamic acid decarboxylase-65 (GAD-65), GAD-67, insulinoma-associated protein 2 (IA-2), insulinoma-associated protein 2β (IA-2β), ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S100p, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5.

In some embodiments, the tolerogenic antigens are specific for one or more of the following autoimmune disorders: rheumatoid arthritis, multiple sclerosis diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, and inflammatory bowel disease.

In some embodiments, the plurality of tolerogenic antigens include one or more of tolerogenic antigens selected from thyroglobulin (TG), thyroid peroxidase (TPO), thyrotropin receptor (TSHR), sodium iodine symporter (NIS), megalin, thyroid autoantigens including TSHR, insulin-like growth factor 1 receptor, calcium sensitive receptor, 21-hydroxylase, 17α-hydroxylase, and P450 side chain cleavage enzyme (P450scc), ACTH receptor, P450c21, P450c17, FSH receptor, α-enolase, pituitary gland-specific protein factor (PGSF) 1a and 2, and type 2 iodothyronine deiodinase, myelin basic protein, myelin oligodendrocyte glycoprotein, proteolipid protein, collagen II, H⁺, K⁺-ATPase, tissue transglutaminase and gliadin, tyrosinase, tyrosinase related protein 1 and 2, acetylcholine receptor, desmoglein 3, 1 and 4, pemphaxin, desmocollins, plakoglobin, perplakin, desmoplakins, acetylcholine receptor, BP180, BP230, plectin, laminin 5, endomysium, tissue transglutaminase, collagen VII, matrix metalloproteinase 1 and 3, the collagen-specific molecular chaperone heat-shock protein 47, fibrillin-1, PDGF receptor, Scl-70, U1 RNP, Th/To, Ku, Jo1, NAG-2, centromere proteins, topoisomerase I, nucleolar proteins, RNA polymerase I, II and III, PM-Slc, fibrillarin, B23, U1snRNP, nuclear antigens SS-A and SS-B, fodrin, poly(ADP-ribose) polymerase, topoisomerase, nuclear proteins including SS-A, high mobility group box 1 (HMGB1), nucleosomes, histone proteins, double-stranded DNA, glomerular basement membrane proteins including collagen IV, cardiac myosin, aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and type 1 interferons interferon alpha, beta and omega.

Such compositions are not limited to specific tolerogenic antigens. In some embodiments, the tolerogenic antigen is a foreign antigen against which a patient has developed an unwanted immune response. In some embodiments, the plurality of tolerogenic antigens are specific for celiac disease. In some embodiments, the tolerogenic antigens are selected form gliadin, glutenin, and fragments thereof capable of inducing an immune response. In some embodiments, the tolerogenic antigens are selected from gliadin (e.g., α-, γ-, and ω-gliadin) or fragments thereof. In some embodiments, the tolerogenic antigens are selected from the group consisting of α, γ, and ω gliadins or fragments thereof. In some embodiments, the tolerogenic antigen includes a polypeptide having at least 90% (e.g., at least 91%, at least 92%, at least 93%, at least 94%, at least 95%, at least 96%, at least 97%, at least 98%, at least 99%) sequence identity to the polypeptide sequence of any one of SEQ ID NOs: 375-580. In some embodiments, tolerogenic antigen includes a polypeptide having at least 95% (e.g., 96%, 97%, 98%, 99%, or 100%) sequence identity to any one of the polypeptide sequences of SEQ ID NOs: 375-580. In some embodiments, the tolerogenic antigen includes a polypeptide having the polypeptide sequence of any one of SEQ ID NOs: 375-580. In some embodiments, the tolerogenic antigen includes two or more (e.g., two, three, four, five, and six) polypeptide sequences having the sequence of any one of SEQ ID NOs: 375-580.

In some embodiments, the tolerogenic antigen is a self antigen against which a subject (e.g., a human patient) has developed an autoimmune response or may develop an autoimmune response. Examples include proinsulin (e.g., for subjects suffering from or at risk of suffering from diabetes), collagens (e.g., for subjects suffering from or at risk of suffering from rheumatoid arthritis), and myelin basic protein (e.g., for subjects suffering from or at risk of suffering from multiple sclerosis). There are many proteins that are human autoimmune proteins, a term referring to various autoimmune diseases wherein the protein or proteins causing the disease are known or can be established by routine testing. Embodiments include testing a patient to identify an autoimmune protein and creating an antigen for use in a molecular fusion and creating immunotolerance to the protein. Embodiments include an antigen, or choosing an antigen from, one or more of the following proteins. In type 1 diabetes mellitus, several main antigens have been identified: insulin, proinsulin, preproinsulin, glutamic acid decarboxylase-65 (GAD-65), GAD-67, insulinoma-associated protein 2 (IA-2), and insulinoma-associated protein 2p (IA-2β); other antigens include ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100β, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor. In Addison's disease, main antigens include 21-hydroxylase, 17α-hydroxylase, and P450 side chain cleavage enzyme (P450scc); other antigens include ACTH receptor, P450c21 and P450c17. In premature ovarian failure, main antigens include FSH receptor and α-enolase. In autoimmune hypophysitis, or pituitary autoimmune disease, main antigens include pituitary gland-specific protein factor (PGSF) 1a and 2; another antigen is type 2 iodothyronine deiodinase. In multiple sclerosis, main antigens include myelin basic protein, myelin oligodendrocyte glycoprotein and proteolipid protein. In rheumatoid arthritis, a main antigen is collagen II. In immunogastritis, a main antigen is H, K-ATPase. In pernicious angemis, a main antigen is intrinsic factor. In celiac disease, main antigens are tissue transglutaminase and gliadin. In vitiligo, a main antigen is tyrosinase, and tyrosinase related protein 1 and 2. In myasthenia gravis, a main antigen is acetylcholine receptor. In pemphigus vulgaris and variants, main antigens are desmoglein 3, 1 and 4; other antigens include pemphaxin, desmocollins, plakoglobin, perplakin, desmoplakins, and acetylcholine receptor. In bullous pemphigoid, main antigens include BP180 and BP230; other antigens include plectin and laminin 5. In dermatitis herpetiformis Duhring, main antigens include endomysium and tissue transglutaminase. In epidermolysis bullosa acquisita, a main antigen is collagen VII. In systemic sclerosis, main antigens include matrix metalloproteinase 1 and 3, the collagen-specific molecular chaperone heat-shock protein 47, fibrillin-1, and PDGF receptor; other antigens include Scl-70, U1 RNP, Th/To, Ku, Jo1, NAG-2, centromere proteins, topoisomerase I, nucleolar proteins, RNA polymerase I, II and III, PM-Slc, fibrillarin, and B23. In mixed connective tissue disease, a main antigen is U1snRNP. In Sjogren's syndrome, the main antigens are nuclear antigens SS-A and SS-B; other antigens include fodrin, poly(ADP-ribose) polymerase and topoisomerase. In systemic lupus erythematosus, main antigens include nuclear proteins including SS-A, high mobility group box 1 (HMGB1), nucleosomes, histone proteins and double-stranded DNA. In Goodpasture's syndrome, main antigens include glomerular basement membrane proteins including collagen IV. In rheumatic heart disease, a main antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

In some cases, the tolerogenic antigen is a foreign antigen against which a patient has developed an unwanted immune response. Examples are food antigens. Embodiments include testing a patient to identify foreign antigen and creating a molecular fusion that includes the antigen and treating the patient to develop immunotolerance to the antigen or food. Examples of such foods and/or antigens are provided. Examples are from peanut: conarachin (Ara h 1), allergen II (Ara h 2), arachis agglutinin, conglutin (Ara h 6); from apple: 31 kda major allergen/disease resistance protein homolog (Mal d 2), lipid transfer protein precursor (Mal d 3), major allergen Mal d 1.03D (Mal d 1): from milk: α-lactalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11 S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen m 2), tropomyosin fast isoform; from wheat and/or other cereals: high molecular weight glutenin, low molecular weight glutenin, alpha- and gamma-gliadin, hordein, secalin, avenin; from strawberry: major strawberry allergy Fra a 1-E (Fra a 1), from banana: profilin (Mus xp 1).

In some embodiments, the tolerogenic antigens are multimeric tolerogenic antigens including the following N-terminal-to-C-terminal structure

(P₄-L₄)ₙ₄-(P₃-L₃)ₙ₃-P₂-(L₁-P₁)ₙ₁

where P₁, P₂, P₃, and P₄ are each independently a tolerogenic antigen;

L₁, L₃, and L₄ are each independently a linker; and

n₁, n₃, and n₄ are each independently 0 or 1, wherein at least one of n₁, n₃, and n₄ are 1.

In some embodiments, n₁ is 1, n₃ is 0, and n₄ is 0, and the tolerogenic antigen includes the following N-terminal-to-C-terminal structure:

P₂-L₁-P₁.

In some embodiments, L₁ is a peptide linker including between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200). In some embodiments, L₁ is a peptide linker including glycine (G) and serine (S) residues. In some embodiments, L₁ is a peptide linker including the amino acid sequence of (GS)ₓ, (GGS)ₓ, or (GGGGS)ₓ, where x is an integer from 1 to 10 (e.g., 1, 2, 3, 4, 5, 6, 7, 8, 9, or 10). In some embodiments, P₁ and P₂ each include different tolerogenic antigens. In some embodiments, P₁ and P₂ each include identical tolerogenic antigens.

In some embodiments, n₁ is 1, n₃ is 1, and n₄ is 0, and the tolerogenic antigen includes the following N-terminal-to-C-terminal structure:

P₃-L₃-P₂-L₁-P₁.

In some embodiments, L₁ and L₃ are each an independently selected peptide linker including between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200). In some embodiments, L₁ and L₃ are each an independently selected peptide linker including glycine (G) and serine (S) residues. In some embodiments, L₁ and L₃ are each an independently selected peptide linker including the amino acid sequence of (GS)ₓ, (GGS)ₓ, or (GGGGS)ₓ, where x is an integer from 1 to 10 (e.g., 1, 2, 3, 4, 5, 6, 7, 8, 9, or 10). In some embodiments, P₁, P₂, and/or P₃ each include different tolerogenic antigens. In some embodiments, P₁, P₂, and P₃ each include identical tolerogenic antigens.

In some embodiments, n₁ is 1, n₃ is 1, and n₄ is 1, and the tolerogenic antigen includes the following N-terminal-to-C-terminal structure:

P₄-L₄-P₃-L₃-P₂-L₁-P₁.

In certain embodiments, L₁ and L₂ are each an independently selected peptide linker including between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200). In some embodiments, L₁, L₂, and L₃ are each an independently selected peptide linker including glycine (G) and serine (S) residues. In some embodiments, L₁, L₂, and L₃ are each an independently selected peptide linker including the amino acid sequence of (GS)ₓ, (GGS)ₓ, or (GGGGS (SEQ ID NO: 219))ₓ, where x is an integer from 1 to 10. In some embodiments, P₁, P₂, P₃, and/or P₄ each include different tolerogenic antigens. In some embodiments, P₁, P₂, P₃, and P₄ each include identical tolerogenic antigens.

In some embodiments, the number of tolerogenic antigens associated with a specific nanoparticle includes a population of between 1 and 30 (e.g., 1-10, 9 to 15, 12 to 18, 15 to 22, 18 to 25, 20 to 27, 22 to 28, or 25 to 30) tolerogenic antigens per nanoparticle. In some embodiments, the number of tolerogenic antigens associated with a specific nanoparticle includes a population of 6 tolerogenic antigens per particle. In other embodiments, the number of tolerogenic antigens associated with a specific nanoparticle includes a population of 8 tolerogenic antigens per particle. In some embodiments, the population of tolerogenic antigens associated with a specific nanoparticle are the same antigen. In some embodiments, the population of tolerogenic antigens associated with a specific nanoparticle includes between 1 and 5 (e.g., 2, 3, 4, and 5) different tolerogenic antigens. In some embodiments, the population of tolerogenic antigens associated with a specific nanoparticle include 3 to 4 different tolerogenic antigens. In some embodiments, the population of tolerogenic antigens are specific to between 1 and 3 different diseases. In certain embodiments, the population of tolerogenic antigens are specific to the same disease.

In some embodiments, the population of tolerogenic antigens associated with a specific nanoparticle includes (i) a first polypeptide population including the amino acid sequence of any one of SEQ ID NOs: 406-588, or a biologically active fragment or variant thereof, (ii) a second polypeptide population including the amino acid sequence of any one of SEQ ID NOs: 406-588, or biologically active fragment or variant thereof, and (iii) a third polypeptide population including the amino acid sequence of any one of SEQ ID NOs: 406-588, or a biologically active fragment or variant thereof.

In some embodiments, the first polypeptide population includes the amino acid sequence of SEQ ID NO: 474, or a biologically active fragment or variant thereof, (ii) the second polypeptide population includes the amino acid sequence of any one of SEQ ID NOs: 406-588, or biologically active fragment or variant thereof, and (iii) the third polypeptide population includes the amino acid sequence of any one of SEQ ID NOs: 406-588, or a biologically active fragment or variant thereof.

In some embodiments, the population of tolerogenic antigens associated with a specific nanoparticle includes (i) the first polypeptide population includes the amino acid sequence of SEQ ID NO: 474, or a biologically active fragment or variant thereof, (ii) the second polypeptide population includes the amino acid sequence of SEQ ID NO: 475, or biologically active fragment or variant thereof, and (iii) the third polypeptide population includes the amino acid sequence of any one of SEQ ID NOs: 406-588, or a biologically active fragment or variant thereof. In some embodiments, the third polypeptide population includes the amino acid sequence of SEQ ID NO: 476, or a biologically active fragment or variant thereof. In some embodiments, the second polypeptide population includes the amino acid sequence of SEQ ID NO: 477, or a biologically active fragment or variant thereof, and/or the third polypeptide population includes the amino acid sequence of SEQ ID NO: 478, or a biologically active fragment or variant thereof.

In some embodiments, the tolerogenic antigen includes a polypeptide having at least 90% (e.g., at least 91%, at least 92%, at least 93%, at least 94%, at least 95%, at least 96%, at least 97%, at least 98%, at least 99%) sequence identity to the polypeptide sequence of SEQ ID NO: 374. In some embodiments, the tolerogenic antigen includes a polypeptide sequence of SEQ ID NO: 374. In some embodiments, the tolerogenic antigen includes a fragment of SEQ ID NO: 373, including 6-12 (e.g., 7, 8, 9, 10, 11, and 12) amino acid residues in length.

In some embodiments, the tolerogenic antigen includes an amide group at the C-terminus. In certain embodiments, the tolerogenic antigen includes a pyroglutamic acid residue at the N-terminus. In another embodiment, the tolerogenic antigen includes an acetyl group at the N-terminus. In some embodiments, the tolerogenic antigen includes a pyroglutamic acid residue at the N-terminus and an amide group at the C-terminus. In some embodiments, the tolerogenic antigen includes an acetyl group at the N-terminus and an amide group at the C-terminus. In certain embodiments, the tolerogenic antigen includes an N-terminus or a C-terminus modified with a cysteine residue bound to a linker. In some embodiments, the tolerogenic antigen includes an N-terminus and a C-terminus modified with cysteine residues bound to a linker.

In some embodiments of any one of the compositions described herein, the population of tolerogenic antigens are conjugated with the nanoparticle phospholipid in such a manner that facilitates strong immune tolerance upon administration to a subject (e.g., a human subject suffering from or at risk of suffering from an autoimmune disorder e.g., MS, celiac disease, rheumatoid arthritis, diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, or inflammatory bowel disease).

In some embodiments, the plurality of tolerogenic antigens are conjugated with the nanoparticle phospholipid via a thiol-reactive and reduction-insensitive linkage between the tolerogenic antigen and the nanoparticle phospholipid. Indeed, a thiol-reactive and reduction-insensitive linkage between the tolerogenic antigen and the nanoparticle phospholipid facilitates strong immune tolerance. In some embodiments, the phospholipid is N-(3-Maleimide-1-oxopropyl)-L-α-phosphatidylethanolamine.

In some embodiments, the tolerogenic antigen is conjugated with the nanoparticle phospholipid via an amine-mediated interaction (e.g., N-(Succinimidyloxy-glutaryl)-L-α-phosphatidylethanolamine, Dioleoyl (DOPE-NHS)). In some embodiments, the amine-mediated interaction is N-(Succinimidyloxy-glutaryl)-L-α-phosphatidylethanolamine, Dioleoyl (DOPE-NHS)). In some embodiments, the amine-mediated interaction is through an amine-reactive phospholipid with self-immolative linkage (e.g., linkers including o-dithiobenzyl, p-dithiobenzyl, beta-dithiobenzyl carbamate moieties, 2,2-dimethyl-4-mercapto-butyric acid, or Disulfide-carbonate-based traceless linker).

In some embodiments, the composition further includes at least one therapeutic agent (e.g., at least one immunomodulatory agent or immunosuppressant (e.g., at least one immunomodulatory agent selected from fingolimod; 2-(1′H-indole-3′-carbonyl)-thiazole-4-carboxylic acid methyl ester (ITE) or related ligands; Trichostatin A; Suberoylanilide hydroxamic acid (SAHA); statins; mTOR inhibitors; TGF-β signaling agents; TGF-β receptor agonists; histone deacetylase inhibitors; corticosteroids; inhibitors of mitochondrial function; NF-κβ inhibitors; adenosine receptor agonists; prostaglandin E2 agonists (PGE2; phosphodiesterase inhibitors; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors; autophagy inhibitors; aryl hydrocarbon receptor inhibitors; proteasome inhibitor I (PSI); oxidized ATPs IDO; vitamin D3; cyclosporins; aryl hydrocarbon receptor inhibitors; resveratrol; azathiopurine (Aza); 6-mercaptopurine (6-MP); 6-thioguanine (6-TG); FK506; sanglifehrin A; salmeterol; mycophenolate mofetil (MMF); aspirin and other COX inhibitors; niflumic acid; estriol; triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNT02424; TH1020; INH-ODN; E6446; AT791; CpG ODN 2088; ODN TTAGGG; COV08-0064; 2R9; GpG oligonucleotides; 2-aminopurine; Amlexanox; Bay11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ))). In particular embodiments, the immunosuppressant is fingolimod; 2-(1′H-indole-3′-carbonyl)-thiazole-4-carboxylic acid methyl ester (ITE) or related ligands; Trichostatin A; and/or Suberoylanilide hydroxamic acid (SAHA). In some embodiments, the at least one therapeutic agent is included within the sHDL nanoparticle. In some embodiments, the sHDL nanoparticle is further admixed with an adjuvant (e.g., CPG, polylC, poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, imiquimod, resiquimod, gardiquimod, 3M-052, SRL172, Virosomes and other Virus-like particles, YF-17D, VEGF trap, beta-glucan, Pam3Cys, Aquila's QS21 stimulon, vadimezan, AsA404 (DMXAA), and any derivative of an adjuvant).

In some embodiments, the composition does not contain an adjuvant.

In another aspect, the disclosure provides a method for treating a subject having or at risk of having one or more autoimmune disorders (e.g., MS, celiac disease, rheumatoid arthritis, diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, and inflammatory bowel disease), including administering an effect amount of the composition of any one of the embodiments described herein to the subject. In some embodiments, the one or more autoimmune disorders is a single autoimmune disorder. In some embodiments, the single autoimmune disorder is celiac disease. In some embodiments, the subject is a human subject. In some embodiments, the method includes administering one more additional therapeutic agents to the subject. In some embodiments, the one or more additional therapeutic agents are administered simultaneously with an effective amount of a composition of any one of the embodiments described herein to the subject. In some embodiments, the one or more additional therapeutic agents are administered at a different time from an effective amount of a composition of any one of the embodiments described herein.

In some embodiments, the one or more additional therapeutic agents are selected from the group consisting of corticosteroids, such as prednisone, betamethasone, clobetasone butyrate; and budesonide, immunosuppressants, such as etanercept, adalimumab, azathioprine, infliximab, cyclosporine, alemtuzumab, and cladribine; and anti-inflammatory agents, such as dapsone and sulfonamide. In some embodiments, the one or more additional therapeutic agents are selected from infliximab, adalimumab, etanercept, dapsone or clobetasone butyrate. In some embodiments, the subject adheres to a gluten-free diet.

Additional embodiments will be apparent to persons skilled in the relevant art based on the teachings contained herein.

### BRIEF DESCRIPTION OF THE DRAWINGS

FIG. 1 is an image depicting T-cell activation.

FIG. 2 is a graph showing the pathological scored for EAE induced with free MOG (100 μg/dose) and HDL-MOG nanodisc (100 μg/dose).

FIGS. 3A-3C show HDL-MOG nanodiscs administered starting day 2 exhibit potent efficacy against EAE. FIG. 3A is a schematic diagram depicting the treatment regimen. FIG. 3B is a graph showing pathological scores for EAE induced with MOG35-55. FIG. 3C is a graph showing pathological scores for EAE induced with MOG1-125.

FIGS. 4A-4C show HDL-MOG nanodiscs administered starting day 15 exhibit potent efficacy against EAE. FIG. 4A is a schematic diagram depicting the treatment regimen. FIG. 4B is a graphs showing pathological scores for EAE induced with MOG35-55. FIG. 4C is a graph showing pathological scores for EAE induced with MOG1-125.

FIGS. 5A-5B are graphs showing HDL-MOG nanodiscs exhibit more potent efficacy than FTY720. Pathological scores for EAE mice treated with HDL-MOG versus FTY720 when the treatment started on day 15 (FIG. 5A) or day 30 (FIG. 5B).

FIGS. 6A-6B show HDL-MOG nanodiscs reduce secretion of inflammatory cytokines in CNS of EAE mice. FIG. 6A is a schematic diagram depicting the treatment regimen. FIG. 6B are graphs showing the amount of GM-CSF, IFN-gamma, and IL-17 released after ex vivo treatment of CNS tissues with MOG35-55 peptide.

FIG. 7 is images of BMDCs and microglia after incubation with HDL-MOG-FITC or MOG-FITC peptide and visualized for antigen uptake. Actin filaments were stained with AlexaFluor 647-Phalloidin, and nuclei were stained with DAPI.

FIGS. 8A-8B are images showing the experimental design and results for Naïve mice (FIG. 8A) or EAE-induced mice (FIG. 8B). EAE-induced mice were administered subcutaneously with PBS, free MOG-TMR, or HDL-MOG-TMR. At the indicated time points, DCs, macrophages, and B cells were isolated from draining inguinal lymph nodes or spinal cord and analyzed for the TMR fluorescence signal.

FIG. 9A is a schematic diagram depicting the treatment regimen of EAE-induced mice administered with HDL-MOG-NOTA-⁶⁴Cu or MOG-NOTA-⁶⁴Cu for a biodistribution study.

FIG. 9B are pictures showing positron emission tomography (PET) imaging over a 24 hour period of EAE-induced mice.

FIG. 9C is a graph showing quantification of ⁶⁴Cu signal in the major organs at 24 hours post injection.

FIG. 10A is a schematic diagram of EAE-induced mice treated with PBS, free MOG, HDL-M30, or HDL-MOG as shown.

FIG. 10B is graphs showing the results from ELISA from CNS collected on day 40, processed into individual cells, restimulated ex vivo with MOG peptide, and quantified for the levels of IL-17, IFN-gamma, and GM-CFS.

FIGS. 10C-10D are graphs depicting the results of intracellular cytokine staining for CNS (FIG. 10C) and splenocytes (FIG. 10D) examined for the frequency of CD4 T cells secreting IL-17, IFN-gamma, and GM-CFS upon ex vivo re-stimulation with or without MOG peptide.

FIG. 11 depicts the experimental design and results of EAE-induced mice treated with PBS, free MOG, HDL-M30, or HDL-MOG as shown. CNS tissues collected on day 40 were examined for the levels of IL-17, IFN-gamma, GM-CFS, and IL-10.

FIG. 12A is a schematic diagram of EAE induced mice treated with PBS, free MOG, HDL-M30, or HDL-MOG as shown. CNS tissues collected on day 40 were examined for the frequency of Tregs.

FIG. 12B-12C are graphs depicting the frequencies of CD25+Foxp3+ Tregs (FIG. 12B) and MOG-tetramer+Foxp3+ Tregs in CNS.

FIG. 13 depicts experimental design for EAE-induced mice treated with PBS or HDL-MOG as shown. Red arrows indicate the days of treatment. In addition, some mice were administered with anti-CD25 IgG on the indicated time points. Mice were monitored over time for the EAE scores.

FIG. 14 are HPLC chromatograms quantifying the amount of FTY720 loaded in for HDL-FTY720, compare with free drug standards.

FIG. 15 are HPLC chromatograms quantifying the amount of ITE loaded in HDL (HDL-ITE) compared with free drug standards.

FIG. 16 are HPLC chromatograms quantifying the amount of TSA loaded in HDL (HDL-TSA) compared with free drug standards.

FIG. 17 are HPLC chromatograms quantifying the amount of SAHA loaded in HDL (HDL-SAHA) compared with free drug standards.

FIG. 18 is a table showing the drug loading efficiencies for FTY72-, ITE, TSA, and SAHA in HDL nanodiscs quantified by HPLC/MS. The size of nanodiscs were measured by Dynamic Light Scattering (DLS).

FIG. 19 is a gel permeation chromatograph depicting HDL-FTY720 compared to blank HDL nanodiscs.

FIG. 20 depicts graphs of HDL loaded with rapamycin (Rapa) analyzed by DLS and gel permeation chromatography, demonstrating that HDL-Rapa exhibited homogenous size distribution with an average hydrodynamic size of ˜10 nm.

FIG. 21 is a graph depicting EAE induced mice on day 0, and mice received i.p. administration of HDL-FTY720 (1 mg/kg of FTY720) on days 14, 21, and 28. Mice were monitored for EAE score.

FIG. 22 is an HPLC chromatogram of HDL nanodiscs loaded with EA peptide, blank HDL, lipid-EA conjugate, and MPB lipid. The amount of EA peptide loaded in HDL was quantified by HPLC-MS.

FIG. 23 is an HPLC chromatogram of HDL nanodiscs loaded with OVA-II peptide, blank HDL, lipid-OVA-II conjugate, and MPB lipid. The amount of OVA-II peptide loaded in HDL was quantified by HPLC-MS.

FIG. 24 is an HPLC chromatogram of HDL nanodiscs loaded with CIA peptide, blank HDL, lipid-CIA conjugate, and MPB lipid. The amount of CIA peptide loaded in HDL was quantified by HPLC-MS.

FIG. 25 is a table showing the antigen conjugation efficiency to form antigen-lipid conjugates (EA, OVA-II, and CIA), antigen-lipid loading efficiency in HDL, and hydrodynamic size of antigen-loaded HDL.

### DEFINITIONS

The term “about” is used herein to mean a value that is ±10% of the recited value.

As used herein, the term “absorbed” refers to a biomacromolecule agent (e.g., antigen, adjuvant, etc.) that is taken into and stably retained in the interior, that is, internal to the outer surface, of a nanoparticle and/or microparticle.

As used herein, by “administering” is meant a method of giving a dosage of a composition described herein (e.g., a nanoparticle or a nanoparticle associated with an antigen) to a subject. The compositions utilized in the methods described herein can be administered by any suitable route, including, for example, by inhalation, nebulization, aerosolization, intranasally, intratracheally, intrabronchially, orally, parenterally (e.g., intravenously, subcutaneously, or intramuscularly), orally, nasally, rectally, topically, or buccally. The compositions utilized in the methods described herein can also be administered locally or systemically. The preferred method of administration can vary depending on various factors (e.g., the components of the composition being administered, and the severity of the condition being treated).

As used herein, the term “admixed” refers to a biomacromolecule agent (e.g., antigen, adjuvant, etc.) that is dissolved, dispersed, or suspended in a nanoparticle and/or microparticle. In some cases, the biomacromolecule agent may be uniformly admixed in the nanoparticle and/or microparticle.

As used herein, the term “adsorbed” refers to the attachment of a biomacromolecule agent (e.g., antigen, adjuvant, etc.) to the external surface of a nanoparticle and/or microparticle. Such adsorption preferably occurs by electrostatic attraction. Electrostatic attraction is the attraction or bonding generated between two or more oppositely charged or ionic chemical groups. Generally, the adsorption is typically reversible.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein can be the full-length, 33-mer polypeptide from α-gliadin (SEQ ID NO: 374) or any fragment thereof or any of the polypeptides disclosed in Table 3 (SEQ ID NOs: 375-405) as epitopes recognized by CD4+ T-cells. Where reference is made to a specific protein herein, the term encompasses the “full-length”, unprocessed protein as well as any form of the protein that results from processing in the cell. The term also encompasses naturally occurring variants of the protein, e.g. splice variants or allelic variants.

As used herein, the terms “autoimmune disorder” and “autoimmune disease”, used herein interchangeably, refers to a medical condition in which a subject's immune system mistakenly attacks the subject's own body.

As used herein, a “combination therapy” or “administered in combination” means that two or more (e.g., 2, 3, 4, 5, 6, 7, 8, 9, 10 or more) different agents or treatments are administered to a subject as part of a defined treatment regimen for a particular disease or condition (e.g., an autoimmune disorder (e.g., MS or celiac disease)). The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder, is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be affected by any appropriate route including, but not limited to, by inhalation, nebulization, aerosolization, intranasally, intratracheally, intrabronchially, orally, parenterally (e.g., intravenously, subcutaneously, or intramuscularly), orally, nasally, rectally, topically, buccally, or by direct absorption through mucous membrane tissues. The therapeutic agents can be administered by the same route or by different routes. For example, a first therapeutic agent of the combination may be administered by intravenous injection while a second therapeutic agent of the combination may be administered orally.

As used herein, the term “complexed” as used herein relates to the non-covalent interaction of a biomacromolecule agent (e.g., antigen, adjuvant, etc.) with a nanoparticle and/or microparticle.

As used herein, the term “conjugated” as used herein indicates a covalent bond association between a biomacromolecule agent (e.g., antigen, adjuvant, etc.) and a nanoparticle and/or microparticle.

As used herein, the term “drug” or “therapeutic agent” is meant to include any molecule, molecular complex, or substance administered to an organism for diagnostic or therapeutic purposes, including medical imaging, monitoring, contraceptive, cosmetic, nutraceutical, pharmaceutical, and prophylactic applications. The term drug is further meant to include any such molecule, molecular complex, or substance that is chemically modified and/or operatively attached to a biologic or biocompatible structure.

As used herein, the term “fragment” refers to less than 100% of the amino acid sequence of a full-length reference protein (e.g., 99%, 90%, 80%, 70%, 60%, 50%, 40%, 30%, 20%, 10%, of the full-length sequence etc.), but including, e.g., 5, 10, 20, 25, 30, 35, 40, 45, 50, 100, 150, 200, 250, 300, 350, or more amino acids. A fragment can be of sufficient length such that a desirable function of the full-length protein is maintained. For example, the regulation of the alternative complement pathway in the fluid phase by fragments of, for example, factor H, is maintained. Such fragments are “biologically active fragments.”

As used herein, the terms “gluten-free” and “gluten-free diet” refer to a diet where cereals from the triticeae grass family, such as wheat, barley, and rye, are not consumed directly or in any food product. A gluten-free diet is a diet where no more than 10-50 mg of gluten are consumed per day.

Gluten-free diets are often employed to minimize or eliminate the effects of celiac disease.

As used herein, the term “HDL” or “high density lipoprotein” refers to high-density lipoprotein. HDL comprises a complex of lipids and proteins in approximately equal amounts that functions as a transporter of cholesterol in the blood. HDL is mainly synthesized in and secreted from the liver and epithelial cells of the small intestine. Immediately after secretion, HDL is in a form of a discoidal particle containing apolipoprotein A-I (also called apoA-1) and phospholipid as its major constituents and is also called nascent HDL. This nascent HDL receives, in blood, free cholesterol from cell membranes of peripheral cells or produced in the hydrolysis course of other lipoproteins, and forms mature spherical HDL while holding, at its hydrophobic center, cholesterol ester converted from said cholesterol by the action of LCAT (lecithin cholesterol acyltransferase). HDL plays an extremely important role in a lipid metabolism process called “reverse cholesterol transport”, which takes, in blood, cholesterol out of peripheral tissues and transports it to the liver. High levels of HDL are associated with a decreased risk of atherosclerosis and coronary heart disease (CHD) as the reverse cholesterol transport is considered one of the major mechanisms for HDL's prophylactic action on atherosclerosis.

As used herein, the term “immunomodulatory agent” refers to a compound that stimulates or suppresses the immune system. As used herein, the term “immunosuppressant” refers to a compound that causes an APC to have an immunosuppressive (e.g., tolerogenic effect). An immunosuppressive effect generally refers to the production or expression of cytokines or other factors by the APC that reduces, inhibits or prevents an undesired immune response or that promotes a desired immune response. When the APC results in an immunosuppressive effect on immune cells that recognize an antigen presented by the APC, the immunosuppressive effect is said to be specific to the presented antigen. Such effect is also referred to herein as a tolerogenic effect. Without being bound by any particular theory, it is thought that the immunosuppressive or tolerogenic effect is a result of the immunosuppressant being delivered to the APC, preferably in the presence of an antigen (e.g., an administered antigen or one that is already present in vivo). Accordingly, the immunosuppressant includes compounds that provide a tolerogenic immune response to an antigen that may or may not be provided in the same composition or a different composition. In one embodiment, the immunosuppressant is one that causes an APC to promote a regulatory phenotype in one or more immune effector cells. For example, the regulatory phenotype may be characterized by the inhibition of the production, induction, stimulation or recruitment of antigen-specific CD8+ T cells, the production, induction, stimulation or recruitment of Treg cells, etc. This may be the result of the conversion of CD8+ T cells or B cells to a regulatory phenotype. This may also be the result of induction of FoxP3 in other immune cells, such as CD4+ T cells, macrophages and iNKT cells. In one embodiment, the immunosuppressant is one that affects the response of the APC after it processes an antigen. In another embodiment, the immunosuppressant is not one that interferes with the processing of the antigen. In a further embodiment, the immunosuppressant is not an apoptotic-signaling molecule. In another embodiment, the immunosuppressant is not a phospholipid.

Immunomodulatory agents include, but are not limited to, statins; mTOR inhibitors, such as rapamycin or a rapamycin analog; TGF-β signaling agents; TGF-β receptor agonists; histone deacetylase inhibitors, such as Trichostatin A; corticosteroids; inhibitors of mitochondrial function, such as rotenone; P₃₈ inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol; triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNT02424; TH1020; INH-ODN; E6446; AT791; CpG ODN 2088; ODN TTAGGG; COV08-0064; 2R9; GpG oligonucleotides; 2-aminopurine; Amlexanox; Bay11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ). In particular embodiments, the immunosuppressant is FTY720 (also known as fingolimod) (Chung and Harung, Clin. Neuropharmacol 33: 91-101, 2010), AhR activation by 2-(1′H-indole-3′-carbonyl)-thiazole-4-carboxylic acid methyl ester (ITE) or related ligands (Yeste A, et al. Proc. Natl. Acad. Sci. USA 109: 11270-11275, 2012; Quintana F. J., et al Proc. Natl. Acad. Sci. USA 107: 20768-20773, 2010), Trichostatin A (TSA) (Reilly C. M. et al. J. Autoimmun 31: 123-130. 2008). Suberoylanilide hydroxamic acid (SAHA), a histone deacetylase inhibitor, (Lucas J. L., et al. Cell Immunol 257: 97-104, 2009) and/or Rapamycin (Rapa) (Maldonado, R. A., et al Proc. Natl. Acad. Sci. USA 112:E156-165, 2015). In embodiments, the immunosuppressant may comprise any of the agents provided herein.

The immunosuppressant can be a compound that directly provides the immunosuppressive (e.g., tolerogenic) effect on APCs or it can be a compound that provides the immunosuppressive (e.g., tolerogenic) effect indirectly (i.e., after being processed in some way after administration). Immunosuppressants, therefore, include prodrug forms of any of the compounds provided herein.

Immunosuppressants also include nucleic acids that encode the peptides, polypeptides or proteins provided herein that result in an immunosuppressive (e.g., tolerogenic) immune response. In embodiments, therefore, the immunosuppressant is a nucleic acid that encodes a peptide, polypeptide or protein that results in an immunosuppressive (e.g., tolerogenic) immune response, and it is the nucleic acid that is coupled to the sHDL nanoparticle.

The nucleic acid may be DNA or RNA, such as mRNA. In embodiments, the compositions comprise a complement, such as a full-length complement, or a degenerate (due to degeneracy of the genetic code) of any of the nucleic acids provided herein. In embodiments, the nucleic acid is an expression vector that can be transcribed when transfected into a cell line. In embodiments, the expression vector may comprise a plasmid, retrovirus, or an adenovirus amongst others. Nucleic acids can be isolated or synthesized using standard molecular biology approaches, for example by using a polymerase chain reaction to produce a nucleic acid fragment, which is then purified and cloned into an expression vector. Additional techniques useful in the practice of this invention may be found in Current Protocols in Molecular Biology 2007 by John Wiley and Sons, Inc.; Molecular Cloning: A Laboratory Manual (Third Edition) Joseph Sambrook, Peter MacCallum Cancer Institute, Melbourne, Australia; David Russell, University of Texas Southwestern Medical Center, Dallas, Cold Spring Harbor.

Other exemplary immunosuppressants include, but are not limited, small molecule drugs, natural products, antibodies (e.g., antibodies against CD20, CD3, CD4), biologics-based drugs, carbohydrate-based drugs, nanoparticles, liposomes, RNAi, antisense nucleic acids, aptamers, methotrexate, NSAIDs; fingolimod; natalizumab; alemtuzumab; anti-CD3; tacrolimus (FK506), etc. Further immunosuppressants, are known to those of skill in the art, and the invention is not limited in this respect.

As used herein, the term “in vitro” refers to an artificial environment and to processes or reactions that occur within an artificial environment. In vitro environments can consist of, but are not limited to, test tubes and cell culture.

The term “in vivo” refers to the natural environment (e.g., an animal or a cell) and to processes or reaction that occur within a natural environment.

As used here, the term “lipids” or “lipid molecules” refer to fatty substances that are insoluble in water and include fats, oils, waxes, and related compounds. They may be either made in the blood (endogenous) or ingested in the diet (exogenous). Lipids are essential for normal body function and whether produced from an exogenous or endogenous source, they must be transported and then released for use by the cells. The production, transportation, and release of lipids for use by the cells is referred to as lipid metabolism. While there are several classes of lipids, two major classes are cholesterol and triglycerides. Cholesterol may be ingested in the diet and manufactured by the cells of most organs and tissues in the body, primarily in the liver. Cholesterol can be found in its free form or, more often, combined with fatty acids forming what is known as cholesterol esters. As used herein, “lipid” or “lipid molecule” refers to any lipophilic compound. Non-limiting examples of lipid compounds include fatty acids, cholesterol, phospholipids, complex lipids, and derivatives or analogs thereof. They are usually divided into at least three classes: (1) “simple lipids,” which include fats and oils as well as waxes; (2) “compound lipids,” which include phospholipids and glycolipids; and (3) “derived lipids” such as steroids. Lipids or lipid molecules suitable for use in the present invention include both membrane-forming lipids and non-membrane-forming lipids.

As used herein the term, “lipoproteins” refer to spherical compounds that are structured so that water-insoluble lipids are contained in a partially water-soluble shell. Depending on the type of lipoprotein, the contents include varying amounts of free and esterified cholesterol, triglycerides, and apoproteins or apolipoproteins. There are five major types of lipoproteins, which differ in function and in their lipid and apoprotein content and are classified according to increasing density: (i) chylomicrons and chylomicron remnants, (ii) very low density lipoproteins (“VLDL”), (iii) intermediate-density lipoproteins (“IDL”), (iv) low-density lipoproteins (“LDL”), and (v) high-density lipoproteins (“HDL”). Cholesterol circulates in the bloodstream as particles associated with lipoproteins.

The term “non-naturally occurring amino acid,” as used herein, means an alpha amino acid that is not naturally produced or found in a mammal. Examples of non-naturally occurring amino acids include D-amino acids; an amino acid having an acetylaminomethyl group attached to a sulfur atom of a cysteine; a pegylated amino acid; the omega amino acids of the formula NH₂(CH₂)ₙCOOH where n is 2-6, neutral nonpolar amino acids, such as sarcosine, t-butyl alanine, t-butyl glycine, N-methyl isoleucine, and norleucine; oxymethionine; phenylglycine; citrulline; methionine sulfoxide; cysteic acid; ornithine; diaminobutyric acid; 3-aminoalanine; 3-hydroxy-D-proline; 2,4-diaminobutyric acid; 2-aminopentanoic acid; 2-aminooctanoic acid, 2-carboxy piperazine; piperazine-2-carboxylic acid, 2-amino-4-phenylbutanoic acid; 3-(2-naphthyl)alanine, and hydroxyproline. Other amino acids are α-aminobutyric acid, α-amino-α-methylbutyrate, aminocyclopropane-carboxylate, aminoisobutyric acid, aminonorbornyl-carboxylate, L-cyclohexylalanine, cyclopentylalanine, L-N-methylleucine, L-N-methylmethionine, L-N-methylnorvaline, L-N-methylphenylalanine, L-N-methylproline, L-N-methylserine, L-N-methyltryptophan, D-ornithine, L-N-methylethylglycine, L-norleucine, α-methyl-aminoisobutyrate, α-methylcyclohexylalanine, D-α-methylalanine, D-α-methylarginine, D-α-methylasparagine, D-α-methylaspartate, D-α-methylcysteine, D-α-methylglutamine, D-α-methylhistidine, D-α-methylisoleucine, D-α-methylleucine, D-α-methyllysine, D-α-methylmethionine, D-α-methylornithine, D-α-methylphenylalanine, D-α-methylproline, D-α-methylserine, D-N-methylserine, D-α-methylthreonine, D-α-methyltryptophan, D-α-methyltyrosine, D-α-methylvaline, D-N-methylalanine, D-N-methylarginine, D-N-methylasparagine, D-N-methylaspartate, D-N-methylcysteine, D-N-methylglutamine, D-N-methylglutamate, D-N-methylhistidine, D-N-methylisoleucine, D-N-methylleucine, D-N-methyllysine, N-methylcyclohexylalanine, D-N-methylornithine, N-methylglycine, N-methylaminoisobutyrate, N-(1-methylpropyl)glycine, N-(2-methylpropyl)glycine, D-N-methyltryptophan, D-N-methyltyrosine, D-N-methylvaline, γ-aminobutyric acid, L-t-butylglycine, L-ethylglycine, L-homophenylalanine, L-α-methylarginine, L-α-methylaspartate, L-α-methylcysteine, L-a-methylglutamine, L-α-methylhistidine, L-α-methylisoleucine, L-α-methylleucine, L-α-methylmethionine, L-α-methylnorvaline, L-α-methylphenylalanine, L-α-methylserine, L-α-methyltryptophan, L-α-methylvaline, N-(N-(2,2-diphenylethyl) carbamylmethylglycine, 1-carboxy-1-(2,2-diphenyl-ethylamino) cyclopropane, 4-hydroxyproline, ornithine, 2-aminobenzoyl (anthraniloyl), D-cyclohexylalanine, 4-phenyl-phenylalanine, L-citrulline, α-cyclohexylglycine, L-1,2,3,4-tetrahydroisoquinoline-3-carboxylic acid, L-thiazolidine-4-carboxylic acid, L-homotyrosine, L-2-furylalanine, L-histidine (3-methyl), N-(3-guanidinopropyl)glycine, O-methyl-L-tyrosine, 0-glycan-serine, meta-tyrosine, nor-tyrosine, L-N,N′,N″-trimethyllysine, homolysine, norlysine, N-glycan asparagine, 7-hydroxy-1,2,3,4-tetrahydro-4-fluorophenylalanine, 4-methylphenylalanine, bis-(2-picolyl)amine, pentafluorophenylalanine, indoline-2-carboxylic acid, 2-aminobenzoic acid, 3-amino-2-naphthoic acid, asymmetric dimethylarginine, L-tetrahydroisoquinoline-1-carboxylic acid, D-tetrahydroisoquinoline-1-carboxylic acid, 1-amino-cyclohexane acetic acid, D/L-allylglycine, 4-aminobenzoic acid, 1-amino-cyclobutane carboxylic acid, 2 or 3 or 4-aminocyclohexane carboxylic acid, 1-amino-1-cyclopentane carboxylic acid, 1-aminoindane-1-carboxylic acid, 4-amino-pyrrolidine-2-carboxylic acid, 2-aminotetraline-2-carboxylic acid, azetidine-3-carboxylic acid, 4-benzyl-pyrolidine-2-carboxylic acid, tert-butylglycine, b-(benzothiazolyl-2-yl)-alanine, b-cyclopropyl alanine, 5,5-dimethyl-1,3-thiazolidine-4-carboxylic acid, (2R,4S)4-hydroxypiperidine-2-carboxylic acid, (2S,4S) and (2S,4R)-4-(2-naphthylmethoxy)-pyrolidine-2-carboxylic acid, (2S,4S) and (2S,4R)4-phenoxy-pyrrolidine-2-carboxylic acid, (2R,5S) and (2S,5R)-5-phenyl-pyrrolidine-2-carboxylic acid, (2S,4S)-4-amino-1-benzoyl-pyrrolidine-2-carboxylic acid, t-butylalanine, (2S,5R)-5-phenyl-pyrrolidine-2-carboxylic acid, 1-aminomethyl-cyclohexane-acetic acid, 3,5-bis-(2-amino)ethoxy-benzoic acid, 3,5-diamino-benzoic acid, 2-methylamino-benzoic acid, N-methylanthranylic acid, L-N-methylalanine, L-N-methylarginine, L-N-methylasparagine, L-N-methylaspartic acid, L-N-methylcysteine, L-N-methylglutamine, L-N-methylglutamic acid, L-N-methylhistidine, L-N-methylisoleucine, L-N-methyllysine, L-N-methylnorleucine, L-N-methylornithine, L-N-methylthreonine, L-N-methyltyrosine, L-N-methylvaline, L-N-methyl-t-butylglycine, L-norvaline, α-methyl-γ-aminobutyrate, 4,4′-biphenylalanine, α-methylcylcopentylalanine, α-methyl-α-napthylalanine, α-methylpenicillamine, N-(4-aminobutyl)glycine, N-(2-aminoethyl)glycine, N-(3-aminopropyl)glycine, N-amino-α-methylbutyrate, α-napthylalanine, N-benzylglycine, N-(2-carbamylethyl)glycine, N-(carbamylmethyl)glycine, N-(2-carboxyethyl)glycine, N-(carboxymethyl)glycine, N-cyclobutylglycine, N-cyclodecylglycine, N-cycloheptylglycine, N-cyclohexylglycine, N-cyclodecylglycine, N-cylcododecylglycine, N-cyclooctylglycine, N-cyclopropylglycine, N-cycloundecylglycine, N-(2,2-diphenylethyl)glycine, N-(3,3-diphenylpropyl)glycine, N-(3-guanidinopropyl)glycine, N-(1-hydroxyethyl)glycine, N-(hydroxyethyl))glycine, N-(imidazolylethyl))glycine, N-(3-indolylyethyl)glycine, N-methyl-γ-aminobutyrate, D-N-methylmethionine, N-methylcyclopentylalanine, D-N-methylphenylalanine, D-N-methylproline, D-N-methylthreonine, N-(1-methylethyl)glycine, N-methyl-napthylalanine, N-methylpenicillamine, N-(p-hydroxyphenyl)glycine, N-(thiomethyl)glycine, penicillamine, L-α-methylalanine, L-α-methylasparagine, L-α-methyl-t-butylglycine, L-methylethylglycine, L-α-methylglutamate, L-a-methylhomophenylalanine, N-(2-methylthioethyl)glycine, L-α-methyllysine, L-α-methylnorleucine, L-a-methylornithine, L-α-methylproline, L-α-methylthreonine, L-α-methyltyrosine, L-N-methyl-homophenylalanine, N-(N-(3,3-diphenylpropyl) carbamylmethylglycine, L-pyroglutamic acid, D-pyroglutamic acid, O-methyl-L-serine, O-methyl-L-homoserine, 5-hydroxylysine, α-carboxyglutamate, phenylglycine, L-pipecolic acid (homoproline), L-homoleucine, L-lysine (dimethyl), L-2-naphthylalanine, L-dimethyldopa or L-dimethoxy-phenylalanine, L-3-pyridylalanine, L-histidine (benzoyloxymethyl), N-cycloheptylglycine, L-diphenylalanine, O-methyl-L-homotyrosine, L-p-homolysine, O-glycan-threoine, Ortho-tyrosine, L-N,N′-dimethyllysine, L-homoarginine, neotryptophan, 3-benzothienylalanine, isoquinoline-3-carboxylic acid, diaminopropionic acid, homocysteine, 3,4-dimethoxyphenylalanine, 4-chlorophenylalanine, L-1,2,3,4-tetrahydronorharman-3-carboxylic acid, adamantylalanine, symmetrical dimethylarginine, 3-carboxythiomorpholine, D-1,2,3,4-tetrahydronorharman-3-carboxylic acid, 3-aminobenzoic acid, 3-amino-1-carboxymethyl-pyridin-2-one, 1-amino-1-cyclohexane carboxylic acid, 2-aminocyclopentane carboxylic acid, 1-amino-1-cyclopropane carboxylic acid, 2-aminoindane-2-carboxylic acid, 4-amino-tetrahydrothiopyran-4-carboxylic acid, azetidine-2-carboxylic acid, b-(benzothiazol-2-yl)-alanine, neopentylglycine, 2-carboxymethyl piperidine, b-cyclobutyl alanine, allylglycine, diaminopropionic acid, homo-cyclohexyl alanine, (2S,4R)-4-hydroxypiperidine-2-carboxylic acid, octahydroindole-2-carboxylic acid, (2S,4R) and (2S,4R)-4-(2-naphthyl), pyrrolidine-2-carboxylic acid, nipecotic acid, (2S,4R) and (2S,4S)-4-(4-phenylbenzyl) pyrrolidine-2-carboxylic acid, (3S)-1-pyrrolidine-3-carboxylic acid, (2S,4S)-4-tritylmercapto-pyrrolidine-2-carboxylic acid, (2S,4S)-4-mercaptoproline, t-butylglycine, N,N-bis(3-aminopropyl)glycine, 1-amino-cyclohexane-1-carboxylic acid, N-mercaptoethylglycine, and selenocysteine. In some embodiments, amino acid residues may be charged or polar. Charged amino acids include alanine, lysine, aspartic acid, or glutamic acid, or non-naturally occurring analogs thereof. Polar amino acids include glutamine, asparagine, histidine, serine, threonine, tyrosine, methionine, or tryptophan, or non-naturally occurring analogs thereof. It is specifically contemplated that in some embodiments, a terminal amino group in the amino acid may be an amido group or a carbamate group.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

The terms “peptide”, “polypeptide” and “protein” are used interchangeably herein to refer to polymers of amino acids (e.g., naturally occurring amino acids and non-natural amino acids) of any length. The terms also encompass an amino acid polymer that has been modified; for example, disulfide bond formation, glycosylation, acetylation, phosphorylation, lipidation, or conjugation with a labeling component.

By “pharmaceutical composition” is meant any composition that contains a therapeutically or biologically active agent (e.g., a nanoparticle containing 1-30 (e.g., 2-30, 3-30, 4-30, 5-30, 6-30, 7-30, or 8-30 tolerogenic antigens)) that is suitable for administration to a subject. The biologically active agent includes a nanoparticle that contains 1-30 (e.g., 8-30 tolerogenic antigens per nanoparticle). The 1-30 tolerogenic antigens associated with a specific nanoparticle may all have the same sequence identities, or the 1-30 tolerogenic antigens associated with a specific nanoparticle may contain between 1 and 5 different populations of tolerogenic antigens having different sequence identities. Any of these formulations can be prepared by well-known and accepted methods in the art. See, for example, Remington: The Science and Practice of Pharmacy (21st ed.), ed. A. R. Gennaro, Lippincott Williams & Wilkins, 2005, and Encyclopedia of Pharmaceutical Technology, ed. J. Swarbrick, Informa Healthcare, 2006, each of which is hereby incorporated by reference.

By “pharmaceutically acceptable diluent, excipient, carrier, or adjuvant” is meant a diluent, excipient, carrier, or adjuvant which is physiologically acceptable to the subject while retaining the therapeutic properties of the pharmaceutical composition with which it is administered.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include environmental material such as surface matter, soil, water, crystals, and industrial samples. Such examples are not however to be construed as limiting the sample types applicable to the present invention.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the terms “synthetic HDL,” “sHDL,” “reconstituted HDL”, or “rHDL” refer to a particle structurally analogous to native HDL, composed of a lipid or lipids in association with at least one of the proteins of HDL, preferably ApoA-I, or a mimetic thereof. Typically, the components of sHDL may be derived from blood or produced by recombinant technology.

By “therapeutically effective amount” is meant the amount of a composition administered to improve, inhibit, or ameliorate a condition of a subject, or a symptom of a disorder or disease, e.g., celiac disease, in a clinically relevant manner. Any improvement in the subject is considered sufficient to achieve treatment. Preferably, an amount sufficient to treat is an amount that reduces, inhibits, or prevents the occurrence or one or more symptoms of the disease or disorder (e.g., celiac disease) or is an amount that reduces the severity of, or the length of time during which a subject suffers from one or more symptoms of the disease or disorder, for example, celiac disease, (e.g., by at least about 10%, about 20%, or about 30%, more preferably by at least about 50%, about 60%, or about 70%, and most preferably by at least about 80%, about 90%, about 95%, about 99%, or more, relative to a control subject that is not treated with a composition described herein). An effective amount of the pharmaceutical composition used to practice the methods described herein (e.g., the treatment of celiac disease) varies depending upon the manner of administration and the age, body weight, and general health of the subject being treated. A physician or researcher can decide the appropriate amount and dosage regimen.

As used herein, the term “tolerogenic antigen” refers to a molecule that is capable of binding to an antibody or to an antigen receptor on a T cell, especially one that induces an immune response.

As used herein, the term “solvent” refers to a medium in which a reaction is conducted. Solvents may be liquid but are not limited to liquid form. Solvent categories include but are not limited to nonpolar, polar, protic, and aprotic.

### DETAILED DESCRIPTION OF THE INVENTION

The present invention relates to nanoparticles associated with a plurality of tolerogenic antigens (e.g., between 1-30 (e.g., between 2-30, 3-30, 4-30, 5-30, 6-30, 7-30, or 8-30 tolerogenic antigens per nanoparticle)) implicated in celiac disease in such a manner that facilitates strong immune tolerance upon administration to a subject (e.g., a human subject suffering from or at risk of suffering from an autoimmune disorder e.g., MS or celiac disease). The present invention further relates to methods for synthesizing such nanoparticles associated with tolerogenic antigens implicated in autoimmune disorder (e.g., MS or celiac disease), as well as systems and methods utilizing such nanoparticles to treat a subject suffering from an autoimmune disorder (e.g., MS or celiac disease).

### Nanoparticles

The present invention is not limited to specific types or kinds of nanoparticles associated with (e.g., complexed, conjugated, encapsulated, absorbed, adsorbed, admixed) tolerogenic antigens for treating, preventing, or ameliorating various types of autoimmune disorders (e.g., celiac disease).

Examples of nanoparticles include, but are not limited to, fullerenes (a.k.a. C₆₀, C₇₀, C₇₆, C₈₀, C₈₄), endohedral metallofullerenes (EMI's) buckyballs, which contain additional atoms, ions, or clusters inside their fullerene cage), trimetallic nitride templated endohedral metallofullerenes (TNT EMEs, high-symmetry four-atom molecular cluster endohedrals, which are formed in a trimetallic nitride template within the carbon cage), single-walled and multi-walled carbon nanotubes, branched and dendritic carbon nanotubes, gold nanorods, silver nanorods, single-walled and multi-walled boron/nitrate nanotubes, carbon nanotube peapods (nanotubes with internal metallo-fullerenes and/or other internal chemical structures), carbon nanohorns, carbon nanohorn peapods, liposomes, nanoshells, dendrimers, quantum dots, superparamagnetic nanoparticles, nanorods, and cellulose nanoparticles. The particle embodiment can also include microparticles with the capability to enhance effectiveness or selectivity. Other non-limiting exemplary nanoparticles include glass and polymer micro- and nano-spheres, biodegradable PLGA micro- and nano-spheres, gold, silver, carbon, and iron nanoparticles.

In some embodiments, the nanoparticle is a modified micelle. In these embodiments, the modified micelle comprises polyol polymers modified to contain a hydrophobic polymer block. The term “hydrophobic polymer block” as used in the present disclosure indicates a segment of the polymer that on its own would be hydrophobic. The term “micelle” as used herein refers to an aggregate of molecules dispersed in a liquid. A typical micelle in aqueous solution forms an aggregate with the hydrophilic “head” regions in contact with surrounding solvent, sequestering the hydrophobic single tail regions in the micelle centre. In some embodiments the head region may be, for example, a surface region of the polyol polymer while the tail region may be, for example, the hydrophobic polymer block region of the polyol polymer.

The invention further encompasses use of particles on the micrometer scale in addition to the nanometer scale. Where microparticles are used, it is preferred that they are relatively small, on the order of 1-50 micrometers. For ease of discussion, the use herein of “nanoparticles” encompasses true nanoparticles (sizes of from about 1 nm to about 1000 nm), microparticles (e.g., from about 1 micrometer to about 50 micrometers), or both.

Examples of nanoparticles include, by way of example and without limitation, paramagnetic nanoparticles, superparamagnetic nanoparticles, metal nanoparticles, fullerene-like materials, inorganic nanotubes, dendrimers, dendrimers with covalently attached metal chelates, nanofibers, nanohorns, nano-onions, nanorods, nanoropes, and quantum dots. In some embodiments, a nanoparticle is a metal nanoparticle (for example, a nanoparticle of gold, palladium, platinum, silver, copper, nickel, cobalt, iridium, or an alloy of two or more thereof). Nanoparticles can include a core or a core and a shell, as in core-shell nanoparticles.

In some embodiments, the nanoparticles are sHDL nanoparticles. Generally, sHDL nanoparticles are composed of a mixture of HDL apolipoprotein and an amphipathic lipid.

The present invention is not limited to use of a particular type or kind of HDL apolipoprotein. HDL apolipoproteins include, for example apolipoprotein A-I (apo A-I), apolipoprotein A-II (apo A-II), apolipoprotein A4 (apo A4), apolipoprotein Cs (apo Cs), apolipoprotein M (apo M), and apolipoprotein E (apo E). In some embodiments, the HDL apolipoprotein is selected from preproapoliprotein, preproApoA-I, proApoA-1, ApoA-I, preproApoA-II, proApoA-II, ApoA-II, apolipoprotein A-II xxx (apo A-II-xxx), preproApoA-IV, proApoA-IV, ApoA-IV, ApoA-V, preproApoE, proApoE, ApoE, preproApoA-IMilano, proApoA-IMilano, ApoA-IMilano, preproApoA-IParis, proApoA-IParis, ApoA-IParis, and peptide mimetics of these proteins mixtures thereof. Preferably, the carrier particles are composed of ApoA-I or ApoA-II, however the use of other lipoproteins including apolipoprotein A4, apolipoprotein Cs or apolipoprotein E may be used alone or in combination to formulate carrier particle mixtures for delivery of therapeutic agents. In some embodiments, mimetics of such HDL apolipoproteins are used.

ApoA-I is synthesized by the liver and small intestine as preproapolipoprotein which is secreted as a proprotein that is rapidly cleaved to generate a mature polypeptide having 243 amino acid residues. ApoA-I consists mainly of 6 to 8 different 22 amino acid repeats, and 2 different 11 amino acid repeats, each of which has the helical wheel signature of an amphipathic a helix, spaced by a linker moiety which is often proline, and, in some cases, consists of a stretch made up of several residues. ApoA-I forms three types of stable complexes with lipids: small, lipid-poor complexes referred to as pre-beta-1 HDL; flattened discoidal particles containing polar lipids (phospholipid and cholesterol) referred to as pre-beta-2 HDL; and spherical particles containing both polar and nonpolar lipids, referred to as spherical or mature HDL (HDL₃ and HDL₂). Most HDL in the circulating population contain both ApoA-I and ApoA-II (the second major HDL protein).

In some embodiments, ApoA-I agonists or mimetics are provided. In some embodiments, such ApoA-I mimetics are capable of forming amphipathic α-helices that mimic the activity of ApoA-I, and have specific activities approaching or exceeding that of the native molecule. In some, the ApoA-I mimetics are peptides or peptide analogues that: form amphipathic helices (in the presence of lipids), bind lipids, form pre-p-like or HDL-like complexes, activate lecithin: cholesterol acyltransferase (LCAT), increase serum levels of HDL fractions, and promote cholesterol efflux.

The present invention is not limited to use of a particular ApoA-I mimetic. In some embodiments, any of the ApoA-I mimetics described in Srinivasa, et al., 2014 Curr. Opinion Lipidology Vol. 25(4): 304-308 are utilized. In some embodiments, any of the ApoA-I mimetics described in U.S. Patent Application Publication Nos. 20110046056 and 20130231459 are utilized.

In some embodiments, the “22A” ApoA-I mimetic is used (PVLDLFRELLNELLEALKQKLK) (SEQ ID NO: 4) (see, e.g., U.S. Pat. No. 7,566,695). In some embodiments, any of the following ApoA-I mimetics shown in Table 1 as described in U.S. Pat. No. 7,566,695 are utilized:

TABLE 1
ApoA-I mimetics

| SEQ ID NO | AMINO ACID SEQUENCE |
|---|---|
| (SEQ ID NO: 1) | PVLDLFRELLNELLEZLKQKLK |
| (SEQ ID NO: 2) | GVLDLFRELLNELLEALKQKLKK |
| (SEQ ID NO: 3) | PVLDLFRELLNELLEWLKQKLK |
| (SEQ ID NO: 4) | PVLDLFRELLNELLEALKQKLK |
| (SEQ ID NO: 5) | PVLDLFRELLNELLEALKQKLKK |
| (SEQ ID NO: 6) | PVLDLFRELLNEXLEALKQKLK |
| (SEQ ID NO: 7) | PVLDLFKELLNELLEALKQKLK |
| (SEQ ID NO: 8) | PVLDLFRELLNEGLEALKQKLK |
| (SEQ ID NO: 9) | PVLDLFRELGNELLEALKQKLK |
| (SEQ ID NO: 10) | PVLDLFRELLNELLEAZKQKLK |
| (SEQ ID NO: 11) | PVLDLFKELLQELLEALKQKLK |
| (SEQ ID NO: 12) | PVLDLFRELLNELLEAGKQKLK |
| (SEQ ID NO: 13) | GVLDLFRELLNEGLEALKQKLK |
| (SEQ ID NO: 14) | PVLDLFRELLNELLEALOQOLO |
| (SEQ ID NO: 15) | PVLDLFRELWNELLEALKQKLK |
| (SEQ ID NO: 16) | PVLDLLRELLNELLEALKQKLK |
| (SEQ ID NO: 17) | PVLELFKELLQELLEALKQKLK |
| (SEQ ID NO: 18) | GVLDLFRELLNELLEALKQKLK |
| (SEQ ID NO: 19) | PVLDLFRELLNEGLEALKQKLK |
| (SEQ ID NO: 20) | PVLDLFREGLNELLEALKQKLK |
| (SEQ ID NO: 21) | PVLDLFRELLNELLEALKQKLK |
| (SEQ ID NO: 22) | PVLDLFRELLNELLEGLKQKLK |
| (SEQ ID NO: 23) | PLLELFKELLQELLEALKQKLK |
| (SEQ ID NO: 24) | PVLDLFRELLNELLEALQKKLK |
| (SEQ ID NO: 25) | PVLDFFRELLNEXLEALKQKLK |
| (SEQ ID NO: 26) | PVLDLFRELLNELLELLKQKLK |
| (SEQ ID NO: 27) | PVLDLFRELLNELZEALKQKLK |
| (SEQ ID NO: 28) | PVLDLFRELLNELWEALKQKLK |
| (SEQ ID NO: 29) | AVLDLFRELLNELLEALKQKLK |
| (SEQ ID NO: 30) | PVLDLFRELLNELLEALKQKLK¹ |
| (SEQ ID NO: 31) | PVLDLFLELLNEXLEALKQKLK |
| (SEQ ID NO: 32) | XVLDLFRELLNELLEALKQKLK |
| (SEQ ID NO: 33) | PVLDLFREKLNELLEALKQKLK |
| (SEQ ID NO: 34) | PVLDZFRELLNELLEALKQKLK |
| (SEQ ID NO: 35) | PVLDWFRELLNELLEALKQKLK |
| (SEQ ID NO: 36) | PLLELLKELLQELLEALKQKLK |
| (SEQ ID NO: 37) | PVLDLFREWLNELLEALKQKLK |
| (SEQ ID NO: 38) | PVLDLFRELLNEXLEAWKQKLK |
| (SEQ ID NO: 39) | PVLDLFRELLEELLKALKKKLK |
| (SEQ ID NO: 40) | PVLDLFNELLRELLEALQKKLK |
| (SEQ ID NO: 41) | PVLDLWRELLNEXLEALKQKLK |
| (SEQ ID NO: 42) | PVLDEFREKLNEXWEALKQKLK |
| (SEQ ID NO: 43) | PVLDEFREKLWEXLEALKQKLK |
| (SEQ ID NO: 44) | PVLDEFREKLNEXLEALKQKLK |
| (SEQ ID NO: 45) | PVLDEFREKLNEXLEALKQKLK |
| (SEQ ID NO: 46) | PVLDLFREKLNEXLEALKQKLK |
| (SEQ ID NO: 47) | ~VLDLFRELLNEGLEALKQKLK |
| (SEQ ID NO: 48) | PVLDLFRELLNELLEALKQKLK |
| (SEQ ID NO: 49) | PVLDLFRNLLEKLLEALEQKLK |
| (SEQ ID NO: 50) | PVLDLFRELLWEXLEALKQKLK |
| (SEQ ID NO: 51) | PVLDLFWELLNEXLEALKQKLK |
| (SEQ ID NO: 52) | PVWDEFREKLNEXLEALKQKLK |
| (SEQ ID NO: 53) | VVLDLFRELLNELLEALKQKLK |
| (SEQ ID NO: 54) | PVLDLFRELLNEWLEALKQKLK |
| (SEQ ID NO: 55) | P~~~LFRELLNELLEALKQKLK |
| (SEQ ID NO: 56) | PVLDLFRELLNELLEALKQKKK |
| (SEQ ID NO: 57) | PVLDLFRNLLEELLKALEQKLK |
| (SEQ ID NO: 58) | PVLDEFREKLNEXLEALKQKL~ |
| (SEQ ID NO: 59) | LVLDLFRELLNELLEALKQKLK |
| (SEQ ID NO: 60) | PVLDLFRELLNELLEALKQ~~~ |
| (SEQ ID NO: 61) | PVLDEFRWKLNEXLEALKQKLK |
| (SEQ ID NO: 62) | PVLDEWREKLNEXLEALKQKLK |
| (SEQ ID NO: 63) | PVLDFFREKLNEXLEALKQKLK |
| (SEQ ID NO: 64) | PWLDEFREKLNEXLEALKQKLK |
| (SEQ ID NO: 65) | ~VLDEFREKLNEXLEALKQKLK |
| (SEQ ID NO: 66) | PVLDLFRNLLEELLEALQKKLK |
| (SEQ ID NO: 67) | ~VLDLFRELLNELLEALKQKLK |
| (SEQ ID NO: 68) | PVLDEFRELLKEXLEALKQKLK |
| (SEQ ID NO: 69) | PVLDEFRKKLNEXLEALKQKLK |
| (SEQ ID NO: 70) | PVLDEFRELLYEXLEALKQKLK |
| (SEQ ID NO: 71) | PVLDEFREKLNELXEALKQKLK |
| (SEQ ID NO: 72) | PVLDLFRELLNEXLWALKQKLK |
| (SEQ ID NO: 73) | PVLDEFWEKLNEXLEALKQKLK |
| (SEQ ID NO: 74) | PVLDKFREKLNEXLEALKQKLK |
| (SEQ ID NO: 75) | PVLDEFREKLNEELEALKQKLK |
| (SEQ ID NO: 76) | PVLDEFRELLFEXLEALKQKLK |
| (SEQ ID NO: 77) | PVLDEFREKLNKXLEALKQKLK |
| (SEQ ID NO: 78) | PVLDEFRDKLNEXLEALKQKLK |
| (SEQ ID NO: 79) | PVLDEFRELLNELLEALKQKLK |
| (SEQ ID NO: 80) | PVLDLFERLLNELLEALQKKLK |
| (SEQ ID NO: 81) | PVLDEFREKLNWXLEALKQKLK |
| (SEQ ID NO: 82) | ~~LDEFREKLNEXLEALKQKLK |
| (SEQ ID NO: 83) | PVLDEFREKLNEXLEALWQKLK |
| (SEQ ID NO: 84) | PVLDEFREKLNELLEALKQKLK |
| (SEQ ID NO: 85) | P~LDLFRELLNELLEALKQKLK |
| (SEQ ID NO: 86) | PVLELFERLLDELLNALQKKLK |
| (SEQ ID NO: 87) | PLLELLKELLQELLEALKQKLK |
| (SEQ ID NO: 88) | PVLDKFRELLNEXLEALKQKLK |
| (SEQ ID NO: 89) | PVLDEFREKLNEXLWALKQKLK |
| (SEQ ID NO: 90) | ~~~DEFREKLNEXLEALKQKLK |
| (SEQ ID NO: 91) | PVLDEFRELLNEXLEALKQKLK |
| (SEQ ID NO: 92) | PVLDEFRELYNEXLEALKQKLK |
| (SEQ ID NO: 93) | PVLDEFREKLNEXLKALKQKLK |
| (SEQ ID NO: 94) | PVLDEFREKLNEALEALKQKLK |
| (SEQ ID NO: 95) | PVLDLFRELLNLXLEALKQKLK |
| (SEQ ID NO: 96) | PVLDLFRELLNEXLEALKQKLK |
| (SEQ ID NO: 97) | PVLDLFRELLNELLE~~~~~~~ |
| (SEQ ID NO: 98) | PVLDLFRELLNEELEALKQKLK |
| (SEQ ID NO: 99) | KLKQKLAELLENLLERFLDLVP |
| (SEQ ID NO: 100) | PVLDLFRELLNELLEALKQKLK |
| (SEQ ID NO: 101) | PVLDLFRELLNWXLEALKQKLK |
| (SEQ ID NO: 102) | PVLDLFRELLNLXLEALKEKLK |
| (SEQ ID NO: 103) | PVLDEFRELLNEELEALKQKLK |
| (SEQ ID NO: 104) | pLLNELLEALKQKLK~~~~~~~ |
| (SEQ ID NO: 105) | PAADAFREAANEAAEAAKQKAK |
| (SEQ ID NO: 106) | PVLDLFREKLNEELEALKQKLK |
| (SEQ ID NO: 107) | KLKQKLAELLENLLERFLDLVP |
| (SEQ ID NO: 108) | PVLDLFRWLLNEXLEALKQKLK |
| (SEQ ID NO: 109) | PVLDEFREKLNERLEALKQKLK |
| (SEQ ID NO :110) | PVLDEFREKLNEXXEALKQKLK |
| (SEQ ID NO: 111) | PVLDEFREKLWEXWEALKQKLK |
| (SEQ ID NO: 112) | PVLDEFREKLNEXSEALKQKLK |
| (SEQ ID NO: 113) | PVLDEFREKLNERLEALKQKLK |
| (SEQ ID NO: 114) | PVLDEFREKLNEXMEALKQKLK |
| (SEQ ID NO: 115) | PKLDEFREKLNEXLEALKQKLK |
| (SEQ ID NO: 116) | PHLDEFREKLNEXLEALKQKLK |
| (SEQ ID NO: 117) | PELDEFREKLNEXLEALKQKLK |
| (SEQ ID NO: 118) | PVLDEFREKLNEXLEALEQKLK |
| (SEQ ID NO: 119) | PVLDEFREKLNEELEAXKQKLK |
| (SEQ ID NO: 120) | PVLDEFREKLNEELEXLKQKLK |
| (SEQ ID NO: 121) | PVLDEFREKLNEELEALWQKLK |
| (SEQ ID NO: 122) | PVLDEFREKLNEELEWLKQKLK |
| (SEQ ID NO: 123) | QVLDLFRELLNELLEALKQKLK |
| (SEQ ID NO: 124) | PVLDLFOELLNELLEALOQOLO |
| (SEQ ID NO: 125) | NVLDLFRELLNELLEALKQKLK |
| (SEQ ID NO: 126) | PVLDLFRELLNELGEALKQKLK |
| (SEQ ID NO: 127) | PVLDLFRELLNELLELLKQKLK |
| (SEQ ID NO: 128) | PVLDLFRELLNELLEFLKQKLK |
| (SEQ ID NO: 129) | PVLELFNDLLRELLEALQKKLK |
| (SEQ ID NO: 130) | PVLELFNDLLRELLEALKQKLK |
| (SEQ ID NO: 131) | PVLELFKELLNELLDALRQKLK |
| (SEQ ID NO: 132) | PVLDLFRELLENLLEALQKKLK |
| (SEQ ID NO: 133) | PVLELFERLLEDLLQALNKKLK |
| (SEQ ID NO: 134) | PVLELFERLLEDLLKALNOKLK |
| (SEQ ID NO: 135) | DVLDLFRELLNELLEALKQKLK |
| (SEQ ID NO: 136) | PALELFKDLLQELLEALKQKLK |
| (SEQ ID NO: 137) | PVLDLFRELLNEGLEAZKQKLK |
| (SEQ ID NO: 138) | PVLDLFRELLNEGLEWLKQKLK |
| (SEQ ID NO: 139) | PVLDLFRELWNEGLEALKQKLK |
| (SEQ ID NO: 140) | PVLDLFRELLNEGLEALOQOLO |
| (SEQ ID NO: 141) | PVLDFFRELLNEGLEALKQKLK |
| (SEQ ID NO: 142) | PVLELFRELLNEGLEALKQKLK |
| (SEQ ID NO: 143) | PVLDLFRELLNEGLEALKQKLK* |
| (SEQ ID NO: 144) | PVLELFENLLERLLDALQKKLK |
| (SEQ ID NO: 145) | GVLELFENLLERLLDALQKKLK |
| (SEQ ID NO: 146) | PVLELFENLLERLLDALQKKLK |
| (SEQ ID NO: 147) | PVLELFENLLERLFDALQKKLK |
| (SEQ ID NO: 148) | PVLELFENLLERLGDALQKKLK |
| (SEQ ID NO: 149) | PVLELFENLWERLLDALQKKLK |
| (SEQ ID NO: 150) | PLLELFENLLERLLDALQKKLK |
| (SEQ ID NO: 151) | PVLELFENLGERLLDALQKKLK |
| (SEQ ID NO: 152) | PVFELFENLLERLLDALQKKLK |
| (SEQ ID NO: 153) | AVLELFENLLERLLDALQKKLK |
| (SEQ ID NO: 154) | PVLELFENLLERGLDALQKKLK |
| (SEQ ID NO: 155) | PVLELFLNLWERLLDALQKKLK |
| (SEQ ID NO: 156) | PVLELFLNLLERLLDALQKKLK |
| (SEQ ID NO: 157) | PVLEFFENLLERLLDALQKKLK |
| (SEQ ID NO: 158) | PVLELFLNLLERLLDWLQKKLK |
| (SEQ ID NO: 159) | PVLDLFENLLERLLDALQKKLK |
| (SEQ ID NO: 160) | PVLELFENLLERLLDWLQKKLK |
| (SEQ ID NO: 161) | PVLELFENLLERLLEALQKKLK |
| (SEQ ID NO: 162) | PVLELFENWLERLLDALQKKLK |
| (SEQ ID NO: 163) | PVLELFENLLERLWDALQKKLK |
| (SEQ ID NO: 164) | PVLELFENLLERLLDAWQKKLK |
| (SEQ ID NO: 165) | PVLELFENLLERLLDLLQKKLK |
| (SEQ ID NO: 166) | PVLELFLNLLEKLLDALQKKLK |
| (SEQ ID NO: 167) | PVLELFENGLERLLDALQKKLK |
| (SEQ ID NO: 168) | PVLELFEQLLEKLLDALQKKLK |
| (SEQ ID NO: 169) | PVLELFENLLEKLLDALQKKLK |
| (SEQ ID NO: 170) | PVLELFENLLEOLLDALQOOLO |
| (SEQ ID NO: 171) | PVLELFENLLEKLLDLLQKKLK |
| (SEQ ID NO: 172) | PVLELFLNLLERLGDALQKKLK |
| (SEQ ID NO: 173) | PVLDLFDNLLDRLLDLLNKKLK |
| (SEQ ID NO: 174) | PVLELFENLLERLLDALQKKLK |
| (SEQ ID NO: 175) | PVLELFENLLERLLELLNKKLK |
| (SEQ ID NO: 176) | PVLELWENLLERLLDALQKKLK |
| (SEQ ID NO: 177) | GVLELFLNLLERLLDALQKKLK |
| (SEQ ID NO: 178) | PVLELFDNLLEKLLEALQKKLR |
| (SEQ ID NO: 179) | PVLELFDNLLERLLDALQKKLK |
| (SEQ ID NO: 180) | PVLELFDNLLDKLLDALQKKLR |
| (SEQ ID NO: 181) | PVLELFENLLERWLDALQKKLK |
| (SEQ ID NO: 182) | PVLELFENLLEKLLEALQKKLK |
| (SEQ ID NO: 183) | PLLELFENLLEKLLDALQKKLK |
| (SEQ ID NO: 184) | PVLELFLNLLERLLDAWQKKLK |
| (SEQ ID NO: 185) | PVLELFENLLERLLDALQOOLO |
| (SEQ ID NO: 186) | PVLELFEQLLERLLDALQKKLK |
| (SEQ ID NO: 187) | PVLELFENLLERLLDALNKKLK |
| (SEQ ID NO: 188) | PVLELFENLLDRLLDALQKKLK |
| (SEQ ID NO: 189) | DVLELFENLLERLLDALQKKLK |
| (SEQ ID NO: 190) | PVLEFWDNLLDKLLDALQKKLR |
| (SEQ ID NO: 191) | PVLDLLRELLEELKQKLK* |
| (SEQ ID NO: 192) | PVLDLFKELLEELKQKLK* |
| (SEQ ID NO: 193) | PVLDLFRELLEELKQKLK* |
| (SEQ ID NO: 194) | PVLELFRELLEELKQKLK* |
| (SEQ ID NO: 195) | PVLELFKELLEELKQKLK* |
| (SEQ ID NO: 196) | PVLDLFRELLEELKNKLK* |
| (SEQ ID NO: 197) | PLLDLFRELLEELKQKLK* |
| (SEQ ID NO: 198) | GVLDLFRELLEELKQKLK* |
| (SEQ ID NO: 199) | PVLDLFRELWEELKQKLK* |
| (SEQ ID NO: 200) | NVLDLFRELLEELKQKLK* |
| (SEQ ID NO: 201) | PLLDLFKELLEELKQKLK* |
| (SEQ ID NO: 202) | PALELFKDLLEELRQKLR* |
| (SEQ ID NO: 203) | AVLDLFRELLEELKQKLK* |
| (SEQ ID NO: 204) | PVLDFFRELLEELKQKLK* |
| (SEQ ID NO: 205) | PVLDLFREWLEELKQKLK* |
| (SEQ ID NO: 206) | PLLELLKELLEELKQKLK* |
| (SEQ ID NO: 207) | PVLELLKELLEELKQKLK* |
| (SEQ ID NO: 208) | PALELFKDLLEELRQRLK* |
| (SEQ ID NO: 209) | PVLDLFRELLNELLQKLK |
| (SEQ ID NO: 210) | PVLDLFRELLEELKQKLK |
| (SEQ ID NO: 211) | PVLDLFRELLEELOQOLO* |
| (SEQ ID NO: 212) | PVLDLFOELLEELOQOLK* |
| (SEQ ID NO: 213) | PALELFKDLLEEFRQRLK* |
| (SEQ ID NO: 214) | PVLDLFRELLEELKQKLK* |
| (SEQ ID NO: 215) | PVLDLFRELLEEWKQKLK* |
| (SEQ ID NO: 216) | PVLELFKELLEELKQKLK |
| (SEQ ID NO: 217) | PVLDLFRELLELLKQKLK |
| (SEQ ID NO: 218) | PVLDLFRELLNELLQKLK* |
| (SEQ ID NO: 219) | PVLDLFRELLNELWQKLK |
| (SEQ ID NO: 220) | PVLDLFRELLEELQKKLK |
| (SEQ ID NO: 221) | DVLDLFRELLEELKQKLK* |
| (SEQ ID NO: 222) | PVLDAFRELLEALLQLKK |
| (SEQ ID NO: 223) | PVLDAFRELLEALAQLKK |
| (SEQ ID NO: 225) | PVLDAFRELAEALAQLKK |
| (SEQ ID NO: 226) | PVLDAFRELGEALLQLKK |
| (SEQ ID NO: 227) | PVLDLFRELGEELKQKLK* |
| (SEQ ID NO: 228) | PVLDLFREGLEELKQKLK* |
| (SEQ ID NO: 229) | PVLDLFRELLEEGKQKLK* |
| (SEQ ID NO: 230) | PVLELFERLLEDLQKKLK |
| (SEQ ID NO: 231) | PVLDLFRELLEKLEQKLK |
| (SEQ ID NO: 232) | PLLELFKELLEELKQKLK* |
| (SEQ ID NO: 233) | LDDLLQKWAEAFNQLLKK |
| (SEQ ID NO: 234) | EWLKAFYEKVLEKLKELF* |
| (SEQ ID NO: 235) | EWLEAFYKKVLEKLKELF* |
| (SEQ ID NO: 236) | DWLKAFYDKVAEKLKEAF* |
| (SEQ ID NO: 237) | DWFKAFYDKVFEKFKEFF |
| (SEQ ID NO: 238) | GIKKFLGSIWKFIKAFVG |
| (SEQ ID NO: 239) | DWFKAFYDKVAEKFKEAF |
| (SEQ ID NO: 240) | DWLKAFYDKVAEKLKEAF |
| (SEQ ID NO: 241) | DWLKAFYDKVFEKFKEFF |
| (SEQ ID NO: 242) | EWLEAFYKKVLEKLKELP |
| (SEQ ID NO: 243) | DWFKAFYDKFFEKFKEFF |
| (SEQ ID NO: 244) | EWLKAFYEKVLEKLKELF |
| (SEQ ID NO: 245) | EWLKAEYEKVEEKLKELF* |
| (SEQ ID NO: 246) | EWLKAEYEKVLEKLKELF* |
| (SEQ ID NO: 247) | EWLKAFYKKVLEKLKELF* |
| (SEQ ID NO: 248) | PVLDLFRELLEQKLK* |
| (SEQ ID NO: 249) | PVLDLFRELLEELKQK* |
| (SEQ ID NO: 250) | PVLDLFRELLEKLKQK* |
| (SEQ ID NO: 251) | PVLDLFRELLEKLQK* |
| (SEQ ID NO: 252) | PVLDLFRELLEALKQK* |
| (SEQ ID NO: 253) | PVLDLFENLLERLKQK* |
| (SEQ ID NO: 254) | PVLDLFRELLNELKQK* |

*indicates peptides that are N-terminal acetylated and C-terminal amidated; indicates peptides that are N-terminal dansylated; sp indicates peptides that exhibited solubility problems under the experimental conditions; X is Aib; Z is Nai; O is Orn; and ~ indicates deleted amino acids.
In some embodiments, an ApoA-I mimetic having the following sequence as described in U.S. Pat. No. 6,743,778 is utilized: Asp Trp Leu Lys Ala Phe Tyr Asp Lys Val Ala Glu Lys Leu Lys Glu Ala Phe (SEQ ID NO: 255).
In some embodiments, any of the following ApoA-I mimetics shown in Table 2 as described in U.S. Patent Application Publication No. 2003/0171277 are utilized:

TABLE 2
ApoA-I mimetics

| SEQ ID | |
|---|---|
| NO | AMINO ACID SEQUENCE |
| (SEQ ID | D-W-L-K-A-F-Y-D-K-V-A-E-K-L-K-E-A-F |
| NO: 256) | |
| (SEQ ID | Ac-D-W-L-K-A-F-Y-D-K-V-A-E-K-L-K-E-A-F-NH₂ |
| NO: 257) | |
| (SEQ ID | Ac-D-W-F-K-A-F-Y-D-K-V-A-E-K-L-K-E-A-F-NH₂ |
| NO: 258) | |
| (SEQ ID | Ac-D-W-L-K-A-F-Y-D-K-V-A-E-K-F-K-E-A-F-NH₂ |
| NO: 259) | |
| (SEQ ID | Ac-D-W-F-K-A-F-Y-D-K-V-A-E-K-F-K-E-A-F-NH₂ |
| NO: 260) | |
| (SEQ ID | Ac-D-W-L-K-A-F-Y-D-K-V-F-E-K-F-K-E-F-F-NH₂ |
| NO: 261) | |
| (SEQ ID | Ac-D-W-L-K-A-F-Y-D-K-F-F-E-K-F-K-E-F-F-NH₂ |
| NO: 262) | |
| (SEQ ID | Ac-D-W-F-K-A-F-Y-D-K-F-F-E-K-F-K-E-F-F-NH₂ |
| NO: 263) | |
| (SEQ ID | Ac-D-W-L-K-A-F-Y-D-K-V-A-E-K-L-K-E-F-F-NH₂ |
| NO: 264) | |
| (SEQ ID | Ac-D-W-L-K-A-F-Y-D-K-V-F-E-K-F-K-E-A-F-NH₂ |
| NO: 265) | |
| (SEQ ID | Ac-D-W-L-K-A-F-Y-D-K-V-F-E-K-L-K-E-F-F-NH₂ |
| NO: 266) | |
| (SEQ ID | Ac-D-W-L-K-A-F-Y-D-K-V-A-E-K-F-K-E-F-F-NH₂ |
| NO: 267) | |
| (SEQ ID | Ac-D-W-L-K-A-F-Y-D-K-V-F-E-K-F-K-E-F-F-NH₂ |
| NO: 268) | |
| (SEQ ID | Ac-E-W-L-K-L-F-Y-E-K-V-L-E-K-F-K-E-A-F-NH₂ |
| NO: 269) | |
| (SEQ ID | Ac-E-W-L-K-A-F-Y-D-K-V-A-E-K-F-K-E-A-F-NH₂ |
| NO: 270) | |
| (SEQ ID | Ac-E-W-L-K-A-F-Y-D-K-V-A-E-K-L-K-E-F-F-NH₂ |
| NO: 271) | |
| (SEQ ID | Ac-E-W-L-K-A-F-Y-D-K-V-F-E-K-F-K-E-A-F-NH₂ |
| NO: 272) | |
| (SEQ ID | Ac-E-W-L-K-A-F-Y-D-K-V-F-E-K-L-K-E-F-F-NH₂ |
| NO: 273) | |
| (SEQ ID | Ac-E-W-L-K-A-F-Y-D-K-V-A-E-K-F-K-E-F-F-NH₂ |
| NO: 274) | |
| (SEQ ID | Ac-E-W-L-K-A-F-Y-D-K-V-F-E-K-F-K-E-F-F-NH₂ |
| NO: 275) | |
| (SEQ ID | Ac-A-F-Y-D-K-V-A-E-K-L-K-E-A-F-NH₂ |
| NO: 276) | |
| (SEQ ID | Ac-A-F-Y-D-K-V-A-E-K-F-K-E-A-F-NH₂ |
| NO: 277) | |
| (SEQ ID | Ac-A-F-Y-D-K-V-A-E-K-F-K-E-A-F-NH₂ |
| NO: 278) | |
| (SEQ ID | Ac-A-F-Y-D-K-F-F-E-K-F-K-E-F-F-NH₂ |
| NO: 279) | |
| (SEQ ID | Ac-A-F-Y-D-K-F-F-E-K-F-K-E-F-F-NH₂ |
| NO: 280) | |
| (SEQ ID | Ac-A-F-Y-D-K-V-A-E-K-F-K-E-A-F-NH₂ |
| NO: 281) | |
| (SEQ ID | Ac-A-F-Y-D-K-V-A-E-K-L-K-E-F-F-NH₂ |
| NO: 282) | |
| (SEQ ID | Ac-A-F-Y-D-K-V-F-E-K-F-K-E-A-F-NH₂ |
| NO: 283) | |
| (SEQ ID | Ac-A-F-Y-D-K-V-F-E-K-L-K-E-F-F-NH₂ |
| NO: 284) | |
| (SEQ ID | Ac-A-F-Y-D-K-V-A-E-K-F-K-E-F-F-NH₂ |
| NO: 285) | |
| (SEQ ID | Ac-K-A-F-Y-D-K-V-F-E-K-F-K-E-F-NH₂ |
| NO: 286) | |
| (SEQ ID | Ac-L-F-Y-E-K-V-L-E-K-F-K-E-A-F-NH₂ |
| NO: 287) | |
| (SEQ ID | Ac-A-F-Y-D-K-V-A-E-K-F-K-E-A-F-NH₂ |
| NO: 288) | |
| (SEQ ID | Ac-A-F-Y-D-K-V-A-E-K-L-K-E-F-F-NH₂ |
| NO: 289) | |
| (SEQ ID | Ac-A-F-Y-D-K-V-F-E-K-F-K-E-A-F-NH₂ |
| NO: 290) | |
| (SEQ ID | Ac-A-F-Y-D-K-V-F-E-K-L-K-E-F-F-NH₂ |
| NO: 291) | |
| (SEQ ID | Ac-A-F-Y-D-K-V-A-E-K-F-K-E-F-F-NH₂ |
| NO: 292) | |
| (SEQ ID | Ac-A-F-Y-D-K-V-F-E-K-F-K-E-F-F-NH₂ |
| NO: 293) | |
| (SEQ ID | Ac-D-W-L-K-A-L-Y-D-K-V-A-E-K-L-K-E-A-L-NH₂ |
| NO: 294) | |
| (SEQ ID | Ac-D-W-F-K-A-F-Y-E-K-V-A-E-K-L-K-E-F-F-NH₂ |
| NO: 295) | |
| (SEQ ID | Ac-D-W-F-K-A-F-Y-E-K-F-F-E-K-F-K-E-F-F-NH₂ |
| NO: 296) | |
| (SEQ ID | Ac-E-W-L-K-A-L-Y-E-K-V-A-E-K-L-K-E-A-L-NH₂ |
| NO: 297) | |
| (SEQ ID | Ac-E-W-L-K-A-F-Y-E-K-V-A-E-K-L-K-E-A-F-NH₂ |
| NO: 298) | |
| (SEQ ID | Ac-E-W-F-K-A-F-Y-E-K-V-A-E-K-L-K-E-F-F-NH₂ |
| NO: 299) | |
| (SEQ ID | Ac-E-W-L-K-A-F-Y-E-K-V-F-E-K-F-K-E-F-F-NH₂ |
| NO: 300) | |
| (SEQ ID | Ac-E-W-L-K-A-F-Y-E-K-F-F-E-K-F-K-E-F-F-NH₂ |
| NO: 301) | |
| (SEQ ID | Ac-E-W-F-K-A-F-Y-E-K-F-F-E-K-F-K-E-F-F-NH₂ |
| NO: 302) | |
| (SEQ ID | Ac-D-F-L-K-A-W-Y-D-K-V-A-E-K-L-K-E-A-W-NH₂ |
| NO: 303) | |
| (SEQ ID | Ac-E-F-L-K-A-W-Y-E-K-V-A-E-K-L-K-E-A-W-NH₂ |
| NO: 304) | |
| (SEQ ID | Ac-D-F-W-K-A-W-Y-D-K-V-A-E-K-L-K-E-W-W-NH₂ |
| NO: 305) | |
| (SEQ ID | Ac-E-F-W-K-A-W-Y-E-K-V-A-E-K-L-K-E-W-W-NH₂ |
| NO: 306) | |
| (SEQ ID | Ac-D-K-L-K-A-F-Y-D-K-V-F-E-W-A-K-E-A-F-NH₂ |
| NO: 307) | |
| (SEQ ID | Ac-D-K-W-K-A-V-Y-D-K-F-A-E-A-F-K-E-F-L-NH₂ |
| NO: 308) | |
| (SEQ ID | Ac-E-K-L-K-A-F-Y-E-K-V-F-E-W-A-K-E-A-F-NH₂ |
| NO: 309) | |
| (SEQ ID | Ac-E-K-W-K-A-V-Y-E-K-F-A-E-A-F-K-E-F-L-NH₂ |
| NO: 310) | |
| (SEQ ID | Ac-D-W-L-K-A-F-V-D-K-F-A-E-K-F-K-E-A-Y-NH₂ |
| NO: 311) | |
| (SEQ ID | Ac-E-K-W-K-A-V-Y-E-K-F-A-E-A-F-K-E-F-L-NH₂ |
| NO: 312) | |
| (SEQ ID | Ac-D-W-L-K-A-F-V-Y-D-K-V-F-K-L-K-E-F-F-NH₂ |
| NO: 313) | |
| (SEQ ID | Ac-E-W-L-K-A-F-V-Y-E-K-V-F-K-L-K-E-F-F-NH₂ |
| NO: 314) | |
| (SEQ ID | Ac-D-W-L-R-A-F-Y-D-K-V-A-E-K-L-K-E-A-F-NH₂ |
| NO: 315) | |
| (SEQ ID | Ac-E-W-L-R-A-F-Y-E-K-V-A-E-K-L-K-E-A-F-NH₂ |
| NO: 316) | |
| (SEQ ID | Ac-D-W-L-K-A-F-Y-D-R-V-A-E-K-L-K-E-A-F-NH₂ |
| NO: 317) | |
| (SEQ ID | Ac-E-W-L-K-A-F-Y-E-R-V-A-E-K-L-K-E-A-F-NH₂ |
| NO: 318) | |
| (SEQ ID | Ac-D-W-L-K-A-F-Y-D-K-V-A-E-R-L-K-E-A-F-NH₂ |
| NO: 319) | |
| (SEQ ID | Ac-E-W-L-K-A-F-Y-E-K-V-A-E-R-L-K-E-A-F-NH₂ |
| NO: 320) | |
| (SEQ ID | Ac-D-W-L-K-A-F-Y-D-K-V-A-E-K-L-R-E-A-F-NH₂ |
| NO: 321) | |
| (SEQ ID | Ac-E-W-L-K-A-F-Y-E-K-V-A-E-K-L-R-E-A-F-NH₂ |
| NO: 322) | |
| (SEQ ID | Ac-D-W-L-K-A-F-Y-D-R-V-A-E-R-L-K-E-A-F-NH₂ |
| NO: 323) | |
| (SEQ ID | Ac-E-W-L-K-A-F-Y-E-R-V-A-E-R-L-K-E-A-F-NH₂ |
| NO: 324) | |
| (SEQ ID | Ac-D-W-L-R-A-F-Y-D-K-V-A-E-K-L-R-E-A-F-NH₂ |
| NO: 325) | |
| (SEQ ID | Ac-E-W-L-R-A-F-Y-E-K-V-A-E-K-L-R-E-A-F-NH₂ |
| NO: 326) | |
| (SEQ ID | Ac-D-W-L-R-A-F-Y-D-R-V-A-E-K-L-K-E-A-F-NH₂ |
| NO: 327) | |
| (SEQ ID | Ac-E-W-L-R-A-F-Y-E-R-V-A-E-K-L-K-E-A-F-NH₂ |
| NO: 328) | |
| (SEQ ID | Ac-D-W-L-K-A-F-Y-D-K-V-A-E-R-L-R-E-A-F-NH₂ |
| NO: 329) | |
| (SEQ ID | Ac-E-W-L-K-A-F-Y-E-K-V-A-E-R-L-R-E-A-F-NH₂ |
| NO: 330) | |
| (SEQ ID | Ac-D-W-L-R-A-F-Y-D-K-V-A-E-R-L-K-E-A-F-NH₂ |
| NO: 331) | |
| (SEQ ID | Ac-E-W-L-R-A-F-Y-E-K-V-A-E-R-L-K-E-A-F-NH₂ |
| NO: 332) | |

In some embodiments, an Apo A-I mimetic having the following sequence as described in U.S. Patent Application Publication No. 2006/0069030 is utilized: F-A-E-K-F-K-E-A-V-K-D-Y-F-A-K-F-W-D (SEQ ID NO:333).

In some embodiments, an Apo A-I mimetic having the following sequence as described in U.S. Patent Application Publication No. 2009/0081293 is utilized: DWFKAFYDKVAEKFKEAF (SEQ ID NO: 334); DWLKAFYDKVAEKLKEAF (SEQ ID NO: 335); PALEDLRQGLLPVLESFKVFLSALEEYTKKLNTQ (SEQ ID NO: 336).

In some embodiments, an Apo A-I mimetic having one of the following sequences is utilized:

(SEQ ID NO: 341)
WDRVKDLATVYVDVLKDSGRDYVSQF,
(SEQ ID NO: 342)
LKLLDNWDSVTSTFSKLREOL,
(SEQ ID NO: 343)
PVTOEFWONLEKETEGLROEMS,
(SEQ ID NO: 344)
KDLEEVKAKVQ,
(SEQ ID NO: 345)
KDLEEVKAKVO,
(SEQ ID NO: 346)
PYLDDFQKKWQEEMELYRQKVE,
(SEQ ID NO: 347)
PLRAELQEGARQKLHELOEKLS,
(SEQ ID NO: 348)
PLGEEMRDRARAHVDALRTHLA,
(SEQ ID NO: 349)
PYSDELRQRLAARLEALKENGG,
(SEQ ID NO: 350)
ARLAEYHAKATEHLSTLSEKAK,
(SEQ ID NO: 351)
PALEDLROGLL,
(SEQ ID NO: 352)
PVLESFKVSFLSALEEYTKKLN,
(SEQ ID NO: 353)
PVLESFVSFLSALEEYTKKLN,
(SEQ ID NO: 352)
PVLESFKVSFLSALEEYTKKLN,
(SEQ ID NO: 354)
TVLLLTICSLEGALVRRQAKEPCV,
(SEQ ID NO: 355)
QTVTDYGKDLME,
(SEQ ID NO: 356)
KVKSPELOAEAKSYFEKSKE,
(SEQ ID NO: 357)
VLTLALVAVAGARAEVSADOVATV,
(SEQ ID NO: 358)
NNAKEAVEHLOKSELTOOLNAL,
(SEQ ID NO: 359)
LPVLVWLSIVLEGPAPAOGTPDVSS,
(SEQ ID NO: 360)
LPVLVWLSIVLEGPAPAQGTPDVSS,
(SEQ ID NO: 361)
ALDKLKEFGNTLEDKARELIS,
(SEQ ID NO: 362)
WALLALLASARASEAEDASLL,
(SEQ ID NO: 363)
HLRKLRKRLLRDADDLQKRLAVYOA,
(SEQ ID NO: 364)
AQAWGERLRARMEEMGSRTRDR,
(SEQ ID NO: 365)
LDEVKEQVAEVRAKLEEQAQ,
(SEQ ID NO: 236)
DWLKAFYDKVAEKLKEAF,
(SEQ ID NO: 366)
DWLKAFYDKVAEKLKEAFPDWAKAAYDKAAEKAKEAA,
(SEQ ID NO: 367)
PVLDLFRELLNELLEALKQKL,
(SEQ ID NO: 368)
PVLDLFRELLNELLEALKQKLA,
(SEQ ID NO: 4)
PVLDLFRELLNELLEALKQKLK,
(SEQ ID NO: 369)
PVLDLFRELLNELLEALKQKLA,
(SEQ ID NO: 370)
PVLDLFRELLNELLEALKKLLK,
(SEQ ID NO: 371)
PVLDLFRELLNELLEALKKLLA,
(SEQ ID NO: 372)
PLLDLFRELLNELLEALKKLLA,
and
(SEQ ID NO: 373)
EVRSKLEEWFAAFREFAEEFLARLKS.

Amphipathic lipids include, for example, any lipid molecule which has both a hydrophobic and a hydrophilic moiety. Examples include phospholipids or glycolipids. Examples of phospholipids which may be used in the sHDL-tolerogenic antigen nanoparticles include but are not limited to 1,2-dilauroyl-sn-glycero-3-phosphocholine; 1,2-dimyristoyl-sn-glycero-3-phosphocholine; 1,2-dipalmitoyl-sn-glycero-3-phosphocholine; 1,2-distearoyl-sn-glycero-3-phosphocholine; 1,2-diarachidoyl-sn-glycero-3-phosphocholine; 1,2-dibehenoyl-sn-glycero-3-phosphocholine; 1,2-dilignoceroyl-sn-glycero-3-phosphocholine; 1,2-dimyristoleoyl-sn-glycero-3-phosphocholine; 1,2-dimyristelaidoyl-sn-glycero-3-phosphocholine; 1,2-dipalmitoleoyl-sn-glycero-3-phosphocholine; 1,2-dipalmitelaidoyl-sn-glycero-3-phosphocholine; 1,2-dipetroselenoyl-sn-glycero-3-phosphocholine; 1,2-dioleoyl-sn-glycero-3-phosphocholine; 1,2-dielaidoyl-sn-glycero-3-phosphocholine; 1,2-dieicosenoyl-sn-glycero-3-phosphocholine; 1,2-dinervonoyl-sn-glycero-3-phosphocholine; 1,2-dilauroyl-sn-glycero-3-phosphoethanolamine; 1,2-dimyristoyl-sn-glycero-3-phosphoethanolamine; 1,2-dipentadecanoyl-sn-glycero-3-phosphoethanolamine; 1,2-dipalmitoyl-sn-glycero-3-phosphoethanolamine; 1,2-distearoyl-sn-glycero-3-phosphoethanolamine; 1,2-dipalmitoleoyl-sn-glycero-3-phosphoethanolamine; 1,2-dielaidoyl-sn-glycero-3-phosphoethanolamine; 1,2-dioleoyl-sn-glycero-3-phosphoethanolamine; dioleoyl-sn-glycero-3-phosphoethanolamine-N-[3-(2-pyridyldithio) propionate]; 1,2-dipalmitoyl-sn-glycero-3-phosphothioethanol; 1,2-di-(9Z-octadecenoyl)-sn-glycero-3-phosphoethanolamine-N-[4-(p-maleimidophenyl)butyramide]; 1,2-dihexadecanoyl-sn-glycero-3-phosphoethanolamine-N-[4-(p-maleimidophenyl)butyramide]; 1,2-dihexadecanoyl-sn-glycero-3-phosphoethanolamine-N-[4-(p-maleimidomethyl)cyclohexane-carboxamide]; 1,2-di-(9Z-octadecenoyl)-sn-glycero-3-phosphoethanolamine-N-[4-(p-maleimidomethyl)cyclohexane-carboxamide]; N-[(3-Maleimide-1-oxopropyl)aminopropyl polyethyleneglycol-carbamyl] distearoylphosphatidyl-ethanolamine; N-[(3-Maleimide-1-oxopropyl)aminopropyl polyethyleneglycol-carbamyl] distearoylphosphatidyl-ethanolamine; N-(3-Maleimide-1-oxopropyl)-L-α-phosphatidylethanolamine, Distearoyl; N-[(3-Maleimide-1-oxopropyl)aminopropyl polyethyleneglycol-carbamyl] distearoylphosphatidyl-ethanolamine; N-(3-Maleimide-1-oxopropyl)-L-α-phosphatidylethanolamine, Dimyristoy; N-(3-Maleimide-1-oxopropyl)-L-α-phosphatidylethanolamine, Dioleoyl; N-(3-Maleimide-1-oxopropyl)-L-α-phosphatidylethanolamine, Dipalmitoyl; N-(3-Maleimide-1-oxopropyl)-L-α-phosphatidylethanolamine, 1-Palmitoyl-2-oleoyl; phosphatidylcholine; phosphatidylinositol; phosphatidylserine; phosphatidylethanolamine; N-(Succinimidyloxy-glutaryl)-L-α-phosphatidylethanolamine, Distearoyl; N-(Succinimidyloxy-glutaryl)-L-α-phosphatidylethanolamine, Dioleoyl; N-(Succinimidyloxy-glutaryl)-L-α-phosphatidylethanolamine, 1-Palmitoyl-2-oleoyl; N-(Succinimidyloxy-glutaryl)-L-α-phosphatidylethanolamine, Dipalmitoyl; N-(Succinimidyloxy-glutaryl)-L-α-phosphatidylethanolamine, Dimyristoyl; 3-(N-succinimidyloxyglutaryl)aminopropyl, and polyethyleneglycol-carbamyl distearoylphosphatidyl-ethanolamine; N-(3-oxopropoxy polyethyleneglycol)carbamyl-distearoyl-ethanolamine.

In some embodiments, the sHDL nanoparticles have a molar ratio of phospholipid/HDL apolipoprotein from 2 to 250 (e.g., 10 to 200, 20 to 100, 20 to 50, 30 to 40).

Generally, the sHDL nanoparticles so formed are spherical or discoidal and have a diameter of from about 5 nm to about 20 nm (e.g., 4-75 nm, 4-60 nm, 4-50 nm, 4-22 nm, 6-18 nm, 8-15 nm, 8-10 nm, etc.). In some embodiments, the sHDL nanoparticles are subjected to size exclusion chromatography to yield a more homogeneous preparation.

Such compositions are not limited to specific tolerogenic antigens implicated in autoimmune disease (e.g., MS or celiac disease).

### Tolerogenic Antigens

The present invention includes compositions and methods for treating autoimmune disease (e.g., MS or celiac disease) including nanoparticles associated with a plurality of tolerogenic antigens (e.g., between 1-30 tolerogenic antigens (e.g., 8-30 tolerogenic antigens per nanoparticle) implicated in autoimmune disease (e.g., MS or celiac disease), as well as methods utilizing such nanoparticles. In the present invention, the tolerogenic antigens are antigens that have been identified to play a role in autoimmune disease (e.g., MS or celiac disease). In some embodiments, the tolerogenic antigen is between about 3 amino acids and about 50 amino acids in length (e.g., 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length). In some embodiments, the tolerogenic antigen is a single tolerogenic antigen between about 3 and about 50 amino acids in length.

In celiac disease, the main antigens are tissue transglutaminase and gliadin (e.g., α-, γ-, and ω-gliadin). Any antigen identified as a tissue transglutaminase or a gliadin antigen may be used.

In some embodiments, the antigen associated with the nanoparticle includes a gliadin polypeptide, such as the full-length gliadin polypeptide or any epitopes of the gliadin polypeptide. In some embodiments, the antigen associated with the nanoparticle includes a 33-mer polypeptide from α2-gliadin. In some embodiments, the 33-mer gliadin polypeptide has at least 90% (at least 91%, at least 92%, at least 93%, at least 94%, at least 95%, at least 96%, at least 97%, at least 98%, at least 99%, or 100%) sequence identity to the polypeptide sequence of LQLQPFPQPELPYPQPELPYPQPELPYPQPQPF (SEQ ID NO: 374). In some embodiments, the antigen associated with the nanoparticles includes an epitope of the 33-mer gliadin polypeptide. The epitope of the 33-mer gliadin polypeptide may be a polypeptide of any length shorter than the 33-mer polypeptide, for example the epitope may include between 25 and 3 (e.g., 24, 23, 22, 21, 20, 19, 18, 17, 16, 15, 14, 13, 12, 11, 10, 9, 8, 7, 6, 5, 4 or 3) amino acid residues, between 20 and 5 (e.g., 19, 18, 17, 16, 15, 14, 13, 12, 11, 10, 9, 8, 7, 6, or 5) amino acids residues, between 12 and 6 (e.g., 12, 11, 10, 9, 8, 7 or 6) amino acid residues, or 9 amino acids in length. Further examples of epitopes of the 33-gliadin that may be associated with the nanoparticles include any one of the epitopes described in Table 3, including SEQ ID NOs: 375-405. In some embodiments, the tolerogenic antigen associated with the nanoparticle may include any one of the antigens described in Table 4, including SEQ ID NOs: 406-580. In some embodiments, the antigen associated with the nanoparticles includes a polypeptide sequence having at least 85% (e.g., at least 86%, at least 87%, at least 88%, at least 89%, at least 90%, at least 95%, or 100%) sequence identity to any one of SEQ ID NOs: 375-580. In some embodiments, the tolerogenic antigen associated with the nanoparticle may include an antigen including two or more (e.g., 2, 3, 4, 5, or 6) polypeptides having the polypeptide sequences of any two of SEQ ID NOs: 375-580. In some embodiments, the plurality of tolerogenic antigens (e.g., between 1-30 (e.g., 6-30, or 8-30 (e.g., 8, 9, 10, 11, 12, 13, 14, 15, 16, 17, 18, 19, 20, 21, 22, 23, 24, 25, 26, 27, 28, 29, or 30)) tolerogenic antigens per nanoparticle) associated with the nanoparticles have the same identity as every other tolerogenic antigen associated with the nanoparticle. In some embodiments, the plurality of tolerogenic antigens associated with the nanoparticles includes a population of between 2-10 (e.g., 2, 3, 4, 5, 6, 7, 8, 9, or 10) different antigen sequences implicated in the same disease; for example, the nanoparticles may be associated with between 3-8 (e.g., 3, 4, 5, 6, 7, or 8), 4-6 (e.g., 4, 5, or 6), or 3-4 different polypeptide antigen sequences. In some embodiments, the nanoparticles may be associated with (i) a first polypeptide population comprising the amino acid sequence of any one of SEQ ID NOs: 406-580, or a biologically active fragment or variant thereof, (ii) a second polypeptide population comprising the amino acid sequence of any one of SEQ ID NOs: 406-580, or biologically active fragment or variant thereof, and (iii) a third polypeptide population comprising the amino acid sequence of any one of SEQ ID NOs: 406-580, or a biologically active fragment or variant thereof. In some instances, the first, second, and third polypeptide populations have different amino acid sequences. In some embodiments, the nanoparticles may be associated with (i) a first polypeptide comprising the amino acid sequence LQPFPQPELPYPQPQ (SEQ ID NO: 474), or a biologically active fragment or variant thereof, (ii) a second polypeptide comprising the amino acid sequence QPFPQPEQPFPWQP (SEQ ID NO: 475), or a biologically active fragment or variant thereof, and (iii) a third polypeptide comprising the amino acid sequence PEQPIPEQPQPYPQQ (SEQ ID NO: 476), or a biologically active fragment or variant thereof. In some embodiments, the nanoparticles may be associated with (i) a first polypeptide comprising the amino acid sequence LQPFPQPELPYPQPQ (SEQ ID NO: 474), or a biologically active fragment or variant thereof, (ii) a second polypeptide comprising the amino acid sequence PQQPFPQPEQPFPWQP (SEQ ID NO: 477), or a biologically active fragment or variant thereof, and (iii) a third polypeptide comprising the amino acid sequence FPEQPIPEQPQPYPQQ (SEQ ID NO: 478), or a biologically active fragment or variant thereof. In some embodiments, the nanoparticles may be associated with (i) a first polypeptide comprising the amino acid sequence ELQPFPQPELPYPQPQ (SEQ ID NO: 506), or a biologically active fragment or variant thereof, (ii) a second polypeptide comprising the amino acid sequence EQPFPQPEQPFPWQP (SEQ ID NO: 507), or a biologically active fragment or variant thereof, and (iii) a third polypeptide comprising the amino acid sequence EPEQPIPEQPQPYPQQ (SEQ ID NO: 508), or a biologically active fragment or variant thereof. In some embodiments, the tolerogenic antigens having polypeptide sequences of SEQ ID NOs: 506, 507, and 508 include an N-terminus pyroglutamic acid (pyroE). In some embodiments described herein, the tolerogenic antigens having polypeptide sequences of SEQ ID NOs: 506, 507, and 508 include a C-terminus amide group. In some embodiments described herein, the tolerogenic antigens having polypeptide sequences of SEQ ID NOs: 506, 507, and 508 include a N-terminus pyroE residue and a C-terminus amide group. In some embodiments, the nanoparticles may be associated with (i) a first polypeptide comprising the amino acid sequence QLQPFPQPELPYPQPQ (SEQ ID NO: 509), or a biologically active fragment or variant thereof, (ii) a second polypeptide comprising the amino acid sequence QQPFPQPEQPFPWQP (SEQ ID NO: 510), or a biologically active fragment or variant thereof, and (iii) a third polypeptide comprising the amino acid sequence FPEQPIPEQPQPYPQQ (SEQ ID NO: 511), or a biologically active fragment or variant thereof. In some embodiments, the tolerogenic antigens having polypeptide sequences of SEQ ID NOs: 509, 510, and 511 include an N-terminus acetyl group. In some embodiments described herein, the tolerogenic antigens having polypeptide sequences of SEQ ID NOs: 509, 510, and 511 include a C-terminus amide group. In some embodiments described herein, the tolerogenic antigens having polypeptide sequences of SEQ ID NOs: 509, 510, and 511 include a N-terminus acetyl group and a C-terminus amide group. In any of the embodiments described herein, the population of antigens associated with the nanoparticle may be fully or partially deamidated. In some embodiments described herein, the tolerogenic antigens associated with the nanoparticle may include an N-terminus pyroglutamic acid (pyroE). In some embodiments described herein, the tolerogenic antigens associated with the nanoparticle may include an N-terminus acetyl group. In some embodiments described herein, the tolerogenic antigens associated with the nanoparticle may include an N-terminus amide group. In some embodiments described herein, the tolerogenic antigens associated with the nanoparticle may include a C-terminus amide group.

TABLE 3
Celiac Disease Relevant T-cell epitopes
recognized by CD4⁺ T cells

| SEQ ID NO: | Sequence |
|---|---|
| 375 | PFPQPELPY |
| 376 | PYPQPELPY |
| 377 | PQPELPYPQ |
| 378 | FRPEQPYPQ |
| 379 | PQQSFPEQQ |
| 380 | IQPEQPAQL |
| 381 | QQPEQPYPQ |
| 382 | SQPEQEFPQ |
| 383 | PQPEQEFPQ |
| 384 | QQPEQPFPQ |
| 385 | PQPEQPFCQ |
| 386 | QQPFPEQPQ |
| 387 | PFPQPEQPF |
| 388 | PQPEQPFPW |
| 389 | PFSEQEQPV |
| 390 | FSQQQESPF |
| 391 | QQPIPEQPQ |
| 392 | PQPEQPFPQ |
| 393 | PIPEQPQPY |
| 394 | EQPIPEQPQ |
| 395 | PQPEQPFPQ |
| 396 | PYPEQEEPF |
| 397 | PYPEQEQPF |
| 398 | PFSEQEQPV |
| 399 | EGSFQPSQE |
| 400 | EQPQQPFPQ |
| 401 | EQPQQPYPE |
| 402 | QGYYPTSPQ |
| 403 | EGSFQPSQE |
| 404 | PQQSFPEQE |
| 405 | QGYYPTSPQ |

TABLE 4
Tolerogenic Antigens

| SEQ | |
|---|---|
| ID | |
| NO: | Sequence |
| 406 | QPFPQPQQPFPW |
| 407 | QPFPQPQQPIPV |
| 408 | QPFPQPEQPFPW |
| 409 | PFPQPEQPIPV |
| 410 | QPFPQPELPFPQ |
| 411 | LPYPQPQLPYPQ |
| 412 | LPYPQPELPYPQ |
| 413 | QPFPQPQLPYPQ |
| 414 | QPFPQPELPYPQ |
| 415 | QPFPQPQQPFSQ |
| 416 | QPFPQPEQPFSQ |
| 417 | QPFPQPQQPFCQ |
| 418 | QPFPQPEQPFCQ |
| 419 | QPFPQPQLPYSQ |
| 420 | QPFPQPELPYSQ |
| 421 | LQQQCSPVAMPQRLAR |
| 422 | QPFPQPQLPYLQ |
| 423 | QPFPQPELPYLQ |
| 424 | QQFIQPQQPFPQ |
| 425 | QQFIQPEQPFPQ |
| 426 | LERPWQQQPLPP |
| 427 | LERPWQEQPLPP |
| 428 | PIPQQPEQPFPL |
| 429 | QGQQGYYPISPQQSGQ |
| 430 | QGQPGYYPTSPQQIGQ |
| 431 | PGQGQSGYYPTSPQQS |
| 432 | PQQTFPQQPQLP |
| 433 | PQQTFPEQPQLP |
| 434 | GQGQSGYYPTSPQQSG |
| 435 | QYEVIRSLVLRTLPNM |
| 436 | QVDPSGQVQWPQ |
| 437 | QVDPSGEVQWPQ |
| 438 | QPFPQPQQPFPL |
| 439 | QPFPQPEQPFPL |
| 440 | QPFPQPQQPIPY |
| 441 | QPFPQPEQPIPY |
| 442 | PQQPVPQQPQPY |
| 443 | PQQPVPEQPQPY |
| 444 | PQPFPQQPIPQQPQPY |
| 445 | QQPIPQQPQPY |
| 446 | QQPIPEQPQPY |
| 447 | QQFPQPQQPFPQ |
| 448 | QQFPQPEQPFPQ |
| 449 | PQQPIPQQPQPYPQQP |
| 450 | QQPFPQQPFPQQPQPY |
| 451 | QPFPQPQQPFSW |
| 452 | QPFPQPEQPFSW |
| 453 | PQQPFPQQPQPYPQQP |
| 454 | QPFPQPQQPIPQ |
| 455 | QPFPQPEQPIPQ |
| 456 | QPFPQPQQPFPQ |
| 457 | QPFPQPEQPFPQ |
| 458 | QPFPQPQQPTPI |
| 459 | QPFPQPEQPTPI |
| 460 | PAPIQPQQPFPQ |
| 461 | PAPIQPEQPFPQ |
| 462 | PQQPFPQQPEQI |
| 463 | PQQPFPEQPEQI |
| 464 | PQQPFPQQPQQI |
| 465 | PQQPFPEQPQQI |
| 466 | PFPQQPEQIISQ |
| 467 | PFPQQPEQIISQ |
| 468 | PFPQQPEQIIPQ |
| 469 | PFPQQPEQIIPQ |
| 470 | QPFPQPQQQLPL |
| 471 | QPFPQPEQQLPL |
| 472 | LFPLPQQPFPQ |
| 473 | LFPLPEQPFPQ |
| 474 | LQPFPQPELPYPQPQ |
| 475 | QPFPQPEQPFPWQP |
| 476 | PEQPIPEQPQPYPQQ |
| 477 | PQQPFPQPEQPFPWQP |
| 478 | FPEQPIPEQPQPYPQQ |
| 479 | PEQPIPEQPQPYPQQ |
| 480 | PQPFLPQLPYPQ |
| 481 | QAFPQPQQTFPH |
| 482 | TPIQPQQPFPQ |
| 483 | PFPLQPQQPFPQ |
| 484 | PFTQPQQPTPI |
| 485 | QPFPQLQQPQQP |
| 486 | VAHAIIMHQQQQQQQE |
| 487 | SYPVQPQQPFPQ |
| 488 | PQQPQPFPQQPVPQQP |
| 489 | PFPWQPQQPFPQ |
| 490 | PFPLQPQQPFPQ |
| 491 | QQPFQPQQPFPQ |
| 492 | NPLQPQQPFPLQPQPP |
| 493 | PLQPQQPFPLQPQPPQ |
| 494 | PNPLQPQQPFPLQ |
| 495 | TIPQQPQQPFPL |
| 496 | SFSQQPQQPFPL |
| 497 | SFSEQPQQPFPL |
| 498 | YSPYQPQQPFPQ |
| 499 | QLPLQPQQPFPQ |
| 500 | QQPQQPFPLQPQQPVP |
| 501 | IIPQQPQQPFPL |
| 502 | PEQIIPQQPQQP |
| 503 | FLLQPQQPFSQ |
| 504 | IISQQPQQPFPL |
| 505 | PFPQRPQQPFPQ |
| 506 | ELQPFPQPELPYPQPQ |
| 507 | EQPFPQPEQPFPWQP |
| 508 | EPEQPIPEQPQPYPQQ |
| 509 | QLQPFPQPELPYPQPQ |
| 510 | QQPFPQPEQPFPWQP |
| 511 | FPEQPIPEQPQPYPQQ |
| 512 | PELP |
| 513 | QPELPYP |
| 514 | PQPELPY |
| 515 | FPQPELP |
| 516 | PELPYPQP |
| 517 | QPELPYPQ |
| 518 | PQPELPYP |
| 519 | FPQPELPY |
| 520 | PFPQPELP |
| 521 | PELPYPQPQ |
| 522 | QPELPYPQP |
| 523 | FPQPELPYP |
| 524 | PFPQPELPY |
| 525 | QPFPQPELP |
| 526 | QPELPYPQPQ |
| 527 | PQPELPYPQP |
| 528 | FPQPELPYPQ |
| 529 | PFPQPELPYP |
| 530 | QPFPQPELPY |
| 531 | LQPFPQPELP |
| 532 | PQPELPYPQPQ |
| 533 | FPQPELPYPQP |
| 534 | PFPQPELPYPQ |
| 535 | QPFPQPELPYP |
| 536 | LQPFPQPELPY |
| 537 | FPQPELPYPQPQ |
| 538 | PFPQPELPYPQP |
| 539 | LQPFPQPELPYP |
| 540 | PFPQPELPYPQPQ |
| 541 | LQPFPQPELPYPQ |
| 542 | QPEQPF |
| 543 | QPEQPFP |
| 544 | PQPEQPF |
| 545 | QPEQPFPW |
| 546 | PQPEQPFP |
| 547 | FPQPEQPF |
| 548 | QPEQPFPWQ |
| 549 | FPQPEQPFP |
| 550 | QPEQPFPWQP |
| 551 | PQPEQPFPWQ |
| 552 | FPQPEQPFPW |
| 553 | PFPQPEQPFP |
| 554 | QPFPQPEQPF |
| 555 | PQPEQPFPWQP |
| 556 | FPQPEQPFPWQ |
| 557 | PFPQPEQPFPW |
| 558 | QPFPQPEQPFP |
| 559 | FPQPEQPFPWQP |
| 560 | PFPQPEQPFPWQ |
| 561 | PFPQPEQPFPWQP |
| 562 | QPFPQPEQPFPWQ |
| 563 | PIPEQPQ |
| 564 | PIPEQPQP |
| 565 | QPIPEQPQ |
| 566 | QPIPEQPQP |
| 567 | PIPEQPQPYP |
| 568 | QPIPEQPQPY |
| 569 | EQPIPEQPQP |
| 570 | PEQPIPEQPQ |
| 571 | PIPEQPQPYPQQ |
| 572 | QPIPEQPQPYPQ |
| 573 | EQPIPEQPQPYP |
| 574 | PEQPIPEQPQPY |
| 575 | QPIPEQPQPYPQQ |
| 576 | EQPIPEQPQPYPQ |
| 577 | PEQPIPEQPQPYP |
| 578 | EQPIPEQPQPYPQQ |
| 579 | PEQPIPEQPQPYPQ |
| 580 | PDLP |
| 581 | PELPYPQ |
| 582 | QPFPQPELPYPQP |
| 583 | QPFPQPELPYPQPQ |
| 584 | LQPFPQPELPYPQP |
| 585 | PIPEQPQPYPQ |
| 586 | QPIPEQPQPYP |
| 587 | EQPIPEQPQPY |
| 588 | PEQPIPEQPQP |

In some embodiments, the tolerogenic antigen is a biologically active fragment of SEQ ID NO: 474. In some instances, the biologically active fragment of SEQ ID NO: 474 includes a polypeptide comprising the sequence of SEQ ID NO: 512. In some instances, the biologically active fragment of SEQ ID NO: 474 includes a polypeptide comprising the sequence of SEQ ID NO: 580.

In some instances, the tolerogenic antigen is a biologically active fragment of SEQ ID NO: 475. In some instances, the biologically active fragment of SEQ ID NO: 475 includes a polypeptide comprising the sequence of SEQ ID NO: 542.

In some embodiments, the tolerogenic antigen is a biologically active fragment of SEQ ID NO: 476. In some instances, the biologically active fragment of SEQ ID NO: 476 includes a polypeptide comprising the sequence of SEQ ID NO: 563.

In some embodiments, the tolerogenic antigen comprises a polypeptide comprising the amino acid sequence PFPQPELPY (SEQ ID NO: 375). In some embodiments, the tolerogenic antigen comprises a polypeptide comprising the amino acid sequence PYPQPELPY (SEQ ID NO: 376). In some embodiments, the tolerogenic antigen comprises a polypeptide comprising the amino acid sequence PQPELPYPQ (SEQ ID NO: 377). In some embodiments, the tolerogenic antigen comprises a polypeptide comprising the amino acid sequence FRPEQPYPQ (SEQ ID NO: 378). In some embodiments, the tolerogenic antigen comprises a polypeptide comprising the amino acid sequence PQQSFPEQQ (SEQ ID NO: 379). In some embodiments, the tolerogenic antigen comprises a polypeptide comprising the amino acid sequence IQPEQPAQL (SEQ ID NO: 380). In some embodiments, the tolerogenic antigen comprises a polypeptide comprising the amino acid sequence QQPEQPYPQ (SEQ ID NO: 381). In some embodiments, the tolerogenic antigen comprises a polypeptide comprising the amino acid sequence SQPEQEFPQ (SEQ ID NO: 382). In some embodiments, the tolerogenic antigen comprises a polypeptide comprising the amino acid sequence PQPEQEFPQ (SEQ ID NO: 383). In some embodiments, the tolerogenic antigen comprises a polypeptide comprising the amino acid sequence QQPEQPFPQ (SEQ ID NO: 384). In some embodiments, the tolerogenic antigen comprises a polypeptide comprising the amino acid sequence PQPEQPFCQ (SEQ ID NO: 385). In some embodiments, the tolerogenic antigen comprises a polypeptide comprising the amino acid sequence QQPFPEQPQ (SEQ ID NO: 386). In some embodiments, the tolerogenic antigen comprises a polypeptide comprising the amino acid sequence PFPQPEQPF (SEQ ID NO: 387). In some embodiments, the tolerogenic antigen comprises a polypeptide comprising the amino acid sequence PQPEQPFPW (SEQ ID NO: 388). In some embodiments, the tolerogenic antigen comprises a polypeptide comprising the amino acid sequence PFSEQEQPV (SEQ ID NO: 389). In some embodiments, the tolerogenic antigen comprises a polypeptide comprising the amino acid sequence FSQQQESPF (SEQ ID NO: 390). In some embodiments, the tolerogenic antigen comprises a polypeptide comprising the amino acid sequence QQPIPEQPQ (SEQ ID NO: 391). In some embodiments, the tolerogenic antigen comprises a polypeptide comprising the amino acid sequence PQPEQPFPQ (SEQ ID NO: 392). In some embodiments, the tolerogenic antigen comprises a polypeptide comprising the amino acid sequence PIPEQPQPY (SEQ ID NO: 393). In some embodiments, the tolerogenic antigen comprises a polypeptide comprising the amino acid sequence EQPIPEQPQ (SEQ ID NO: 394). In some embodiments, the tolerogenic antigen comprises a polypeptide comprising the amino acid sequence PQPEQPFPQ (SEQ ID NO: 395). In some embodiments, the tolerogenic antigen comprises a polypeptide comprising the amino acid sequence PYPEQEEPF (SEQ ID NO: 396). In some embodiments, the tolerogenic antigen comprises a polypeptide comprising the amino acid sequence PYPEQEQPF (SEQ ID NO: 397). In some embodiments, the tolerogenic antigen comprises a polypeptide comprising the amino acid sequence PFSEQEQPV (SEQ ID NO: 398). In some embodiments, the tolerogenic antigen comprises a polypeptide comprising the amino acid sequence EGSFQPSQE (SEQ ID NO: 399). In some embodiments, the tolerogenic antigen comprises a polypeptide comprising the amino acid sequence EQPQQPFPQ (SEQ ID NO: 400). In some embodiments, the tolerogenic antigen comprises a polypeptide comprising the amino acid sequence EQPQQPYPE (SEQ ID NO: 401). In some embodiments, the tolerogenic antigen comprises a polypeptide comprising the amino acid sequence QGYYPTSPQ (SEQ ID NO: 402). In some embodiments, the tolerogenic antigen comprises a polypeptide comprising the amino acid sequence EGSFQPSQE (SEQ ID NO: 403). In some embodiments, the tolerogenic antigen comprises a polypeptide comprising the amino acid sequence PQQSFPEQE (SEQ ID NO: 404). In some embodiments, the tolerogenic antigen comprises a polypeptide comprising the amino acid sequence QGYYPTSPQ (SEQ ID NO: 405). In some embodiments, the tolerogenic antigen comprises a polypeptide comprising the amino acid sequence QPFPQPQQPFPW (SEQ ID NO: 406). In some embodiments, the tolerogenic antigen comprises a polypeptide comprising the amino acid sequence QPFPQPQQPIPV (SEQ ID NO: 407). In some embodiments, the tolerogenic antigen comprises a polypeptide comprising the amino acid sequence QPFPQPEQPFPW (SEQ ID NO: 408). In some embodiments, the tolerogenic antigen comprises a polypeptide comprising the amino acid sequence PFPQPEQPIPV (SEQ ID NO: 409). In some embodiments, the tolerogenic antigen comprises a polypeptide comprising the amino acid sequence QPFPQPELPFPQ (SEQ ID NO: 410). In some embodiments, the tolerogenic antigen comprises a polypeptide comprising the amino acid sequence LPYPQPQLPYPQ (SEQ ID NO: 411). In some embodiments, the tolerogenic antigen comprises a polypeptide comprising the amino acid sequence LPYPQPELPYPQ (SEQ ID NO: 412). In some embodiments, the tolerogenic antigen comprises a polypeptide comprising the amino acid sequence QPFPQPQLPYPQ (SEQ ID NO: 413). In some embodiments, the tolerogenic antigen comprises a polypeptide comprising the amino acid sequence QPFPQPELPYPQ (SEQ ID NO: 414). In some embodiments, the tolerogenic antigen comprises a polypeptide comprising the amino acid sequence QPFPQPQQPFSQ (SEQ ID NO: 415). In some embodiments, the tolerogenic antigen comprises a polypeptide comprising the amino acid sequence QPFPQPEQPFSQ (SEQ ID NO: 416). In some embodiments, the tolerogenic antigen comprises a polypeptide comprising the amino acid sequence QPFPQPQQPFCQ (SEQ ID NO: 417). In some embodiments, the tolerogenic antigen comprises a polypeptide comprising the amino acid sequence QPFPQPEQPFCQ (SEQ ID NO: 418). In some embodiments, the tolerogenic antigen comprises a polypeptide comprising the amino acid sequence QPFPQPQLPYSQ (SEQ ID NO: 419). In some embodiments, the tolerogenic antigen comprises a polypeptide comprising the amino acid sequence QPFPQPELPYSQ (SEQ ID NO: 420). In some embodiments, the tolerogenic antigen comprises a polypeptide comprising the amino acid sequence LQQQCSPVAMPQRLAR (SEQ ID NO: 421). In some embodiments, the tolerogenic antigen comprises a polypeptide comprising the amino acid sequence QPFPQPQLPYLQ (SEQ ID NO: 422). In some embodiments, the tolerogenic antigen comprises a polypeptide comprising the amino acid sequence QPFPQPELPYLQ (SEQ ID NO: 423). In some embodiments, the tolerogenic antigen comprises a polypeptide comprising the amino acid sequence QQFIQPQQPFPQ (SEQ ID NO: 424). In some embodiments, the tolerogenic antigen comprises a polypeptide comprising the amino acid sequence QQFIQPEQPFPQ (SEQ ID NO: 425). In some embodiments, the tolerogenic antigen comprises a polypeptide comprising the amino acid sequence LERPWQQQPLPP (SEQ ID NO: 426). In some embodiments, the tolerogenic antigen comprises a polypeptide comprising the amino acid sequence LERPWQEQPLPP (SEQ ID NO: 427). In some embodiments, the tolerogenic antigen comprises a polypeptide comprising the amino acid sequence PIPQQPEQPFPL (SEQ ID NO: 428). In some embodiments, the tolerogenic antigen comprises a polypeptide comprising the amino acid sequence QGQQGYYPISPQQSGQ (SEQ ID NO: 429). In some embodiments, the tolerogenic antigen comprises a polypeptide comprising the amino acid sequence QGQPGYYPTSPQQIGQ (SEQ ID NO: 430). In some embodiments, the tolerogenic antigen comprises a polypeptide comprising the amino acid sequence PGQGQSGYYPTSPQQS (SEQ ID NO: 431). In some embodiments, the tolerogenic antigen comprises a polypeptide comprising the amino acid sequence PQQTFPQQPQLP (SEQ ID NO: 432). In some embodiments, the tolerogenic antigen comprises a polypeptide comprising the amino acid sequence PQQTFPEQPQLP (SEQ ID NO: 433). In some embodiments, the tolerogenic antigen comprises a polypeptide comprising the amino acid sequence GQGQSGYYPTSPQQSG (SEQ ID NO: 434). In some embodiments, the tolerogenic antigen comprises a polypeptide comprising the amino acid sequence QYEVIRSLVLRTLPNM (SEQ ID NO: 435). In some embodiments, the tolerogenic antigen comprises a polypeptide comprising the amino acid sequence QVDPSGQVQWPQ (SEQ ID NO: 436). In some embodiments, the tolerogenic antigen comprises a polypeptide comprising the amino acid sequence QVDPSGEVQWPQ (SEQ ID NO: 437). In some embodiments, the tolerogenic antigen comprises a polypeptide comprising the amino acid sequence QPFPQPQQPFPL (SEQ ID NO: 438). In some embodiments, the tolerogenic antigen comprises a polypeptide comprising the amino acid sequence QPFPQPEQPFPL (SEQ ID NO: 439). In some embodiments, the tolerogenic antigen comprises a polypeptide comprising the amino acid sequence QPFPQPQQPIPY (SEQ ID NO: 440). In some embodiments, the tolerogenic antigen comprises a polypeptide comprising the amino acid sequence QPFPQPEQPIPY (SEQ ID NO: 441). In some embodiments, the tolerogenic antigen comprises a polypeptide comprising the amino acid sequence PQQPVPQQPQPY (SEQ ID NO: 442). In some embodiments, the tolerogenic antigen comprises a polypeptide comprising the amino acid sequence PQQPVPEQPQPY (SEQ ID NO: 443). In some embodiments, the tolerogenic antigen comprises a polypeptide comprising the amino acid sequence PQPFPQQPIPQQPQPY (SEQ ID NO: 444). In some embodiments, the tolerogenic antigen comprises a polypeptide comprising the amino acid sequence QQPIPQQPQPY (SEQ ID NO: 445). In some embodiments, the tolerogenic antigen comprises a polypeptide comprising the amino acid sequence QQPIPEQPQPY (SEQ ID NO: 446). In some embodiments, the tolerogenic antigen comprises a polypeptide comprising the amino acid sequence QQFPQPQQPFPQ (SEQ ID NO: 447). In some embodiments, the tolerogenic antigen comprises a polypeptide comprising the amino acid sequence QQFPQPEQPFPQ (SEQ ID NO: 448). In some embodiments, the tolerogenic antigen comprises a polypeptide comprising the amino acid sequence PQQPIPQQPQPYPQQP (SEQ ID NO: 449). In some embodiments, the tolerogenic antigen comprises a polypeptide comprising the amino acid sequence QQPFPQQPFPQQPQPY (SEQ ID NO: 450). In some embodiments, the tolerogenic antigen comprises a polypeptide comprising the amino acid sequence QPFPQPQQPFSW (SEQ ID NO: 451). In some embodiments, the tolerogenic antigen comprises a polypeptide comprising the amino acid sequence QPFPQPEQPFSW (SEQ ID NO: 452). In some embodiments, the tolerogenic antigen comprises a polypeptide comprising the amino acid sequence PQQPFPQQPQPYPQQP (SEQ ID NO: 453). In some embodiments, the tolerogenic antigen comprises a polypeptide comprising the amino acid sequence QPFPQPQQPIPQ (SEQ ID NO: 454). In some embodiments, the tolerogenic antigen comprises a polypeptide comprising the amino acid sequence QPFPQPEQPIPQ (SEQ ID NO: 455). In some embodiments, the tolerogenic antigen comprises a polypeptide comprising the amino acid sequence QPFPQPQQPFPQ (SEQ ID NO: 456). In some embodiments, the tolerogenic antigen comprises a polypeptide comprising the amino acid sequence QPFPQPEQPFPQ (SEQ ID NO: 457). In some embodiments, the tolerogenic antigen comprises a polypeptide comprising the amino acid sequence QPFPQPQQPTPI (SEQ ID NO: 458). In some embodiments, the tolerogenic antigen comprises a polypeptide comprising the amino acid sequence QPFPQPEQPTPI (SEQ ID NO: 459). In some embodiments, the tolerogenic antigen comprises a polypeptide comprising the amino acid sequence PAPIQPQQPFPQ (SEQ ID NO: 460). In some embodiments, the tolerogenic antigen comprises a polypeptide comprising the amino acid sequence PAPIQPEQPFPQ (SEQ ID NO: 461). In some embodiments, the tolerogenic antigen comprises a polypeptide comprising the amino acid sequence PQQPFPQQPEQI (SEQ ID NO: 462). In some embodiments, the tolerogenic antigen comprises a polypeptide comprising the amino acid sequence PQQPFPEQPEQI (SEQ ID NO: 463). In some embodiments, the tolerogenic antigen comprises a polypeptide comprising the amino acid sequence PQQPFPQQPQQI (SEQ ID NO: 464). In some embodiments, the tolerogenic antigen comprises a polypeptide comprising the amino acid sequence PQQPFPEQPQQI (SEQ ID NO: 465). In some embodiments, the tolerogenic antigen comprises a polypeptide comprising the amino acid sequence PFPQQPEQIISQ (SEQ ID NO: 466). In some embodiments, the tolerogenic antigen comprises a polypeptide comprising the amino acid sequence PFPQQPEQIISQ (SEQ ID NO: 467). In some embodiments, the tolerogenic antigen comprises a polypeptide comprising the amino acid sequence PFPQQPEQIIPQ (SEQ ID NO: 468). In some embodiments, the tolerogenic antigen comprises a polypeptide comprising the amino acid sequence PFPQQPEQIIPQ (SEQ ID NO: 469). In some embodiments, the tolerogenic antigen comprises a polypeptide comprising the amino acid sequence QPFPQPQQQLPL (SEQ ID NO: 470). In some embodiments, the tolerogenic antigen comprises a polypeptide comprising the amino acid sequence QPFPQPEQQLPL (SEQ ID NO: 471). In some embodiments, the tolerogenic antigen comprises a polypeptide comprising the amino acid sequence LFPLPQQPFPQ (SEQ ID NO: 472). In some embodiments, the tolerogenic antigen comprises a polypeptide comprising the amino acid sequence LFPLPEQPFPQ (SEQ ID NO: 473). In some embodiments, the tolerogenic antigen comprises a polypeptide comprising the amino acid sequence LQPFPQPELPYPQPQ (SEQ ID NO: 474). In some embodiments, the tolerogenic antigen comprises a polypeptide comprising the amino acid sequence QPFPQPEQPFPWQP (SEQ ID NO: 475). In some embodiments, the tolerogenic antigen comprises a polypeptide comprising the amino acid sequence PEQPIPEQPQPYPQQ (SEQ ID NO: 476). In some embodiments, the tolerogenic antigen comprises a polypeptide comprising the amino acid sequence PQQPFPQPEQPFPWQP (SEQ ID NO: 477). In some embodiments, the tolerogenic antigen comprises a polypeptide comprising the amino acid sequence FPEQPIPEQPQPYPQQ (SEQ ID NO: 478). In some embodiments, the tolerogenic antigen comprises a polypeptide comprising the amino acid sequence PEQPIPEQPQPYPQQ (SEQ ID NO: 479). In some embodiments, the tolerogenic antigen comprises a polypeptide comprising the amino acid sequence PQPFLPQLPYPQ (SEQ ID NO: 480). In some embodiments, the tolerogenic antigen comprises a polypeptide comprising the amino acid sequence QAFPQPQQTFPH (SEQ ID NO: 481). In some embodiments, the tolerogenic antigen comprises a polypeptide comprising the amino acid sequence TPIQPQQPFPQ (SEQ ID NO: 482). In some embodiments, the tolerogenic antigen comprises a polypeptide comprising the amino acid sequence PFPLQPQQPFPQ (SEQ ID NO: 483). In some embodiments, the tolerogenic antigen comprises a polypeptide comprising the amino acid sequence PFTQPQQPTPI (SEQ ID NO: 484). In some embodiments, the tolerogenic antigen comprises a polypeptide comprising the amino acid sequence QPFPQLQQPQQP (SEQ ID NO: 485). In some embodiments, the tolerogenic antigen comprises a polypeptide comprising the amino acid sequence VAHAIIMHQQQQQQQE (SEQ ID NO: 486). In some embodiments, the tolerogenic antigen comprises a polypeptide comprising the amino acid sequence SYPVQPQQPFPQ (SEQ ID NO: 487). In some embodiments, the tolerogenic antigen comprises a polypeptide comprising the amino acid sequence PQQPQPFPQQPVPQQP (SEQ ID NO: 488). In some embodiments, the tolerogenic antigen comprises a polypeptide comprising the amino acid sequence PFPWQPQQPFPQ (SEQ ID NO: 489). In some embodiments, the tolerogenic antigen comprises a polypeptide comprising the amino acid sequence PFPLQPQQPFPQ (SEQ ID NO: 490). In some embodiments, the tolerogenic antigen comprises a polypeptide comprising the amino acid sequence QQPFQPQQPFPQ (SEQ ID NO: 491). In some embodiments, the tolerogenic antigen comprises a polypeptide comprising the amino acid sequence NPLQPQQPFPLQPQPP (SEQ ID NO: 492). In some embodiments, the tolerogenic antigen comprises a polypeptide comprising the amino acid sequence PLQPQQPFPLQPQPPQ (SEQ ID NO: 493). In some embodiments, the tolerogenic antigen comprises a polypeptide comprising the amino acid sequence PNPLQPQQPFPLQ (SEQ ID NO: 494). In some embodiments, the tolerogenic antigen comprises a polypeptide comprising the amino acid sequence TIPQQPQQPFPL (SEQ ID NO: 495). In some embodiments, the tolerogenic antigen comprises a polypeptide comprising the amino acid sequence SFSQQPQQPFPL (SEQ ID NO: 496). In some embodiments, the tolerogenic antigen comprises a polypeptide comprising the amino acid sequence SFSEQPQQPFPL (SEQ ID NO: 497). In some embodiments, the tolerogenic antigen comprises a polypeptide comprising the amino acid sequence YSPYQPQQPFPQ (SEQ ID NO: 498). In some embodiments, the tolerogenic antigen comprises a polypeptide comprising the amino acid sequence QLPLQPQQPFPQ (SEQ ID NO: 499). In some embodiments, the tolerogenic antigen comprises a polypeptide comprising the amino acid sequence QQPQQPFPLQPQQPVP (SEQ ID NO: 500). In some embodiments, the tolerogenic antigen comprises a polypeptide comprising the amino acid sequence IIPQQPQQPFPL (SEQ ID NO: 501). In some embodiments, the tolerogenic antigen comprises a polypeptide comprising the amino acid sequence PEQIIPQQPQQP (SEQ ID NO: 502). In some embodiments, the tolerogenic antigen comprises a polypeptide comprising the amino acid sequence FLLQPQQPFSQ (SEQ ID NO: 503). In some embodiments, the tolerogenic antigen comprises a polypeptide comprising the amino acid sequence IISQQPQQPFPL (SEQ ID NO: 504). In some embodiments, the tolerogenic antigen comprises a polypeptide comprising the amino acid sequence PFPQRPQQPFPQ (SEQ ID NO: 505). In some embodiments, the tolerogenic antigen comprises a polypeptide comprising the amino acid sequence ELQPFPQPELPYPQPQ (SEQ ID NO: 506). In some embodiments, the tolerogenic antigen comprises a polypeptide comprising the amino acid sequence EQPFPQPEQPFPWQP (SEQ ID NO: 507). In some embodiments, the tolerogenic antigen comprises a polypeptide comprising the amino acid sequence EPEQPIPEQPQPYPQQ (SEQ ID NO: 508). In some embodiments, the tolerogenic antigen comprises a polypeptide comprising the amino acid sequence QLQPFPQPELPYPQPQ (SEQ ID NO: 509). In some embodiments, the tolerogenic antigen comprises a polypeptide comprising the amino acid sequence QQPFPQPEQPFPWQP (SEQ ID NO: 510). In some embodiments, the tolerogenic antigen comprises a polypeptide comprising the amino acid sequence FPEQPIPEQPQPYPQQ (SEQ ID NO: 511). In some embodiments, the tolerogenic antigen comprises a polypeptide comprising the amino acid sequence PELP (SEQ ID NO: 512). In some embodiments, the tolerogenic antigen comprises a polypeptide comprising the amino acid sequence QPELPYP (SEQ ID NO: 513). In some embodiments, the tolerogenic antigen comprises a polypeptide comprising the amino acid sequence PQPELPY (SEQ ID NO: 514). In some embodiments, the tolerogenic antigen comprises a polypeptide comprising the amino acid sequence FPQPELP (SEQ ID NO: 515). In some embodiments, the tolerogenic antigen comprises a polypeptide comprising the amino acid sequence PELPYPQP (SEQ ID NO: 516). In some embodiments, the tolerogenic antigen comprises a polypeptide comprising the amino acid sequence QPELPYPQ (SEQ ID NO: 517). In some embodiments, the tolerogenic antigen comprises a polypeptide comprising the amino acid sequence PQPELPYP (SEQ ID NO: 518). In some embodiments, the tolerogenic antigen comprises a polypeptide comprising the amino acid sequence FPQPELPY (SEQ ID NO: 519). In some embodiments, the tolerogenic antigen comprises a polypeptide comprising the amino acid sequence PFPQPELP (SEQ ID NO: 520). In some embodiments, the tolerogenic antigen comprises a polypeptide comprising the amino acid sequence PELPYPQPQ (SEQ ID NO: 521). In some embodiments, the tolerogenic antigen comprises a polypeptide comprising the amino acid sequence QPELPYPQP (SEQ ID NO: 522). In some embodiments, the tolerogenic antigen comprises a polypeptide comprising the amino acid sequence FPQPELPYP (SEQ ID NO: 523). In some embodiments, the tolerogenic antigen comprises a polypeptide comprising the amino acid sequence PFPQPELPY (SEQ ID NO: 524). In some embodiments, the tolerogenic antigen comprises a polypeptide comprising the amino acid sequence QPFPQPELP (SEQ ID NO: 525). In some embodiments, the tolerogenic antigen comprises a polypeptide comprising the amino acid sequence QPELPYPQPQ (SEQ ID NO: 526). In some embodiments, the tolerogenic antigen comprises a polypeptide comprising the amino acid sequence PQPELPYPQP (SEQ ID NO: 527). In some embodiments, the tolerogenic antigen comprises a polypeptide comprising the amino acid sequence FPQPELPYPQ (SEQ ID NO: 528). In some embodiments, the tolerogenic antigen comprises a polypeptide comprising the amino acid sequence PFPQPELPYP (SEQ ID NO: 529). In some embodiments, the tolerogenic antigen comprises a polypeptide comprising the amino acid sequence QPFPQPELPY (SEQ ID NO: 530). In some embodiments, the tolerogenic antigen comprises a polypeptide comprising the amino acid sequence LQPFPQPELP (SEQ ID NO: 531). In some embodiments, the tolerogenic antigen comprises a polypeptide comprising the amino acid sequence PQPELPYPQPQ (SEQ ID NO: 532). In some embodiments, the tolerogenic antigen comprises a polypeptide comprising the amino acid sequence FPQPELPYPQP (SEQ ID NO: 533). In some embodiments, the tolerogenic antigen comprises a polypeptide comprising the amino acid sequence PFPQPELPYPQ (SEQ ID NO: 534). In some embodiments, the tolerogenic antigen comprises a polypeptide comprising the amino acid sequence QPFPQPELPYP (SEQ ID NO: 535). In some embodiments, the tolerogenic antigen comprises a polypeptide comprising the amino acid sequence LQPFPQPELPY (SEQ ID NO: 536). In some embodiments, the tolerogenic antigen comprises a polypeptide comprising the amino acid sequence FPQPELPYPQPQ (SEQ ID NO: 537). In some embodiments, the tolerogenic antigen comprises a polypeptide comprising the amino acid sequence PFPQPELPYPQP (SEQ ID NO: 538). In some embodiments, the tolerogenic antigen comprises a polypeptide comprising the amino acid sequence LQPFPQPELPYP (SEQ ID NO: 539). In some embodiments, the tolerogenic antigen comprises a polypeptide comprising the amino acid sequence PFPQPELPYPQPQ (SEQ ID NO: 540). In some embodiments, the tolerogenic antigen comprises a polypeptide comprising the amino acid sequence LQPFPQPELPYPQ (SEQ ID NO: 541). In some embodiments, the tolerogenic antigen comprises a polypeptide comprising the amino acid sequence QPEQPF (SEQ ID NO: 542). In some embodiments, the tolerogenic antigen comprises a polypeptide comprising the amino acid sequence QPEQPFP (SEQ ID NO: 543). In some embodiments, the tolerogenic antigen comprises a polypeptide comprising the amino acid sequence PQPEQPF (SEQ ID NO: 544). In some embodiments, the tolerogenic antigen comprises a polypeptide comprising the amino acid sequence QPEQPFPW (SEQ ID NO: 545). In some embodiments, the tolerogenic antigen comprises a polypeptide comprising the amino acid sequence PQPEQPFP (SEQ ID NO: 546). In some embodiments, the tolerogenic antigen comprises a polypeptide comprising the amino acid sequence FPQPEQPF (SEQ ID NO: 547). In some embodiments, the tolerogenic antigen comprises a polypeptide comprising the amino acid sequence QPEQPFPWQ (SEQ ID NO: 548). In some embodiments, the tolerogenic antigen comprises a polypeptide comprising the amino acid sequence FPQPEQPFP (SEQ ID NO: 549). In some embodiments, the tolerogenic antigen comprises a polypeptide comprising the amino acid sequence QPEQPFPWQP (SEQ ID NO: 550). In some embodiments, the tolerogenic antigen comprises a polypeptide comprising the amino acid sequence PQPEQPFPWQ (SEQ ID NO: 551). In some embodiments, the tolerogenic antigen comprises a polypeptide comprising the amino acid sequence FPQPEQPFPW (SEQ ID NO: 552). In some embodiments, the tolerogenic antigen comprises a polypeptide comprising the amino acid sequence PFPQPEQPFP (SEQ ID NO: 553). In some embodiments, the tolerogenic antigen comprises a polypeptide comprising the amino acid sequence QPFPQPEQPF (SEQ ID NO: 554). In some embodiments, the tolerogenic antigen comprises a polypeptide comprising the amino acid sequence PQPEQPFPWQP (SEQ ID NO: 555). In some embodiments, the tolerogenic antigen comprises a polypeptide comprising the amino acid sequence FPQPEQPFPWQ (SEQ ID NO: 556). In some embodiments, the tolerogenic antigen comprises a polypeptide comprising the amino acid sequence PFPQPEQPFPW (SEQ ID NO: 557). In some embodiments, the tolerogenic antigen comprises a polypeptide comprising the amino acid sequence QPFPQPEQPFP (SEQ ID NO: 558). In some embodiments, the tolerogenic antigen comprises a polypeptide comprising the amino acid sequence FPQPEQPFPWQP (SEQ ID NO: 559). In some embodiments, the tolerogenic antigen comprises a polypeptide comprising the amino acid sequence PFPQPEQPFPWQ (SEQ ID NO: 560). In some embodiments, the tolerogenic antigen comprises a polypeptide comprising the amino acid sequence PFPQPEQPFPWQP (SEQ ID NO: 561). In some embodiments, the tolerogenic antigen comprises a polypeptide comprising the amino acid sequence QPFPQPEQPFPWQ (SEQ ID NO: 562). In some embodiments, the tolerogenic antigen comprises a polypeptide comprising the amino acid sequence PIPEQPQ (SEQ ID NO: 563). In some embodiments, the tolerogenic antigen comprises a polypeptide comprising the amino acid sequence PIPEQPQP (SEQ ID NO: 564). In some embodiments, the tolerogenic antigen comprises a polypeptide comprising the amino acid sequence QPIPEQPQ (SEQ ID NO: 565). In some embodiments, the tolerogenic antigen comprises a polypeptide comprising the amino acid sequence QPIPEQPQP (SEQ ID NO: 566). In some embodiments, the tolerogenic antigen comprises a polypeptide comprising the amino acid sequence PIPEQPQPYP (SEQ ID NO: 567). In some embodiments, the tolerogenic antigen comprises a polypeptide comprising the amino acid sequence QPIPEQPQPY (SEQ ID NO: 568). In some embodiments, the tolerogenic antigen comprises a polypeptide comprising the amino acid sequence EQPIPEQPQP (SEQ ID NO: 569). In some embodiments, the tolerogenic antigen comprises a polypeptide comprising the amino acid sequence PEQPIPEQPQ (SEQ ID NO: 570). In some embodiments, the tolerogenic antigen comprises a polypeptide comprising the amino acid sequence PIPEQPQPYPQQ (SEQ ID NO: 571). In some embodiments, the tolerogenic antigen comprises a polypeptide comprising the amino acid sequence QPIPEQPQPYPQ (SEQ ID NO: 572). In some embodiments, the tolerogenic antigen comprises a polypeptide comprising the amino acid sequence EQPIPEQPQPYP (SEQ ID NO: 573). In some embodiments, the tolerogenic antigen comprises a polypeptide comprising the amino acid sequence PEQPIPEQPQPY (SEQ ID NO: 574). In some embodiments, the tolerogenic antigen comprises a polypeptide comprising the amino acid sequence QPIPEQPQPYPQQ (SEQ ID NO: 575). In some embodiments, the tolerogenic antigen comprises a polypeptide comprising the amino acid sequence EQPIPEQPQPYPQ (SEQ ID NO: 576). In some embodiments, the tolerogenic antigen comprises a polypeptide comprising the amino acid sequence PEQPIPEQPQPYP (SEQ ID NO: 577). In some embodiments, the tolerogenic antigen comprises a polypeptide comprising the amino acid sequence EQPIPEQPQPYPQQ (SEQ ID NO: 578). In some embodiments, the tolerogenic antigen comprises a polypeptide comprising the amino acid sequence PEQPIPEQPQPYPQ (SEQ ID NO: 579). In some embodiments, the tolerogenic antigen comprises a polypeptide comprising the amino acid sequence PDLP (SEQ ID NO: 580). In some embodiments, the tolerogenic antigen comprises a polypeptide comprising the amino acid sequence PELPYPQ (SEQ ID NO: 581). In some embodiments, the tolerogenic antigen comprises a polypeptide comprising the amino acid sequence QPFPQPELPYPQP (SEQ ID NO: 582). In some embodiments, the tolerogenic antigen comprises a polypeptide comprising the amino acid sequence QPFPQPELPYPQPQ (SEQ ID NO: 583). In some embodiments, the tolerogenic antigen comprises a polypeptide comprising the amino acid sequence LQPFPQPELPYPQP (SEQ ID NO: 584). In some embodiments, the tolerogenic antigen comprises a polypeptide comprising the amino acid sequence PIPEQPQPYPQ (SEQ ID NO: 585). In some embodiments, the tolerogenic antigen comprises a polypeptide comprising the amino acid sequence QPIPEQPQPYP (SEQ ID NO: 586). In some embodiments, the tolerogenic antigen comprises a polypeptide comprising the amino acid sequence EQPIPEQPQPY (SEQ ID NO: 587). In some embodiments, the tolerogenic antigen comprises a polypeptide comprising the amino acid sequence PEQPIPEQPQP (SEQ ID NO: 588).

In some embodiments, such tolerogenic antigens include human allograft transplantation antigens. Examples of such human allograft transplantation antigens include, but are not limited to, the subunits of the various MHC class I and MHC class II haplotype proteins, and single-amino-acid polymorphisms on minor blood group antigens including RhCE, Kell, Kidd, Duffy and Ss.

In some embodiments, the tolerogenic antigen is a self antigen against which a subject (e.g., a human patient) has developed an autoimmune response or may develop an autoimmune response. Examples include proinsulin (e.g., for subjects suffering from or at risk of suffering from diabetes), collagens (e.g., for subjects suffering from or at risk of suffering from rheumatoid arthritis), and myelin basic protein (e.g., for subjects suffering from or at risk of suffering from multiple sclerosis). There are many proteins that are human autoimmune proteins, a term referring to various autoimmune diseases wherein the protein or proteins causing the disease are known or can be established by routine testing. Embodiments include testing a patient to identify an autoimmune protein and creating an antigen for use in a molecular fusion and creating immunotolerance to the protein. Embodiments include an antigen, or choosing an antigen from, one or more of the following proteins. In type 1 diabetes mellitus, several main antigens have been identified: insulin, proinsulin, preproinsulin, glutamic acid decarboxylase-65 (GAD-65), GAD-67, insulinoma-associated protein 2 (IA-2), and insulinoma-associated protein 2p (IA-2β); other antigens include ICA69, ICA12 (SOX-13), carboxypeptidase H, Imogen 38, GLIMA 38, chromogranin-A, HSP-60, carboxypeptidase E, peripherin, glucose transporter 2, hepatocarcinoma-intestine-pancreas/pancreatic associated protein, S100p, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5. In autoimmune diseases of the thyroid, including Hashimoto's thyroiditis and Graves' disease, main antigens include thyroglobulin (TG), thyroid peroxidase (TPO) and thyrotropin receptor (TSHR); other antigens include sodium iodine symporter (NIS) and megalin. In thyroid-associated ophthalmopathy and dermopathy, in addition to thyroid autoantigens including TSHR, an antigen is insulin-like growth factor 1 receptor. In hypoparathyroidism, a main antigen is calcium sensitive receptor. In Addison's disease, main antigens include 21-hydroxylase, 17α-hydroxylase, and P450 side chain cleavage enzyme (P450scc); other antigens include ACTH receptor, P450c21 and P450c17. In premature ovarian failure, main antigens include FSH receptor and α-enolase. In autoimmune hypophysitis, or pituitary autoimmune disease, main antigens include pituitary gland-specific protein factor (PGSF) 1a and 2; another antigen is type 2 iodothyronine deiodinase. In multiple sclerosis, main antigens include myelin basic protein, myelin oligodendrocyte glycoprotein and proteolipid protein. In rheumatoid arthritis, a main antigen is collagen II. In immunogastritis, a main antigen is H, K-ATPase. In pernicious angemis, a main antigen is intrinsic factor. In celiac disease, main antigens are tissue transglutaminase and gliadin. In vitiligo, a main antigen is tyrosinase, and tyrosinase related protein 1 and 2. In myasthenia gravis, a main antigen is acetylcholine receptor. In pemphigus vulgaris and variants, main antigens are desmoglein 3, 1 and 4; other antigens include pemphaxin, desmocollins, plakoglobin, perplakin, desmoplakins, and acetylcholine receptor. In bullous pemphigoid, main antigens include BP180 and BP230; other antigens include plectin and laminin 5. In dermatitis herpetiformis Duhring, main antigens include endomysium and tissue transglutaminase. In epidermolysis bullosa acquisita, a main antigen is collagen VII. In systemic sclerosis, main antigens include matrix metalloproteinase 1 and 3, the collagen-specific molecular chaperone heat-shock protein 47, fibrillin-1, and PDGF receptor; other antigens include Scl-70, U1 RNP, Th/To, Ku, Jo1, NAG-2, centromere proteins, topoisomerase I, nucleolar proteins, RNA polymerase I, II and III, PM-Slc, fibrillarin, and B23. In mixed connective tissue disease, a main antigen is U1snRNP. In Sjogren's syndrome, the main antigens are nuclear antigens SS-A and SS-B; other antigens include fodrin, poly(ADP-ribose) polymerase and topoisomerase. In systemic lupus erythematosus, main antigens include nuclear proteins including SS-A, high mobility group box 1 (HMGB1), nucleosomes, histone proteins and double-stranded DNA. In Goodpasture's syndrome, main antigens include glomerular basement membrane proteins including collagen IV. In rheumatic heart disease, a main antigen is cardiac myosin. Other autoantigens revealed in autoimmune polyglandular syndrome type 1 include aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and the type 1 interferons interferon alpha, beta and omega.

In some cases, the tolerogenic antigen is a foreign antigen against which a patient has developed an unwanted immune response. Examples are food antigens. Embodiments include testing a patient to identify foreign antigen and creating a molecular fusion that comprises the antigen and treating the patient to develop immunotolerance to the antigen or food. Examples of such foods and/or antigens are provided. Examples are from peanut: conarachin (Ara h 1), allergen II (Ara h 2), arachis agglutinin, conglutin (Ara h 6); from apple: 31 kDa major allergen/disease resistance protein homolog (Mal d 2), lipid transfer protein precursor (Mal d 3), major allergen Mal d 1.03D (Mal d 1): from milk: α-actalbumin (ALA), lactotransferrin; from kiwi: actinidin (Act c 1, Act d 1), phytocystatin, thaumatin-like protein (Act d 2), kiwellin (Act d 5); from mustard: 2S albumin (Sin a 1), 11S globulin (Sin a 2), lipid transfer protein (Sin a 3), profilin (Sin a 4); from celery: profilin (Api g 4), high molecular weight glycoprotein (Api g 5); from shrimp: Pen a 1 allergen (Pen a 1), allergen Pen m 2 (Pen m 2), tropomyosin fast isoform; from wheat and/or other cereals: high molecular weight glutenin, low molecular weight glutenin, alpha- and gamma-gliadin, hordein, secalin, avenin; from strawberry: major strawberry allergy Era a 1-E (Era a 1), from banana: profilin (Mus xp 1). In some embodiments, the tolerogenic antigens are antigenic peptides of any one of SEQ ID NOs: 589-742 (Table 5).

TABLE 5
Tolerogenic Antigens cont.

| SEQ ID | | |
|---|---|---|
| NO: | Sequence | Origin |
| 589 | Ser Gln Gln Pro Tyr Leu Gln Leu Gln | Gliadin |
| 590 | Ala Leu Ala Leu Val Arg Met Leu Ile | ATP-binding casset transporter multidrug |
| | | resistance-associate protein 7 |
| 591 | Tyr Leu Leu Pro Arg Arg Gly Pro Arg Leu | HCV core |
| 592 | Ala Ile Ser Pro Arg Thr Leu Asn Ala | p24 protein of HIV-1 |
| 593 | Val Met Ala Pro Arg Thr Leu Ile Leu | (HCMV) UL40 or HLA-Cw3 |
| 594 | Ser Gln Ala Pro Leu Pro Cys Val Leu | Epstein-Barr virus (EBC) BZLF-1 |
| 595 | Gln Met Arg Pro Val Ser Arg Val Leu | Leader sequence of human HSP60 |
| 596 | His Ala Val Ser Glu Gly Thr Lys Ala Val | Histone H2B type 1-L |
| | Thr Lys Tyr Thr Ser Ser | |
| 597 | Pro Ala Glu Thr Ala Thr Pro Ala Pro Val | Histone H1.5 |
| | Glu Lys Ser Pro Ala Lys | |
| 598 | Ala Tyr Val Arg Leu Ala Pro Asp Tyr Asp | 60S ribosomal protein L23a |
| | Ala Leu Asp Val Ala Asn | |
| 599 | His Ala Val Ser Glu Gly Thr Lys Ala Val | Histone H2B type 1-J |
| | Thr Lys Tyr Thr Ser Ala | |
| 600 | Ala Val Ser Asp Gly Val Ile Lys Val Phe | Cofilin-1 |
| | Asn Asp Met Lys Val Arg | |
| 601 | His Ala Val Ser Glu Gly Thr Lys Ala Val | Histone H2B type 1-L |
| | Thr Lys Tyr Thr Ser Ser | |
| 602 | Gln Leu Leu Gln Ala Asn Pro Ile Leu Glu | Myoson-9 |
| | Ala Phe Gly Asn Ala Lys | |
| 603 | Lys Ser Ala Asp Thr Leu TrpAsp Ile Gln | L-lactate dehydrogenase B chain |
| | Lys Asp Leu Lys Asp Leu | |
| 604 | Ala Tyr Val Arg Leu Ala Pro Asp Tyr Asp | 60S ribosomal protein L23a |
| | Ala Leu Asp Val Ala Asn | |
| 605 | His Ala Val Ser Glu Gly Thr Lys Ala Val | Histone H2B type 1-J |
| | Thr Lys Tyr Thr Ser Ala | |
| 606 | Thr Gly Leu Ile Lys Gly Ser Gly Thr Ala | Pyruvate kinase isozymes M1/M2 |
| | Glu Val Glu Leu Lys Lys | |
| 607 | Val Ser Asp Gly Val Ile Lys Val Phe Asn | Cofilin-1 |
| | Asp Met Lys Val Arg Lys | |
| 608 | Ala Ser Gly Asn Tyr Ala Thr Val Ile Ser | 60S ribosomal protein L8 |
| | His Asn Pro Glu Thr Lys | |
| 609 | Thr Ala Glu Ile Leu Glu Leu Ala Gly Asn | Histone H2Atype 1-D |
| | Ala Ala Arg Asp Asn Lys | |
| 610 | His Ala Val Ser Glu Gly Thr Lys Ala Val | Histone H2Atype 1-J |
| | Thr Lys Tyr Thr Ser Ala | |
| 611 | Pro Ala Pro Val Glu Lys Ser Pro Ala Lys | Histone H1.5 |
| | Lys Lys Ala Thr Lys | |
| 612 | Ser Ala Asp Thr Leu TrpAsp Ile Gln Lys | L-lactate dehydrogenase B chain |
| | Asp Leu Lys Asp Leu | |
| 613 | Thr Gly Leu Ile Lys Gly Ser Gly Thr Ala | Pyruvate kinase isozymes M1/M2 |
| | Glu Val Glu Leu Lys | |
| 614 | Lys Ser Ala Asp Thr Leu Trp Gly Ile Gln | L-lactate dehydrogenase A chain |
| | Lys Glu Leu Gln Phe | |
| 615 | His Gly Ser Tyr Glu Asp Ala Val His Ser | T-complex protein 1 subunit alpha |
| | Gly Ala Leu Asn Asp | |
| 616 | Ser Asp Gly Val Ile Lys Val Phe Asn Asp | Cofilin-1 |
| | Met Lys Val Arg Lys | |
| 617 | Ala Gly Asn Leu Gly Gly Gly Val Val Thr | 60S ribosomal protein L22 |
| | Ile Glu Arg Ser Lys | |
| 618 | Ala Gln Ala Ala Ala Pro Ala Ser Val Pro | 60S ribosomal protein L29 |
| | Ala Gln Ala Pro Lys | |
| 619 | Pro Arg Lys Ile Glu Glu Ile Lys Asp Phe | 60S ribosomal protein L38 |
| | Leu Leu Thr Ala Arg | |
| 620 | Ser Glu Gly Thr Lys Ala Val Thr Lys Tyr | Histone H2B type 1-L |
| | Thr Ser Ser Lys | |
| 621 | Val Leu Lys Gln Val His Pro Asp Thr Gly | Histone H2B type 1-L |
| | Ile Ser Ser Lys | |
| 622 | Ser Trp Thr Ala Ala Asp Thr Ala Ala Gln | HLA class 1 histocompatibility antigen, Cw-1 |
| | Ile Thr Gln Arg | alpha chain |
| 623 | Phe Ile Ser Val Gly Tyr Val Asp Asp Thr | HLA class 1 histocompatibility antigen, Cw-1 |
| | Gln Phe Val Arg | alpha chain |
| 624 | Asn Ile Asp Asp Gly Thr Ser Asp Arg Pro | 60S ribosomal protein L27 |
| | Tyr Ser His Ala | |
| 625 | Val Leu Lys Gln Val His Pro Asp Thr Gly | Histone H2B type 1-J |
| | Ile Ser Ser Lys | |
| 626 | Arg Lys Thr Val Thr Ala Met Asp Val Val | Histone H4 |
| | Tyr Ala Leu Lys | |
| 627 | Ser Ala Asp Thr Leu Trp Gly Ile Gln Lys | L-lactate dehydrogenase A chain |
| | Glu Leu Gln Phe | |
| 628 | Ala Ser Ala Glu Thr Val Asp Pro Ala Ser | Fascin |
| | Leu Trp Glu Tyr | |
| 629 | Thr Val Val Asn Lys Asp Val Phe Arg | 60S ribosomal protein L27 |
| | Asp Pro Ala Leu | |
| 630 | Lys Thr Val Thr Ala Met Asp Val Val Tyr | Histone H4 |
| | Ala Leu Lys | |
| 631 | Glu Gly Ile Pro Ala Leu Asp Asn Phe Leu | Elongation factor 2 |
| | Asp Lys Leu | |
| 632 | Arg Val Thr Ile Met Pro Lys Asp Ile Gln | Histone H3.3C |
| | Leu Ala Arg | |
| 633 | Pro Val Ala Val Met Ala Glu Ser Ala Phe | COP9 signalosome complex subunit 8 |
| | Ser Phe Lys | |
| 634 | Gln Thr Val Ala Val Gly Val Ile Lys Ala | Elongation factor 1-alpha 1 |
| | Val Asp Lys | |
| 635 | Ile Leu Glu Leu Ala Gly Asn Ala Ala Arg | Histone H2Atype 1-D |
| | Asp Asn Lys | |
| 636 | Gly Thr Gly Ala Ser Gly Ser Phe Lys Leu | Histone H1.5 |
| | Asn Lys | |
| 637 | Lys Gln Val His Pro Asp Thr Gly Ile Ser | Histone H2B Type 1-J |
| | Ser Lys | |
| 638 | Val Gly Gly Thr Ser Asp Val Glu Val Asn | 60 kDa heat shock protein, mitochondrial |
| | Glu Lys | |
| 639 | Asn Ser Val Val Glu Ala Ser Glu Ala Ala | 14-3-3 protein eta |
| | Tyr Lys | |
| 640 | Ala Leu Arg Tyr Pro Met Ala Val Gly Leu | 60S ribosomal protein L36 |
| | Asn Lys | |
| 641 | Ser Leu Val Ser Lys Gly Thr Leu Val Gln | Histone H1.5 |
| | Thr Lys | |
| 642 | Pro Glu Leu Ala Lys Ser Ala Pro Ala Pro | Histone H2B type 1-L |
| | Lys | |
| 643 | Ser Glu Met Glu Val Gln Asp Ala Glu Leu | Proliferation-associated protein 2G4 |
| | Lys | |
| 644 | Gln Thr Tyr Ser Thr Glu Pro Asn Asn | 60S ribosomal protein L28 |
| | Leu Lys | |
| 645 | Pro Met Phe Ile Val Asn Thr Asn Val Pro | Macrophage migration inhibitory factor |
| | Arg | |
| 646 | Ala Gly Phe Ala Gly Asp Asp Ala Pro Arg | Actin, cytoplasmic 1 |
| 647 | Arg Val Asn Ala Gly Thr Leu Ala Val Leu | von Willebrand factor A domain-containing |
| | | protein 8 |
| 648 | Ile Gly Gln Ser Lys Val Phe Phe Arg | Myoson-9 |
| 649 | Thr Ala Glu Ile Leu Glu Leu Ala Gly Asn | Histone H2Atype 1-D |
| | Ala Ala Arg Asp Asn Lys | |
| 650 | Ile Leu Glu Leu Ala Gly Asn Ala Ala Arg | Histone H2Atype 1-D |
| | Asp Asn Lys | |
| 651 | Ala Leu Ala Gly Cys His Leu Glu Asp Thr | Polyamine-modulated factor 1-binding |
| | Gln Arg Lys Leu Gln Lys Gly | protein 1 |
| 652 | Met Gln Leu Ile Thr Arg Gly Lys Gly Ala | lsthmin-1 |
| | Gly Thr Pro Asn Leu Ile | |
| 653 | Lys Met Lys Leu Arg Asn Thr Val His | Taste receptor type 2 member 50 |
| | Leu Ser Tyr Leu Thr Val | |
| 654 | Cys Arg Ala Ser Gln Thr Ile Ser Ser Tyr | Ig kappa chain V-I region OU |
| | Leu Asp Trp Tyr Gln | |
| 655 | Pro Ala Ala Leu Thr Asn Lys Gly Asn Thr | Intraflagellar transport protein 88 homologue |
| | Val Phe Ala | |
| 656 | Trp Thr Pro Gly Pro Ser Ala Gly Val Thr | Mucin-19 |
| | Gly Ile Ala | |
| 657 | Ile Leu Arg Thr Ile Gly Lys Glu Ala Phe | Trafficking protein particle complex subunit 8 |
| 658 | Arg Ser Cys Gly Tyr Ala Cys Thr Ala | Isthmin-1 |
| 659 | Phe Pro Asn Gly Phe Ser Phe Ile His | von Willebrand factor A, EGF, and pentraxin |
| | | domain-containing protein 1 |
| 660 | Ser His Gly Pro Tyr Ile Lys Leu Ile | Major facilitator superfamily domain- |
| | | containing protein 2A |
| 661 | Ala Gln Ala Ala Ala Pro Ala Ser Val Pro | 60S ribosomal protein L29 |
| | Ala Gln Ala Pro Lys | |
| 662 | Ala Tyr Val Arg Leu Ala Pro Asp Tyr Asp | 60S ribosomal protein L23a |
| | Ala Leu Asp Val Ala Asn Lys | |
| 663 | Ala Tyr Val Arg Leu Ala Pro Asp Tyr Asp | 60S ribosomal protein L23a |
| | Ala Leu Asp Val Ala Asn | |
| 664 | Ala Ser Gly Asn Tyr Ala Thr Val Ile Ser | 60S ribosomal protein L8 |
| | His Asn Pro Glu Thr Lys | |
| 665 | Ala Gly Asn Leu Gly Gly Gly Val Val Thr | 60S ribosomal protein L22 |
| | Ile Glu Arg Ser Lys | |
| 666 | Pro Arg Lys Ile Glu Glu Ile Lys Asp Phe | 60S ribosomal protein L38 |
| | Leu Leu Thr Ala Arg | |
| 667 | Asn Ile Asp Asp Gly Thr Ser Asp Arg Pro | 60S ribosomal protein L27 |
| | Tyr Ser His Ala | |
| 668 | Thr Val Val Asn Lys Asp Val Phe Arg | 60S ribosomal protein L27 |
| | Asp Pro Ala Leu | |
| 669 | Ala Leu Arg Tyr Pro Met Ala Val Gly Leu | 60S ribosomal protein L36 |
| | Asn Lys | |
| 670 | Gln Thr Tyr Ser Thr Glu Pro Asn Asn | 60S ribosomal protein L28 |
| | Leu Lys | |
| 671 | Pro Glu Leu Ala Lys Ser Ala Pro Ala Pro | Histone H2B type 1-L |
| | Lys | |
| 672 | Lys Gln Val His Pro Asp Thr Gly Ile Ser | Histone H2B type 1-J |
| | Ser Lys | |
| 673 | Val Leu Lys Gln Val His Pro Asp Thr Gly | Histone H2B type 1-J |
| | Ile Ser Ser Lys | |
| 674 | Ser Glu Gly Thr Lys Ala Val Thr Lys Tyr | Histone H2B type 1-L |
| | Thr Ser Ser Lys | |
| 675 | His Ala Val Ser Glu Gly Thr Lys Ala Val | Histone H2B type 1-J |
| | Thr Lys Tyr Thr Ser Ala | |
| 676 | His Ala Val Ser Glu Gly Thr Lys Ala Val | Histone H2B type 1-L |
| | Thr Lys Tyr Thr Ser Ser | |
| 677 | His Ala Val Ser Glu Gly Thr Lys Ala Val | Histone H2B type 1-J |
| | Thr Lys Tyr Thr Ser Ala Lys | |
| 678 | His Ala Val Ser Glu Gly Thr Lys Ala Val | Histone H2B type 1-L |
| | Thr Lys Tyr Thr Ser Ser Lys | |
| 679 | His Ala Val Ser Glu Gly Thr Lys Ala Val | Histone H2B type 1-J |
| | Thr Lys Tyr Thr Ser Ala | |
| 680 | Ala Gly Phe Ala Gly Asp Asp Ala Pro Arg | Actin, cytoplasmic 1 |
| 681 | Ser Gln Ala Pro Leu Pro Cys Val Leu | Epstein-Barr virus (EBC) BZLF-1 |
| 682 | Val Met Ala Pro Arg Thr Leu Phe Leu | HLA-G leader peptide |
| 683 | Pro Lys Lys Thr Glu Ser His His Lys Ala | Histone H2Atype 3 |
| | Lys Gly Lys | |
| 684 | Ala Ala Val Leu Glu Tyr Leu | Histone H2A type 2-B |
| 685 | Ala Gln Ala Ala Ala Pro Ala Ser Val Pro | 60S ribosomal protein L29 |
| | Ala Gln Ala Pro Lys Arg Thr Gln Ala Pro | |
| | Thr Lys Ala Ser Glu | |
| 686 | Lys Leu Glu Lys Glu Glu Glu Glu Gly Ile | High mobility group protein HMG-I/HMG-Y |
| | Ser Gln Glu Ser Ser Glu Glu Glu Gln | |
| 687 | Gly Asp Arg Ser Glu Asp Phe Gly Val | Annexin A1 |
| | Asn Glu Asp Leu Ala Asp Ser Asp Ala | |
| | Arg | |
| 688 | Val Ala Pro Glu Glu His Pro Val Leu Leu | Actin, cytoplasmic 1 |
| | Thr Glu Ala Pro Leu Asn Pro Lys | |
| 689 | Ser Thr Ala Gly Asp Thr His Leu Gly Gly | Heat shock cognate 71 kDa protein |
| | Glu Asp Phe Asp Asn Arg | |
| 690 | Lys Val Pro Gln Val Ser Thr Pro Thr Leu | Serum albumin |
| | Val Glu Val Ser Arg | |
| 691 | Pro Asp Pro Ala Lys Ser Ala Pro Ala Pro | Histone H2B type 1-H |
| | Lys Lys Gly Ser Lys | |
| 692 | Leu Gln Ala Glu Ile Glu Gly Leu Lys Gly | Keratin, type II cytoskeletal 8 |
| | Gln Arg | |
| 693 | Pro Asp Pro Ala Lys Ser Ala Pro Ala Pro | Histone H2B type 1-H |
| | Lys | |
| 694 | Pro Glu Leu Ala Lys Ser Ala Pro Ala Pro | Histone H2B type 1-L |
| | Lys | |
| 695 | Pro Glu Pro Val Lys Ser Ala Pro Val Pro | Histone H2B Type 1-M |
| | Lys | |
| 696 | Ala Ala Pro Ala Thr Arg Ala Ala Leu | Solute carrier family 15 member 4 |
| 697 | Ser Ala Pro Ser Arg Ala Thr Ala Leu | BTB/POZ domain-containing protein |
| | | KCTD18 |
| 698 | Ile Leu Asn Phe Pro Pro Pro Pro | Caprin-2 |
| 699 | Ile Ala Pro Thr Gly His Ser Leu | Septin-6 |
| 700 | Ile Ser Pro His Gly Asn Ala Leu | ATP-dependent Clp protease ATP-binding |
| | | subunit clpx-like, mitochondrial |
| 701 | Pro Asp Pro Ala Lys Ser Ala Pro Ala Pro | Histone H2B type 1-H |
| | Lys Lys Gly Ser Lys | |
| 702 | Pro Asp Pro Ala Lys Ser Ala Pro Ala Pro | Histone H2B type 1-H |
| | Lys | |
| 703 | Pro Glu Leu Ala Lys Ser Ala Pro Ala Pro | Histone H2B type 1-L |
| | Lys | |
| 704 | Pro Glu Pro Val Lys Ser Ala Pro Val Pro | Histone H2B type 1-M |
| | Lys | |
| 705 | Val Ala Pro Glu Glu His Pro Val Leu Leu | Actin, cytoplasmic 1 |
| | Thr Glu Ala Pro Leu Asn Pro Lys | |
| 706 | Pro Leu Leu Ala Leu Leu Ala Leu Trp | Insulin |
| | Gly Pro Asp Pro | |
| 707 | Pro Lys Thr Arg Arg Glu Ala Glu Val Gly | Insulin |
| | Gln | |
| 708 | Arg Arg Glu Ala Glu Asp Leu Gln Gly Ser | Insulin |
| | Leu | |
| 709 | Arg Arg Glu Ala Glu Asp Leu Glu Gly Ser | Insulin |
| | Leu | |
| 710 | Asp Leu Gln Val Gly Gln Val Glu Leu Gly | Insulin |
| | Gly Gly Pro | |
| 711 | Asp Leu Gln Val Gly Glu Val Glu Leu Gly | Insulin |
| | Gly Gly Pro | |
| 712 | Cys Gly Ser His Leu Val Glu Ala Leu Tyr | Insulin |
| | Leu Val Cys | |
| 713 | Met Asn Ile Leu Leu Glu Tyr Val Val Lys | Insulin |
| | Ser | |
| 714 | Asn Met Phe Thr Tyr Glu Ile Ala Pro Val | Insulin |
| | Phe Val Leu Leu Glu Tyr Val | |
| 715 | Asn Val Cys Phe Trp Tyr Ile Pro Pro Ser | Insulin |
| | Leu Arg Thr Leu Glu Asp Asn | |
| 716 | Phe Asn Gln Leu Ser Thr Gly Leu Asp | Insulin |
| | Met Val Gly Leu Ala Ala Asp Trp | |
| 717 | Gly Arg Thr Gly Thr Tyr Ile Leu Ile Asp | Insulin |
| | Met Val Leu Asn Arg Met Ala | |
| 718 | Pro Lys Ala Ala Arg Pro Pro Val Thr Pro | Insulin |
| | Val Leu Leu Glu Lys Lys Ser | |
| 719 | Leu Leu Ala Leu Leu Ala Leu Trp Gly | Insulin |
| | Pro Asp | |
| 720 | Lys Lys Lys Lys Tyr Val Ser Ile Asp Val | Thyroid Stimulating Hormone Receptor |
| | Thr Leu Gln Gln Leu Glu Ser His Lys Lys | |
| | Lys | |
| 721 | Gly Leu Lys Met Phe Pro Asp Leu Thr | Thyroid Stimulating Hormone Receptor |
| | Lys Val Tyr Ser Thr Asp | |
| 722 | H-Pro-Arg-His-Arg-Asp-Thr-Gly-Ile-Leu- | Myelin Basic Protein |
| | Asp-Ser-Ile- Gly-Arg-Phe-NH2 | |
| 723 | H-Glu-Asn-Pro-Val-Val-His-Phe-Phe- | Myelin Basic Protein |
| | Lys-Asn-1 le-VaI- Th r-Pro-Arg-Th r-Pro- | |
| | NH2 | |
| 724 | H-Ala-Ser-Asp-Tyr-Lys-Ser-Ala-His-Lys- | Myelin Basic Protein |
| | Gly-Phe-Lys- Gly-Val-Asp-NH2 | |
| 725 | H-Gly-Phe-Lys-Gly-Val-Asp-Ala-GIn-Gly- | Myelin Basic Protein |
| | Thr-Leu-Ser- Lys-Ile-Phe-NH2 | |
| 726 | Thr Gln Gln Ile Arg Leu Gln Ala Glu Ile | Apo E |
| | Phe Gln Ala Arg | |
| 727 | Ala Gln Gln Ile Arg Leu Gln Ala Glu Ala | Apo E |
| | Phe Gln Ala Arg | |
| 728 | Thr Ala Gln Ile Arg Leu Gln Ala Glu Ile | Apo E |
| | Phe Gln Ala Arg | |
| 729 | Thr Gln Ala Ile Arg Leu Gln Ala Glu Ile | Apo E |
| | Phe Gln Ala Arg | |
| 730 | Thr Gln Gln Ala Arg Leu Gln Ala Glu Ile | Apo E |
| | Phe Gln Ala Arg | |
| 731 | Thr Gln Gln Ile Ala Leu Gln Ala Glu Ile | Apo E |
| | Phe Gln Ala Arg | |
| 732 | Gln Thr Gln Gln Ile Arg Leu Gln Ala Glu | Apo E |
| | Ile Phe Gln Ala Arg | |
| 733 | Gln Gln Ile Arg Leu Gln Ala Glu Ile Phe | Apo E |
| | Gln Ala Arg | |
| 734 | Asn Ile Asp Ala Leu Asn Glu Asn Lys | p-lactoglobulin |
| 735 | Asn Val Leu Val Leu Asp Thr Asp Tyr | p-lactoglobulin |
| | Lys Lys | |
| 736 | Asn Thr Pro Glu Val Asp Asp Glu Ala | p-lactoglobulin |
| | Leu Glu Lys Phe Asp Lys | |
| 737 | Leu Glu Asp Ala Arg Arg Leu Lys Ala Ile | A cl repressor protein |
| | Tyr Glu Lys Lys Lys | |
| 738 | His Ser Leu Gly Lys Trp Leu Gly His Pro | Myelin proteolipid protein |
| | Asp Lys Phe | |
| 739 | Asn Thr Trp Thr Thr Cys Gln Ser Ile Ala | Myelin proteolipid protein |
| | Phe Pro Ser Lys | |
| 740 | Ile Ala Ala Thr Tyr Asn Phe Ala Val Leu | Myelin proteolipid protein |
| | Lys Leu Met Gly Arg Gly | |
| 741 | Val His Phe Phe Lys Asn Ile Val Thr Pro | Major basic protein |
| | Arg Thr Pro | |
| 742 | Asp Glu Gly Gly Tyr Thr Cys Phe Phe | Myelin oligodendrocyte glycoprotein |
| | Arg Asp His Ser Tyr Gln | |

In some instances, the autoimmune disease is Type 1 diabetes and the tolerogenic antigen is derived from Carboxypeptidase H, Chromagranin A, Glutamate decarboxylase, Imogen-38, Insulin, Insulinoma antigen-2 and 2β, Islet-specific glucose-6-phosphatase catalytic subunit related protein (IGRP), pancreatic beta-cell antigens or Proinsulin.

In some instances, the autoimmune disease is MS and the tolerogenic antigen is derived from α-enolase, aquaporin-4, β-arrestin, myelin basic protein, myelin oligodendrocytic glycoprotein, proteolipid protein, or S100-β.

In some instances, the autoimmune disease is rheumatoid arthritis and the tolerogenic antigen is derived from citrullinated protein, collagen II, heat shock proteins, gpI30-RAPS, or human cartilage glycoprotein 39.

In some instances, the autoimmune disease is systemic lupus erythematosus and the tolerogenic antigen is derived from La antigen, nucleosome histones and ribonucleoproteins (snRNP), phospholipid-p-2 glycoprotein I complex, poly(ADP-ribose) polymerase, glycoprotein gp70, or Sm antigens of U-1 small ribonucleoprotein complex.

In some instances, the autoimmune disease is scleroderma and the tolerogenic antigen is derived from fibrillarin or small nucleolar protein (snoRNP).

In some embodiments, the autoimmune disease is Graves' disease and the tolerogenic antigen is derived from thyroid stimulating factor receptor (TSH-R).

In some instances, the autoimmune disease is biliary cirrhosis and the tolerogenic antigen is derived from pyruvate dehydrogenase dihydrolipoamide acetyltransferase (PCD-E2).

In some embodiments, the autoimmune disease is alopecia areata and the tolerogenic antigen is derived from hair follicle antigens.

In some instances, the autoimmune disease is ulcerative colitis and the tolerogenic antigen is derived from human tropomyosin isoform 5 (hTM5).

In some instances, the tolerogenic antigen is derived from an antigen selected from a group consisting of 17-hydroxylase, 21-hydroxylase, ADAMTS13, Annexin A5, apoH, AQP4, aromatic acid carboxylase, Basement Membrane Collagen Type IV, BP-1, BP-2, carbonic anhydrase, carboxypeptidase H, cardiolipin, cardiolipin, chromogranin A, complement component 3, Desmoglein 3, enolase, epidermal transglutaminase, GD1a, gliadin, glutamate receptor, Glutamic acid decarboxylase, glycoproteins IIb-IIIa or Ib-IX, GMCSF, gpIIb-IIIa or 1b-IX, GQ1b, GQ1b, histidine-tRNA, histones, HPA-1a, HPA-5b, HSP60, HSP70, HSP90, Hu, IA-2beta, IAPP, ICA69, IFN-gamma, IGRP, IL-1, insulin, insulinoma antigen-2, interferon omega, Jo1, keratin, Kir4.1, LA, LKM-1, LKM-1, LKM-2, LKM-3, LP, major peripheral myelin protein P0, Mi-2, muscarinic acetylcholine receptor M1, MuSK protein; hypocretin, myelin associated glycoprotein (MAG), myelin basic protein (MBP), myelin oligodendrocyte glycoprotein (MOG), myelin-associated oligodendrocytic basic protein cardiac myosin, myeloperoxidase, neurofilaments, nicotinic acetylcholine receptor, orexin, outer surface protein (OSP), β62, phosphatidylserine, proteolipid protein (PLP), pyruvate dehydrogenase, Q-type calcium channel, Ro, sc170, signal recognition peptide, SMA, soluble liver antigen, sp100, synaptogagmin, thyroglobulin, thyroid peroxidase, tissue transglutaminase, TNF-alpha, topoisomerase, transglutaminase, type XVII collagen, U1-RNP, voltage-gated calcium channels, Yo, ZnT8, β2 glycoprotein I, or β2 glycoprotein I.

Tolerogenic antigens may further include, but are not limited to, hInsB₁₀₋₁₈ (HLVEALYLV (SEQ ID NO: 743)), hIGRP₂₂₈₋₂₃₆ (LNIDLLWSV (SEQ ID NO: 744)), hIGRP₂₆₅₋₂₇₃ (VLFGLGFAI (SEQ ID NO: 745)), IGRP₂₀₆₋₂₁₄ (VYLKTNVFL (SEQ ID NO: 746)), NRP-A7 (KYNKANAFL (SEQ ID NO: 747)), NRP-14 (KYNIANVFL (SEQ ID NO: 748)), NRP-V7 (KYNKANVFL (SEQ ID NO: 749)), YAI/db (FQDENYLYL (SEQ ID NO: 750)) and/or INS B15-23 (LYLVCGERG (SEQ ID NO: 751)), as well as peptides and proteins disclosed in U.S. Publication 20050202032.

In certain aspects, a peptide antigen for use in the treatment of type 1 diabetes is GAD65₁₁₄₁₂₃, VMNILLQYVV (SEQ ID NO: 752); GAD65₅₃₆₋₅₄₅, RMMEYGTTMV (SEQ ID NO: 753); GFAP₁₄₃₋₁₅₁, NLAQTDLATV (SEQ ID NO: 754); GFAP₂₁₄₋₂₂₂, QLARQQVHV (SEQ ID NO: 755); IA-2₁₇₂₋₁₈₀, SLSPLQAEL (SEQ ID NO: 756); IA-2₄₈₂₋₄₉₀, SLAAGVKLL (SEQ ID NO: 757); IA-2₈₀₅₋₈₁₃, VIVMLTPLV (SEQ ID NO: 758); ppIAPP₅₋₁₃, KLQVFLIVL (SEQ ID NO: 759); ppIAPP₉₋₁₇, FLIVLSVAL (SEQ ID NO: 760); IGRP₁₅₂₋₁₆₀, FLWSVFMLI (SEQ ID NO: 761); IGRP₂₁₁₋₂₁₉, NLFLFLFAV (SEQ ID NO: 762); IGRP₂₁₅₋₂₂₃, FLFAVGFYL (SEQ ID NO: 763); IGRP₂₂₂₋₂₃₀, YLLLRVLNI (SEQ ID NO: 764); IGRP₂₂₈₋₂₃₆, LNIDLLWSV (SEQ ID NO: 744); IGRP₂₆₅₋₂₇₃, VLFGLGFAI (SEQ ID NO: 745); IGRP₂₉₃₋₃₀₁, RLLCALTSL (SEQ ID NO: 765); Pro-insulin_{L2-10}, ALWMRLLPL (SEQ ID NO: 766); Pro-insulin_{L3-11}, LWMRLLPLL (SEQ ID NO: 767); Pro-insulin_{L6-14}, RLLPLLALL (SEQ ID NO: 768); Pro-insulin_{B5-14}, HLCGSHLVEA (SEQ ID NO: 769); Pro-insulin_{B10-18}, HLVEALYLV (SEQ ID NO: 743); Proinsulin_{B14-22}, ALYLVCGER (SEQ ID NO: 770); Pro-insulin_{B15-24}, LYLVCGERGF (SEQ ID NO: 771); Pro-insulin_{B17-25}, LVCGERGFF (SEQ ID NO: 772); Pro-insulin_{B18-27}, VCGERGFFYT (SEQ ID NO: 773); Pro-insulin_{B20-27}, GERGFFYT (SEQ ID NO: 774); Pro-insulin_{B21-29}, ERGFFYTPK (SEQ ID NO: 775); Pro-insulin_{B25-C1}, FYTPKTRRE (SEQ ID NO: 776); Proinsulin_{B27-C5}, TPKTRREAEDL (SEQ ID NO: 777); Pro-insulin_{C20-28}, SLQPLALEG (SEQ ID NO: 778); Pro-insulin_{C25-33}, ALEGSLQKR (SEQ ID NO: 779); Pro-insulin_{C29-A5}, SLQKRGIVEQ (SEQ ID NO: 780); Pro-insulin_{A1-10}, GIVEQCCTSI (SEQ ID NO: 781); Pro-insulin_{A2-10}, IVEQCCTSI (SEQ ID NO: 782); Pro-insulin_{A12-20}, SLYQLENYC (SEQ ID NO: 783), or combinations thereof.

In still further aspects, tolerogenic antigens associated with multiple sclerosis (MS) can be used and include: MAG₂₈₇₋₂₉₅, SLLLELEEV (SEQ ID NO: 784); MAG₅₀₉₋₅₁₇, LMWAKIGPV (SEQ ID NO: 785); MAG₅₅₆₋₅₆₄, VLFSSDFRI (SEQ ID NO: 786); MBP₁₁₀₋₁₁₈, SLSRFSWGA (SEQ ID NO: 787); MOG₁₁₄₋₁₂₂, KVEDPFYWV (SEQ ID NO: 788); MOG₁₆₆₋₁₇₅, RTFDPHFLRV (SEQ ID NO: 789); MOG₁₇₂₋₁₈₀, FLRVPCWKI (SEQ ID NO: 790); MOG₁₇₉₋₁₈₈, KITLFVIVPV (SEQ ID NO: 791); MOG₁₈₈₋₁₉₆, VLGPLVALI (SEQ ID NO: 792); MOG₁₈₁₋₁₈₉, TLFVIVPVL (SEQ ID NO: 793); MOG₂₀₅₋₂₁₄, RLAGQFLEEL (SEQ ID NO: 794); PLP₈₀₋₈₈, FLYGALLLA (SEQ ID NO: 795), or combinations thereof.

In some instances, tolerogenic antigens associated with systemic lupus erythematosus can be used including, but not limited to, FIEWNKLRFRQGLEW (SEQ ID NO: 796). In some instances, the tolerogenic antigen comprising a polypeptide having the sequence of SEQ ID NO: 796 includes at least one amino acid moiety that is a D-amino acid.

Multimeric Tolerogenic Antigens

In certain embodiments, a tolerogenic antigen provided herein is a multimeric tolerogenic antigen. In one example, a multimeric tolerogenic antigen includes two or more tolerogenic antigens (e.g., a tolerogenic antigen described herein) connected by way or a linker (e.g., a peptide linker). In some instances, the tolerogenic antigen includes the following N-terminal-to-C-terminal structure:

(P₄-L₄)ₙ₄-(P₃-L₃)ₙ₃-P₂-(L₁-P₁)ₙ₁

Wherein P₁, P₂, P₃, and P₄ are each independently selected from any tolerogenic antigen described herein (e.g., any tolerogenic antigen from tables 3-5); L₁, L₃, and L₄ are each independently a linker; and n₁, n₃, and n₄ are each independently 0 or 1, wherein at least one of n₁, n₃, and n₄ are 1.

In some instances, n₁ is 1, n₃ is 0, and n₄ is 0, and the tolerogenic antigen includes the following N-terminal-to-C-terminal structure:

P₂-L₁-P₁.

In some instances, the peptide linker includes between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200)). In some instances, the peptide linker comprises glycine (Gly) and serine (Ser) amino acids. In some instances, the peptide linker includes the amino acid sequence of any one of (GS)ₓ, (GGS)ₓ, (GGGGS (SEQ ID NO: 797))ₓ, (GGSG)ₓ, (SGGG)ₓ, wherein x is an integer from 1 to 10. In certain embodiments the linker includes the amino acid sequence of (GGGGS (SEQ ID NO: 797))ₓ, wherein x is an integer from 2-5. In some instances, P₂ and P₁ are different tolerogenic antigens. In some instances, P₂ and P₁ are identical tolerogenic antigens.

In some instances, n₁ is 1, n₃ is 1, and n₄ is 0, and the tolerogenic antigen comprises the following N-terminal-to-C-terminal structure:

P₃-L₃-P₂-L₁-P₁.

In some instances, each peptide linker independently includes between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200)). In some instances, the peptide linker comprises glycine (Gly) and serine (Ser) amino acids. In some instances, the peptide linker includes the amino acid sequence of any one of (GS)ₓ, (GGS)ₓ, (GGGGS (SEQ ID NO: 797))ₓ, (GGSG)ₓ, (SGGG)ₓ, wherein x is an integer from 1 to 10. In certain embodiments the linker includes the amino acid sequence of (GGGGS (SEQ ID NO: 797))ₓ, wherein x is an integer from 2-5. In some instances, P₃, P₂, and/or P₁ are different tolerogenic antigens. In some instances, P₃, P₂, and/or P₁ are identical tolerogenic antigens.

In some instances, n₁ is 1, n₃ is 1, and n₄ is 1, and the tolerogenic antigen comprises the following N-terminal-to-C-terminal structure:

P₄-L₄-P₃-L₃-P₂-L₁-P₁.

In some instances, each peptide linker independently includes between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200)). In some instances, the peptide linker comprises glycine (Gly) and serine (Ser) amino acids. In some instances, the peptide linker includes the amino acid sequence of any one of (GS)ₓ, (GGS)ₓ, (GGGGS (SEQ ID NO: 797))ₓ, (GGSG)ₓ, (SGGG)ₓ, wherein x is an integer from 1 to 10. In certain embodiments the linker includes the amino acid sequence of (GGGGS (SEQ ID NO: 797))ₓ, wherein x is an integer from 2-5. In some instances, P₄, P₃, P₂, and/or P₁ are different tolerogenic antigens. In some instances, P₄, P₃, P₂, and/or P₁ are identical tolerogenic antigens.

In some embodiments, the tolerogenic antigen is conjugated with the nanoparticle phospholipid in such a manner that facilitates strong immune tolerance upon administration to a subject (e.g., a human subject suffering from or at risk of suffering from an autoimmune disorder (e.g., MS or celiac disease)).

In some embodiments, the tolerogenic antigen is conjugated with the nanoparticle phospholipid via a thiol-reactive and reduction-insensitive linkage between the tolerogenic antigen and the nanoparticle phospholipid. Indeed, a thiol-reactive and reduction-insensitive linkage between the tolerogenic antigen and the nanoparticle phospholipid facilitates strong immune tolerance. In some embodiments, the phospholipid is e.g., N-(3-Maleimide-1-oxopropyl)-L-α-phosphatidylethanolamine.

In some embodiments, the tolerogenic antigen is conjugated with the nanoparticle phospholipid via an amine-mediated interaction. For example, in some embodiments, the amine-mediated interaction is through an amine-reactive phospholipid (e.g., N-(Succinimidyloxy-glutaryl)-L-α-phosphatidylethanolamine, Dioleoyl (DOPE-NHS)). In some embodiments, the amine-mediated interaction is through an amine-reactive phospholipid with self-immolative linkage (e.g., linkers including o-dithiobenzyl, p-dithiobenzyl, beta-dithiobenzyl carbamate moieties, 2,2-dimethyl-4-mercapto-butyric acid, or Disulfide-carbonate-based traceless linker).

In some embodiments, the number of tolerogenic antigens associated with a specific nanoparticle is any amount that facilitates strong immune tolerance upon administration to a subject (e.g., a human subject suffering from or at risk of suffering from an autoimmune disorder (e.g., MS or celiac disease). In some embodiments, the amount of tolerogenic antigens associated with a specific nanoparticle is between 1 and 30 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30).

Tolerogenic antigens can be prepared by a number of techniques known in the art, depending on that nature of the molecule. Polynucleotide, polypeptide, and carbohydrate antigens can be isolated from cells of the species to be treated in which they are enriched. Short peptides are conveniently prepared by amino acid synthesis. Longer proteins of known sequence can be prepared by synthesizing an encoding sequence or PCR-amplifying an encoding sequence from a natural source or vector, and then expressing the encoding sequence in a suitable bacterial or eukaryotic host cell.

### Linkers

In some embodiments of the composition described herein, the nanoparticle with a plurality of tolerogenic antigens includes a linker between the tolerogenic antigens and the nanoparticle. In some embodiments, the linker refers to a covalent linkage or connection between the tolerogenic antigen and a phospholipid group of the nanoparticle. In some embodiments, the N-terminus and/or C-terminus of the tolerogenic antigen is modified with a terminal cysteine residue, which is attached to a linker. In some embodiments, the N-terminus and/or C-terminus of the tolerogenic antigen is modified with a terminal C(S)ₙ polypeptide, wherein n is between 1-10 (e.g., 2, 3, 4, 5, 6, 7, 8, 9, or 10) serine residues, and wherein the terminal serine residue is attached to a linker. In some embodiments, the N-terminus and/or C-terminus of the tolerogenic antigen is modified with a terminal CSS polypeptide, which is attached to a linker. In some embodiments, the linker is a thiol reactive crosslinker. In some embodiments, the linker is a maleimide linker. In some embodiments, the linker is a pyridyl linker. In some embodiments, the N-terminus and/or C-terminus of the tolerogenic antigen is modified with a terminal cysteine residue, which is attached to a maleimide linker. In some embodiments, the N-terminus and/or C-terminus of the tolerogenic antigen is modified with a terminal cysteine residue, which is attached to a pyridyl linker. In some embodiments, the N-terminus and/or C-terminus of the tolerogenic antigen is modified with a terminal CSS polypeptide, which is attached to a maleimide linker. In some embodiments, the N-terminus and/or C-terminus of the tolerogenic antigen is modified with a terminal CSS polypeptide, which is attached to a pyridyl linker. The linker may be attached to a modified nucleoside or nucleotide (e.g., Cys and Ser) on the nucleobase or sugar moiety at a first end and to a payload, e.g., a lipid e.g., a phospholipid, at a second end.

Linkers may be chemical linkers, which are known to one of skill in the art. Linkers may alternately be peptide linkers. The linker may be of sufficient length as to not interfere with a polypeptide sequence or a lipid moiety. Examples of chemical groups that can be incorporated into the linker include, but are not limited to, alkyl, alkenyl, alkynyl, amido, amino, ether, thioether, ester, alkylene, heteroalkylene, aryl, or heterocyclyl groups, each of which can be optionally substituted. A linker may include a synthetic group derived from, e.g., a synthetic polymer (e.g., a polyethylene glycol (PEG) polymer). In some embodiments, a linker may include one or more amino acid residues, such as D- or L-amino acid residues. Further examples of useful linkers include those that contain electrophiles, such as Michael acceptors (e.g., maleimides), activated esters, electron-deficient carbonyl compounds, and aldehydes, among others, suitable for reaction with nucleophilic substituents present within antibodies, antigen-binding fragments, proteins, peptides, and small molecules, such as amine and thiol moieties.

In the present invention, a linker between multimeric tolerogenic antigens (e.g. L₁, L₃, and/or L₄) can be polypeptide including between 2 and 200 amino acids (e.g., between 5 and 50 (e.g., between 5 and 20, 15 and 30, 25 and 40, or 35 and 50), between 45 and 100 (e.g., between 45 and 60, 55 and 70, 65 and 80, 75 and 90, or 85 and 100), 95 and 150 (e.g., between 95 and 110, 105 and 120, 115 and 130, 125 and 140, or 135 and 150), or 145 and 200 amino acids (e.g., between 145 and 160, 155 and 170, 165 and 180, 175 and 190, or 185 and 200)). In some embodiments, a linker between multimeric tolerogenic antigens (e.g. L₁, L₃, and/or L₄) is a polypeptide containing at least 12 amino acids, such as 12-200 amino acids (e.g., 12-200, 12-180, 12-160, 12-140, 12-120, 12-100, 12-90, 12-80, 12-70, 12-60, 12-50, 12-40, 12-30, 12-20, 12-19, 12-18, 12-17, 12-16, 12-15, 12-14, or 12-13 amino acids) (e.g., 14-200, 16-200, 18-200, 20-200, 30-200, 40-200, 50-200, 60-200, 70-200, 80-200, 90-200, 100-200, 120-200, 140-200, 160-200, 180-200, or 190-200 amino acids). In some embodiments, a linker between multimeric tolerogenic antigens (e.g. L₁, L₃, and/or L₄) is a polypeptide containing 12-30 amino acids (e.g., 12, 13, 14, 15, 16, 17, 18, 19, 20, 21, 22, 23, 24, 25, 26, 27, 28, 29, or 30 amino acids).

Suitable peptide linkers are known in the art, and include, for example, peptide linkers containing flexible amino acid residues such as glycine and serine. In certain embodiments, a linker can contain motifs, e.g., multiple or repeating motifs, of GS, GGS, GGGGS (SEQ ID NO: 797), GGSG (SEQ ID NO: 798), or SGGG (SEQ ID NO: 799). In certain embodiments, a linker can contain 2 to 12 amino acids including motifs of GS, e.g., GS, GSGS (SEQ ID NO: 800), GSGSGS (SEQ ID NO: 801), GSGSGSGS (SEQ ID NO: 802), GSGSGSGSGS (SEQ ID NO: 803), or GSGSGSGSGSGS (SEQ ID NO: 804). In certain other embodiments, a linker can contain 3 to 12 amino acids including motifs of GGS, e.g., GGS, GGSGGS (SEQ ID NO: 805), GGSGGSGGS (SEQ ID NO: 806), and GGSGGSGGSGGS (SEQ ID NO: 807). In yet other embodiments, a linker can contain 4 to 12 amino acids including motifs of GGSG (SEQ ID NO: 808), e.g., GGSGGGSG (SEQ ID NO: 809), or GGSGGGSGGGSG (SEQ ID NO: 810). In other embodiments, a linker can contain motifs of GGGGS (SEQ ID NO: 797), e.g., GGGGSGGGGSGGGGS (SEQ ID NO:811). In certain embodiments, a linker is SGGGSGGGSGGGSGGGSGGG (SEQ ID NO: 812).

In preferred embodiments, a peptide linker (e.g., L₁, L₃, and/or L₄) is a peptide linker including the amino acid sequence of any one of (GS)ₓ, (GGS)ₓ, (GGGGS)ₓ, (GGSG)ₓ, (SGGG)ₓ, wherein x is an integer from 1 to 50 (e.g., 1-40, 1-30, 1-20, 1-10, or 1-5). In preferred embodiments, the peptide linker has the amino acid sequence (GGGGS)ₓ, wherein x is an integer selected from 1, 2, 3, 4, 5, 6, 7, 8, 9, or 10.

In some embodiments, a peptide linker contains only glycine residues, e.g., at least 4 glycine residues (e.g., 4-200, 4-180, 4-160, 4-140, 4-40, 4-100, 4-90, 4-80, 4-70, 4-60, 4-50, 4-40, 4-30, 4-20, 4-19, 4-18, 4-17, 4-16, 4-15, 4-14, 4-13, 4-12, 4-11, 4-10, 4-9, 4-8, 4-7, 4-6 or 4-5 glycine residues) (e.g., 4-200, 6-200, 8-200, 10-200, 12-200, 14-200, 16-200, 18-200, 20-200, 30-200, 40-200, 50-200, 60-200, 70-200, 80-200, 90-200, 100-200, 120-200, 140-200, 160-200, 180-200, or 190-200 glycine residues). In certain embodiments, a linker has 4-30 glycine residues (e.g., 4, 5, 6, 7, 8, 9, 10, 11, 12, 13, 14, 15, 16, 17, 18, 19, 20, 21, 22, 23, 24, 25, 26, 27, 28, 29, or 30 glycine residues). In some embodiments, a linker containing only glycine residues may not be glycosylated (e.g., O-linked glycosylation, also referred to as O-glycosylation) or may have a decreased level of glycosylation (e.g., a decreased level of O-glycosylation) (e.g., a decreased level of O-glycosylation with glycans such as xylose, mannose, sialic acids, fucose (Fuc), and/or galactose (Gal) (e.g., xylose)) as compared to, e.g., a linker containing one or more serine residues.

In some embodiments, a linker containing only glycine residues may not be O-glycosylated (e.g., O-xylosylation) or may have a decreased level of O-glycosylation (e.g., a decreased level of O-xylosylation) as compared to, e.g., a linker containing one or more serine residues.

In some embodiments, a linker containing only glycine residues may not undergo proteolysis or may have a decreased rate of proteolysis as compared to, e.g., a linker containing one or more serine residues.

In certain embodiments, a linker can contain motifs of GGGG (SEQ ID NO: 813), e.g., GGGGGGGG (SEQ ID NO: 814), GGGGGGGGGGGG (SEQ ID NO: 815), GGGGGGGGGGGGGGGG (SEQ ID NO: 816), or GGGGGGGGGGGGGGGGGGGG (SEQ ID NO: 817). In certain embodiments, a linker can contain motifs of GGGGG (SEQ ID NO: 818), e.g., GGGGGGGGGG (SEQ ID NO: 819), GGGGGGGGGGGGGGG (SEQ ID NO: 820, or GGGGGGGGGGGGGGGGGGGG (SEQ ID NO: 821).

In certain embodiments, a linker is GGGGGGGGGGGGGGGGGGGG (SEQ ID NO: 822).

In other embodiments, a linker can also contain amino acids other than I cine and serine, e.g.,

(SEQ ID NO: 823)
GENLYFQSGG,
(SEQ ID NO: 757)
SACYCELS,
(SEQ ID NO: 824)
RSIAT,
(SEQ ID NO: 825)
RPACKIPNDLKQKVMNH,
(SEQ ID NO: 826)
GGSAGGSGSGSSGGSSGASGTGTAGGTGSGSGTGSG,
(SEQ ID NO: 827)
AAANSSIDLISVPVDSR,
or
(SEQ ID NO: 828)
GGSGGGSEGGGSEGGGSEGGGSEGGGSEGGGSGGGS.

Tolerogenic Antigen Variants

In certain embodiments, amino acid sequence variants of the tolerogenic antigens of the invention are contemplated. For example, it may be desirable to improve the tolerogenic antigenicity and/or other biological properties of the tolerogenic antigens. Amino acid sequence variants of a tolerogenic antigen may be prepared by introducing appropriate modifications into the nucleotide sequence encoding the tolerogenic antigen, or by peptide synthesis. Such modifications include, for example, deletions from, and/or insertions into and/or substitutions of residues within the amino acid sequences of the tolerogenic antigens. Any combination of deletion, insertion, and substitution can be made to arrive at the final construct, provided that the final construct possesses the desired characteristics, for example, inducing antigen tolerance.

In certain embodiments, tolerogenic antigen variants having one or more amino acid substitutions are provided. Conservative substitutions are shown in Table 6 under the heading of “preferred substitutions.” More substantial changes are provided in Table 6 under the heading of “exemplary substitutions,” and as further described below in reference to amino acid side chain classes. Amino acid substitutions may be introduced into a tolerogenic antigen of interest and the products screened for a desired activity, for example, retained/improved tolerogenic antigenicity.

TABLE 6
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, lie;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

A useful method for identification of residues or regions of a tolerogenic antigen that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, And Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Tolerogenic antigen variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues.

### Methods of Treating Autoimmune Disorders

As noted, in certain embodiments, the present invention provides methods for treating an autoimmune disorder (e.g., MS celiac disease) through administering to a subject (e.g., a human subject suffering from or at risk of suffering from an autoimmune disorder e.g., celiac disease) a composition comprising nanoparticles associated with tolerogenic antigens implicated in autoimmune disease (e.g., MS or celiac disease) via a linkage (e.g., between 1-30 tolerogenic antigens per nanoparticle), as described herein.

The immune system can be classified into two functional subsystems: the innate and the acquired immune system. The innate immune system is the first line of defense against infections, and most potential pathogens are rapidly neutralized by this system before they can cause, for example, a noticeable infection. The acquired immune system reacts to molecular structures, referred to as antigens, of the intruding organism. There are two types of acquired immune reactions, which include the humoral immune reaction and the cell-mediated immune reaction. In the humoral immune reaction, antibodies secreted by B cells into bodily fluids bind to pathogen-derived antigens, leading to the elimination of the pathogen through a variety of mechanisms, e.g. complement-mediated lysis. In the cell-mediated immune reaction, T-cells capable of destroying other cells are activated. For example, if proteins associated with a disease (e.g., MS or celiac disease) are present in a cell, they are fragmented proteolytically to peptides within the cell. Specific cell proteins then attach themselves to the antigen or peptide formed in this manner and transport them to the surface of the cell, where they are presented to the molecular defense mechanisms, in particular T-cells, of the body. Cytotoxic T cells recognize these antigens and kill the cells that harbor the antigens.

The molecules that transport and present peptides on the cell surface are referred to as proteins of the major histocompatibility complex (MHC), which is known as the human leukocyte antigen (HLA) complex in humans. MHC proteins are classified into two types, referred to as MHC class I and MHC class II. The structures of the proteins of the two MHC classes are very similar; however, they have very different functions. Proteins of MHC class I are present on the surface of almost all cells of the body, including most tumor cells. MHC class I proteins are loaded with antigens that usually originate from endogenous proteins or from pathogens present inside cells and are then presented to naive or cytotoxic T-lymphocytes (CTLs). MHC class II proteins are present on dendritic cells, B-lymphocytes, macrophages, and other antigen-presenting cells. They mainly present peptides, which are processed from external antigen sources, i.e. outside of the cells, to T-helper (Th) cells. Most of the peptides bound by the MHC class I proteins originate from cytoplasmic proteins produced in the healthy host cells of an organism itself and do not normally stimulate an immune reaction. Accordingly, cytotoxic T-lymphocytes that recognize such self-peptide-presenting MHC molecules of class I are deleted in the thymus (central tolerance) or, after their release from the thymus, are deleted or inactivated, i.e. tolerized (peripheral tolerance). MHC molecules are capable of stimulating an immune reaction when they present peptides to non-tolerized T-lymphocytes. Cytotoxic T-lymphocytes have both T-cell receptors (TCR) and CD8 molecules on their surface. T-Cell receptors are capable of recognizing and binding peptides complexed with the molecules of MHC class I. Each cytotoxic T-lymphocyte expresses a unique T-cell receptor which is capable of binding specific MHC/peptide complexes.

In some embodiments, the compositions and methods described herein for treating celiac disease including nanoparticles associated with a plurality of tolerogenic antigens (e.g., between 1-30 tolerogenic antigens per particle) implicated in celiac disease. In the case of celiac disease, the MHC class II protein haplotypes, HLA-DQ2 and HLA-DQ8, have been implicated in the pathogenesis of the disease. While having the HLA-DQ2 and HLA-DQ8 haplotypes does not necessitate that the subject also has celiac disease, these haplotypes are required for celiac disease to occur. The reason for this is that HLA-DQ2 and HLA-DQ8 have a greater affinity for deamidated polypeptides. The 33-mer gliadin polypeptide is deamidated by the TG2 enzyme, and, therefore, the deamidated 33-mer gliadin polypeptide binds to HLA-DQ2 or HLA-DQ8 to form the HLA-DQ2-gliadin or HLA-DQ8-gliadin complex. This complex activates the host-gluten-specific CD₄⁺ T-cells, which stimulate B-cells, which then produce anti-gliadin and anti-TG2 antibodies. T-cell activation causes cytokine production, leading to inflammation and damage to the small intestine, along with an increase in IFNγ production, leading to mucosal intestinal legions.

To prevent an autoimmune response, the tolerogenic antigens attach themselves to the HLA-DQ2 or HLA-DQ8 by competitive affinity binding within the endoplasmic reticulum, before they are presented on the cell surface. Here, the affinity of an individual peptide antigen is directly linked to its amino acid sequence and the presence of specific binding motifs in defined positions within the amino acid sequence. If the sequence of such a peptide is known, it is possible to manipulate the immune system against diseased cells using, for example, peptide vaccines.

In some embodiments of any of the methods described herein, the subject suffering from an autoimmune disease (e.g., celiac disease) adheres to a strict, gluten-free diet. In some embodiments of any of the methods described herein, the subject suffering from celiac disease does not adhere to a gluten-free diet.

Furthermore, experiments conducted during the course of developing embodiments for the present invention determined that inverse vaccination with nanoparticles carrying tolerogenic antigens linked (e.g., by way of a thiol-reactive or a self-immolative linker) with lipid moieties is an effective strategy for treatment of autoimmune conditions (e.g., MS). It was further demonstrated that such therapeutic compositions are optimized with a ratio of 1-30 (e.g., 6-30, 7-30, or 8-30) tolerogenic antigens per nanoparticle.

Such methods are not limited to treating a specific type of autoimmune disorder. Examples of such autoimmune disorders include, but are not limited to, rheumatoid arthritis, multiple sclerosis diabetes (e.g., type 1 diabetes mellitus), autoimmune diseases of the thyroid (e.g., Hashimoto's thyroiditis, Graves' disease), thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, and inflammatory bowel disease.

In some embodiments, such methods for treating or preventing autoimmune disorders further comprise administering (e.g., simultaneously or at different times) additional therapeutic agents. Examples of such therapeutic agents include, but are not limited to, disease-modifying antirheumatic drugs (e.g., leflunomide, methotrexate, sulfasalazine, hydroxychloroquine), biologic agents (e.g., rituximab, infliximab, etanercept, adalimumab, golimumab), nonsteroidal anti-inflammatory drugs (e.g., ibuprofen, celecoxib, ketoprofen, naproxen, piroxicam, diclofenac), analgesics (e.g., acetaminophen, tramadol), immunomodulators (e.g., anakinra, abatacept), glucocorticoids (e.g., prednisone, methylprednisone), TNF-α inhibitors (e.g., adalimumab, certolizumab pegol, etanercept, golimumab, infliximab), IL-1 inhibitors, and metalloprotease inhibitors. In some embodiments, the therapeutic agents include, but are not limited to, infliximab, adalimumab, etanercept, or parenteral gold or oral gold. In some instances, the therapeutic agent is an immunomodulatory agent or immunosuppressant (e.g., statins; mTOR inhibitors, such as rapamycin or a rapamycin analog; TGF-β signaling agents; TGF-β receptor agonists; histone deacetylase inhibitors, such as Trichostatin A; corticosteroids; inhibitors of mitochondrial function, such as rotenone; P38 inhibitors; NF-κβ inhibitors, such as 6Bio, Dexamethasone, TCPA-1, IKK VII; adenosine receptor agonists; prostaglandin E2 agonists (PGE2), such as Misoprostol; phosphodiesterase inhibitors, such as phosphodiesterase 4 inhibitor (PDE4), such as Rolipram; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors, such as TGX-221; autophagy inhibitors, such as 3-Methyladenine; aryl hydrocarbon receptor inhibitors; proteasome inhibitor I (PSI); and oxidized ATPs, such as P2X receptor blockers. Immunosuppressants also include IDO, vitamin D3, cyclosporins, such as cyclosporine A, aryl hydrocarbon receptor inhibitors, resveratrol, azathiopurine (Aza), 6-mercaptopurine (6-MP), 6-thioguanine (6-TG), FK506, sanglifehrin A, salmeterol, mycophenolate mofetil (MMF), aspirin and other COX inhibitors, niflumic acid, estriol, triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNT02424; TH1020; INH-ODN; E6446; AT791; CpG ODN 2088; ODN TTAGGG; COV08-0064; 2R9; GpG oligonucleotides; 2-aminopurine; Amlexanox; Bay11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ)). In particular embodiments, the immunosuppressant is fingolimod; 2-(1′H-indole-3′-carbonyl)-thiazole-4-carboxylic acid methyl ester (ITE) or related ligands; Trichostatin A; and/or Suberoylanilide hydroxamic acid (SAHA).

Such methods are not limited to a particular manner of administering the composition comprising nanoparticles associated with tolerogenic antigens. Indeed, any acceptable method known to one of ordinary skill in the art may be used to administer such a composition to the subject. The administration may be localized (i.e., to a particular region, physiological system, tissue, organ, or cell type) or systemic. Such compositions can be administered by a number of routes including, but not limited to oral, inhalation (nasal or pulmonary), intravenous, intraperitoneal, intramuscular, transdermal, subcutaneous, topical, sublingual, or rectal means. Injections can be e.g., intravenous, intradermal, subcutaneous, intramuscular, or intraperitoneal. In some embodiments, the injections can be given at multiple locations.

Administration of the formulations may be accomplished by any acceptable method which allows an effective amount of the composition to achieve its desired effect. The particular mode selected will depend upon factors such as the particular formulation, the severity of the state of the subject being treated, and the dosage required to induce an effective immune response. As generally used herein, an “effective amount” is that amount which is able to induce an immune response in the treated subject. The actual effective amounts of such compositions can vary according to the specific antigen or combination thereof being utilized, the particular composition formulated, the mode of administration, and the age, weight, condition of the individual being vaccinated, as well as the route of administration and the disease or disorder.

### Nanoparticle Characterization

The nanoparticles of the present invention may be characterized for size and uniformity by any suitable analytical techniques. These include, but are not limited to, atomic force microscopy (AFM), electrospray-ionization mass spectroscopy, MALDI-TOF mass spectroscopy, LC-MS/MS, ¹³C nuclear magnetic resonance spectroscopy, high performance liquid chromatography (HPLC), size exclusion chromatography (SEC) (equipped with multi-angle laser light scattering, dual UV and refractive index detectors), capillary electrophoresis, and get electrophoresis. These analytical methods assure the uniformity of the sHDL nanoparticle population and are important in the production quality control for eventual use in in vivo applications.

In some embodiments, gel permeation chromatography (GPC), which can separate sHDL nanoparticles from liposomes and free ApoA-I mimetic peptide, is used to analyze the sHDL-Tolerogenic antigen nanoparticles. In some embodiments, the size distribution and zeta-potential are determined by dynamic light scattering (DLS) using, for example, a Malven Nanosizer instrument.

### Pharmaceutical Compositions

Where clinical applications are contemplated, in some embodiments of the present invention, the sHDL nanoparticles are prepared as part of a pharmaceutical composition in a form appropriate for the intended application. Generally, this entails preparing compositions that are essentially free of pyrogens, as well as other impurities that could be harmful to humans or animals. However, in some embodiments of the present invention, a straight sHDL nanoparticle formulation may be administered using one or more of the routes described herein.

In preferred embodiments, the sHDL nanoparticles are used in conjunction with appropriate salts and buffers to render delivery of the compositions in a stable manner to allow for uptake by target cells. Buffers also are employed when the sHDL nanoparticles are introduced into a patient. Aqueous compositions comprise an effective amount of the sHDL nanoparticles to cells dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. Except insofar as any conventional media or agent is incompatible with the vectors or cells of the present invention, its use in therapeutic compositions is contemplated. Supplementary active ingredients may also be incorporated into the compositions.

In some embodiments of the present invention, the active compositions include classic pharmaceutical preparations. Administration of these compositions according to the present invention is via any common route so long as the target tissue is available via that route. This includes oral, nasal, buccal, rectal, vaginal, or topical. Alternatively, administration may be by orthotopic, intradermal, subcutaneous, intramuscular, intraperitoneal, or intravenous injection.

The active sHDL nanoparticles may also be administered parenterally or intraperitoneally or intratumorally. Solutions of the active compounds as free base or pharmacologically acceptable salts are prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it may be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active sHDL nanoparticles in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, sHDL nanoparticles are administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution is suitably buffered, if necessary, and the liquid diluent first rendered isotonic with sufficient saline or glucose. These particular aqueous solutions are especially suitable for intravenous, intramuscular, subcutaneous, and intraperitoneal administration. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). In some embodiments of the present invention, the active particles or agents are formulated within a therapeutic mixture to comprise about 0.0001 to 1.0 milligrams, or about 0.001 to 0.1 milligrams, or about 0.1 to 1.0 or even about 10 milligrams per dose or so. Multiple doses may be administered.

Additional formulations that are suitable for other modes of administration include vaginal suppositories and pessaries. A rectal pessary or suppository may also be used. Suppositories are solid dosage forms of various weights and shapes, usually medicated, for insertion into the rectum, vagina, or the urethra. After insertion, suppositories soften, melt or dissolve in the cavity fluids. In general, for suppositories, traditional binders and carriers may include, for example, polyalkylene glycols or triglycerides; such suppositories may be formed from mixtures containing the active ingredient in the range of 0.5% to 10%, preferably 1%-2%. Vaginal suppositories or pessaries are usually globular or oviform and weighing about 5 g each. Vaginal medications are available in a variety of physical forms, e.g., creams, gels, or liquids, which depart from the classical concept of suppositories. The sHDL nanoparticles also may be formulated as inhalants.

### Kits

In some embodiments, the present invention also provides kits comprising compositions comprising nanoparticles associated with tolerogenic antigens via a thiol-reactive and reduction-insensitive linkage as described herein. In some embodiments, the kits comprise one or more of the reagents and tools necessary to generate sHDL nanoparticles, and methods of using such sHDL nanoparticles.

### Examples

The following example is provided in order to demonstrate and further illustrate certain preferred embodiments and aspects of the present invention and are not to be construed as limiting the scope thereof.

### Example 1. Production of sHDL Nanodsic with Myelin-Based Peptides for the Treatment of Subjects with Multiple Sclerosis

This example demonstrates that myelin-based peptide-conjugated nanodiscs promote immunological tolerance against multiple sclerosis/experimental autoimmune encephalomyelitis (EAE).

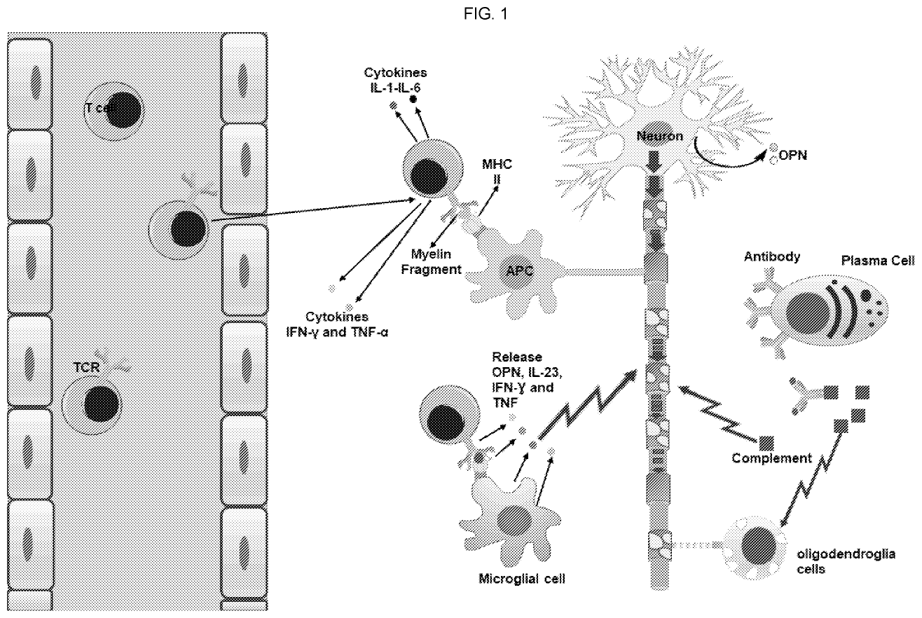

Multiple sclerosis (MS) is an autoimmune disease caused by the autoimmune response against axons and myelin sheaths of the central nervous system (CNS), leading to axonal loss and demyelination (FIG. 1). Current treatments for MS are mainly based on immunosuppressive therapies that have unintended side effects on global immune responses and cause significant toxicity. The following experiments were conducted to develop a delivery platform that can efficiently deliver MS antigens in a tolerogenic manner. In summary, such experiments generated Myelin Oligodendrocyte Glycoprotein (MOG) peptide antigen into synthetic high density lipoprotein (HDL) nanodisc, and demonstrated that HDL-MOG can exert potent efficacy against in experimental autoimmune encephalomyelitis (EAE), which is a widely used murine model of MS.

HDL nanodisc was prepared as previously described. Briefly, DMPC was dissolved in chloroform, which was evaporated with nitrogen flow and under vacuum for at least 1 h. The resulting lipid film was hydrated in 10 mM sodium phosphate buffer and sonicated in a bath sonicator for 10 min. ApoA1 mimetic peptide 22A dissolved in endotoxin free water was added to the above mixture (22A:DMPC=1:2 weight ratio) to obtain nanodiscs. To load MOG antigen peptide in nanodiscs, each antigen peptide was reacted with 4-(4-maleimidophenyl)-butyric DOPE for 3 h in dimethylformamide (DMF), which was removed by freeze-drying after dilution 10× with endotoxin-free water. The lipid-peptide conjugates were dissolved in DMSO and added to pre-formed sHDL and incubated for 30 min at room temperature. Unreacted MOG peptides were removed by using Zeba Spin Desalting columns (Pierce) following the manufacturer's instructions. The conjugation efficiency of MOG peptide was determined by LC-MS and gel permeation chromatography (GPC). EAE was induced in female C57BL/6 mice by inoculation with 100 μg of MOG in complete Freund's adjuvant on day 0 following the administration of pertussis toxin. Mice were then injected subcutaneously at tail base for therapeutic study with sHDL-MOG, MOG or PBS on indicated days. Mice were weighed and scored daily based on the following guideline: 0, no sign of disease; 1, loss of tone in the tail; 2, hind limb paresis; 3, hind limb paralysis; 4, tetraplegia; and 5, moribund.

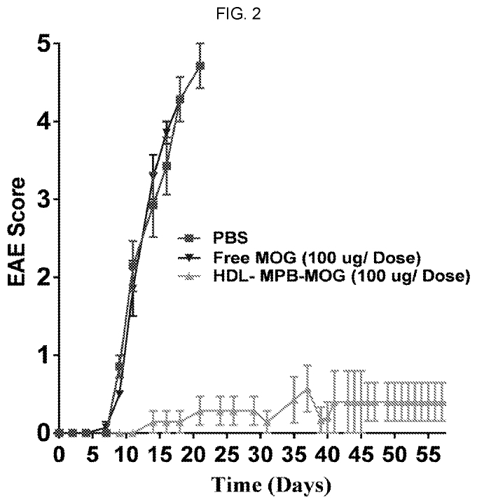

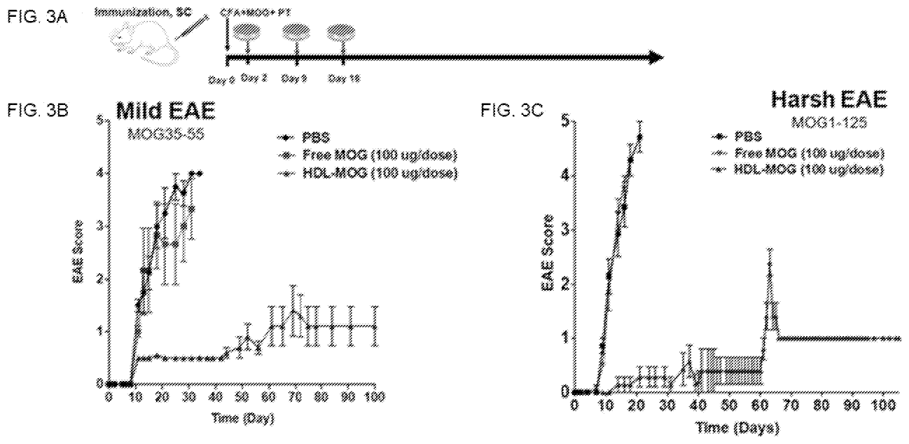

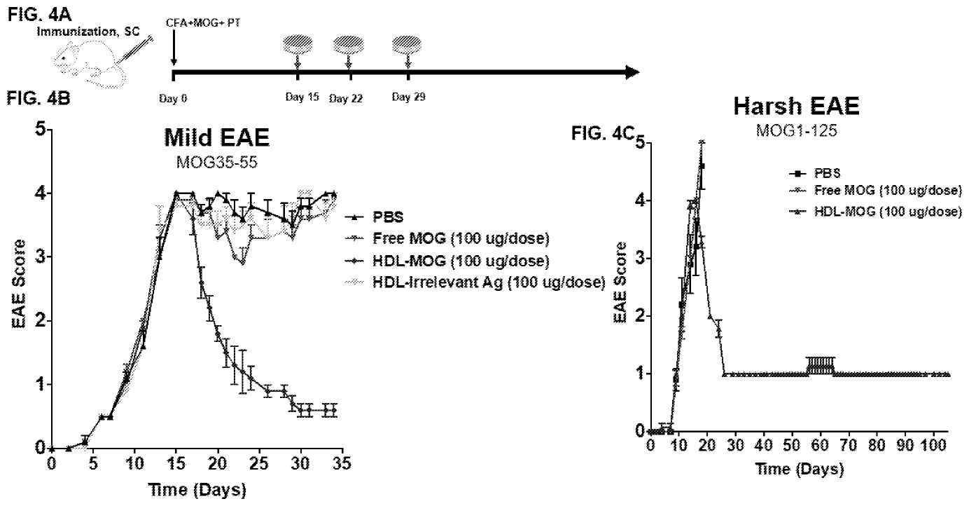

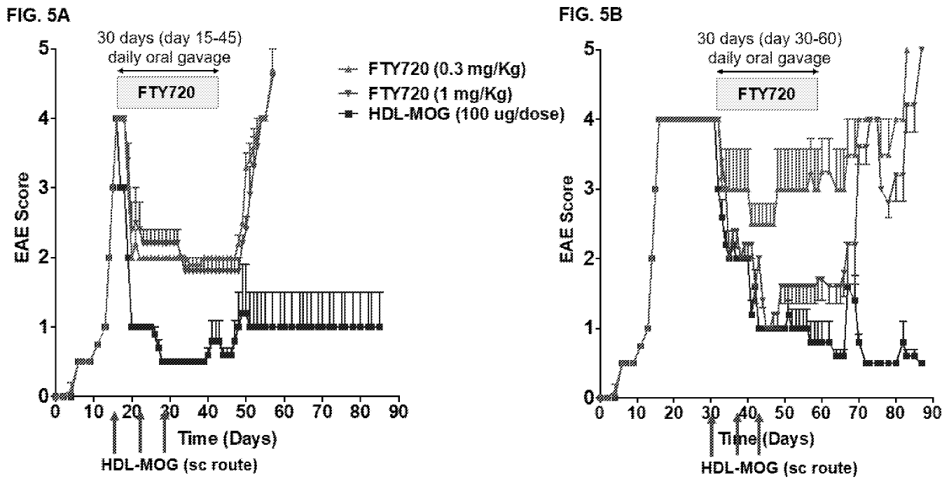

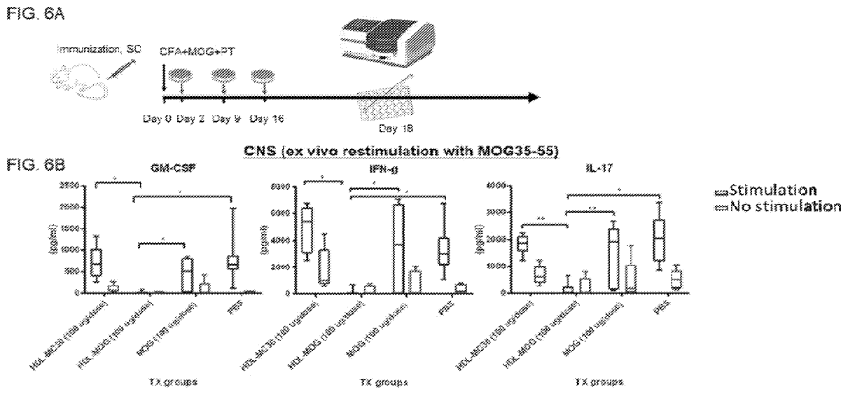

MOG-modified sHDL exhibited uniform disc-like morphology with an average diameter of 11±1 nm. The loading efficiency of MOG35-55 in HDL was ˜90% as quantified by HPLC/MS. Experiments first induced EAE on day 0 using either MOG35-55 (“mild EAE”) or MOG1-125 (“harsh EAE”), and treated animals on days 2, 9, and 16 (FIGS. 2 and 3A). SC administration of HDL-MOG significantly inhibited the symptoms of EAE, with the average pathological scores remaining below 1 for both mild and harsh EAE conditions (FIGS. 2, 3B, and 3C). On the other hand, mice administered with PBS or free MOG peptide were moribund within 20 days, with all animals progressing to the pathological scores of 5 (FIGS. 3B and 3C). Next, experiments were conducted that induced EAE on day 0 and delayed HDL-MOG treatments until day 15 when mice exhibited pathological scores of 4 (FIG. 4A). HDL-MOG treatments given s.c. on days 15, 22, and 29 significantly reversed the symptoms of EAE with mice showing the EAE score of 1 by day 35 for both mild and harsh EAE conditions (FIGS. 4B and 4C). Next, experiments directly compared the efficacy of HDL-MOG to FTY720, also known as Fingolimod, which is a FDA-approved therapeutic widely used by MS patients. FTY720 was orally administered daily for 30 days, starting either day 15 (FIG. 5A) or day 30 (FIG. 5B). When HDL-MOG was given s.c. on days 15, 22, and 29, mice exhibited significantly reduced EAE symptoms and scores remained below 1 for 85 days (FIG. 5A). In contrast, mice treated for 30 days with FTY720 had EAE scores remaining above 2, but when FTY720 treatment stopped, mice showed severe EAE symptoms and had to be euthanized by day 55 (FIG. 5A). Similarly, when HDL-MOG was given s.c. on days 30, 37, and 44, mice exhibited significantly reduced EAE symptoms and scores remained below 1 for 85 days (FIG. 5A). In contrast, mice treated for 30 days with FTY720 starting day 30 had decreased EAE score to 3 and 1 for FTY720 dose of 0.3 mg/Kg and 1 mg/Kg, respectively (FIG. 5B). However, when FTY720 treatment stopped, mice showed severe EAE symptoms and had to be euthanized by day 90. Lastly, this mechanistic study showed that HDL-MOG treatment decreased the production of IFN-γ and IL-17 among lymphocytes located in the central nervous system, compared with treatment with free MOG peptide (FIG. 6), suggesting that this HDL-based strategy may have inhibited the autoantigen-specific Th1 and Th17 responses known to be critical in the pathogenesis of EAE and MS.

### Example 2. Visualized Cellular Uptake of HDL-MOG or Free MOG Peptide by Bone Marrow-Derived Dendritic Cells (BMDCs) and Microglia

To obtain bone marrow-derived dendritic cells (BMDCs), bone marrow was flushed from femurs and tibia bone of 5 to 6 weeks old C57BL/6 mice. Bone marrow cells were plated at 1×10⁶ cells per dish in RPMI 1640 supplemented with 10% FBS, 55 μM β-mercaptoethanol, 5 ng/ml of GM-CSF, and 100 U/ml penicillin (BMDC media). On day three and five, one half of the culture media was replaced with fresh media. After 8 days, BMDCs were harvested and plated at 1×10⁶ cells per well in 12-well plates.

Mixed glial cell cultures were harvested from the cerebral cortex of newborn (P0-2) C57BL6/J mice as described previously. Cortices and brain stem were separated, and the blood vessels and meninges were carefully isolated. Then, the tissues were digested by enzymatic dissociation (0.05% trypsin-EDTA and 25 mg/ml DNase I), washed two times with 1% BSA in PBS. Cells were resuspended in DMEM (10% FBS, 1% penicillin/streptomycin, 0.5 mM 2-mercaptoethanol). Mixed glial cells were cultured in poly-D-lysine-coated flasks and grown at 37° C. and 5% CO2. After 10 days, microglia cells were isolated from the underlying astrocytic layer by shaking of the flask. The cell culture contained more than 95% microglial (CD11 b+, CD45+) cells as analyzed by flow cytometry.

Internalization of fluorescent HDL-MOG by BMDCs or microglia were visualized using confocal microscopy. BMDCs or microglia were seeded at 1×10⁶ cells on 35 mm Petri dishes (MatTek) and incubated with the mixture of free CSS-MOG-K(FITC), or sHDL-CSS-MOG-K(FITC). Cells were then washed three times with PBS, fixed with 4% paraformaldehyde, washed, and permeabilized with 0.1% Triton-X solution. Actin filaments were stained with AlexaFluor 647-Phalloidin, MHC-II was stained with MHC-II-Texas red, and the nuclei were stained with DAPI. The samples were imaged using a 63× oil-immersion lens on a Nikon A-I spectral confocal microscope.

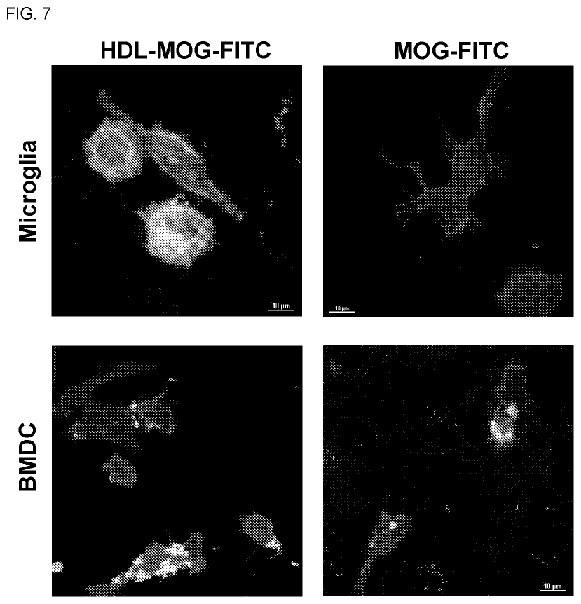

As shown in FIG. 7 BMDCs and microglia avidly internalized HDL-MOG-FITC. In contrast, we observed minimal signal of free MOG-FITC peptide in BMDCs and microglia. This suggest that APCs, such as DCs and microglia, phagocytose sHDL-MOG with high efficiency.

### Example 3. Biodistribution of HDL-MOG and Free MOG Peptide after SC Administration in Mice

Tetramethylrhodamine (TMR, excitation/emission ˜540/560 nm) modified MOG peptide (CSSGWYRSPFSRVVHL-TMR, MOG-TMR SEQ ID NO: 829) was prepared by reacting TMR-NHS and MOG peptide according to the manufacturer's instruction. MOG-TMR was purified using HPLC and reacted with DOPE-MAL in DMF to produce DOPE-Mal-MOG-TMR (MOG-TMR). Next, the DMF solution was diluted with water, freeze-dried, dissolved in DMSO, and added to previously made HDL to produce HDL-MOG-TMR. Conjugation of MOG-TMR to DOPE-MAL and incorporation of MOG-TMR in HDL were measured by HPLC/MS, as indicated above. For the lymph node draining studies, naive female C57BL/6 mice or EAE-induced mice were administered s.c. at tail base with HDL-MOG-TMR or free MOG-TMR containing antigen peptide (100 μg per mouse) in 100 μl volume. After 24, 96, and 196 hr, animals were euthanized, organs were harvested, and TMR signal was measured with an IVIS optical imaging system (Caliper Life Sciences). Inguinal lymph nodes and spinal cord were cut into small pieces and passed through a 70-μm cell strainer, washed two times, and stained with the indicated antibodies, followed by flow cytometry analysis.

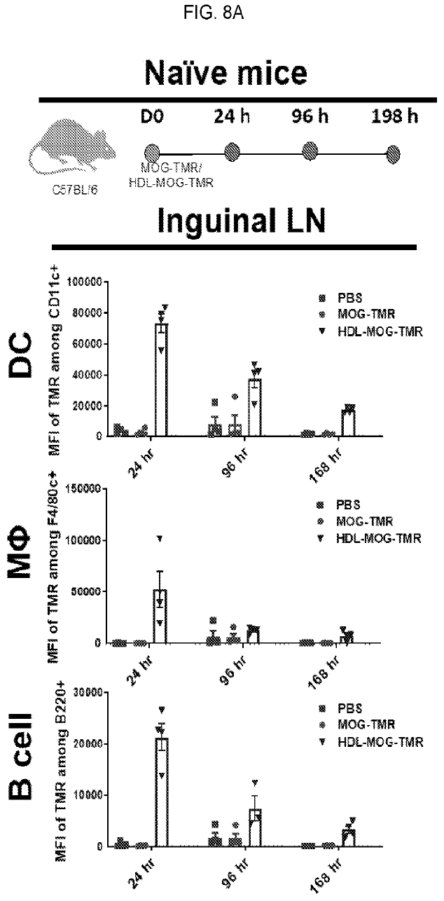

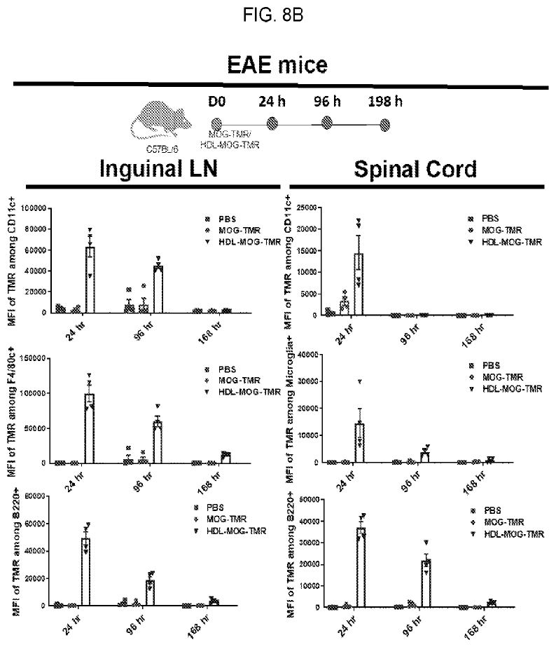

SC administration of free MOG-TMR peptide in naïve mice resulted in minimal TMR signal in inguinal dLNs after one day (FIG. 8A). The low signal intensity can be attributed to the systemic dissemination of small molecular-weight peptides. In contrast, sHDL-MOG-TMR exhibited significantly increased TMR signal in dLNs (FIG. 8A), with significant cellular uptake of Ag by CD11c⁺ DCs, B220⁺ B cells, and F4/80⁺ macrophages. Similarly, in EAE-induced mice, we also observed significantly enhanced uptake of Ag after administration of HDL-MOG-TMR, compared with free MOG-TMR peptide (FIG. 8B). Interestingly, we detected accumulation of HDL-MOG-TMR in spinal cord in EAE-induced mice, leading to their uptake among CD11c⁺ DCs, B220⁺ B cells, and F4/80⁺ macrophages. In contrast, we did not observe accumulation of HDL-MOG-TMR in spinal cord in naïve mice, indicating that EAE-mediated damage of blood brain barrier allowed infiltration of HDL-MOG into the central nervous system (CNS).

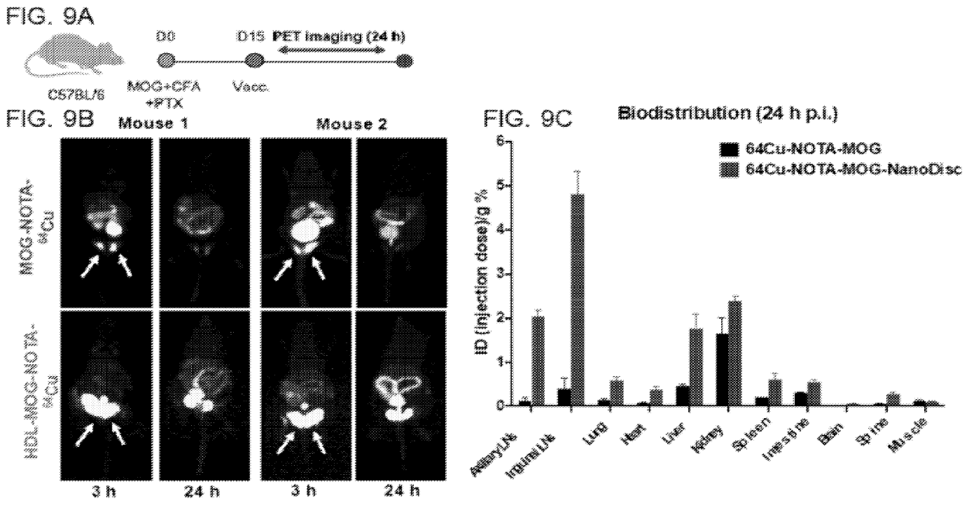

To further quantify biodistribution of HDL-MOG, we employed positron emission tomography (PET) imaging. Copper-64 (⁶⁴Cu) was synthesized with an onsite cyclotron (GE PETtrace) method. ⁶⁴CuCl2 (74 MBq) was diluted in 0.3 mL of 0.1 M sodium acetate buffer (pH 5.0) and mixed with 0.5 mg of HDL-MOG. The mixing was conducted at 37° C. for 30 min with constant shaking. Subsequently, 5 μL 0.1 M EDTA (ethylenediaminetetraacetic acid) was added into the solution and shaken for 5 min to remove non-specifically bound ⁶⁴Cu. The resulting HDL-MOG-NOTA-⁶⁴Cu was purified by centrifugation filtration (10 kDa). C57BL/6 mice were then administered s.c. with 5-8 MBq of HDL-MOG-NOTA-⁶⁴Cu or MOG-NOTA-⁶⁴Cu, and PET imaging was performed overtime using a microPET/microCT Inveon rodent model scanner (Siemens Medical Solutions USA, Inc.) (FIG. 9A). Quantitative PET data for the major organs were presented as the percentage injected dose per gram of tissue (% ID g-1). Subcutaneous (s.c.) administration of free ⁶⁴Cu-tagged MOG resulted in rapid systemic dissemination with minimal antigen remaining at the injection site or in lymph nodes by 24 hr (FIG. 9B). On the other hand, s.c. administration of HDL-MOG-NOTA-⁶⁴Cu resulted in significant accumulation of ⁶⁴Cu-tagged MOG in multiple draining LNs as well as at the injection site at 24 hr time point (FIG. 9B). Mice were euthanized at 24 hr and radioactivity of major organs were quantified (FIG. 9C). In agreement with the PET imaging dataset, we detected significantly higher ⁶⁴Cu signal in draining lymph nodes and other issues (including spine) for the HDL-MOG-NOTA-⁶⁴Cu group, compared with MOG-NOTA-⁶⁴Cu. These results suggest that HDL significantly enhances antigen delivery to draining lymph nodes and that a subset of HDL forms a depot at the injection site, allowing for continuous, systemic delivery to the major organs.

### Example 4. The Impact of HDL-MOG Treatment on Inflammatory Cytokines and Inflammatory Cytokine-Producers in EAE Mice

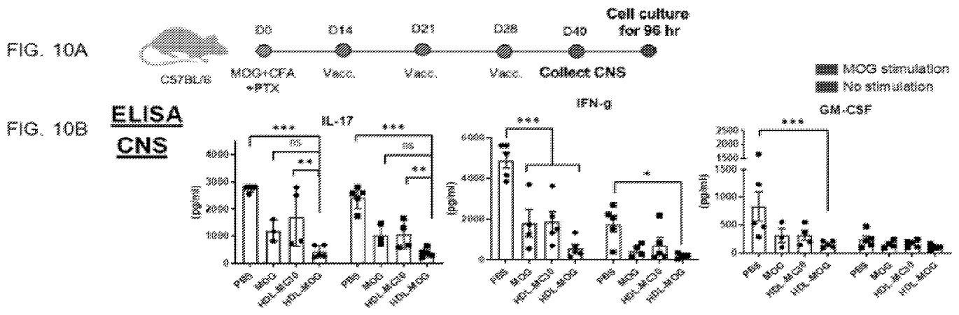

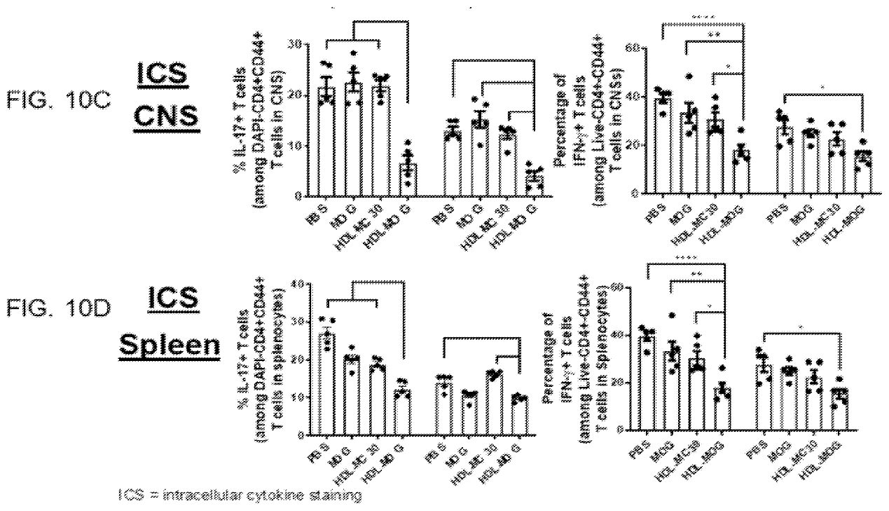

Briefly, female C57BL/6 mice were injected subcutaneously with an emulsion of MOG₃₅₋₅₅ (MEVGWYRSPFSRVVHLYRNGK, SEQ ID NO: 830) in complete Freund's adjuvant (CFA) and intraperitoneal (i.p.) injection of pertussis toxin (120 ng/dose on days 0 and 2). EAE-induced mice were treated with PBS, free MOG, HDL-MOG, or HDL-M30 (irrelevant CD4+ T cell epitope from B16F10 tumor cells) and CNS tissues were harvested on day 40 (FIG. 10A). CNS tissues were harvested from mice after intracardiac perfusion with PBS. Spinal cord was harvested, homogenized in 10 ml of PBS containing 1% BSA, and pelleted at 800×g for 5 min. Cell pellets were resuspended in 3 ml of collagenase A (1 mg/ml) and DNase I (1 mg/ml) in HBSS and incubated in a 37° C. water bath for 30 min. Samples were pelleted at 800×g, resuspended in 27% Percoll, and centrifuged for 10 min at 800×g. The myelin/debris layer and Percoll were removed, and the cell pellet was stained and analyzed by flow cytometric analysis. Splenic immune cells were isolated by homogenization through a 70 m strainer (BD Falcon). RBCs were lysed using ACK lysis buffer. Cells were washed with 25 ml of PBS, centrifuged at 800×g, resuspended in FACS buffer, and stained with antibodies. For the cellular surface staining, cells were resuspended in PBS with a fixable viability dye (BV510) for 10 min. Then, cells were washed two times with FACs buffer and resuspended in Fc Block (anti-CD16/32; 100 ng/ml). For ex vivo re-stimulation, cells were incubated for 96 hr with MOG₃₅₋₅₅ before brefeldin A (BFA) (5 μg/ml) was added for 4 h. For intracellular staining, cells were stained for surface markers, fixed/permeabilized, and stained with antibody for 30 min on the ice. After 2× washing, cells were resuspended in FACS buffer and analyzed by flow cytometric analysis. Data were collected with a Cytek Aurora flow cytometer using FCS express software (V7).

On day 40, CNS tissues were isolated and pulsed ex vivo with MOG₃₅₋₅₅ peptide, followed by measurement of IL-17, IFN-gamma, and GM-CSF by ELISA (FIG. 10B). Animals treated with PBS, free MOG peptide, or HDL-M30 had high levels of IL-17, IFN-gamma, and GM-CSF in CNS (FIG. 10B), indicating strong inflammation. In stark contrast, HDL-MOG-treated animals had significantly reduced levels of IL-17, IFN-gamma, and GM-CSF in CNS (FIG. 10B), suggesting antigen-specific immune tolerance induced by HDL-MOG treatments. In parallel, we performed intracellular cytokine staining on CD4+ T cells from CNS cells (FIG. 10C) or splenocytes (FIG. 10D) ex vivo stimulated with or without free MOG peptide. As in FIG. 10B, treatments with PBS, free MOG peptide, or HDL-M30 resulted in high frequencies of CD4+ T cells producing IL-17, IFN-gamma, and GM-CSF in both CNS and spleen. In stark contrast, HDL-MOG-treated mice had significantly reduced frequency of CD4+ T cells producing IL-17, IFN-gamma, and GM-CSF in both CNS and spleen (FIGS. 10C-10D).

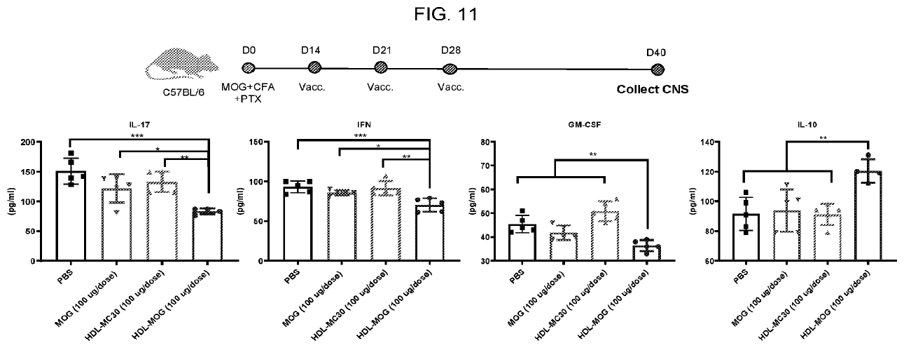

To further validate these results, we treated EAE mice as shown in FIG. 11, collected fresh CNS tissues on day 40, and performed ELISA without ex vivo re-stimulation. Correlating with the results shown in FIGS. 4A-4D, HDL-MOG treatments led to significant reduction in the concentrations of IL-17, IFN-gamma, and GM-CSF, while increasing IL-10 levels in the CNS (FIG. 11). However, mice treated with free MOG or HDL-M30 had similar levels of IL-17, IFN-gamma, GM-CSF, and IL-10 as in PBS-treated EAE mice.

Taken together, these studies showed that HDL-MOG treatments led to antigen-specific immune tolerance in both the systemic compartment as well as in the CNS—the peripheral site of inflammation.

### Example 5. The Impact of HDL-MOG Treatment on Regulatory T Cells (Tregs) in EAE Mice

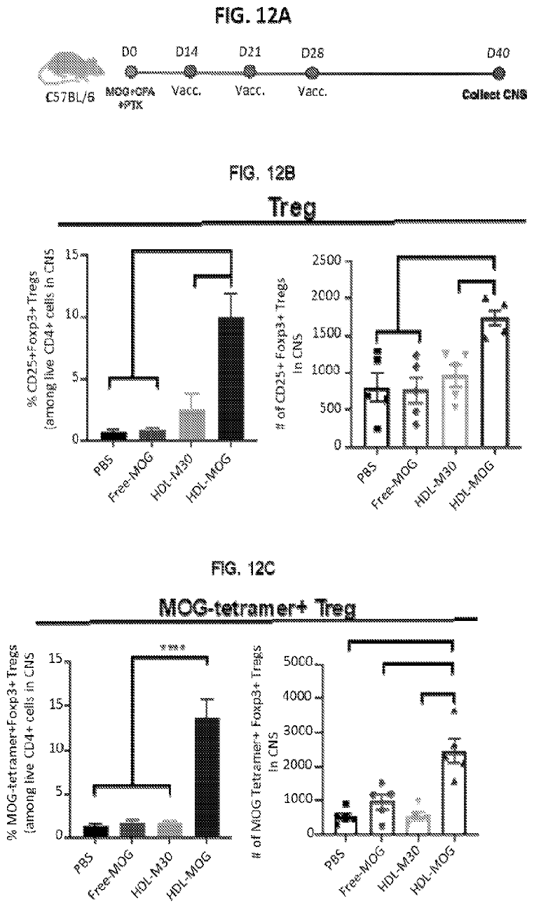

EAE-induced mice were treated as shown in FIG. 12A, and the frequency of Tregs were quantified in the CNS. Cells from CNS were stained by anti-CD25, anti-CD4, and MOG-tetramer, followed by fixation/permeabilization and intracellular staining with anti-Foxp3. Stained cells were then analyzed by flow cytometry. HDL-MOG treatments significantly increased the frequency of CD25+Foxp3+ Tregs in the CNS (FIG. 12B), and we also validated this using MOG-tetramer (FIG. 12C), indicating that HDL-MOG induces MOG-specific Tregs in CNS. On the other hand, mice treated with free MOG peptide or HDL-M30 had basal level of Tregs as in PBS-treated EAE mice (FIGS. 12A-12B).

### Example 6. We Studied the Impact of Regulatory T Cells (Tregs) on the Treatment Outcomes of HDL-MOG

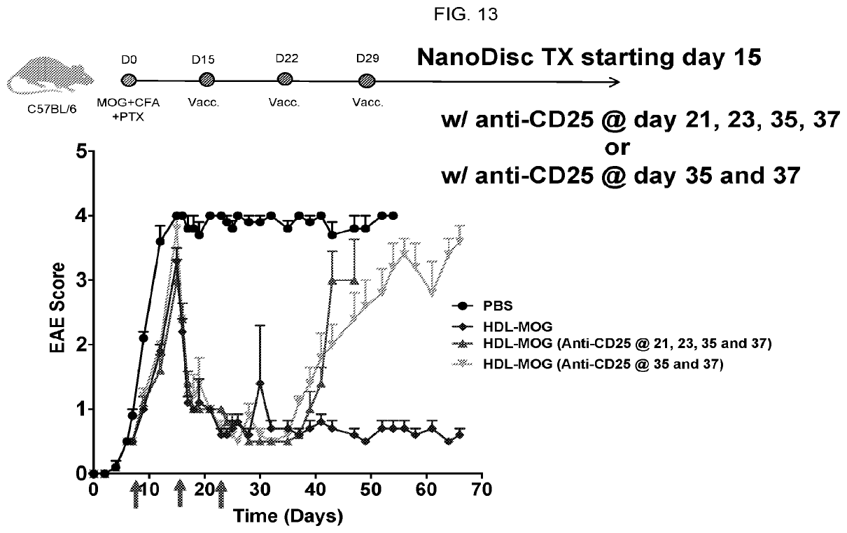

EAE-induced mice were administered s.c. with PBS or HDL-MOG on days 15, 22, and 29, and a subset of animals also received i.p. administration of anti-25 IgG to deplete Tregs at the specified time points (FIG. 13). EAE-mice treated with HDL-MOG exhibited drastic improvement in the EAE symptoms (FIG. 13) as demonstrated before. When anti-CD25 was administered on days 35 and 37, mice quickly relapsed and exhibited EAE>3 score by day 50 (FIG. 13), indicating that anti-CD25-mediated depletion of Tregs triggered the relapse. Interestingly, administration of anti-CD25 on days 21, 23, 35, and 37 resulted in the similar outcomes as in mice given anti-CD25 only on days 35 and 37. These results suggest that Tregs play crucial roles in HDL-MOG-mediated immune tolerance and that Tregs are critical for the long-term control of the disease.

### Example 7. sHDL Nanodisc with Various Immunomodulatory Drugs

We formulated HDL nanodiscs loaded with various immunomodulatory drugs. In particular, FTY720, ITE, TSA, SAHA, and rapamycin (Rapa) have been reported to exhibit strong immunomodulatory properties. FTY720 (also known as fingolimod) is an oral drug that can sequester T cells in lymphoid tissues (Chung and Harung, Clin. Neuropharmacol 33: 91-101, 2010). AhR activation by 2-(1′H-indole-3′-carbonyl)-thiazole-4-carboxylic acid methyl ester (ITE) or related ligands have been reported expand Tregs and promote immune tolerance (Yeste A, et al. Proc. Natl. Acad. Sci. USA 109: 11270-11275, 2012; Quintana F. J., et al Proc. Natl. Acad. Sci. USA 107: 20768-20773, 2010). Trichostatin A (TSA) can increase the frequency of Tregs and increase immunosuppressive functions of Tregs (Reilly C. M. et al. J. Autoimmun 31: 123-130. 2008). Suberoylanilide hydroxamic acid (SAHA), a histone deacetylase inhibitor, has been shown to induce Tregs (Lucas J. L., et al. Cell Immunol 257: 97-104, 2009). Rapamycin (Rapa) has been shown to induce immunosuppression when co-administered with biologics, including foreign enzymes (Maldonado, R. A., et al Proc. Natl. Acad. Sci. USA 112:E156-165, 2015).

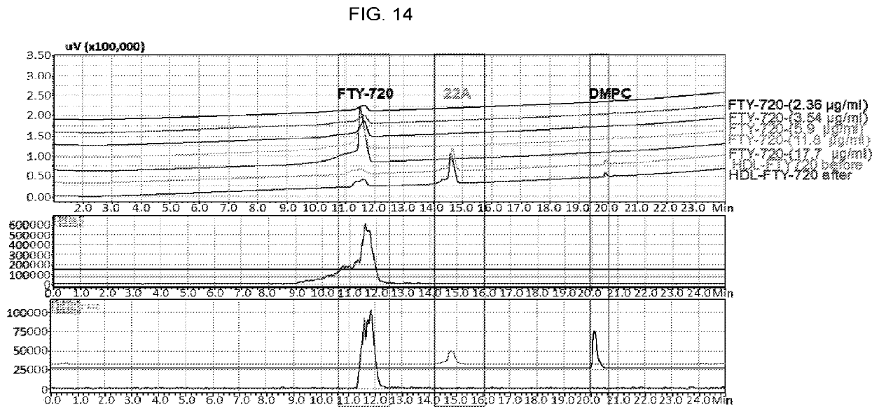

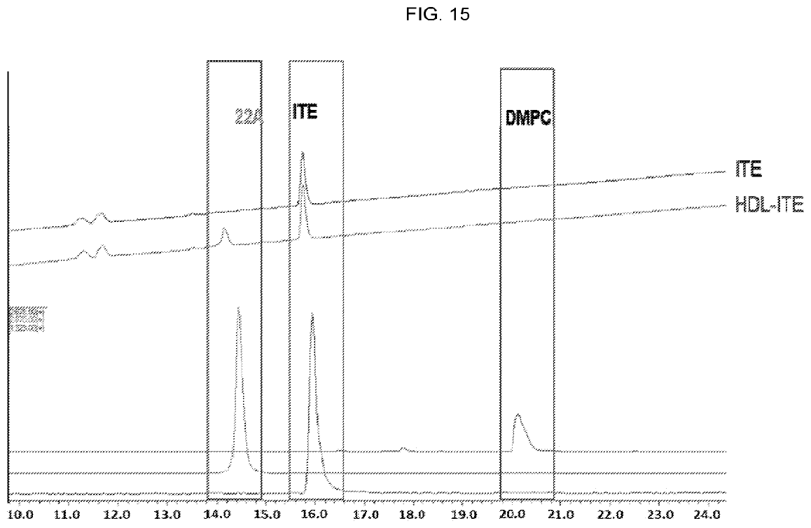

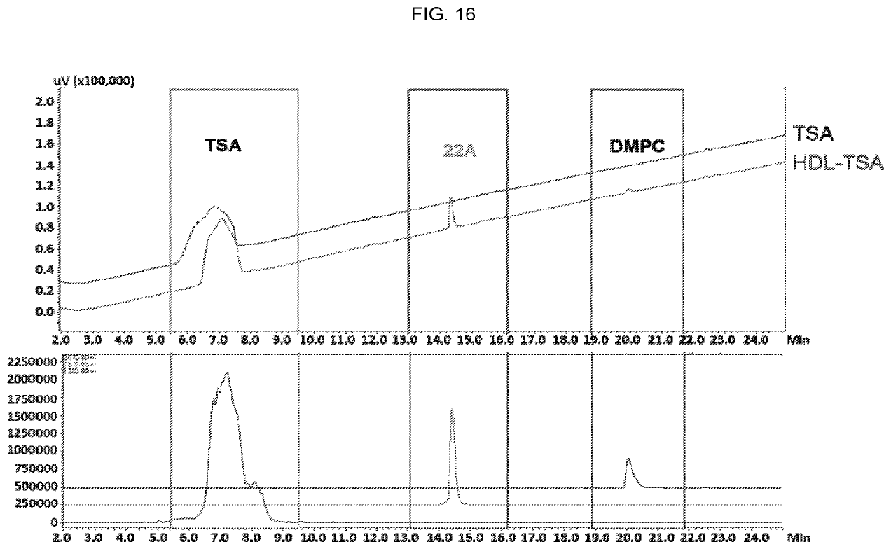

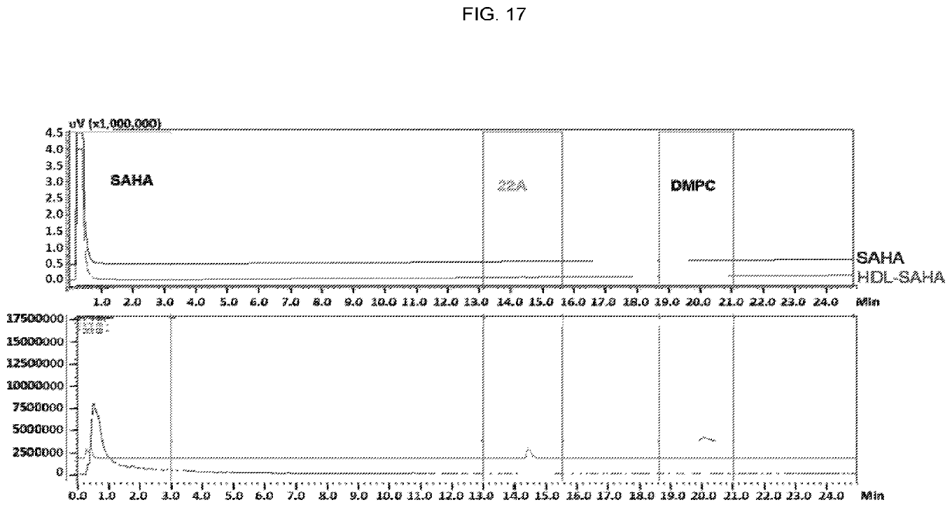

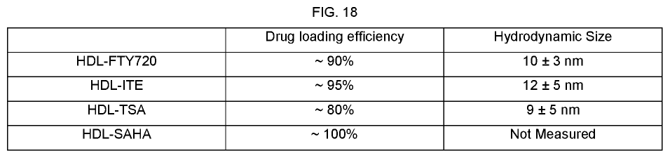

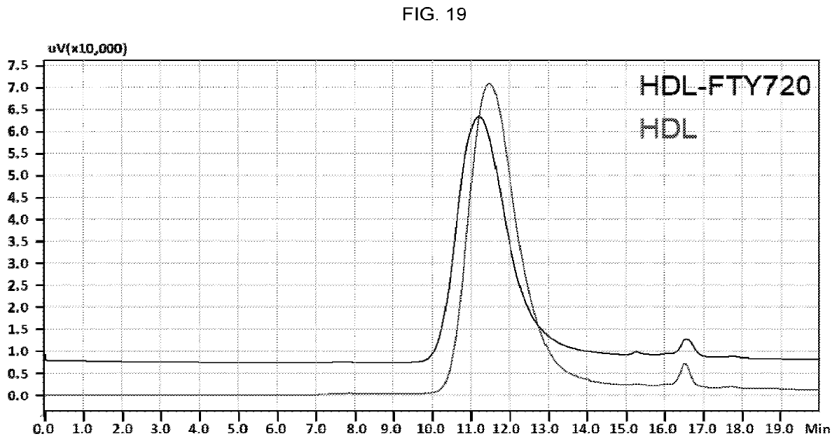

To produce HDL nanodiscs carrying immunomodulatory drugs, 22A, and DMPC were dissolved in acetic acid and freeze-dried overnight as shown above. Drugs (FTY720, ITE, TSA, or SAHA) were dissolved in chloroform and added to lyophilized powder, followed by drying overnight in vacuum oven. Dried samples were rehydrated by 10 mM phosphate buffer. After three heat-cooling cycles, HDL-loaded with drugs were performed. The concentration of each drug was measured by HPLC-MS as shown for FTY720, ITE, TSA, or SAHA (FIGS. 14-17, respectively). Free drugs were used to generate standard curves. As shown in FIG. 18, we achieved highly efficient drug encapsulate efficiencies of ˜90%, ˜95%, ˜80%, and ˜100% for FTY720, ITE, TSA, and SAHA, respectively. Samples were also analyzed by dynamic light scattering (DLS) for the particle sizes. As shown in FIG. 18, HDL-FTY720, HDL-ITE, and HDL-TSA exhibited hydrodynamic size of ˜10 nm, 12 nm, and 9 nm, respectively. We also analyzed HDL-FTY720 samples using gel permeation chromatography (GPC) (FIG. 19). GPC chromatograms showed that HDL-FTY720 was relatively homogeneous and eluted earlier thank blank HDL nanodiscs, indicating successful encapsulation of FTY720 in HDL nanodiscs.

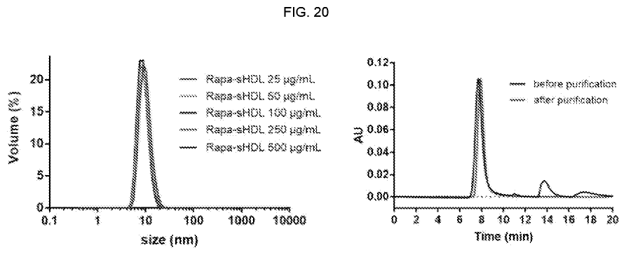

We have also encapsulated Rapa into HDL. As analyzed by dynamic light scattering and gel permeation chromatography, HDL-Rapa exhibited a homogeneous size distribution with an average hydrodynamic size of ˜10 nm (FIG. 20).

### Example 8. Efficacy of HDL-FTY720 in EAE Mouse Model

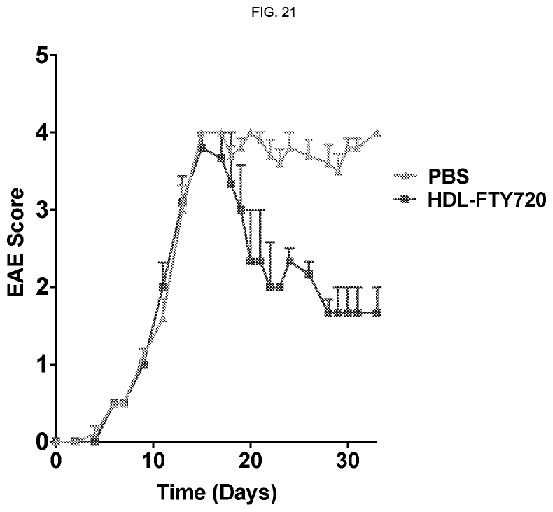

We examined the therapeutic potential of HDL-FTY720 in the EAE model. EAE was initiated as described above. Briefly, EAE was induced in mice as indicated above. Mice received i.p. administration of PBS or HDL-FTY720 (1 mg/kg) on days 14, 21, and 28. Mice were scored daily and assigned EAE scores as indicated above. EAE-induced mice treated with HDL-FTY720 quickly improved symptoms and the EAE score of 4 at the peak the disease on day 14 decreased to EAE score of <2 by day 30 (FIG. 21). These results suggest that HDL-FTY720 may be used for systemic delivery of FTY720.

### Example 9. sHDL with CD4+ T Cell Epitopes

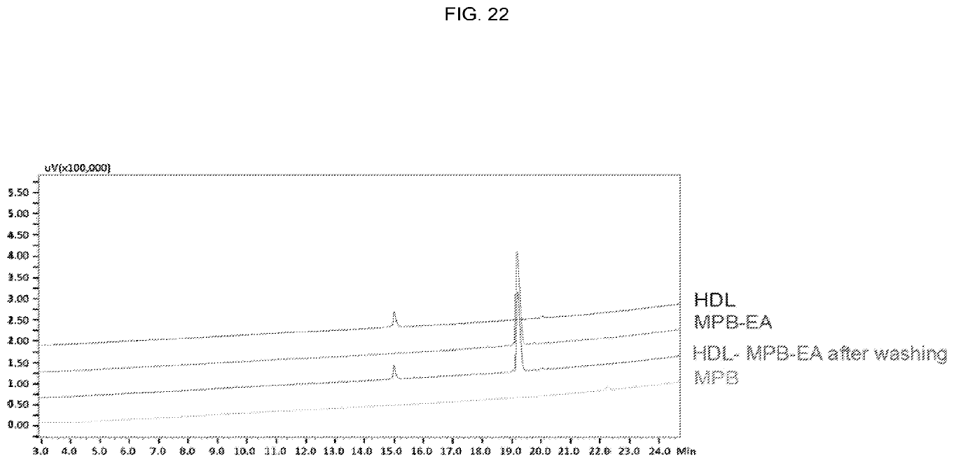

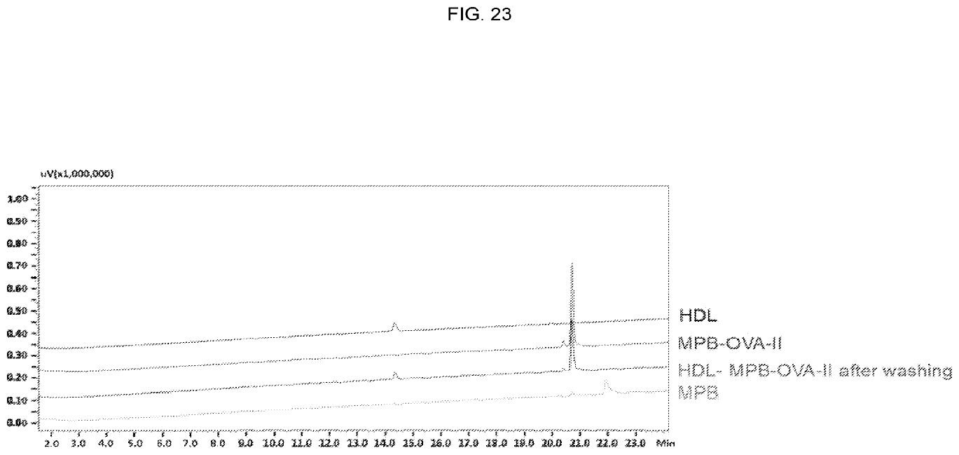

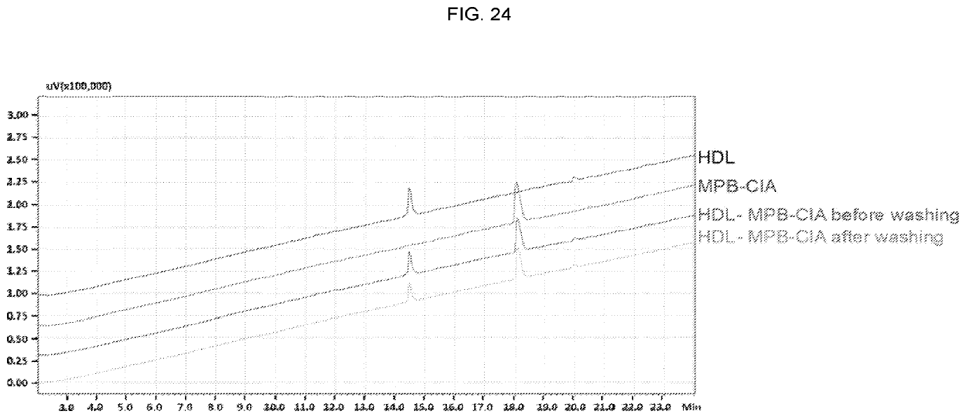

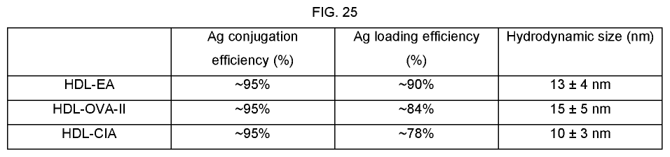

We synthesized HDL nanodiscs loaded with other CD4+ T cell epitopes for validation of our approach. We have synthesized phospholipids conjugated to either Ea chain 52-68 peptide (EA, ASFEAQGALANIAVDKA (SEQ ID NO: 831)), ovalbumin-II 323-339 peptide (OVA-II, ISQAVHAAHAEINEAGR (SEQ ID NO: 832)), and type-II collagen 250-270 peptide (CIA, GPKGQTGKPGIAGFKGEQGPK (SEQ ID NO: 833)), each modified with CSS-peptide at N-terminus. Antigen-lipid conjugates were loaded into HDL nanodiscs as described above and analyzed by HPLC/MS and DLS. EA, OVA-II, and CIA peptides were conjugated to MPB-phospholipids with ˜95% conjugation efficiency (FIGS. 22-25). Antigen-lipid conjugates were efficiently loaded into HDL nanodiscs with ˜90%, ˜84%, and ˜78% loading efficiency for EA, OVA-II, and CIA peptides, respectively (FIGS. 22-25). HDL-EA, HDL-OVA-II, and HDL-CIA exhibited hydrodynamic sizes of 13, 15, and 10 nm, respectively (FIGS. 22-25).

### Example 10. Production of sHDL Nanodiscs with Gliadin Peptides for the Treatment of Subjects with Celiac Disease

An HDL nanodisc is prepared by dissolving dipalmitoylphosphatidylcholine (DMPC) in chloroform followed by evaporating the chloroform using nitrogen flow under vacuum for at least 1 hour. The resulting lipid film is rehydrated in 10 mM sodium phosphate buffer and sonicated in a bath sonicator for 10 minutes. ApoA1 mimetic peptide 22A, dissolved in endotoxin free water, is then added to the above mixture in a ratio of 1:2 (w/w) of ApoA1 mimetic peptide 22A to DMPC in order to obtain nanodiscs. Gliadin peptides are loaded on the nanodiscs by reacting each antigen peptide with 4-(4-maleimidophenyl)-butyric DOPE for 3 hours in dimethylformamide (DMF). The DMF is removed by freeze-drying after the reaction mixture is diluted 10× with endotoxin-free water. The lipid-peptide conjugates are dissolved in DMSO and added to pre-formed sHDL and incubated for 30 minutes at room temperature. Unreacted gliadin peptides are removed using Zeba Spin Desalting columns (Pierce) following the manufacturer's instructions. The conjugation efficiency of gliadin peptide is determined using LC-MS and gel permeation chromatography (GPC). Nanodiscs loaded with gliadin peptides provide efficient means to deliver gliadin peptides to dendritic cells and other antigen-presenting cells upon in vivo administration. Nanodiscs allow for antigen processing and presentation on antigen-presenting cells so that immune tolerance against celiac disease is induced. Patients suffering from celiac disease may be treated with these nanodiscs via subcutaneous administration. Patients may be administered a weekly dosage for up to 6 weeks or longer, followed by maintenance doses after 3 months or longer.

### Other Embodiments

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

1. A composition comprising an sHDL nanoparticle associated with a plurality of tolerogenic antigens in such a manner that the resulting composition is capable of facilitating strong immune tolerance to antigens associated with an autoimmune disease upon administration to a subject, wherein the sHDL nanoparticle comprises a mixture of at least one phospholipid and at least one HDL apolipoprotein or apolipoprotein mimetic.

2. The composition of claim 1, wherein the phospholipid is selected from the group consisting of 1,2-dilauroyl-sn-glycero-3-phosphocholine; 1,2-dimyristoyl-sn-glycero-3-phosphocholine; 1,2-dipalmitoyl-sn-glycero-3-phosphocholine; 1,2-distearoyl-sn-glycero-3-phosphocholine; 1,2-diarachidoyl-sn-glycero-3-phosphocholine; 1,2-dibehenoyl-sn-glycero-3-phosphocholine; 1,2-dilignoceroyl-sn-glycero-3-phosphocholine; 1,2-dimyristoleoyl-sn-glycero-3-phosphocholine; 1,2-dimyristelaidoyl-sn-glycero-3-phosphocholine; 1,2-dipalmitoleoyl-sn-glycero-3-phosphocholine; 1,2-dipalmitelaidoyl-sn-glycero-3-phosphocholine; 1,2-dipetroselenoyl-sn-glycero-3-phosphocholine; 1,2-dioleoyl-sn-glycero-3-phosphocholine; 1,2-dielaidoyl-sn-glycero-3-phosphocholine; 1,2-dieicosenoyl-sn-glycero-3-phosphocholine; 1,2-dinervonoyl-sn-glycero-3-phosphocholine; 1,2-dilauroyl-sn-glycero-3-phosphoethanolamine; 1,2-dimyristoyl-sn-glycero-3-phosphoethanolamine; 1,2-dipentadecanoyl-sn-glycero-3-phosphoethanolamine; 1,2-dipalmitoyl-sn-glycero-3-phosphoethanolamine; 1,2-distearoyl-sn-glycero-3-phosphoethanolamine; 1,2-dipalmitoleoyl-sn-glycero-3-phosphoethanolamine; 1,2-dielaidoyl-sn-glycero-3-phosphoethanolamine; 1,2-dioleoyl-sn-glycero-3-phosphoethanolamine; dioleoyl-sn-glycero-3-phosphoethanolamine-N-[3-(2-pyridyldithio) propionate]; 1,2-dipalmitoyl-sn-glycero-3-phosphothioethanol; 1,2-di-(9Z-octadecenoyl)-sn-glycero-3-phosphoethanolamine-N-[4-(p-maleimidophenyl)butyramide]; 1,2-dihexadecanoyl-sn-glycero-3-phosphoethanolamine-N-[4-(p-maleimidophenyl)butyramide]; 1,2-dihexadecanoyl-sn-glycero-3-phosphoethanolamine-N-[4-(p-maleimidomethyl)cyclohexane-carboxamide]; 1,2-di-(9Z-octadecenoyl)-sn-glycero-3-phosphoethanolamine-N-[4-(p-maleimidomethyl)cyclohexane-carboxamide]; N-[(3-Maleimide-1-oxopropyl)aminopropyl polyethyleneglycol-carbamyl] distearoylphosphatidyl-ethanolamine; N-[(3-Maleimide-1-oxopropyl)aminopropyl polyethyleneglycol-carbamyl] distearoylphosphatidyl-ethanolamine; N-(3-Maleimide-1-oxopropyl)-L-α-phosphatidylethanolamine, Distearoyl; N-[(3-Maleimide-1-oxopropyl)aminopropyl polyethyleneglycol-carbamyl] distearoylphosphatidyl-ethanolamine; N-(3-Maleimide-1-oxopropyl)-L-α-phosphatidylethanolamine, Dimyristoy; N-(3-Maleimide-1-oxopropyl)-L-α-phosphatidylethanolamine, Dioleoyl; N-(3-Maleimide-1-oxopropyl)-L-α-phosphatidylethanolamine, Dipalmitoyl; N-(3-Maleimide-1-oxopropyl)-L-α-phosphatidylethanolamine, 1-Palmitoyl-2-oleoyl; phosphatidylcholine; phosphatidylinositol; phosphatidylserine; phosphatidylethanolamine; N-(Succinimidyloxy-glutaryl)-L-α-phosphatidylethanolamine, Distearoyl; N-(Succinimidyloxy-glutaryl)-L-α-phosphatidylethanolamine, Dioleoyl; N-(Succinimidyloxy-glutaryl)-L-α-phosphatidylethanolamine, 1-Palmitoyl-2-oleoyl; N-(Succinimidyloxy-glutaryl)-L-α-phosphatidylethanolamine, Dipalmitoyl; N-(Succinimidyloxy-glutaryl)-L-α-phosphatidylethanolamine, Dimyristoyl; 3-(N-succinimidyloxyglutaryl)aminopropyl, and polyethyleneglycol-carbamyl distearoylphosphatidyl-ethanolamine; N-(3-oxopropoxy polyethyleneglycol)carbamyl-distearoyl-ethanolamine.

3. The composition of claim 1, wherein the HDL apolipoprotein component is selected from the group consisting of apolipoprotein A-I (apoA-1), apolipoprotein A-II (apoA-II), apolipoprotein A-II xxx (apoA-II-xxx), apolipoprotein A4 (apoA4), apolipoprotein Cs (apoCs), apolipoprotein E (apoE), apolipoprotein A-I milano (apoA-I-milano), apolipoprotein A-I paris (apoA-I-paris), apolipoprotein M (apoM), an HDL apolipoprotein mimetic, preproapoliprotein, preproApoA-I, proApoA I, preproApoA-II, proApoA II, preproApoA-IV, proApoA-IV, ApoA-V, preproApoE, proApoE, preproApoA I_{Milano}, proApoA-I_{Milano}, preproApoA-I_{Paris}, proApoA-I_{Paris}, and mixtures thereof.

4. The composition of claim 1, wherein the apolipoprotein mimetic is described by any of SEQ ID NOs: 1-336 and WDRVKDLATVYVDVLKDSGRDYVSQF (SEQ ID NO:341), LKLLDNWDSVTSTFSKLREOL (SEQ ID NO:342), PVTOEFWDNLEKETEGLROEMS (SEQ ID NO:343), KDLEEVKAKVQ (SEQ ID NO: 344), KDLEEVKAKVO (SEQ ID NO: 345), PYLDDFQKKWQEEMELYRQKVE (SEQ ID NO: 346), PLRAELQEGARQKLHELOEKLS (SEQ ID NO: 347), PLGEEMRDRARAHVDALRTHLA (SEQ ID NO: 348), PYSDELRQRLAARLEALKENGG (SEQ ID NO: 349), ARLAEYHAKATEHLSTLSEKAK (SEQ ID NO: 350), PALEDLROGLL (SEQ ID NO: 351), PVLESFKVSFLSALEEYTKKLN (SEQ ID NO:352), PVLESFVSFLSALEEYTKKLN (SEQ ID NO:353), PVLESFKVSFLSALEEYTKKLN (SEQ ID NO:352), TVLLLTICSLEGALVRRQAKEPCV (SEQ ID NO: 354) QTVTDYGKDLME (SEQ ID NO:355), KVKSPELOAEAKSYFEKSKE (SEQ ID NO:356), VLTLALVAVAGARAEVSADOVATV (SEQ ID NO:357), NNAKEAVEHLOKSELTOOLNAL (SEQ ID NO:358), LPVLVWLSIVLEGPAPAOGTPDVSS (SEQ ID NO:359), LPVLVVVLSIVLEGPAPAQGTPDVSS (SEQ ID NO:360), ALDKLKEFGNTLEDKARELIS (SEQ ID NO: 361), VVALLALLASARASEAEDASLL (SEQ ID NO:362), HLRKLRKRLLRDADDLQKRLAVYOA (SEQ ID NO:363), AQAWGERLRARMEEMGSRTRDR (SEQ ID NO:364), LDEVKEQVAEVRAKLEEQAQ (SEQ ID NO:365), DWLKAFYDKVAEKLKEAF (SEQ ID NO:236), DWLKAFYDKVAEKLKEAFPDWAKAAYDKAAEKAKEAA (SEQ ID NO:366), PVLDLFRELLNELLEALKQKL (SEQ ID NO:367), PVLDLFRELLNELLEALKQKLA (SEQ ID NO:368), PVLDLFRELLNELLEALKQKLK (SEQ ID NO:4), PVLDLFRELLNELLEALKQKLA (SEQ ID NO:369), PVLDLFRELLNELLEALKKLLK (SEQ ID NO:370), PVLDLFRELLNELLEALKKLLA (SEQ ID NO:371), PLLDLFRELLNELLEALKKLLA (SEQ ID NO:372), and EVRSKLEEWFAAFREFAEEFLARLKS (SEQ ID NO: 373).

5. The composition of any one of claims 1-4, wherein the plurality of tolerogenic antigens are tolerogenic antigens comprising between 3 amino acids and 50 amino acids in length.

6. The compositions of any one of claims 1-5, wherein the plurality of tolerogenic antigens are tolerogenic antigens comprising a polypeptide comprising a nucleic acid sequence of any one of SEQ ID NOs: 375-796.

7. The composition of any one of claims 1-6, wherein the plurality of tolerogenic antigens are human allograft transplantation antigens.

8. The composition of claim 7, wherein the human allograft transplantation antigens are selected from subunits of the various MHC class I and MHC class II haplotype proteins, and single-amino-acid polymorphisms on minor blood group antigens including RhCE, Kell, Kidd, Duffy and Ss.

9. The composition of claim 1, wherein the plurality of tolerogenic antigens are specific for type 1 diabetes mellitus.

10. The composition of claim 9, wherein the tolerogenic antigens are selected from insulin, proinsulin, preproinsulin, glutamic acid decarboxylase-65 (GAD-65), GAD-67, insulinoma-associated protein 2 (IA-2), insulinoma-associated protein 2p (IA-2β), ICA69, ICA12 (SOX-13), carboxypeptidase H, Imogen 38, GLIMA 38, chromogranin-A, HSP-60, caboxypeptidase E, peripherin, glucose transporter 2, hepatocarcinoma-intestine-pancreas/pancreatic associated protein, S100p, glial fibrillary acidic protein, regenerating gene II, pancreatic duodenal homeobox 1, dystrophia myotonica kinase, islet-specific glucose-6-phosphatase catalytic subunit-related protein, and SST G-protein coupled receptors 1-5.

11. The composition of claim 1, wherein the tolerogenic antigens are specific for one or more of the following autoimmune disorders: rheumatoid arthritis, multiple sclerosis, diabetes, autoimmune diseases of the thyroid, thyroid-associated ophthalmopathy and dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, and inflammatory bowel disease.

12. The composition of claim 1, wherein the plurality of tolerogenic antigens comprises one or more of tolerogenic antigens selected from thyroglobulin (TG), thyroid peroxidase (TPO), thyrotropin receptor (TSHR), sodium iodine symporter (NIS), megalin, thyroid autoantigens including TSHR, insulin-like growth factor 1 receptor, calcium sensitive receptor, 21-hydroxylase, 17α-hydroxylase, and P450 side chain cleavage enzyme (P450scc), ACTH receptor, P450c21, P450c17, FSH receptor, α-enolase, pituitary gland-specific protein factor (PGSF) 1a and 2, and type 2 iodothyronine deiodinase, myelin basic protein, myelin oligodendrocyte glycoprotein, proteolipid protein, collagen II, H⁺, K⁺-ATPase, tissue transglutaminase and gliadin, tyrosinase, tyrosinase related protein 1 and 2, acetylcholine receptor, desmoglein 3, 1 and 4, pemphaxin, desmocollins, plakoglobin, perplakin, desmoplakins, acetylcholine receptor, BP180, BP230, plectin, laminin 5, endomysium, tissue transglutaminase, collagen VII, matrix metalloproteinase 1 and 3, the collagen-specific molecular chaperone heat-shock protein 47, fibrillin-1, PDGF receptor, Scl-70, U1 RNP, Th/To, Ku, Jo1, NAG-2, centromere proteins, topoisomerase I, nucleolar proteins, RNA polymerase I, II and III, PM-Slc, fibrillarin, B23, U1snRNP, nuclear antigens SS-A and SS-B, fodrin, poly(ADP-ribose) polymerase, topoisomerase, nuclear proteins including SS-A, high mobility group box 1 (HMGB1), nucleosomes, histone proteins, double-stranded DNA, glomerular basement membrane proteins including collagen IV, cardiac myosin, aromatic L-amino acid decarboxylase, histidine decarboxylase, cysteine sulfinic acid decarboxylase, tryptophan hydroxylase, tyrosine hydroxylase, phenylalanine hydroxylase, hepatic P450 cytochromes P4501A2 and 2A6, SOX-9, SOX-10, calcium-sensing receptor protein, and type 1 interferons interferon alpha, beta and omega.

13. The composition of any one of claims 1-6, wherein the plurality of tolerogenic antigens are specific for celiac disease.

14. The composition of claim 13, wherein the tolerogenic antigens are selected from gliadin, glutenin, and fragments thereof capable of inducing an immune response.

15. The composition of claim 14, wherein the tolerogenic antigens are selected from gliadin or fragments thereof.

16. The composition of claim 15, wherein the tolerogenic antigens are selected from the group consisting of α, γ, and ω gliadins or fragments thereof.

17. The composition of claim 15 or 16, wherein the tolerogenic antigen comprises a polypeptide having at least 90% sequence identity to the polypeptide sequence of any one of SEQ ID NOs: 375-580.

18. The composition of claim 17, wherein the tolerogenic antigen comprises a polypeptide having at least 90% sequence identity to any one of the polypeptide sequences of SEQ ID NOs: 375-580.

19. The composition of claim 18, wherein the tolerogenic antigen comprises a polypeptide having the polypeptide sequence of any one of SEQ ID NOs: 375-580.

20. The composition of claim 19, wherein the tolerogenic antigen comprises two or more polypeptide sequences having the sequence of any one of SEQ ID NOs: 375-580.

21. The composition of any one of claims 1-4, wherein the tolerogenic antigens are multimeric tolerogenic antigens comprising the following N-terminal-to-C-terminal structure
(P₄-L₄)ₙ₄-(P₃-L₃)ₙ₃-P₂-(L₁-P₁)ₙ₁
wherein P₁, P₂, P₃, and P₄ are each independently a tolerogenic antigen;
L₁, L₃, and L₄ are each independently a linker; and
n₁, n₃, and n₄ are each independently 0 or 1, wherein at least one of n₁, n₃, and n₄ are 1.

22. The composition of claim 21, wherein n₁ is 1, n₃ is 0, and n₄ is 0, and the tolerogenic antigen comprises the following N-terminal-to-C-terminal structure:
P₂-L₁-P₁.

23. The composition of claim 22, wherein L₁ is a peptide linker comprising between 2 and 200 amino acids.

24. The composition of claim 23, wherein L₁ is a peptide linker comprising between 5 and 50 amino acids.

25. The composition of claim 23 or 24, wherein L₁ is a peptide linker comprising glycine (G) and serine (S) residues.

26. The composition of any one of claims 22-25, wherein L₁ is a peptide linker comprising the amino acid sequence of (GS)ₓ, (GGS)ₓ, or (GGGGS)ₓ, wherein x is an integer from 1 to 10.

27. The composition of any one of claims 22-26, wherein P₁ and P₂ each comprise different tolerogenic antigens.

28. The composition of any one of claims 22-26, wherein P₁ and P₂ each comprise identical tolerogenic antigens.

29. The composition of claim 21, wherein n₁ is 1, n₃ is 1, and n₄ is 0, and the tolerogenic antigen comprises the following N-terminal-to-C-terminal structure:
P₃-L₃-P₂-L₁-P₁.

30. The composition of claim 29, wherein L₁ and L₃ are each an independently selected peptide linker comprising between 2 and 200 amino acids.

31. The composition of claim 30, wherein L₁ and L₃ are each an independently selected peptide linker comprising between 5 and 50 amino acids.

32. The composition of claim 30 or 31, wherein L₁ and L₃ are each an independently selected peptide linker comprising glycine (G) and serine (S) residues.

33. The composition of any one of claims 29-32, wherein L₁ and L₃ are each an independently selected peptide linker comprising the amino acid sequence of (GS)ₓ, (GGS)ₓ, or (GGGGS)ₓ, wherein x is an integer from 1 to 10.

34. The composition of any one of claims 29-33, wherein P₁, P₂, and/or P₃ each comprise different tolerogenic antigens.

35. The composition of any one of claims 28-33, wherein P₁, P₂, and P₃ each comprise identical tolerogenic antigens.

36. The composition of claim 21, wherein n₁ is 1, n₃ is 1, and n₄ is 1, and the tolerogenic antigen comprises the following N-terminal-to-C-terminal structure:
P₄-L₄-P₃-L₃-P₂-L₁-P₁.

37. The composition of claim 36, wherein L₁ and L₂ are each an independently selected peptide linker comprising between 2 and 200 amino acids.

38. The composition of claim 37, wherein L₁, L₂, and L₃ are each an independently selected peptide linker comprising between 5 and 50 amino acids.

39. The composition of claim 37 or 38, wherein L₁, L₂, and L₃ are each an independently selected peptide linker comprising glycine (G) and serine (S) residues.

40. The composition of any one of claims 36-39, wherein L₁, L₂, and L₃ are each an independently selected peptide linker comprising the amino acid sequence of (GS)ₓ, (GGS)ₓ, or (GGGGS (SEQ ID NO: 219))ₓ, wherein x is an integer from 1 to 10.

41. The composition of any one of claims 36-40, wherein P₁, P₂, P₃, and/or P₄ each comprise different tolerogenic antigens.

42. The composition of any one of claims 36-40, wherein P₁, P₂, P₃, and P₄ each comprise identical tolerogenic antigens.

43. The composition of any one of claims 1-42, wherein the number of tolerogenic antigens associated with a specific nanoparticle includes a population of between 1 and 30 tolerogenic antigens per nanoparticle.

44. The composition of claim 43, wherein the number of tolerogenic antigens associated with a specific nanoparticle includes a population of between 1 and 10 tolerogenic antigens per particle.

45. The composition of claim 43 or 44, wherein the number of tolerogenic antigens associated with a specific nanoparticle includes a population of 6 tolerogenic antigens per particle.

46. The composition of claim 43 or 44, wherein the number of tolerogenic antigens associated with a specific nanoparticle includes a population of 8 tolerogenic antigens per particle.

47. The composition of any one of claim 44, wherein the population of tolerogenic antigens associated with a specific nanoparticle are the same tolerogenic antigen.

48. The composition of any one of claims 43-46, where the population of tolerogenic antigens associated with a specific nanoparticle comprises between 1 and 5 different tolerogenic antigens.

49. The composition of claim 48, wherein the population of tolerogenic antigens associated with a specific nanoparticle include 3 to 4 different tolerogenic antigens.

50. The composition of claim 48, wherein the population of tolerogenic antigens are specific to between 1 and 3 different diseases.

51. The composition of claim 48, wherein the population of tolerogenic antigens are specific to the same disease.

52. The composition of any one of claims 1-20, wherein the population of tolerogenic antigens associated with a specific nanoparticle comprises (i) a first polypeptide population comprising the amino acid sequence of any one of SEQ ID NOs: 406-588, or a biologically active fragment or variant thereof, (ii) a second polypeptide population comprising the amino acid sequence of any one of SEQ ID NOs: 406-588, or biologically active fragment or variant thereof, and (iii) a third polypeptide population comprising the amino acid sequence of any one of SEQ ID NOs: 406-588, or a biologically active fragment or variant thereof.

53. The composition of claim 51, wherein the first polypeptide population comprises the amino acid sequence of SEQ ID NO: 474, or a biologically active fragment or variant thereof, (ii) the second polypeptide population comprises the amino acid sequence of any one of SEQ ID NOs: 406-588, or biologically active fragment or variant thereof, and (iii) the third polypeptide population comprises the amino acid sequence of any one of SEQ ID NOs: 406-588, or a biologically active fragment or variant thereof.

54. The composition of either claim 51 or 52, wherein the population of tolerogenic antigens associated with a specific nanoparticle comprises (i) the first polypeptide population comprises the amino acid sequence of SEQ ID NO: 474, or a biologically active fragment or variant thereof, (ii) the second polypeptide population comprises the amino acid sequence of SEQ ID NO: 475, or biologically active fragment or variant thereof, and (iii) the third polypeptide population comprises the amino acid sequence of any one of SEQ ID NOs: 406-588, or a biologically active fragment or variant thereof.

55. The composition of claim 54, wherein the third polypeptide population comprises the amino acid sequence of SEQ ID NO: 476, or a biologically active fragment or variant thereof.

56. The composition of claim 53, wherein the second polypeptide population comprises the amino acid sequence of SEQ ID NO: 477, or a biologically active fragment or variant thereof, and/or the third polypeptide population comprises the amino acid sequence of SEQ ID NO: 478, or a biologically active fragment or variant thereof.

57. The composition of claim 52, wherein the first polypeptide population comprises the amino acid sequence of SEQ ID NO: 506, or a biologically active fragment or variant thereof, (ii) the second polypeptide population comprises the amino acid sequence of any one of SEQ ID NOs: 406-588, or biologically active fragment or variant thereof, and (iii) the third polypeptide population comprises the amino acid sequence of any one of SEQ ID NOs: 406-588, or a biologically active fragment or variant thereof.

58. The composition of claim 57, wherein the population of tolerogenic antigens associated with a specific nanoparticle comprises (i) the first polypeptide population comprises the amino acid sequence of SEQ ID NO: 506, or a biologically active fragment or variant thereof, (ii) the second polypeptide population comprises the amino acid sequence of SEQ ID NO: 507, or biologically active fragment or variant thereof, and (iii) the third polypeptide population comprises the amino acid sequence of any one of SEQ ID NOs: 406-588, or a biologically active fragment or variant thereof.

59. The composition of claim 58, wherein the third polypeptide population comprises the amino acid sequence of SEQ ID NO: 508, or a biologically active fragment or variant thereof.

60. The composition of claim 17, wherein the tolerogenic antigen comprises a polypeptide having at least 90% sequence identity to the polypeptide sequence of SEQ ID NO: 374.

61. The composition of claim 60, wherein the tolerogenic antigen comprises a polypeptide having at least 95% sequence identity to the polypeptide of SEQ ID NO: 374.

62. The composition of claim 61, wherein the tolerogenic antigen comprises a polypeptide having at least 97% sequence identity to the polypeptide of SEQ ID NO: 374.

63. The composition of claim 62, wherein the tolerogenic antigen comprises a polypeptide sequence of SEQ ID NO: 374.

64. The composition of claim 63, wherein the tolerogenic antigen comprises a fragment of SEQ ID NO: 374, comprising 6-12 amino acid residues in length.

65. The composition of any one of claims 1-64, wherein the tolerogenic antigen comprises an amide group at the C-terminus.

66. The composition of any one of claims 1-65, wherein the tolerogenic antigen comprises a pyroglutamic acid residue at the N-terminus.

67. The composition of any one of claims 1-65, wherein the tolerogenic antigen comprises an acetyl group at the N-terminus.

68. The composition of any one of claims 1-66, wherein the tolerogenic antigen comprises a pyroglutamic acid residue at the N-terminus and an amide group at the C-terminus.

69. The composition of any one of claims 1-65 and 68, wherein the tolerogenic antigen comprises an acetyl group at the N-terminus and an amide group at the C-terminus.

70. The composition of any one of claims 1-69, wherein the tolerogenic antigen comprises an N-terminus or a C-terminus modified with a cysteine residue bound to a linker.

71. The composition of any one of claims 1-70, wherein the tolerogenic antigen comprises an N-terminus and a C-terminus modified with cysteine residues bound to a linker

72. The composition of any one of claims 1-71, wherein the plurality of tolerogenic antigens are conjugated with the nanoparticle phospholipid in such a manner that facilitates strong immune tolerance upon administration to a subject.

73. The composition of any one of claims 1-72, wherein the plurality of tolerogenic antigens are conjugated with the nanoparticle phospholipid via a thiol-reactive and reduction-insensitive linkage between each tolerogenic antigen and the nanoparticle phospholipid.

74. The composition of claim 73, wherein the nanoparticle phospholipid is N-(3-Maleimide-1-oxopropyl)-L-α-phosphatidylethanolamine.

75. The composition of claim 74, wherein the population of tolerogenic antigens are conjugated with the nanoparticle phospholipid an amine-mediated interaction.

76. The composition of claim 75, wherein the nanoparticle phospholipid is N-(Succinimidyloxy-glutaryl)-L-α-phosphatidylethanolamine, Dioleoyl (DOPE-NHS).

77. The composition of claim 75 or 76, wherein the amine-mediated interaction is through an amine-reactive phospholipid with self-immolative linkage.

78. The composition of any one of claims 1-77, wherein the composition further comprises at least one therapeutic agent.

79. The composition of claim 78, wherein the at least one therapeutic agent is at least one immunosuppressant or immunomodulatory agent.

80. The composition of claim 79, wherein the at least one immunosuppressant or immunomodulatory agent is selected from the group comprising fingolimod; 2-(1′H-indole-3′-carbonyl)-thiazole-4-carboxylic acid methyl ester (ITE) or related ligands; Trichostatin A; Suberoylanilide hydroxamic acid (SAHA); statins; mTOR inhibitors; TGF-β signaling agents; TGF-β receptor agonists; histone deacetylase inhibitors; corticosteroids; inhibitors of mitochondrial function; NF-κβ inhibitors; adenosine receptor agonists; prostaglandin E2 agonists (PGE2; phosphodiesterase inhibitors; proteasome inhibitors; kinase inhibitors; G-protein coupled receptor agonists; G-protein coupled receptor antagonists; glucocorticoids; retinoids; cytokine inhibitors; cytokine receptor inhibitors; cytokine receptor activators; peroxisome proliferator-activated receptor antagonists; peroxisome proliferator-activated receptor agonists; histone deacetylase inhibitors; calcineurin inhibitors; phosphatase inhibitors; PI3 KB inhibitors; autophagy inhibitors; aryl hydrocarbon receptor inhibitors; proteasome inhibitor I (PSI); oxidized ATPs IDO; vitamin D3; cyclosporins; aryl hydrocarbon receptor inhibitors; resveratrol; azathiopurine (Aza); 6-mercaptopurine (6-MP); 6-thioguanine (6-TG); FK506; sanglifehrin A; salmeterol; mycophenolate mofetil (MMF); aspirin and other COX inhibitors; niflumic acid; estriol; triptolide; OPN-305, OPN-401; Eritoran (E5564); TAK-242; Cpn10; NI-0101; 1A6; AV411; IRS-954 (DV-1079); IMO-3100; CPG-52363; CPG-52364; OPN-305; ATNC05; NI-0101; IMO-8400; Hydroxychloroquine; CU-CPT22; C29; Ortho-vanillin; SSL3 protein; OPN-305; 5 SsnB; Vizantin; (+)-N-phenethylnoroxymorphone; VB3323; Monosaccharide 3; (+)-Naltrexone and (+)-naloxone; HT52; HTB2; Compound 4a; CNT02424; TH1020; INH-ODN; E6446; AT791; CpG ODN 2088; ODN TTAGGG; COV08-0064; 2R9; GpG oligonucleotides; 2-aminopurine; Amlexanox; Bay11-7082; BX795; CH-223191; Chloroquine; CLI-095; CU-CPT9a; Cyclosporin A; CTY387; Gefitnib; Glybenclamide; H-89; H-131; Isoliquiritigenin; MCC950; MRT67307; OxPAPC; Parthenolide; Pepinh-MYD; Pepinh-TRIF; Polymyxin B; R406; RU.521; VX-765; YM201636; Z-VAD-FMK; and AHR-specific ligands; including but not limited to 2,3,7,8-tetrachloro-dibenzo-p-dioxin (TCDD); tryptamine (TA); and 6 formylindolo[3,2 b]carbazole (FICZ).

81. The composition of any one of claims 78-80, wherein the at least one therapeutic agent is comprised within the sHDL nanoparticle.

82. The composition of any one of claims 1-81, wherein the sHDL nanoparticle is further admixed with an adjuvant.

83. The composition of claim 81, wherein the adjuvant is selected from a list comprising CPG, polylC, poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®, vector system, PLGA microparticles, imiquimod, resiquimod, gardiquimod, 3M-052, SRL172, Virosomes and other Virus-like particles, YF-17D, VEGF trap, beta-glucan, Pam3Cys, Aquila's QS21 stimulon, vadimezan, AsA404 (DMXAA), and any derivative of an adjuvant.

84. The composition of any one of claims 1-80, wherein the composition does not contain an adjuvant.

85. A method of treating a subject having or at risk of having one or more autoimmune disorders, comprising administering an effective amount of the composition of any one of claims 1-84 to the subject.

86. The method of claim 85, wherein the one or more autoimmune disorders are selected from a list comprising: rheumatoid arthritis, multiple sclerosis, diabetes, autoimmune diseases of the thyroid, thyroid-associated ophthalmopathy, thyroid-associated dermopathy, hypoparathyroidism, Addison's disease, premature ovarian failure, autoimmune hypophysitis, pituitary autoimmune disease, immunogastritis, pernicious angemis, celiac disease, vitiligo, myasthenia gravis, pemphigus vulgaris and variants, bullous pemphigoid, dermatitis herpetiformis Duhring, epidermolysis bullosa acquisita, systemic sclerosis, mixed connective tissue disease, Sjogren's syndrome, systemic lupus erythematosus, Goodpasture's syndrome, rheumatic heart disease, autoimmune polyglandular syndrome type 1, Aicardi-Goutières syndrome, Acute pancreatitis Age-dependent macular degeneration, Alcoholic liver disease, Liver fibrosis, Metastasis, Myocardial infarction, Nonalcoholic steatohepatitis (NASH), Parkinson's disease, Polyarthritis/fetal and neonatal anemia, Sepsis, and inflammatory bowel disease.

87. The method of claim 85, wherein the one or more autoimmune disorders is a single autoimmune disorder.

88. The method of claim 87, wherein the single autoimmune disorder is celiac disease.

89. The method of any one of claims 85-88, wherein the subject is a human subject.
